SEQUENCE LISTING (373 sequences)

SEQ ID NO: 1 - protein, 474 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 2 - DNA, 1422 bp, Artificial Sequence
  Description of Artificial Sequence Synthetic polynucleotide
cagatcgtcctgacacagagcccagctatcatgtcagcaagccccggcgagaaagtcacaatgacttgctcagccagctcctctgtgagctacatgaactggtatcagcagaaaagcggaacctcccccaagagatggatctacgacacatccaagctggcctctggagtgcctgctcacttcaggggcagcggctctgggaccagttattcactgacaatttccggcatggaggccgaagatgccgctacctactattgccagcagtggagttcaaacccattcacttttggatctggcaccaagctggaaattaatggcggaggaggctccggaggaggagggtctggaggaggaggaagtcaggtgcagctgcagcagtccggagcagagctggctcgaccaggagctagtgtgaaaatgtcctgtaaggcaagcggctacaccttcacacggtataccatgcattgggtgaaacagagacccgggcagggactggaatggatcgggtacattaatcctagccgaggatacacaaactacaaccagaagtttaaagacaaggccactctgaccacagataagagctcctctaccgcttatatgcagctgagttcactgacatctgaggacagtgcagtgtactattgcgccaggtactatgacgatcactactgtctggattattggggccaggggactaccctgacagtgagctccgcagccgaacctaaatctagtgacaagactcatacctgccccccttgtccagcaccagaggctgcaggaggaccttccgtgttcctgtttccacccaaaccaaaggatactctgatgatctcccggacacctgaagtcacttgcgtggtcgtgagcgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggcactgcctgccccaatcgagaagacaattagcaaagcaaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggcaag

SEQ ID NO: 3 - protein, 106 aa, Artificial Sequence [sequence omitted]

SEQ ID NO: 4 - DNA, 318 bp, Artificial Sequence
  Description of Artificial Sequence Synthetic polynucleotide
cagatcgtcctgacacagagcccagctatcatgtcagcaagccccggcgagaaagtcacaatgacttgctcagccagctcctctgtgagctacatgaactggtatcagcagaaaagcggaacctcccccaagagatggatctacgacacatccaagctggcctctggagtgcctgctcacttcaggggcagcggctctgggaccagttattcactgacaatttccggcatggaggccgaagatgccgctacctactattgccagcagtggagttcaaacccattcacttttggatctggcaccaagctggaaattaat

SEQ ID NO: 5 - protein, 15 aa, Artificial Sequence
  Description of Artificial Sequence Synthetic peptide
GGGGSGGGGSGGGGS

SEQ ID NO: 6 - DNA, 45 bp, Artificial Sequence
  Description of Artificial Sequence Synthetic oligonucleotide
ggcggaggaggctccggaggaggagggtctggaggaggaggaagt

SEQ ID NO: 7 - protein, 119 aa, Artificial Sequence [sequence omitted]

SEQ ID NO: 8 - DNA, 357 bp, Artificial Sequence
  Description of Artificial Sequence Synthetic polynucleotide
caggtgcagctgcagcagtccggagcagagctggctcgaccaggagctagtgtgaaaatgtcctgtaaggcaagcggctacaccttcacacggtataccatgcattgggtgaaacagagacccgggcagggactggaatggatcgggtacattaatcctagccgaggatacacaaactacaaccagaagtttaaagacaaggccactctgaccacagataagagctcctctaccgcttatatgcagctgagttcactgacatctgaggacagtgcagtgtactattgcgccaggtactatgacgatcactactgtctggattattggggccaggggactaccctgacagtgagctcc

SEQ ID NO: 9 - protein, 17 aa, Artificial Sequence
  Description of Artificial Sequence Synthetic peptide
AAEPKSSDKTHTCPPCP

SEQ ID NO: 10 - DNA, 51 bp, Artificial Sequence
  Description of Artificial Sequence Synthetic oligonucleotide
gcagccgaacctaaatctagtgacaagactcatacctgccccccttgtcca

SEQ ID NO: 11 - protein, 110 aa, Artificial Sequence
  Description of Artificial Sequence Synthetic polypeptide
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK

SEQ ID NO: 12 - DNA, 330 bp, Artificial Sequence
  Description of Artificial Sequence Synthetic polynucleotide
gcaccagaggctgcaggaggaccttccgtgttcctgtttccacccaaaccaaaggatactctgatgatctcccggacacctgaagtcacttgcgtggtcgtgagcgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggcactgcctgccccaatcgagaagacaattagcaaagcaaag

SEQ ID NO: 13 - protein, 106 aa, Artificial Sequence
  Description of Artificial Sequence Synthetic polypeptide
GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 14 - DNA, 318 bp, Artificial Sequence
  Description of Artificial Sequence Synthetic polynucleotide
gggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggc

SEQ ID NO: 15 - protein, 473 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEINGGGGSGGGGSGGGGSQVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 16 - DNA, 1419 bp, Artificial Sequence
  Description of Artificial Sequence Synthetic polynucleotide
cagatcgtcctgactcagagccccgctattatgtccgcttcccctggagaaaaggtcactatgacttgttccgcctctagttccgtctcctacatgaactggtatcagcagaaatctggaacaagtcccaagcgatggatctacgacacttccaagctggcatctggagtgcctgcccacttccgaggcagcggctctgggacaagttattcactgactatttctggcatggaggccgaagatgccgctacatactattgccagcagtggagctccaacccattcacctttggatgtggcacaaagctggagatcaatggcggaggaggctccggaggaggagggtctggaggaggaggaagtcaggtccagctgcagcagagcggagcagaactggctagaccaggagccagtgtgaaaatgtcatgcaaggccagcggctacacattcactcggtataccatgcattgggtgaaacagagaccaggacagtgtctggagtggatcggctacattaatcccagcagggggtacacaaactacaaccagaagtttaaagacaaggcaaccctgaccaccgataagtctagttcaacagcttatatgcagctgagctccctgacttcagaagacagcgctgtgtactattgcgcacgctactatgacgatcactactgtctggattattgggggcagggaactaccctgaccgtgtctagtgcagccgagcctaaatcaagcgacaagacccatacatgccccccttgtccggcgccagaagctgcaggcggaccaagcgtgttcctgtttccacccaaacctaaggatactctgatgattagccgaactcctgaggtcacctgcgtggtcgtgagcgtgtcccacgaggacccagaagtcaagttcaactggtacgtggatggggtcgaagtgcataatgccaaaaccaagcccagggaggaacagtacaactccacttatcgcgtcgtgtctgtcctgaccgtgctgcaccaggactggctgaatggcaaggagtacaaatgtaaggtctcaaataaggctctgcccgcccctatcgaaaaaactatctcaaaggcaaaaggccagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 17 - protein, 106 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEIN

SEQ ID NO: 18 - DNA, 318 bp, Artificial Sequence
  Description of Artificial Sequence Synthetic polynucleotide
cagatcgtcctgactcagagccccgctattatgtccgcttcccctggagaaaaggtcactatgacttgttccgcctctagttccgtctcctacatgaactggtatcagcagaaatctggaacaagtcccaagcgatggatctacgacacttccaagctggcatctggagtgcctgcccacttccgaggcagcggctctgggacaagttattcactgactatttctggcatggaggccgaagatgccgctacatactattgccagcagtggagctccaacccattcacctttggatgtggcacaaagctggagatcaat

SEQ ID NO: 19 - protein, 15 aa, Artificial Sequence
  Description of Artificial Sequence Synthetic peptide
GGGGSGGGGSGGGGS

SEQ ID NO: 20 - DNA, 45 bp, Artificial Sequence; identical to SEQ ID NO: 6
  Description of Artificial Sequence Synthetic oligonucleotide

SEQ ID NO: 21 - protein, 119 aa, Artificial Sequence
  Description of Artificial Sequence Synthetic polypeptide
QVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSS

SEQ ID NO: 22 - DNA, 357 bp, Artificial Sequence
  Description of Artificial Sequence Synthetic polynucleotide
caggtccagctgcagcagagcggagcagaactggctagaccaggagccagtgtgaaaatgtcatgcaaggccagcggctacacattcactcggtataccatgcattgggtgaaacagagaccaggacagtgtctggagtggatcggctacattaatcccagcagggggtacacaaactacaaccagaagtttaaagacaaggcaaccctgaccaccgataagtctagttcaacagcttatatgcagctgagctccctgacttcagaagacagcgctgtgtactattgcgcacgctactatgacgatcactactgtctggattattgggggcagggaactaccctgaccgtgtctagt

SEQ ID NO: 23 - protein, 17 aa, Artificial Sequence
  Description of Artificial Sequence Synthetic peptide
AAEPKSSDKTHTCPPCP

SEQ ID NO: 24 - DNA, 51 bp, Artificial Sequence
  Description of Artificial Sequence Synthetic oligonucleotide
gcagccgagcctaaatcaagcgacaagacccatacatgccccccttgtccg

SEQ ID NO: 25 - protein, 110 aa, Artificial Sequence; identical to SEQ ID NO: 11
  Description of Artificial Sequence Synthetic polypeptide

SEQ ID NO: 26 - DNA, 330 bp, Artificial Sequence
  Description of Artificial Sequence Synthetic polynucleotide
gcgccagaagctgcaggcggaccaagcgtgttcctgtttccacccaaacctaaggatactctgatgattagccgaactcctgaggtcacctgcgtggtcgtgagcgtgtcccacgaggacccagaagtcaagttcaactggtacgtggatggggtcgaagtgcataatgccaaaaccaagcccagggaggaacagtacaactccacttatcgcgtcgtgtctgtcctgaccgtgctgcaccaggactggctgaatggcaaggagtacaaatgtaaggtctcaaataaggctctgcccgcccctatcgaaaaaactatctcaaaggcaaaa

SEQ ID NO: 27 - protein, 106 aa, Artificial Sequence; identical to SEQ ID NO: 13
  Description of Artificial Sequence Synthetic polypeptide

SEQ ID NO: 28 - DNA, 318 bp, Artificial Sequence
  Description of Artificial Sequence Synthetic polynucleotide
ggccagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 29 - protein, 477 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 30 - DNA, 1431 bp, Artificial Sequence
  Description of Artificial Sequence Synthetic polynucleotide
gacatcaaactgcagcagagcggagcagagctggctcgaccaggagccagtgtgaaaatgtcatgcaagaccagcggctacacattcactcggtatacaatgcactgggtgaagcagagaccaggacagggactggaatggatcggatatattaacccttcccgaggctacacaaactacaaccagaagtttaaagacaaggcaactctgaccacagataagagctcctctaccgcctacatgcagctgagttcactgacaagtgaggactcagccgtgtactattgcgctaggtactatgacgatcattactgtctggattattggggacagggcactaccctgactgtcagctccgtggaaggagggagcggaggctccggaggatctggcgggagtggaggcgtggacgatatccagctgacccagtccccagctattatgtccgcatctcccggcgagaaagtcaccatgacatgccgcgcctctagttcagtgagctacatgaactggtatcagcagaaatcaggcactagccccaagagatggatctacgacacctccaaggtcgcttctggggtgccttataggttcagtgggtcaggaagcggcacctcctactctctgacaattagctccatggaggctgaagatgccgctacctactattgtcagcagtggtctagtaatccactgacttttggggcaggaaccaaactggagctgaaggcagccgaacccaaatcaagcgacaagactcacacctgcccaccttgtccagcaccagaagctgcaggaggacctagcgtgttcctgtttccacccaaaccaaaggatacactgatgatcagccggacacctgaggtcacttgcgtggtcgtggacgtgagccacgaggaccccgaagtcaagttcaactggtacgtggacggcgtcgaagtgcataatgccaaaaccaagcctagggaggaacagtacaatagtacatatagagtcgtgtcagtgctgaccgtcctgcatcaggattggctgaacgggaaggagtacaaatgcaaggtgtccaacaaggcactgcctgccccaatcgagaagaccatttctaaagcaaagggccagccccgagaacctcaggtctatgtgctgcctccatcccgggacgagctgacaaaaaaccaggtctctctgctgtgtctggtgaaggggttctacccatctgatattgctgtggagtgggaaagtaatggacagcccgagaacaattatctgacatggccccctgtgctggactccgatggatctttctttctgtacagcaaactgactgtggacaagtccagatggcagcagggcaacgtctttagttgttcagtgatgcacgaggccctgcacaatcattacacccagaaaagcctgtccctgtctcccggcaag

SEQ ID NO: 31 - protein, 119 aa, Artificial Sequence
  Description of Artificial Sequence Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 32 - DNA, 357 bp, Artificial Sequence
  Description of Artificial Sequence Synthetic polynucleotide
gacatcaaactgcagcagagcggagcagagctggctcgaccaggagccagtgtgaaaatgtcatgcaagaccagcggctacacattcactcggtatacaatgcactgggtgaagcagagaccaggacagggactggaatggatcggatatattaacccttcccgaggctacacaaactacaaccagaagtttaaagacaaggcaactctgaccacagataagagctcctctaccgcctacatgcagctgagttcactgacaagtgaggactcagccgtgtactattgcgctaggtactatgacgatcattactgtctggattattggggacagggcactaccctgactgtcagctcc

SEQ ID NO: 33 - protein, 14 aa, Artificial Sequence
  Description of Artificial Sequence Synthetic peptide
GGSGGSGGSGGSGG

SEQ ID NO: 34 - DNA, 42 bp, Artificial Sequence
  Description of Artificial Sequence Synthetic oligonucleotide
ggagggagcggaggctccggaggatctggcgggagtggaggc

SEQ ID NO: 35 - protein, 106 aa, Artificial Sequence
  Description of Artificial Sequence Synthetic polypeptide
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 36 - DNA, 318 bp, Artificial Sequence
  Description of Artificial Sequence Synthetic polynucleotide
gatatccagctgacccagtccccagctattatgtccgcatctcccggcgagaaagtcaccatgacatgccgcgcctctagttcagtgagctacatgaactggtatcagcagaaatcaggcactagccccaagagatggatctacgacacctccaaggtcgcttctggggtgccttataggttcagtgggtcaggaagcggcacctcctactctctgacaattagctccatggaggctgaagatgccgctacctactattgtcagcagtggtctagtaatccactgacttttggggcaggaaccaaactggagctgaag

SEQ ID NO: 37 - protein, 17 aa, Artificial Sequence
  Description of Artificial Sequence Synthetic peptide
AAEPKSSDKTHTCPPCP

SEQ ID NO: 38 - DNA, 51 bp, Artificial Sequence
  Description of Artificial Sequence Synthetic oligonucleotide
gcagccgaacccaaatcaagcgacaagactcacacctgcccaccttgtcca

SEQ ID NO: 39 - protein, 110 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 40 - DNA, 330 bp, Artificial Sequence
  Description of Artificial Sequence Synthetic polynucleotide
gcaccagaagctgcaggaggacctagcgtgttcctgtttccacccaaaccaaaggatacactgatgatcagccggacacctgaggtcacttgcgtggtcgtggacgtgagccacgaggaccccgaagtcaagttcaactggtacgtggacggcgtcgaagtgcataatgccaaaaccaagcctagggaggaacagtacaatagtacatatagagtcgtgtcagtgctgaccgtcctgcatcaggattggctgaacgggaaggagtacaaatgcaaggtgtccaacaaggcactgcctgccccaatcgagaagaccatttctaaagcaaag

SEQ ID NO: 41 - protein, 106 aa, Artificial Sequence; identical to SEQ ID NO: 13
  Description of Artificial Sequence Synthetic polypeptide

SEQ ID NO: 42 - DNA, 318 bp, Artificial Sequence
  Description of Artificial Sequence Synthetic polynucleotide
ggccagccccgagaacctcaggtctatgtgctgcctccatcccgggacgagctgacaaaaaaccaggtctctctgctgtgtctggtgaaggggttctacccatctgatattgctgtggagtgggaaagtaatggacagcccgagaacaattatctgacatggccccctgtgctggactccgatggatctttctttctgtacagcaaactgactgtggacaagtccagatggcagcagggcaacgtctttagttgttcagtgatgcacgaggccctgcacaatcattacacccagaaaagcctgtccctgtctcccggc

SEQ ID NO: 43 - protein, 483 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGCGTKLEIKGGGGSGGGGSGGGGSQVQLQQSGAELVRPGSSVKISCKASGYAFSSYWMNWVKQRPGQCLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 44 - DNA, 1449 bp, Artificial Sequence
  Description of Artificial Sequence Synthetic polynucleotide
gacatccagctgacacagagccccgcaagcctggccgtgagcctgggacagagagccactatttcatgcaaagcctcacagagcgtggactatgatggagacagctatctgaactggtaccagcagatcccaggccagccccctaaactgctgatctacgacgccagcaatctggtgtccggcatcccacccaggttcagtggatcaggcagcgggaccgattttacactgaacattcaccctgtcgagaaggtggacgccgctacctaccattgccagcagtccacagaggacccctggactttcggatgtggcaccaaactggaaatcaagggcgggggaggctcaggaggaggagggagcggaggaggaggcagccaggtgcagctgcagcagagcggagcagaactggtccgacctggaagctccgtgaaaatttcttgcaaggccagtggctatgctttttctagttactggatgaattgggtgaagcagcgaccaggacagtgtctggagtggatcgggcagatttggcctggggatggagacaccaactataatggaaagttcaaaggcaaggcaactctgaccgccgacgaatcaagctccacagcttatatgcagctgtctagtctggctagtgaggattcagcagtgtacttttgcgcccggagagaaaccacaactgtgggcagatactattacgcaatggactactggggccaggggaccacagtcaccgtgtcaagcgcagccgagcccaaatcctctgataagacacacacttgccctccatgtccggcgccagaagctgcaggcggaccttccgtgttcctgtttccccctaaaccaaaggacactctgatgatctctcgcactccagaggtcacctgcgtggtcgtgtccgtgtctcacgaggaccccgaagtcaaattcaactggtatgtggacggggtcgaagtgcataatgccaaaacaaagcctagggaggaacagtataactctacataccgcgtcgtgagtgtcctgactgtgctgcatcaggattggctgaatggcaaggagtacaaatgtaaggtgagcaacaaagcactgcccgcccctatcgaaaaaactattagcaaagcaaaaggacagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 45 - protein, 111 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGCGTKLEIK

SEQ ID NO: 46 - DNA, 333 bp, Artificial Sequence
  Description of Artificial Sequence Synthetic polynucleotide
gacatccagctgacacagagccccgcaagcctggccgtgagcctgggacagagagccactatttcatgcaaagcctcacagagcgtggactatgatggagacagctatctgaactggtaccagcagatcccaggccagccccctaaactgctgatctacgacgccagcaatctggtgtccggcatcccacccaggttcagtggatcaggcagcgggaccgattttacactgaacattcaccctgtcgagaaggtggacgccgctacctaccattgccagcagtccacagaggacccctggactttcggatgtggcaccaaactggaaatcaag

SEQ ID NO: 47 - protein, 15 aa, Artificial Sequence
  Description of Artificial Sequence Synthetic peptide
GGGGSGGGGSGGGGS

SEQ ID NO: 48 - DNA, 45 bp, Artificial Sequence
  Description of Artificial Sequence Synthetic oligonucleotide
ggcgggggaggctcaggaggaggagggagcggaggaggaggcagc

SEQ ID NO: 49 - protein, 124 aa, Artificial Sequence
  Description of Artificial Sequence Synthetic polypeptide
QVQLQQSGAELVRPGSSVKISCKASGYAFSSYWMNWVKQRPGQCLEWIGQIWPGDGDTNYNGKFKGKATLTADESSSTAYMQLSSLASEDSAVYFCARRETTTVGRYYYAMDYWGQGTTVTVSS

SEQ ID NO: 50 - DNA, 372 bp, Artificial Sequence
  Description of Artificial Sequence Synthetic polynucleotide
caggtgcagctgcagcagagcggagcagaactggtccgacctggaagctccgtgaaaatttcttgcaaggccagtggctatgctttttctagttactggatgaattgggtgaagcagcgaccaggacagtgtctggagtggatcgggcagatttggcctggggatggagacaccaactataatggaaagttcaaaggcaaggcaactctgaccgccgacgaatcaagctccacagcttatatgcagctgtctagtctggctagtgaggattcagcagtgtacttttgcgcccggagagaaaccacaactgtgggcagatactattacgcaatggactactggggccaggggaccacagtcaccgtgtcaagc

SEQ ID NO: 51 - protein, 17 aa, Artificial Sequence
  Description of Artificial Sequence Synthetic peptide
AAEPKSSDKTHTCPPCP

SEQ ID NO: 52 - DNA, 51 bp, Artificial Sequence
  Description of Artificial Sequence Synthetic oligonucleotide
gcagccgagcccaaatcctctgataagacacacacttgccctccatgtccg

SEQ ID NO: 53 - protein, 110 aa, Artificial Sequence; identical to SEQ ID NO: 11
  Description of Artificial Sequence Synthetic polypeptide

SEQ ID NO: 54 - DNA, 330 bp, Artificial Sequence
  Description of Artificial Sequence Synthetic polynucleotide
gcgccagaagctgcaggcggaccttccgtgttcctgtttccccctaaaccaaaggacactctgatgatctctcgcactccagaggtcacctgcgtggtcgtgtccgtgtctcacgaggaccccgaagtcaaattcaactggtatgtggacggggtcgaagtgcataatgccaaaacaaagcctagggaggaacagtataactctacataccgcgtcgtgagtgtcctgactgtgctgcatcaggattggctgaatggcaaggagtacaaatgtaaggtgagcaacaaagcactgcccgcccctatcgaaaaaactattagcaaagcaaaa

SEQ ID NO: 55 - protein, 106 aa, Artificial Sequence
  Description of Artificial Sequence Synthetic polypeptide
GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 56 - DNA, 318 bp, Artificial Sequence
  Description of Artificial Sequence Synthetic polynucleotide
ggacagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 57 - protein, 484 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 58 - DNA, 1452 bp, Artificial Sequence
  Description of Artificial Sequence Synthetic polynucleotide
gatattcagctgacacagagtcctgcatcactggctgtgagcctgggacagcgagcaactatctcctgcaaagccagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacaccaactataatggaaagttcaaaggcaaggccacactgactgctgacgagtcaagctccacagcctatatgcagctgtctagtctggcaagcgaggattccgccgtgtacttttgcgctcggagagaaaccacaactgtgggcaggtactattacgctatggactactggggccaggggaccacagtcaccgtgtcaagcgcagccgaacccaaatcctctgataagacccacacatgccctccatgtccagctcctgaggctgcaggaggaccaagcgtgttcctgtttccccctaaacctaaggacacactgatgatctctcggacacccgaagtcacttgtgtggtcgtgagcgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggcactgccagcccccatcgagaagacaatttccaaagcaaagggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgctctggtcagtaaactgactgtggataagtcacggtggcagcagggaaacgtctttagttgttcagtgatgcacgaggcactgcacaatcattacacccagaaaagcctgtccctgtctcccggcaag

SEQ ID NO: 59 - protein, 111 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 60 - DNA, 333 bp, Artificial Sequence
  Description of Artificial Sequence Synthetic polynucleotide
gatattcagctgacacagagtcctgcatcactggctgtgagcctgggacagcgagcaactatctcctgcaaagccagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaag

SEQ ID NO: 61 - protein, 15 aa, Artificial Sequence
  Description of Artificial Sequence Synthetic peptide
GGGGSGGGGSGGGGS

SEQ ID NO: 62 - DNA, 45 bp, Artificial Sequence
  Description of Artificial Sequence Synthetic oligonucleotide
ggaggaggaggcagtggcggaggagggtcaggaggaggaggaagc

SEQ ID NO: 63 - protein, 124 aa, Artificial Sequence
  Description of Artificial Sequence Synthetic polypeptide
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 64 - DNA, 372 bp, Artificial Sequence
  Description of Artificial Sequence Synthetic polynucleotide
caggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacaccaactataatggaaagttcaaaggcaaggccacactgactgctgacgagtcaagctccacagcctatatgcagctgtctagtctggcaagcgaggattccgccgtgtacttttgcgctcggagagaaaccacaactgtgggcaggtactattacgctatggactactggggccaggggaccacagtcaccgtgtcaagc

SEQ ID NO: 65 - protein, 17 aa, Artificial Sequence
  Description of Artificial Sequence Synthetic peptide
AAEPKSSDKTHTCPPCP

SEQ ID NO: 66 - DNA, 51 bp, Artificial Sequence
  Description of Artificial Sequence Synthetic oligonucleotide
gcagccgaacccaaatcctctgataagacccacacatgccctccatgtcca

SEQ ID NO: 67 - protein, 110 aa, Artificial Sequence; identical to SEQ ID NO: 11
  Description of Artificial Sequence Synthetic polypeptide

SEQ ID NO: 68 - DNA, 330 bp, Artificial Sequence
  Description of Artificial Sequence Synthetic polynucleotide
gctcctgaggctgcaggaggaccaagcgtgttcctgtttccccctaaacctaaggacacactgatgatctctcggacacccgaagtcacttgtgtggtcgtgagcgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggcactgccagcccccatcgagaagacaatttccaaagcaaag

SEQ ID NO: 69 - protein, 106 aa, Artificial Sequence; identical to SEQ ID NO: 55
  Description of Artificial Sequence Synthetic polypeptide

SEQ ID NO: 70 - DNA, 318 bp, Artificial Sequence
  Description of Artificial Sequence Synthetic polynucleotide
ggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgctctggtcagtaaactgactgtggataagtcacggtggcagcagggaaacgtctttagttgttcagtgatgcacgaggcactgcacaatcattacacccagaaaagcctgtccctgtctcccggc

SEQ ID NO: 71 - protein, 484 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 72 - DNA, 1452 bp, Artificial Sequence
  Description of Artificial Sequence Synthetic polynucleotide
gacattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacactaactataatggaaagttcaaaggcaaggctacactgactgcagacgagtcaagctccaccgcttatatgcagctgtctagtctggccagcgaggattccgctgtctacttttgcgcacggagagaaaccacaactgtgggcaggtactattacgcaatggactactggggccaggggaccacagtcaccgtgtcaagcgcagccgaacccaaatcctctgataagacccacacatgccctccatgtccagcacctgagctgctgggaggaccaagcgtgttcctgtttccacctaaacctaaggacaccctgatgatctctcggacacccgaagtcacttgtgtggtcgtggatgtgagccacgaggaccctgaagtcaaattcaactggtacgtggatggcgtcgaggtgcataatgccaaaacaaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagaccatttccaaagctaagggccagcctcgagaaccacaggtgtatacatacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgcactggtcagtaaactgacagtggataagtcacggtggcagcagggaaacgtctttagttgttcagtgatgcacgaggccctgcacaatcattacactcagaaaagcctgtccctgtctcccggcaag

SEQ ID NO: 73 - protein, 111 aa, Artificial Sequence; identical to SEQ ID NO: 59
  Description of Artificial Sequence Synthetic polypeptide

SEQ ID NO: 74 - DNA, 333 bp, Artificial Sequence
  Description of Artificial Sequence Synthetic polynucleotide
gacattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaag

SEQ ID NO: 75 - protein, 15 aa, Artificial Sequence
  Description of Artificial Sequence Synthetic peptide
GGGGSGGGGSGGGGS

SEQ ID NO: 76 - DNA, 45 bp, Artificial Sequence; identical to SEQ ID NO: 62
  Description of Artificial Sequence Synthetic oligonucleotide

SEQ ID NO: 77 - protein, 124 aa, Artificial Sequence; identical to SEQ ID NO: 63
  Description of Artificial Sequence Synthetic polypeptide

SEQ ID NO: 78 - DNA, 372 bp, Artificial Sequence
  Description of Artificial Sequence Synthetic polynucleotide
caggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacactaactataatggaaagttcaaaggcaaggctacactgactgcagacgagtcaagctccaccgcttatatgcagctgtctagtctggccagcgaggattccgctgtctacttttgcgcacggagagaaaccacaactgtgggcaggtactattacgcaatggactactggggccaggggaccacagtcaccgtgtcaagc

SEQ ID NO: 79 - protein, 17 aa, Artificial Sequence
  Description of Artificial Sequence Synthetic peptide
AAEPKSSDKTHTCPPCP

SEQ ID NO: 80 - DNA, 51 bp, Artificial Sequence; identical to SEQ ID NO: 66
  Description of Artificial Sequence Synthetic oligonucleotide

SEQ ID NO: 81 - protein, 110 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 82 - DNA, 330 bp, Artificial Sequence
  Description of Artificial Sequence Synthetic polynucleotide
gcacctgagctgctgggaggaccaagcgtgttcctgtttccacctaaacctaaggacaccctgatgatctctcggacacccgaagtcacttgtgtggtcgtggatgtgagccacgaggaccctgaagtcaaattcaactggtacgtggatggcgtcgaggtgcataatgccaaaacaaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagaccatttccaaagctaag

SEQ ID NO: 83 - protein, 106 aa, Artificial Sequence
  Description of Artificial Sequence Synthetic polypeptide
GQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 84 - DNA, 318 bp, Artificial Sequence
  Description of Artificial Sequence Synthetic polynucleotide
ggccagcctcgagaaccacaggtgtatacatacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgcactggtcagtaaactgacagtggataagtcacggtggcagcagggaaacgtctttagttgttcagtgatgcacgaggccctgcacaatcattacactcagaaaagcctgtccctgtctcccggc

SEQ ID NO: 85 - protein, 484 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 86 - DNA, 1452 bp, Artificial Sequence
  Description of Artificial Sequence Synthetic polynucleotide
gatattcagctgacccagagtcctgcatcactggctgtgagcctgggacagcgagcaacaatctcctgcaaagccagtcagtcagtggactatgatggcgactcctatctgaactggtaccagcagatcccagggcagccccctaagctgctgatctacgacgcttcaaatctggtgagcggcatcccaccacgattcagcggcagcggctctggaaccgattttacactgaacattcacccagtcgagaaggtggacgccgctacctaccattgccagcagtctacagaggacccctggactttcggcgggggaaccaaactggaaatcaagggaggaggaggcagtggcggaggagggtcaggaggaggaggaagccaggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacacaaactataatggaaagttcaaaggcaaggccactctgaccgctgacgagtcaagctccactgcttatatgcagctgtctagtctggcaagcgaggattccgccgtctacttttgcgctcggagagaaaccacaactgtgggcaggtactattacgcaatggactactggggccaggggaccacagtcaccgtgtcaagcgcagccgaacccaaatcctctgataagacacacacttgccctccatgtccagcacctgaggctgcaggaggaccaagcgtgttcctgtttccccctaaacctaaggacactctgatgatctctcggactcccgaagtcacctgtgtggtcgtgagcgtgagccacgaggaccctgaagtcaaattcaactggtacgtggatggcgtcgaggtgcataatgccaaaacaaagcctagggaggaacagtataactccacataccgcgtcgtgtctgtcctgactgtgctgcatcaggactggctgaacggaaaggagtacaaatgcaaggtgagcaacaaggcactgccagcccccatcgagaagaccatttccaaagccaagggccagcctcgagaaccacaggtctatgtgctgccacccagccgggacgagctgacaaaaaaccaggtctccctgctgtgtctggtgaagggattctacccttctgatattgctgtggagtgggaaagtaatggccagccagaaaacaattatctgacttggcctccagtgctggattctgacgggagtttctttctgtacagtaaactgaccgtggataagtcacggtggcagcagggaaacgtctttagttgttcagtgatgcacgaggccctgcacaatcattacacccagaaaagcctgtccctgtctcccggcaag

SEQ ID NO: 87 - protein, 111 aa, Artificial Sequence; identical to SEQ ID NO: 59
  Description of Artificial Sequence Synthetic polypeptide

SEQ ID NO: 88 - DNA, 333 bp, Artificial Sequence
  Description of Artificial Sequence Synthetic polynucleotide
gatattcagctgacccagagtcctgcatcactggctgtgagcctgggacagcgagcaacaatctcctgcaaagccagtcagtcagtggactatgatggcgactcctatctgaactggtaccagcagatcccagggcagccccctaagctgctgatctacgacgcttcaaatctggtgagcggcatcccaccacgattcagcggcagcggctctggaaccgattttacactgaacattcacccagtcgagaaggtggacgccgctacctaccattgccagcagtctacagaggacccctggactttcggcgggggaaccaaactggaaatcaag

SEQ ID NO: 89 - protein, 15 aa, Artificial Sequence
  Description of Artificial Sequence Synthetic peptide
GGGGSGGGGSGGGGS

SEQ ID NO: 90 - DNA, 45 bp, Artificial Sequence; identical to SEQ ID NO: 62
  Description of Artificial Sequence Synthetic oligonucleotide

SEQ ID NO: 91 - protein, 124 aa, Artificial Sequence; identical to SEQ ID NO: 63
  Description of Artificial Sequence Synthetic polypeptide

SEQ ID NO: 92 - DNA, 372 bp, Artificial Sequence
  Description of Artificial Sequence Synthetic polynucleotide
caggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacacaaactataatggaaagttcaaaggcaaggccactctgaccgctgacgagtcaagctccactgcttatatgcagctgtctagtctggcaagcgaggattccgccgtctacttttgcgctcggagagaaaccacaactgtgggcaggtactattacgcaatggactactggggccaggggaccacagtcaccgtgtcaagc

SEQ ID NO: 93 - protein, 17 aa, Artificial Sequence
  Description of Artificial Sequence Synthetic peptide
AAEPKSSDKTHTCPPCP

SEQ ID NO: 94 - DNA, 51 bp, Artificial Sequence
  Description of Artificial Sequence Synthetic oligonucleotide
gcagccgaacccaaatcctctgataagacacacacttgccctccatgtcca

SEQ ID NO: 95 - protein, 110 aa, Artificial Sequence; identical to SEQ ID NO: 11
  Description of Artificial Sequence Synthetic polypeptide

SEQ ID NO: 96 - DNA, 330 bp, Artificial Sequence
  Description of Artificial Sequence Synthetic polynucleotide
gcacctgaggctgcaggaggaccaagcgtgttcctgtttccccctaaacctaaggacactctgatgatctctcggactcccgaagtcacctgtgtggtcgtgagcgtgagccacgaggaccctgaagtcaaattcaactggtacgtggatggcgtcgaggtgcataatgccaaaacaaagcctagggaggaacagtataactccacataccgcgtcgtgtctgtcctgactgtgctgcatcaggactggctgaacggaaaggagtacaaatgcaaggtgagcaacaaggcactgccagcccccatcgagaagaccatttccaaagccaag

SEQ ID NO: 97 - protein, 106 aa, Artificial Sequence; identical to SEQ ID NO: 13
  Description of Artificial Sequence Synthetic polypeptide

SEQ ID NO: 98 - DNA, 318 bp, Artificial Sequence
  Description of Artificial Sequence Synthetic polynucleotide
ggccagcctcgagaaccacaggtctatgtgctgccacccagccgggacgagctgacaaaaaaccaggtctccctgctgtgtctggtgaagggattctacccttctgatattgctgtggagtgggaaagtaatggccagccagaaaacaattatctgacttggcctccagtgctggattctgacgggagtttctttctgtacagtaaactgaccgtggataagtcacggtggcagcagggaaacgtctttagttgttcagtgatgcacgaggccctgcacaatcattacacccagaaaagcctgtccctgtctcccggc

SEQ ID NO: 99 - protein, 474 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 100 - DNA, 1422 bp, Artificial Sequence
  Description of Artificial Sequence Synthetic polynucleotide
cagatcgtcctgacacagagcccagcaatcatgtcagccagccccggcgagaaagtcacaatgacttgctcagcaagctcctctgtgagctacatgaactggtatcagcagaaaagcggaacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatctggcaccaagctggaaattaatggcggaggaggctccggaggaggagggtctggaggaggaggaagtcaggtccagctgcagcagtccggagctgagctggcacgaccaggagcaagtgtgaaaatgtcctgtaaggccagcggctacaccttcacacggtataccatgcattgggtgaaacagagacccgggcagggactggaatggatcgggtacattaatcctagccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactgtctggattattggggccaggggactaccctgaccgtgagctccgcagccgaacctaaatctagtgacaagactcatacctgccccccttgtccagcaccagagctgctgggaggaccttccgtgttcctgtttccacccaaaccaaaggatactctgatgatctcccggacacctgaagtcacttgcgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaaggggcagccccgagaacctcaggtgtacactctgcctccatctcgggacgagctgaccaaaaaccaggtcagtctgctgtgtctggtgaagggcttctatccaagcgatattgctgtggagtgggaatccaatgggcagcccgaaaacaattacatgacatggccccctgtcctggactcagatgggagcttctttctgtatagtaaactgactgtggacaagtcacggtggcagcagggaaacgtctttagctgttccgtgatgcatgaggccctgcacaatcattacacccagaaatctctgagtctgtcacccggcaag

SEQ ID NO: 101 - protein, 106 aa, Artificial Sequence [sequence omitted]

SEQ ID NO: 102 - DNA, 318 bp, Artificial Sequence
  Description of Artificial Sequence Synthetic polynucleotide
cagatcgtcctgacacagagcccagcaatcatgtcagccagccccggcgagaaagtcacaatgacttgctcagcaagctcctctgtgagctacatgaactggtatcagcagaaaagcggaacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatctggcaccaagctggaaattaat

SEQ ID NO: 103 - protein, 15 aa, Artificial Sequence
  Description of Artificial Sequence Synthetic peptide
GGGGSGGGGSGGGGS

SEQ ID NO: 104 - DNA, 45 bp, Artificial Sequence; identical to SEQ ID NO: 6
  Description of Artificial Sequence Synthetic oligonucleotide

SEQ ID NO: 105 - protein, 119 aa, Artificial Sequence [sequence omitted]

SEQ ID NO: 106 - DNA, 357 bp, Artificial Sequence
  Description of Artificial Sequence Synthetic polynucleotide
caggtccagctgcagcagtccggagctgagctggcacgaccaggagcaagtgtgaaaatgtcctgtaaggccagcggctacaccttcacacggtataccatgcattgggtgaaacagagacccgggcagggactggaatggatcgggtacattaatcctagccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactgtctggattattggggccaggggactaccctgaccgtgagctcc

SEQ ID NO: 107 - protein, 17 aa, Artificial Sequence
  Description of Artificial Sequence Synthetic peptide
AAEPKSSDKTHTCPPCP

SEQ ID NO: 108 - DNA, 51 bp, Artificial Sequence; identical to SEQ ID NO: 10
  Description of Artificial Sequence Synthetic oligonucleotide

SEQ ID NO: 109 - protein, 110 aa, Artificial Sequence; identical to SEQ ID NO: 81
  Description of Artificial Sequence Synthetic polypeptide

SEQ ID NO: 110 - DNA, 330 bp, Artificial Sequence
  Description of Artificial Sequence Synthetic polynucleotide
gcaccagagctgctgggaggaccttccgtgttcctgtttccacccaaaccaaaggatactctgatgatctcccggacacctgaagtcacttgcgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaag

SEQ ID NO: 111 - protein, 106 aa, Artificial Sequence
  Description of Artificial Sequence Synthetic polypeptide
GQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPG

SEQ ID NO: 112 - DNA, 318 bp, Artificial Sequence
  Description of Artificial Sequence Synthetic polynucleotide
gggcagccccgagaacctcaggtgtacactctgcctccatctcgggacgagctgaccaaaaaccaggtcagtctgctgtgtctggtgaagggcttctatccaagcgatattgctgtggagtgggaatccaatgggcagcccgaaaacaattacatgacatggccccctgtcctggactcagatgggagcttctttctgtatagtaaactgactgtggacaagtcacggtggcagcagggaaacgtctttagctgttccgtgatgcatgaggccctgcacaatcattacacccagaaatctctgagtctgtcacccggc

SEQ ID NO: 113 - protein, 480 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSSSTGGGGSGGGGSGGGGSDIQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 114 - DNA, 1440 bp, Artificial Sequence
  Description of Artificial Sequence Synthetic polynucleotide
caggtccagctgcagcagagcggagcagagctggctcgaccaggagctagtgtgaaaatgtcatgcaaggcaagcggctacaccttcacacggtatactatgcactgggtgaaacagagacccggacagggcctggaatggatcgggtacattaaccctagccgaggatacaccaactacaaccagaagtttaaagacaaggccaccctgaccacagataagagctcctctacagcttatatgcagctgagttcactgacttctgaggacagtgccgtgtactattgtgctcggtactatgacgatcattactccctggattattgggggcagggaactaccctgaccgtgagctcctctagtacaggaggaggaggcagtggaggaggagggtcaggcggaggaggaagcgacatccagattgtgctgacacagtctccagctatcatgtccgcatctcccggcgagaaagtcactatgacctgctccgcctcaagctccgtgtcttacatgaattggtatcagcagaaatcaggaaccagccccaagagatggatctacgacacatccaagctggcatctggagtgcctgcacacttcaggggcagtgggtcaggaactagctattccctgaccattagcggcatggaggccgaagatgccgctacctactattgtcagcagtggtctagtaacccattcacatttggcagcgggactaagctggagatcaatagggcagccgaacccaaatcaagcgacaagacacatacttgccccccttgtccagctccagaagctgcaggaggaccttccgtgttcctgtttccacccaaaccaaaggatacactgatgattagccgcacccctgaggtcacatgcgtggtcgtgagcgtgagccacgaggaccccgaagtcaagttcaactggtacgtggacggcgtcgaagtgcataatgccaaaaccaagcctagggaggaacagtacaacagtacatatagagtcgtgtcagtgctgaccgtcctgcaccaggattggctgaacggcaaggagtacaaatgcaaggtgtccaacaaggcactgcctgccccaatcgagaagaccatttctaaagctaaggggcagccccgagaacctcaggtctacgtgtatcctccatcccgggacgagctgactaaaaaccaggtctctctgacctgtctggtgaagggcttttacccatctgatattgcagtcgagtgggaaagtaatgggcagcccgagaacaattataagacaactccccctgtgctggactccgatgggtctttcgcactggtcagcaaactgacagtggataagtccagatggcagcagggaaacgtcttttcttgtagtgtgatgcatgaagccctgcacaatcattacactcagaaatcactgagcctgtcccccggcaag

SEQ ID NO: 115 - protein, 119 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 116 - DNA, 357 bp, Artificial Sequence
  Description of Artificial Sequence Synthetic polynucleotide
caggtccagctgcagcagagcggagcagagctggctcgaccaggagctagtgtgaaaatgtcatgcaaggcaagcggctacaccttcacacggtatactatgcactgggtgaaacagagacccggacagggcctggaatggatcgggtacattaaccctagccgaggatacaccaactacaaccagaagtttaaagacaaggccaccctgaccacagataagagctcctctacagcttatatgcagctgagttcactgacttctgaggacagtgccgtgtactattgtgctcggtactatgacgatcattactccctggattattgggggcagggaactaccctgaccgtgagctcc

SEQ ID NO: 117 - protein, 15 aa, Artificial Sequence
  Description of Artificial Sequence Synthetic peptide
GGGGSGGGGSGGGGS

SEQ ID NO: 118 - DNA, 45 bp, Artificial Sequence
  Description of Artificial Sequence Synthetic oligonucleotide
ggaggaggaggcagtggaggaggagggtcaggcggaggaggaagc

SEQ ID NO: 119 - protein, 106 aa, Artificial Sequence [sequence omitted]

SEQ ID NO: 120 - DNA, 318 bp, Artificial Sequence
  Description of Artificial Sequence Synthetic polynucleotide
cagattgtgctgacacagtctccagctatcatgtccgcatctcccggcgagaaagtcactatgacctgctccgcctcaagctccgtgtcttacatgaattggtatcagcagaaatcaggaaccagccccaagagatggatctacgacacatccaagctggcatctggagtgcctgcacacttcaggggcagtgggtcaggaactagctattccctgaccattagcggcatggaggccgaagatgccgctacctactattgtcagcagtggtctagtaacccattcacatttggcagcgggactaagctggagatcaat

SEQ ID NO: 121 - protein, 17 aa, Artificial Sequence
  Description of Artificial Sequence Synthetic peptide
AAEPKSSDKTHTCPPCP

SEQ ID NO: 122 - DNA, 51 bp, Artificial Sequence
  Description of Artificial Sequence Synthetic oligonucleotide
gcagccgaacccaaatcaagcgacaagacacatacttgccccccttgtcca

SEQ ID NO: 123 - protein, 110 aa, Artificial Sequence; identical to SEQ ID NO: 11
  Description of Artificial Sequence Synthetic polypeptide

SEQ ID NO: 124 - DNA, 330 bp, Artificial Sequence
  Description of Artificial Sequence Synthetic polynucleotide
gctccagaagctgcaggaggaccttccgtgttcctgtttccacccaaaccaaaggatacactgatgattagccgcacccctgaggtcacatgcgtggtcgtgagcgtgagccacgaggaccccgaagtcaagttcaactggtacgtggacggcgtcgaagtgcataatgccaaaaccaagcctagggaggaacagtacaacagtacatatagagtcgtgtcagtgctgaccgtcctgcaccaggattggctgaacggcaaggagtacaaatgcaaggtgtccaacaaggcactgcctgccccaatcgagaagaccatttctaaagctaag

SEQ ID NO: 125 - protein, 106 aa, Artificial Sequence; identical to SEQ ID NO: 55
  Description of Artificial Sequence Synthetic polypeptide

SEQ ID NO: 126 - DNA, 318 bp, Artificial Sequence
  Description of Artificial Sequence Synthetic polynucleotide
gggcagccccgagaacctcaggtctacgtgtatcctccatcccgggacgagctgactaaaaaccaggtctctctgacctgtctggtgaagggcttttacccatctgatattgcagtcgagtgggaaagtaatgggcagcccgagaacaattataagacaactccccctgtgctggactccgatgggtctttcgcactggtcagcaaactgacagtggataagtccagatggcagcagggaaacgtcttttcttgtagtgtgatgcatgaagccctgcacaatcattacactcagaaatcactgagcctgtcccccggc

SEQ ID NO: 127 - protein, 484 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 128 - DNA, 1452 bp, Artificial Sequence
  Description of Artificial Sequence Synthetic polynucleotide
gatattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacaccaactataatggaaagttcaaaggcaaggctacactgactgcagacgagtcaagctccacagcttatatgcagctgtctagtctggccagcgaggattccgctgtgtacttttgcgcacggagagaaaccacaactgtgggcaggtactattacgcaatggactactggggccaggggaccacagtcaccgtgtcaagcgcagccgaacccaaatcctctgataagacccacacatgccctccatgtccagcacctgagctgctgggaggaccaagcgtgttcctgtttccacctaaacctaaggacacactgatgatctctcggacacccgaagtcacttgtgtggtcgtggatgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagacaatttccaaagctaagggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgcactggtcagtaaactgactgtggataagtcacggtggcagcagggaaacgtctttagttgttcagtgatgcacgaggccctgcacaatcattacacccagaaaagcctgtccctgtctcccggcaag

SEQ ID NO: 129 - protein, 111 aa, Artificial Sequence; identical to SEQ ID NO: 59
  Description of Artificial Sequence Synthetic polypeptide

SEQ ID NO: 130 - DNA, 333 bp, Artificial Sequence
  Description of Artificial Sequence Synthetic polynucleotide
gatattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaag

SEQ ID NO: 131 - protein, 15 aa, Artificial Sequence
  Description of Artificial Sequence Synthetic peptide
GGGGSGGGGSGGGGS

SEQ ID NO: 132 - DNA, 45 bp, Artificial Sequence; identical to SEQ ID NO: 62
  Description of Artificial Sequence Synthetic oligonucleotide

SEQ ID NO: 133 - protein, 124 aa, Artificial Sequence; identical to SEQ ID NO: 63
  Description of Artificial Sequence Synthetic polypeptide

SEQ ID NO: 134 - DNA, 372 bp, Artificial Sequence
  Description of Artificial Sequence Synthetic polynucleotide
caggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacaccaactataatggaaagttcaaaggcaaggctacactgactgcagacgagtcaagctccacagcttatatgcagctgtctagtctggccagcgaggattccgctgtgtacttttgcgcacggagagaaaccacaactgtgggcaggtactattacgcaatggactactggggccaggggaccacagtcaccgtgtcaagc

SEQ ID NO: 135 - protein, 17 aa, Artificial Sequence
  Description of Artificial Sequence Synthetic peptide
AAEPKSSDKTHTCPPCP

SEQ ID NO: 136 - DNA, 51 bp, Artificial Sequence; identical to SEQ ID NO: 66
  Description of Artificial Sequence Synthetic oligonucleotide

SEQ ID NO: 137 - protein, 110 aa, Artificial Sequence; identical to SEQ ID NO: 81
  Description of Artificial Sequence Synthetic polypeptide

SEQ ID NO: 138 - DNA, 330 bp, Artificial Sequence
  Description of Artificial Sequence Synthetic polynucleotide
gcacctgagctgctgggaggaccaagcgtgttcctgtttccacctaaacctaaggacacactgatgatctctcggacacccgaagtcacttgtgtggtcgtggatgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagacaatttccaaagctaag

SEQ ID NO: 139 - protein, 106 aa, Artificial Sequence; identical to SEQ ID NO: 55
  Description of Artificial Sequence Synthetic polypeptide

SEQ ID NO: 140 - DNA, 318 bp, Artificial Sequence
  Description of Artificial Sequence Synthetic polynucleotide
ggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgcactggtcagtaaactgactgtggataagtcacggtggcagcagggaaacgtctttagttgttcagtgatgcacgaggccctgcacaatcattacacccagaaaagcctgtccctgtctcccggc

SEQ ID NO: 141 - protein, 474 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEINGGGGSGGGGSGGGGSQVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 142 - DNA, 1422 bp, Artificial Sequence
  Description of Artificial Sequence Synthetic polynucleotide
cagatcgtcctgacacagtccccagcaatcatgtcagccagccccggggagaaagtcacaatgacttgctcagcaagctcctctgtgagctacatgaactggtatcagcagaaaagcgggacctcccccaagagatggatctacgacacatccaagctggcttctggagtgcctgcacacttcaggggcagcggctctgggaccagttattcactgacaattagcggcatggaggctgaagatgccgctacctactattgccagcagtggagttcaaacccattcacttttggatgtggcaccaagctggaaattaatggcggaggaggctccggaggaggagggtctggaggaggaggaagtcaggtgcagctgcagcagtccggagctgagctggcacgaccaggagcaagtgtgaaaatgtcatgcaaggccagcggctacaccttcacacggtataccatgcattgggtgaaacagagacccggacagtgtctggaatggatcggctacattaatccttctcgagggtacacaaactacaaccagaagtttaaagacaaggctactctgaccacagataagagctcctctaccgcatatatgcagctgagttcactgacatctgaggacagtgccgtgtactattgcgctaggtactatgacgatcactactgtctggattattgggggcagggaactaccctgacagtgagctccgcagccgaacctaaatctagtgacaagactcatacctgccccccttgtccagcaccagagctgctgggaggacctagcgtgttcctgtttccacccaaaccaaaggatactctgatgatctcccggacacctgaagtcacttgcgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggaaaggagtacaaatgcaaggtgagcaacaaggccctgcctgctccaatcgagaagacaattagcaaagccaagggccagccccgagaacctcaggtctacgtgctgcctccatctcgggacgagctgactaaaaaccaggtcagtctgctgtgtctggtgaagggattctatccaagcgatattgctgtggagtgggaatccaatggccagcccgaaaacaattacctgacttggccccctgtcctggactcagatggcagcttctttctgtatagtaaactgaccgtggacaagtcacggtggcagcaggggaacgtctttagctgttccgtgatgcatgaggccctgcacaatcattacacccagaaatctctgagtctgtcacccggcaag

SEQ ID NO: 143 - protein, 106 aa, Artificial Sequence; identical to SEQ ID NO: 17
  Description of Artificial Sequence Synthetic polypeptide

SEQ ID NO: 144 - DNA, 318 bp, Artificial Sequence
  Description of Artificial Sequence Synthetic polynucleotide
cagatcgtcctgacacagtccccagcaatcatgtcagccagccccggggagaaagtcacaatgacttgctcagcaagctcctctgtgagctacatgaactggtatcagcagaaaagcgggacctcccccaagagatggatctacgacacatccaagctggcttctggagtgcctgcacacttcaggggcagcggctctgggaccagttattcactgacaattagcggcatggaggctgaagatgccgctacctactattgccagcagtggagttcaaacccattcacttttggatgtggcaccaagctggaaattaat

SEQ ID NO: 145 - protein, 15 aa, Artificial Sequence
  Description of Artificial Sequence Synthetic peptide
GGGGSGGGGSGGGGS

SEQ ID NO: 146 - DNA, 45 bp, Artificial Sequence; identical to SEQ ID NO: 6
  Description of Artificial Sequence Synthetic oligonucleotide

SEQ ID NO: 147 - protein, 119 aa, Artificial Sequence; identical to SEQ ID NO: 21
  Description of Artificial Sequence Synthetic polypeptide

SEQ ID NO: 148 - DNA, 357 bp, Artificial Sequence
  Description of Artificial Sequence Synthetic polynucleotide
caggtgcagctgcagcagtccggagctgagctggcacgaccaggagcaagtgtgaaaatgtcatgcaaggccagcggctacaccttcacacggtataccatgcattgggtgaaacagagacccggacagtgtctggaatggatcggctacattaatccttctcgagggtacacaaactacaaccagaagtttaaagacaaggctactctgaccacagataagagctcctctaccgcatatatgcagctgagttcactgacatctgaggacagtgccgtgtactattgcgctaggtactatgacgatcactactgtctggattattgggggcagggaactaccctgacagtgagctcc

SEQ ID NO: 149 - protein, 17 aa, Artificial Sequence
  Description of Artificial Sequence Synthetic peptide
AAEPKSSDKTHTCPPCP

SEQ ID NO: 150 - DNA, 51 bp, Artificial Sequence; identical to SEQ ID NO: 10
  Description of Artificial Sequence Synthetic oligonucleotide

SEQ ID NO: 151 - protein, 110 aa, Artificial Sequence; identical to SEQ ID NO: 81
  Description of Artificial Sequence Synthetic polypeptide

SEQ ID NO: 152 - DNA, 330 bp, Artificial Sequence
  Description of Artificial Sequence Synthetic polynucleotide
gcaccagagctgctgggaggacctagcgtgttcctgtttccacccaaaccaaaggatactctgatgatctcccggacacctgaagtcacttgcgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggaaaggagtacaaatgcaaggtgagcaacaaggccctgcctgctccaatcgagaagacaattagcaaagccaag

SEQ ID NO: 153 - protein, 106 aa, Artificial Sequence; identical to SEQ ID NO: 13
  Description of Artificial Sequence Synthetic polypeptide

SEQ ID NO: 154 - DNA, 318 bp, Artificial Sequence
  Description of Artificial Sequence Synthetic polynucleotide
ggccagccccgagaacctcaggtctacgtgctgcctccatctcgggacgagctgactaaaaaccaggtcagtctgctgtgtctggtgaagggattctatccaagcgatattgctgtggagtgggaatccaatggccagcccgaaaacaattacctgacttggccccctgtcctggactcagatggcagcttctttctgtatagtaaactgaccgtggacaagtcacggtggcagcaggggaacgtctttagctgttccgtgatgcatgaggccctgcacaatcattacacccagaaatctctgagtctgtcacccggc

SEQ ID NO: 155 - protein, 474 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEINGGGGSGGGGSGGGGSQVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 156 - DNA, 1422 bp, Artificial Sequence
  Description of Artificial Sequence Synthetic polynucleotide
cagatcgtcctgacacagagcccagcaatcatgtcagccagccccggggagaaagtcacaatgacttgctcagcaagctcctctgtgagctacatgaactggtatcagcagaaaagcgggacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatgtggcaccaagctggaaattaatggcggaggaggctccggaggaggagggtctggaggaggaggaagtcaggtgcagctgcagcagtccggagctgagctggcacgaccaggagcaagtgtgaaaatgtcatgcaaggccagcggctacaccttcacacggtataccatgcattgggtgaaacagagacccggacagtgtctggaatggatcggctacattaatcctagccgagggtacacaaactacaaccagaagtttaaagacaaggctactctgaccacagataagagctcctctaccgcatatatgcagctgagttcactgacatctgaggacagtgccgtgtactattgcgctaggtactatgacgatcactactccctggattattgggggcagggaactaccctgacagtgagctccgcagccgaacctaaatctagtgacaagactcatacctgcccaccttgtccagcaccagagctgctgggcgggccttctgtgttcctgtttccacccaaaccaaaggatactctgatgatctcccggacacctgaagtcacttgtgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggaaaggagtacaaatgcaaggtgagcaacaaggccctgcctgctccaatcgagaagacaattagcaaagccaagggccagccccgagaacctcaggtctacgtgctgcctccatctcgggacgagctgactaaaaaccaggtcagtctgctgtgtctggtgaagggattctatccaagcgatattgctgtggagtgggaatccaatggccagcccgaaaacaattacctgacttggccccctgtcctggactcagatggcagcttctttctgtatagtaaactgaccgtggacaagtcacggtggcagcaggggaacgtctttagctgttccgtgatgcatgaggccctgcacaatcattacacccagaaatctctgagtctgtcacccggcaag

SEQ ID NO: 157 - protein, 106 aa, Artificial Sequence; identical to SEQ ID NO: 17
  Description of Artificial Sequence Synthetic polypeptide

SEQ ID NO: 158 - DNA, 318 bp, Artificial Sequence
  Description of Artificial Sequence Synthetic polynucleotide
cagatcgtcctgacacagagcccagcaatcatgtcagccagccccggggagaaagtcacaatgacttgctcagcaagctcctctgtgagctacatgaactggtatcagcagaaaagcgggacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatgtggcaccaagctggaaattaat

SEQ ID NO: 159 - protein, 15 aa, Artificial Sequence
  Description of Artificial Sequence Synthetic peptide
GGGGSGGGGSGGGGS

SEQ ID NO: 160 - DNA, 45 bp, Artificial Sequence; identical to SEQ ID NO: 6
  Description of Artificial Sequence Synthetic oligonucleotide

SEQ ID NO: 161 - protein, 119 aa, Artificial Sequence
  Description of Artificial Sequence Synthetic polypeptide
QVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYSLDYWGQGTTLTVSS

SEQ ID NO: 162 - DNA, 357 bp, Artificial Sequence
  Description of Artificial Sequence Synthetic polynucleotide
caggtgcagctgcagcagtccggagctgagctggcacgaccaggagcaagtgtgaaaatgtcatgcaaggccagcggctacaccttcacacggtataccatgcattgggtgaaacagagacccggacagtgtctggaatggatcggctacattaatcctagccgagggtacacaaactacaaccagaagtttaaagacaaggctactctgaccacagataagagctcctctaccgcatatatgcagctgagttcactgacatctgaggacagtgccgtgtactattgcgctaggtactatgacgatcactactccctggattattgggggcagggaactaccctgacagtgagctcc

SEQ ID NO: 163 - protein, 17 aa, Artificial Sequence
  Description of Artificial Sequence Synthetic peptide
AAEPKSSDKTHTCPPCP

SEQ ID NO: 164 - DNA, 51 bp, Artificial Sequence
  Description of Artificial Sequence Synthetic oligonucleotide
gcagccgaacctaaatctagtgacaagactcatacctgcccaccttgtcca

SEQ ID NO: 165 - protein, 110 aa, Artificial Sequence; identical to SEQ ID NO: 81
  Description of Artificial Sequence Synthetic polypeptide

SEQ ID NO: 166 - DNA, 330 bp, Artificial Sequence
  Description of Artificial Sequence Synthetic polynucleotide
gcaccagagctgctgggcgggccttctgtgttcctgtttccacccaaaccaaaggatactctgatgatctcccggacacctgaagtcacttgtgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggaaaggagtacaaatgcaaggtgagcaacaaggccctgcctgctccaatcgagaagacaattagcaaagccaag

SEQ ID NO: 167 - protein, 106 aa, Artificial Sequence; identical to SEQ ID NO: 13
  Description of Artificial Sequence Synthetic polypeptide

SEQ ID NO: 168 - DNA, 318 bp, Artificial Sequence; identical to SEQ ID NO: 154
  Description of Artificial Sequence Synthetic polynucleotide

SEQ ID NO: 169 - protein, 504 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 170 - DNA, 1512 bp, Artificial Sequence
  Description of Artificial Sequence Synthetic polynucleotide
gatattcagctgacacagtctccagctagtctggcagtgagcctgggccagcgggctactatcagctgcaaggcaagccagtccgtcgactacgatggggacagctatctgaactggtaccagcagatccccggacagccccctaaactgctgatctacgacgcctcaaatctggtgagcggcatcccacccagattctctggaagtggctcagggaccgattttacactgaacattcaccccgtggagaaggtcgacgccgctacctaccattgccagcagtccactgaggacccctggaccttcggaggaggaacaaagctggaaatcaaaggcggaggaggcagtggaggaggagggagcggaggaggaggaagccaggtgcagctgcagcagagcggagcagaactggtgagacctggaagctccgtcaagatttcctgtaaagcatctggctatgccttttctagttactggatgaattgggtgaagcagaggccaggacagggactggagtggatcggacagatttggcctggggatggagacaccaactacaatggaaagttcaaaggcaaggctaccctgacagcagacgaatcaagctccacagcttacatgcagctgtctagtctggcatcagaggatagcgccgtgtatttttgcgctcggagagaaaccacaactgtcggccgctactattacgccatggactactggggccaggggaccacagtgacagtctcaagcggcgggggaggctccgatatcaagctgcagcagtctggagcagagctggctcgaccaggagccagtgtgaagatgtcatgtaaaaccagcggctatactttcaccaggtacacaatgcactgggtgaaacagcgcccaggacagggcctggaatggatcggatacattaacccctccaggggctataccaactacaatcagaagttcaaggataaagccactctgactaccgacaagtcctctagtaccgcttatatgcagctgtcaagcctgacatccgaggactctgcagtgtattactgcgcccgctattacgacgatcattattgtctggattactgggggcagggaacaactctgactgtgtcctctgtcgaagggggaagtggagggtcaggaggcagcggaggcagcggaggggtggacgatatccagctgacccagtcccctgccattatgagcgcttccccaggcgagaaggtgacaatgacttgcagggctagttcaagcgtctcttatatgaattggtatcagcagaagtctggcactagtcctaaacgatggatctatgacacctccaaagtggcatctggggtcccataccggttctctggcagtgggtcaggaactagctattccctgaccatttcctctatggaggcagaagatgcagccacctattactgtcagcagtggagttcaaatcccctgacatttggcgccgggactaagctggagctgaaacaccatcaccatcaccat

SEQ ID NO: 171 - protein, 111 aa, Artificial Sequence; identical to SEQ ID NO: 59
  Description of Artificial Sequence Synthetic polypeptide

SEQ ID NO: 172 - DNA, 333 bp, Artificial Sequence
  Description of Artificial Sequence Synthetic polynucleotide
gatattcagctgacacagtctccagctagtctggcagtgagcctgggccagcgggctactatcagctgcaaggcaagccagtccgtcgactacgatggggacagctatctgaactggtaccagcagatccccggacagccccctaaactgctgatctacgacgcctcaaatctggtgagcggcatcccacccagattctctggaagtggctcagggaccgattttacactgaacattcaccccgtggagaaggtcgacgccgctacctaccattgccagcagtccactgaggacccctggaccttcggaggaggaacaaagctggaaatcaaa

SEQ ID NO: 173 - protein, 15 aa, Artificial Sequence
  Description of Artificial Sequence Synthetic peptide
GGGGSGGGGSGGGGS

SEQ ID NO: 174 - DNA, 45 bp, Artificial Sequence
  Description of Artificial Sequence Synthetic oligonucleotide
ggcggaggaggcagtggaggaggagggagcggaggaggaggaagc

SEQ ID NO: 175 - protein, 124 aa, Artificial Sequence; identical to SEQ ID NO: 63
  Description of Artificial Sequence Synthetic polypeptide

SEQ ID NO: 176 - DNA, 372 bp, Artificial Sequence
  Description of Artificial Sequence Synthetic polynucleotide
caggtgcagctgcagcagagcggagcagaactggtgagacctggaagctccgtcaagatttcctgtaaagcatctggctatgccttttctagttactggatgaattgggtgaagcagaggccaggacagggactggagtggatcggacagatttggcctggggatggagacaccaactacaatggaaagttcaaaggcaaggctaccctgacagcagacgaatcaagctccacagcttacatgcagctgtctagtctggcatcagaggatagcgccgtgtatttttgcgctcggagagaaaccacaactgtcggccgctactattacgccatggactactggggccaggggaccacagtgacagtctcaagc

SEQ ID NO: 177 - protein, 119 aa, Artificial Sequence; identical to SEQ ID NO: 31
  Description of Artificial Sequence Synthetic polypeptide

SEQ ID NO: 178 - DNA, 357 bp, Artificial Sequence
  Description of Artificial Sequence Synthetic polynucleotide
gatatcaagctgcagcagtctggagcagagctggctcgaccaggagccagtgtgaagatgtcatgtaaaaccagcggctatactttcaccaggtacacaatgcactgggtgaaacagcgcccaggacagggcctggaatggatcggatacattaacccctccaggggctataccaactacaatcagaagttcaaggataaagccactctgactaccgacaagtcctctagtaccgcttatatgcagctgtcaagcctgacatccgaggactctgcagtgtattactgcgcccgctattacgacgatcattattgtctggattactgggggcagggaacaactctgactgtgtcctct

SEQ ID NO: 179 - protein, 14 aa, Artificial Sequence
  Description of Artificial Sequence Synthetic peptide
GGSGGSGGSGGSGG

SEQ ID NO: 180 - DNA, 42 bp, Artificial Sequence
  Description of Artificial Sequence Synthetic oligonucleotide
gggggaagtggagggtcaggaggcagcggaggcagcggaggg

SEQ ID NO: 181 - protein, 106 aa, Artificial Sequence; identical to SEQ ID NO: 35
  Description of Artificial Sequence Synthetic polypeptide

SEQ ID NO: 182 - DNA, 318 bp, Artificial Sequence
  Description of Artificial Sequence Synthetic polynucleotide
gatatccagctgacccagtcccctgccattatgagcgcttccccaggcgagaaggtgacaatgacttgcagggctagttcaagcgtctcttatatgaattggtatcagcagaagtctggcactagtcctaaacgatggatctatgacacctccaaagtggcatctggggtcccataccggttctctggcagtgggtcaggaactagctattccctgaccatttcctctatggaggcagaagatgcagccacctattactgtcagcagtggagttcaaatcccctgacatttggcgccgggactaagctggagctgaaa

SEQ ID NO: 183 - protein, 474 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 184 - DNA, 1422 bp, Artificial Sequence
  Description of Artificial Sequence Synthetic polynucleotide
cagatcgtcctgacacagagcccagcaatcatgtcagccagccccggcgagaaagtcacaatgacttgctcagcaagctcctctgtgagctacatgaactggtatcagcagaaaagcggaacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatctggcaccaagctggaaattaatggcggaggaggctccggaggaggagggtctggaggaggaggaagtcaggtgcagctgcagcagagcggagctgagctggcacgaccaggagcaagtgtgaaaatgtcctgtaaggccagcggctacaccttcacacggtataccatgcattgggtgaaacagagacccgggcagggactggaatggatcgggtacattaatccttcccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactccctggattattggggccaggggactaccctgacagtgagctccgcagccgaacctaaatctagtgacaagactcatacctgccccccttgtccagcaccagagctgctgggaggacctagcgtgttcctgtttccacccaaaccaaaggatactctgatgatctcccggacacctgaagtcacttgtgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggcaag

SEQ ID NO: 185 - protein, 106 aa, Artificial Sequence [sequence omitted]

SEQ ID NO: 186 - DNA, 318 bp, Artificial Sequence; identical to SEQ ID NO: 102
  Description of Artificial Sequence Synthetic polynucleotide

SEQ ID NO: 187 - protein, 15 aa, Artificial Sequence
  Description of Artificial Sequence Synthetic peptide
GGGGSGGGGSGGGGS

SEQ ID NO: 188 - DNA, 45 bp, Artificial Sequence; identical to SEQ ID NO: 6
  Description of Artificial Sequence Synthetic oligonucleotide

SEQ ID NO: 189 - protein, 119 aa, Artificial Sequence; identical to SEQ ID NO: 115
  Description of Artificial Sequence Synthetic polypeptide

SEQ ID NO: 190 - DNA, 357 bp, Artificial Sequence
  Description of Artificial Sequence Synthetic polynucleotide
caggtgcagctgcagcagagcggagctgagctggcacgaccaggagcaagtgtgaaaatgtcctgtaaggccagcggctacaccttcacacggtataccatgcattgggtgaaacagagacccgggcagggactggaatggatcgggtacattaatccttcccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactccctggattattggggccaggggactaccctgacagtgagctcc

SEQ ID NO: 191 - protein, 17 aa, Artificial Sequence
  Description of Artificial Sequence Synthetic peptide
AAEPKSSDKTHTCPPCP

SEQ ID NO: 192 - DNA, 51 bp, Artificial Sequence; identical to SEQ ID NO: 10
  Description of Artificial Sequence Synthetic oligonucleotide

SEQ ID NO: 193 - protein, 110 aa, Artificial Sequence; identical to SEQ ID NO: 81
  Description of Artificial Sequence Synthetic polypeptide

SEQ ID NO: 194 - DNA, 330 bp, Artificial Sequence
  Description of Artificial Sequence Synthetic polynucleotide
gcaccagagctgctgggaggacctagcgtgttcctgtttccacccaaaccaaaggatactctgatgatctcccggacacctgaagtcacttgtgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaag

SEQ ID NO: 195 - protein, 106 aa, Artificial Sequence; identical to SEQ ID NO: 13
  Description of Artificial Sequence Synthetic polypeptide

SEQ ID NO: 196 - DNA, 318 bp, Artificial Sequence; identical to SEQ ID NO: 14
  Description of Artificial Sequence Synthetic polynucleotide

SEQ ID NO: 197 - protein, 474 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 198 - DNA, 1422 bp, Artificial Sequence
  Description of Artificial Sequence Synthetic polynucleotide
cagatcgtcctgacacagagcccagctatcatgtcagcaagccccggcgagaaagtcacaatgacttgctcagccagctcctctgtgagctacatgaactggtatcagcagaaaagcggaacctcccccaagagatggatctacgacacatccaagctggcctctggagtgcctgctcacttcaggggcagcggctctgggaccagttattcactgacaatttccggcatggaggccgaagatgccgctacctactattgccagcagtggagttcaaacccattcacttttggatctggcaccaagctggaaattaatggcggaggaggctccggaggaggagggtctggaggaggaggaagtcaggtgcagctgcagcagagcggagcagagctggctcgaccaggagctagtgtgaaaatgtcctgtaaggcaagcggctacaccttcacacggtataccatgcattgggtgaaacagagacccgggcagggactggaatggatcgggtacattaatccttcccgaggatacacaaactacaaccagaagtttaaagacaaggccactctgaccacagataagagctcctctaccgcttatatgcagctgagttcactgacatctgaggacagtgcagtgtactattgcgccaggtactatgacgatcactactccctggattattggggccaggggactaccctgacagtgagctccgcagccgaacctaaatctagtgacaagactcatacctgccccccttgtccagcaccagaggctgcaggaggacctagcgtgttcctgtttccacccaaaccaaaggatactctgatgatctcccggacacctgaagtcacttgtgtggtcgtgagcgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggcactgcctgccccaatcgagaagacaattagcaaagcaaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggcaag

SEQ ID NO: 199 - protein, 106 aa, Artificial Sequence [sequence omitted]

SEQ ID NO: 200 - DNA, 318 bp, Artificial Sequence; identical to SEQ ID NO: 4
  Description of Artificial Sequence Synthetic polynucleotide

SEQ ID NO: 201 - protein, 15 aa, Artificial Sequence
  Description of Artificial Sequence Synthetic peptide
GGGGSGGGGSGGGGS

SEQ ID NO: 202 - DNA, 45 bp, Artificial Sequence; identical to SEQ ID NO: 6
  Description of Artificial Sequence Synthetic oligonucleotide

SEQ ID NO: 203 - protein, 119 aa, Artificial Sequence; identical to SEQ ID NO: 115
  Description of Artificial Sequence Synthetic polypeptide

SEQ ID NO: 204 - DNA, 357 bp, Artificial Sequence
  Description of Artificial Sequence Synthetic polynucleotide
caggtgcagctgcagcagagcggagcagagctggctcgaccaggagctagtgtgaaaatgtcctgtaaggcaagcggctacaccttcacacggtataccatgcattgggtgaaacagagacccgggcagggactggaatggatcgggtacattaatccttcccgaggatacacaaactacaaccagaagtttaaagacaaggccactctgaccacagataagagctcctctaccgcttatatgcagctgagttcactgacatctgaggacagtgcagtgtactattgcgccaggtactatgacgatcactactccctggattattggggccaggggactaccctgacagtgagctcc

SEQ ID NO: 205 - protein, 17 aa, Artificial Sequence
  Description of Artificial Sequence Synthetic peptide
AAEPKSSDKTHTCPPCP

SEQ ID NO: 206 - DNA, 51 bp, Artificial Sequence; identical to SEQ ID NO: 10
  Description of Artificial Sequence Synthetic oligonucleotide

SEQ ID NO: 207 - protein, 110 aa, Artificial Sequence; identical to SEQ ID NO: 11
  Description of Artificial Sequence Synthetic polypeptide

SEQ ID NO: 208 - DNA, 330 bp, Artificial Sequence
  Description of Artificial Sequence Synthetic polynucleotide
gcaccagaggctgcaggaggacctagcgtgttcctgtttccacccaaaccaaaggatactctgatgatctcccggacacctgaagtcacttgtgtggtcgtgagcgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggcactgcctgccccaatcgagaagacaattagcaaagcaaag

SEQ ID NO: 209 - protein, 106 aa, Artificial Sequence; identical to SEQ ID NO: 13
  Description of Artificial Sequence Synthetic polypeptide

SEQ ID NO: 210 - DNA, 318 bp, Artificial Sequence; identical to SEQ ID NO: 14
  Description of Artificial Sequence Synthetic polynucleotide

SEQ ID NO: 211 - protein, 484 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 212 - DNA, 1452 bp, Artificial Sequence
  Description of Artificial Sequence Synthetic polynucleotide
gatattcagctgacacagagtcctgcatcactggctgtgagcctgggacagcgagcaactatctcctgcaaagccagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacaccaactataatggaaagttcaaaggcaaggccacactgactgctgacgagtcaagctccacagcctatatgcagctgtctagtctggcaagcgaggattccgccgtgtacttttgcgctcggagagaaaccacaactgtgggcaggtactattacgctatggactactggggccaggggaccacagtcaccgtgtcaagcgcagccgaacccaaatcctctgataagacccacacatgccctccatgtccagctcctgaggctgcaggaggaccaagcgtgttcctgtttccccctaaacctaaggacacactgatgatctctcggacacccgaagtcacttgtgtggtcgtggatgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggcactgccagcccccatcgagaagacaatttccaaagcaaagggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgctctggtcagtaaactgactgtggataagtcacggtggcagcagggaaacgtctttagttgttcagtgatgcacgaggcactgcacaatcattacacccagaaaagcctgtccctgtctcccggcaag

SEQ ID NO: 213 - protein, 111 aa, Artificial Sequence; identical to SEQ ID NO: 59
  Description of Artificial Sequence Synthetic polypeptide

SEQ ID NO: 214 - DNA, 333 bp, Artificial Sequence; identical to SEQ ID NO: 60
  Description of Artificial Sequence Synthetic polynucleotide

SEQ ID NO: 215 - protein, 15 aa, Artificial Sequence
  Description of Artificial Sequence Synthetic peptide
GGGGSGGGGSGGGGS

SEQ ID NO: 216 - DNA, 45 bp, Artificial Sequence; identical to SEQ ID NO: 62
  Description of Artificial Sequence Synthetic oligonucleotide

SEQ ID NO: 217 - protein, 124 aa, Artificial Sequence; identical to SEQ ID NO: 63
  Description of Artificial Sequence Synthetic polypeptide

SEQ ID NO: 218 - DNA, 372 bp, Artificial Sequence; identical to SEQ ID NO: 64
  Description of Artificial Sequence Synthetic polynucleotide

SEQ ID NO: 219 - protein, 17 aa, Artificial Sequence
  Description of Artificial Sequence Synthetic peptide
AAEPKSSDKTHTCPPCP

SEQ ID NO: 220 - DNA, 51 bp, Artificial Sequence; identical to SEQ ID NO: 66
  Description of Artificial Sequence Synthetic oligonucleotide

SEQ ID NO: 221 - protein, 110 aa, Artificial Sequence; identical to SEQ ID NO: 39
  Description of Artificial Sequence Synthetic polypeptide

SEQ ID NO: 222 - DNA, 330 bp, Artificial Sequence
  Description of Artificial Sequence Synthetic polynucleotide
gctcctgaggctgcaggaggaccaagcgtgttcctgtttccccctaaacctaaggacacactgatgatctctcggacacccgaagtcacttgtgtggtcgtggatgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggcactgccagcccccatcgagaagacaatttccaaagcaaag

SEQ ID NO: 223 - protein, 106 aa, Artificial Sequence; identical to SEQ ID NO: 55
  Description of Artificial Sequence Synthetic polypeptide

SEQ ID NO: 224 - DNA, 318 bp, Artificial Sequence; identical to SEQ ID NO: 70
  Description of Artificial Sequence Synthetic polynucleotide

SEQ ID NO: 225 - protein, 481 aa, Artificial Sequence
  Description of Artificial Sequence Synthetic polypeptide
DIQLTQSPSSLSASVGDRATITCRASQSVDYEGDSYLNWYQQKPGKAPKLLIYDASNLVSGIPSRFSGSGSGTDFTLTISSVQPEDAATYYCQQSTEDPWTFGCGTKLEIKGGGGSGGGGSGGGGSQVQLVQSGAEVKKPGASVKISCKASGYAFSSYWMNWVRQAPGQCLEWIGQIWPGDGDTNYAQKFQGRATLTADESTSTAYMELSSLRSEDTAVYYCARRETTTVGRYYYAMDYWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 226 - DNA, 1443 bp, Artificial Sequence
  Description of Artificial Sequence Synthetic polynucleotide
gatattcagctgacccagagcccaagctccctgtctgccagtgtgggggatagggctacaatcacttgccgcgcatcacagagcgtggactatgagggcgattcctatctgaactggtaccagcagaagccagggaaagcacccaagctgctgatctacgacgcctctaatctggtgagtggcattccctcaaggttctccggatctggcagtgggactgactttaccctgacaatctctagtgtgcagcccgaggatgccgctacctactattgccagcagtctacagaagacccttggactttcggatgtggcaccaaactggagattaagggaggaggaggcagtggcggaggagggtcaggaggaggaggaagccaggtccagctggtgcagagcggagcagaggtcaagaaacccggagccagcgtgaaaatttcctgcaaggcctctggctatgctttctcaagctactggatgaactgggtgaggcaggcaccaggacagtgtctggaatggatcggacagatttggcctggggacggagataccaattatgctcagaagtttcagggacgcgcaactctgaccgccgatgagtcaacaagcactgcatacatggagctgtcctctctgcgctccgaagacacagccgtgtactattgcgcacggagagaaaccacaactgtgggccgatactattacgcaatggattactggggccaggggaccacagtcactgtgagttcagagcctaaaagctccgacaagacccacacatgcccaccttgtccggcgccagaagcagccggagggcctagcgtgttcctgtttccacccaagccaaaagataccctgatgatcagccggactcctgaggtcacctgcgtggtcgtgtccgtgtctcacgaggacccagaagtcaaattcaactggtatgtggatggcgtcgaagtgcataatgctaagacaaaaccccgagaggaacagtataactccacctaccgggtcgtgtctgtcctgacagtgctgcatcaggactggctgaacggcaaggagtacaagtgcaaagtgagcaacaaggccctgcccgccccaatcgaaaagaccatttccaaggccaaagggcagcctcgcgaacctcaggtctacgtgtaccctccatctagggatgaactgacaaaaaaccaggtcagtctgacttgtctggtgaagggcttctacccaagcgacattgccgtggagtgggaatccaatggccagcccgagaacaattacaagactaccccccctgtgctggacagcgatgggtccttcgctctggtcagtaaactgacagtggataagtcaagatggcagcagggaaatgtctttagttgttcagtgatgcacgaggcactgcacaaccactacacccagaagtcactgtccctgtcacccggc

SEQ ID NO: 227 - protein, 15 aa, Artificial Sequence
  Description of Artificial Sequence Synthetic peptide
GGGGSGGGGSGGGGS

SEQ ID NO: 228 - DNA, 45 bp, Artificial Sequence; identical to SEQ ID NO: 62
  Description of Artificial Sequence Synthetic oligonucleotide

SEQ ID NO: 229 - protein, 110 aa, Artificial Sequence; identical to SEQ ID NO: 11
  Description of Artificial Sequence Synthetic polypeptide

SEQ ID NO: 230 - DNA, 330 bp, Artificial Sequence
  Description of Artificial Sequence Synthetic polynucleotide
gcgccagaagcagccggagggcctagcgtgttcctgtttccacccaagccaaaagataccctgatgatcagccggactcctgaggtcacctgcgtggtcgtgtccgtgtctcacgaggacccagaagtcaaattcaactggtatgtggatggcgtcgaagtgcataatgctaagacaaaaccccgagaggaacagtataactccacctaccgggtcgtgtctgtcctgacagtgctgcatcaggactggctgaacggcaaggagtacaagtgcaaagtgagcaacaaggccctgcccgccccaatcgaaaagaccatttccaaggccaaa

SEQ ID NO: 231 - protein, 106 aa, Artificial Sequence; identical to SEQ ID NO: 55
  Description of Artificial Sequence Synthetic polypeptide

SEQ ID NO: 232 - DNA, 318 bp, Artificial Sequence
  Description of Artificial Sequence Synthetic polynucleotide
gggcagcctcgcgaacctcaggtctacgtgtaccctccatctagggatgaactgacaaaaaaccaggtcagtctgacttgtctggtgaagggcttctacccaagcgacattgccgtggagtgggaatccaatggccagcccgagaacaattacaagactaccccccctgtgctggacagcgatgggtccttcgctctggtcagtaaactgacagtggataagtcaagatggcagcagggaaatgtctttagttgttcagtgatgcacgaggcactgcacaaccactacacccagaagtcactgtccctgtcacccggc

SEQ ID NO: 233 - protein, 483 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGCGTKLEIKGGGGSGGGGSGGGGSQVQLQQSGAELVRPGSSVKISCKASGYAFSSYWMNWVKQRPGQCLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 234 - DNA, 1449 bp, Artificial Sequence
  Description of Artificial Sequence Synthetic polynucleotide
gatattcagctgactcagagtcctgcttcactggcagtgagcctgggacagcgagcaaccatctcctgcaaagctagtcagtcagtggactatgatggagactcctatctgaactggtaccagcagatcccaggccagccccctaagctgctgatctacgacgcctcaaatctggtgagcggcatcccaccacgattcagcggcagcggctctgggactgattttaccctgaacattcacccagtcgagaaggtggacgccgctacataccattgccagcagtctaccgaggacccctggacattcggatgtggcactaaactggaaatcaagggaggaggaggcagtggcggaggagggtcaggaggaggaggaagccaggtgcagctgcagcagagcggagcagagctggtcagaccaggaagctccgtgaaaatttcctgcaaggcatctggctatgccttttctagttactggatgaattgggtgaagcagaggccaggccagtgtctggaatggatcgggcagatttggcccggggatggagacacaaactataatggaaagttcaaaggcaaggctacactgactgcagacgagtcaagctccactgcttatatgcagctgtctagtctggccagcgaggattccgctgtgtacttttgcgcacggagagaaaccacaactgtgggcaggtactattacgcaatggactactggggccaggggaccacagtcaccgtgtcaagcgcagccgaacccaaatcctctgataagacccacacatgccctccatgtccagcacctgagctgctgggaggaccaagcgtgttcctgtttccacctaaacctaaggacactctgatgatctctcggacacccgaagtcacttgtgtggtcgtggatgtgagccacgaggaccctgaagtcaaattcaactggtacgtggatggcgtcgaggtgcataatgccaaaacaaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagaccatttccaaagctaagggccagcctcgagaaccacaggtctatgtgtacccacccagccgggacgagctgaccaaaaaccaggtctccctgacatgtctggtgaaggggttttatccttctgatattgccgtggagtgggaaagtaatggacagccagaaaacaattacaaaactacccctccagtgctggattctgacggcagtttcgcactggtcagtaaactgaccgtggataagtcacggtggcagcaggggaacgtctttagttgttcagtgatgcacgaggccctgcacaatcattacacacagaagagcctgtccctgtctcccggc

SEQ ID NO: 235 - protein, 111 aa, Artificial Sequence; identical to SEQ ID NO: 45
  Description of Artificial Sequence Synthetic polypeptide

SEQ ID NO: 236 - DNA, 333 bp, Artificial Sequence
  Description of Artificial Sequence Synthetic polynucleotide
gatattcagctgactcagagtcctgcttcactggcagtgagcctgggacagcgagcaaccatctcctgcaaagctagtcagtcagtggactatgatggagactcctatctgaactggtaccagcagatcccaggccagccccctaagctgctgatctacgacgcctcaaatctggtgagcggcatcccaccacgattcagcggcagcggctctgggactgattttaccctgaacattcacccagtcgagaaggtggacgccgctacataccattgccagcagtctaccgaggacccctggacattcggatgtggcactaaactggaaatcaag

SEQ ID NO: 237 - protein, 15 aa, Artificial Sequence
  Description of Artificial Sequence Synthetic peptide
GGGGSGGGGSGGGGS

SEQ ID NO: 238 - DNA, 45 bp, Artificial Sequence; identical to SEQ ID NO: 62
  Description of Artificial Sequence Synthetic oligonucleotide

SEQ ID NO: 239 - protein, 124 aa, Artificial Sequence; identical to SEQ ID NO: 49
  Description of Artificial Sequence Synthetic polypeptide

SEQ ID NO: 240 - DNA, 372 bp, Artificial Sequence
  Description of Artificial Sequence Synthetic polynucleotide
caggtgcagctgcagcagagcggagcagagctggtcagaccaggaagctccgtgaaaatttcctgcaaggcatctggctatgccttttctagttactggatgaattgggtgaagcagaggccaggccagtgtctggaatggatcgggcagatttggcccggggatggagacacaaactataatggaaagttcaaaggcaaggctacactgactgcagacgagtcaagctccactgcttatatgcagctgtctagtctggccagcgaggattccgctgtgtacttttgcgcacggagagaaaccacaactgtgggcaggtactattacgcaatggactactggggccaggggaccacagtcaccgtgtcaagc

SEQ ID NO: 241 - protein, 17 aa, Artificial Sequence
  Description of Artificial Sequence Synthetic peptide
AAEPKSSDKTHTCPPCP

SEQ ID NO: 242 - DNA, 51 bp, Artificial Sequence; identical to SEQ ID NO: 66
  Description of Artificial Sequence Synthetic oligonucleotide

SEQ ID NO: 243 - protein, 110 aa, Artificial Sequence; identical to SEQ ID NO: 81
  Description of Artificial Sequence Synthetic polypeptide

SEQ ID NO: 244 - DNA, 330 bp, Artificial Sequence
  Description of Artificial Sequence Synthetic polynucleotide
gcacctgagctgctgggaggaccaagcgtgttcctgtttccacctaaacctaaggacactctgatgatctctcggacacccgaagtcacttgtgtggtcgtggatgtgagccacgaggaccctgaagtcaaattcaactggtacgtggatggcgtcgaggtgcataatgccaaaacaaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagaccatttccaaagctaag

SEQ ID NO: 245 - protein, 106 aa, Artificial Sequence; identical to SEQ ID NO: 55
  Description of Artificial Sequence Synthetic polypeptide

SEQ ID NO: 246 - DNA, 318 bp, Artificial Sequence
  Description of Artificial Sequence Synthetic polynucleotide
ggccagcctcgagaaccacaggtctatgtgtacccacccagccgggacgagctgaccaaaaaccaggtctccctgacatgtctggtgaaggggttttatccttctgatattgccgtggagtgggaaagtaatggacagccagaaaacaattacaaaactacccctccagtgctggattctgacggcagtttcgcactggtcagtaaactgaccgtggataagtcacggtggcagcaggggaacgtctttagttgttcagtgatgcacgaggccctgcacaatcattacacacagaagagcctgtccctgtctcccggc

SEQ ID NO: 247 - protein, 480 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSSSTGGGGSGGGGSGGGGSDIQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 248 - DNA, 1440 bp, Artificial Sequence
  Description of Artificial Sequence Synthetic polynucleotide
caggtccagctgcagcagagcggagctgagctggcacgaccaggagcaagtgtgaaaatgtcatgcaaggccagcggctacaccttcacacggtatactatgcactgggtgaaacagagacccggacagggcctggaatggatcgggtacattaaccctagccgaggatacaccaactacaaccagaagtttaaagacaaggctaccctgaccacagataagagctcctctacagcatatatgcagctgagttcactgacttctgaggacagtgctgtgtactattgtgcacggtactatgacgatcattactccctggattattgggggcagggaactaccctgaccgtgagctcctctagtacaggaggaggaggcagtggaggaggagggtcaggcggaggaggaagcgacatccagattgtgctgacacagtctccagcaatcatgtccgcctctcccggcgagaaagtcactatgacctgctccgcctcaagctccgtgtcttacatgaattggtatcagcagaaatcaggaaccagccccaagagatggatctacgacacatccaagctggcctctggcgtgcctgctcacttcaggggcagtgggtcaggaactagctattccctgaccattagcggcatggaggccgaagatgccgctacctactattgtcagcagtggtctagtaacccattcacatttggcagcgggactaagctggagatcaatagggcagccgaacccaaatcaagcgacaagacacatacttgccccccttgtccagcaccagaactgctgggaggaccttccgtgttcctgtttccacccaaaccaaaggatacactgatgattagccgcacccctgaggtcacatgcgtggtcgtggacgtgagccacgaggaccccgaagtcaagttcaactggtacgtggacggcgtcgaagtgcataatgccaaaaccaagcctagggaggaacagtacaacagtacatatagagtcgtgtcagtgctgaccgtcctgcaccaggattggctgaacggcaaggagtacaaatgcaaggtgtccaacaaggccctgcctgctccaatcgagaagaccatttctaaagcaaaggggcagccccgagaacctcaggtctacgtgtatcctccatcccgggacgagctgactaaaaaccaggtctctctgacctgtctggtgaagggcttttacccatctgatattgctgtcgagtgggaaagtaatgggcagcccgagaacaattataagacaactccccctgtgctggactccgatgggtctttcgccctggtcagcaaactgacagtggataagtccagatggcagcagggaaacgtcttttcttgtagtgtgatgcatgaagctctgcacaatcattacactcagaaatcactgagcctgtcccccggcaag

SEQ ID NO: 249 - protein, 119 aa, Artificial Sequence; identical to SEQ ID NO: 115
  Description of Artificial Sequence Synthetic polypeptide

SEQ ID NO: 250 - DNA, 357 bp, Artificial Sequence
  Description of Artificial Sequence Synthetic polynucleotide
caggtccagctgcagcagagcggagctgagctggcacgaccaggagcaagtgtgaaaatgtcatgcaaggccagcggctacaccttcacacggtatactatgcactgggtgaaacagagacccggacagggcctggaatggatcgggtacattaaccctagccgaggatacaccaactacaaccagaagtttaaagacaaggctaccctgaccacagataagagctcctctacagcatatatgcagctgagttcactgacttctgaggacagtgctgtgtactattgtgcacggtactatgacgatcattactccctggattattgggggcagggaactaccctgaccgtgagctcc

SEQ ID NO: 251 - protein, 15 aa, Artificial Sequence
  Description of Artificial Sequence Synthetic peptide
GGGGSGGGGSGGGGS

SEQ ID NO: 252 - DNA, 45 bp, Artificial Sequence; identical to SEQ ID NO: 118
  Description of Artificial Sequence Synthetic oligonucleotide

SEQ ID NO: 253 - protein, 106 aa, Artificial Sequence [sequence omitted]

SEQ ID NO: 254 - DNA, 318 bp, Artificial Sequence
  Description of Artificial Sequence Synthetic polynucleotide
cagattgtgctgacacagtctccagcaatcatgtccgcctctcccggcgagaaagtcactatgacctgctccgcctcaagctccgtgtcttacatgaattggtatcagcagaaatcaggaaccagccccaagagatggatctacgacacatccaagctggcctctggcgtgcctgctcacttcaggggcagtgggtcaggaactagctattccctgaccattagcggcatggaggccgaagatgccgctacctactattgtcagcagtggtctagtaacccattcacatttggcagcgggactaagctggagatcaat

SEQ ID NO: 255 - protein, 17 aa, Artificial Sequence
  Description of Artificial Sequence Synthetic peptide
AAEPKSSDKTHTCPPCP

SEQ ID NO: 256 - DNA, 51 bp, Artificial Sequence; identical to SEQ ID NO: 122
  Description of Artificial Sequence Synthetic oligonucleotide

SEQ ID NO: 257 - protein, 110 aa, Artificial Sequence; identical to SEQ ID NO: 81
  Description of Artificial Sequence Synthetic polypeptide

SEQ ID NO: 258 - DNA, 330 bp, Artificial Sequence
  Description of Artificial Sequence Synthetic polynucleotide
gcaccagaactgctgggaggaccttccgtgttcctgtttccacccaaaccaaaggatacactgatgattagccgcacccctgaggtcacatgcgtggtcgtggacgtgagccacgaggaccccgaagtcaagttcaactggtacgtggacggcgtcgaagtgcataatgccaaaaccaagcctagggaggaacagtacaacagtacatatagagtcgtgtcagtgctgaccgtcctgcaccaggattggctgaacggcaaggagtacaaatgcaaggtgtccaacaaggccctgcctgctccaatcgagaagaccatttctaaagcaaag

SEQ ID NO: 259 - protein, 106 aa, Artificial Sequence; identical to SEQ ID NO: 55
  Description of Artificial Sequence Synthetic polypeptide

SEQ ID NO: 260 - DNA, 318 bp, Artificial Sequence
  Description of Artificial Sequence Synthetic polynucleotide
gggcagccccgagaacctcaggtctacgtgtatcctccatcccgggacgagctgactaaaaaccaggtctctctgacctgtctggtgaagggcttttacccatctgatattgctgtcgagtgggaaagtaatgggcagcccgagaacaattataagacaactccccctgtgctggactccgatgggtctttcgccctggtcagcaaactgacagtggataagtccagatggcagcagggaaacgtcttttcttgtagtgtgatgcatgaagctctgcacaatcattacactcagaaatcactgagcctgtcccccggc

SEQ ID NO: 261 - protein, 484 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 262 - DNA, 1452 bp, Artificial Sequence
  Description of Artificial Sequence Synthetic polynucleotide
gacattcagctgacccagagtcctgcttcactggcagtgagcctgggacagcgagcaacaatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctggaaccgattttacactgaacattcacccagtcgagaaggtggacgccgctacctaccattgccagcagtctacagaggacccctggactttcggcgggggaaccaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacacaaactataatggaaagttcaaaggcaaggctactctgaccgcagacgagtcaagctccactgcatatatgcagctgtctagtctggccagcgaggattccgctgtctacttttgcgcacggagagaaaccacaactgtgggcaggtactattacgccatggactactggggccaggggaccacagtcaccgtgtcaagcgcagccgaacccaaatcctctgataagacacacacttgccctccatgtccagctcctgagctgctgggaggaccaagcgtgttcctgtttccacctaaacctaaggacactctgatgatctctcggactcccgaagtcacctgtgtggtcgtggatgtgagccacgaggaccctgaagtcaaattcaactggtacgtggatggcgtcgaggtgcataatgccaaaacaaagcctagggaggaacagtataactccacataccgcgtcgtgtctgtcctgactgtgctgcatcaggactggctgaacggaaaggagtacaaatgcaaggtgagcaacaaggccctgccagctcccatcgagaagaccatttccaaagctaagggccagcctcgagaaccacaggtctatgtgctgccacccagccgggacgagctgacaaaaaaccaggtctccctgctgtgtctggtgaagggattctacccttctgatattgcagtggagtgggaaagtaatggccagccagaaaacaattatctgacttggcctccagtgctggattctgacgggagtttctttctgtacagtaaactgaccgtggataagtcacggtggcagcagggaaacgtctttagttgttcagtgatgcacgaggccctgcacaatcattacacccagaaaagcctgtccctgtctcccggcaag

SEQ ID NO: 263 - protein, 111 aa, Artificial Sequence; identical to SEQ ID NO: 59
  Description of Artificial Sequence Synthetic polypeptide

SEQ ID NO: 264 - DNA, 333 bp, Artificial Sequence
  Description of Artificial Sequence Synthetic polynucleotide
gacattcagctgacccagagtcctgcttcactggcagtgagcctgggacagcgagcaacaatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctggaaccgattttacactgaacattcacccagtcgagaaggtggacgccgctacctaccattgccagcagtctacagaggacccctggactttcggcgggggaaccaaactggaaatcaag

SEQ ID NO: 265 - protein, 15 aa, Artificial Sequence
  Description of Artificial Sequence Synthetic peptide
GGGGSGGGGSGGGGS

SEQ ID NO: 266 - DNA, 45 bp, Artificial Sequence; identical to SEQ ID NO: 62
  Description of Artificial Sequence Synthetic oligonucleotide

SEQ ID NO: 267 - protein, 124 aa, Artificial Sequence; identical to SEQ ID NO: 63
  Description of Artificial Sequence Synthetic polypeptide

SEQ ID NO: 268 - DNA, 372 bp, Artificial Sequence
  Description of Artificial Sequence Synthetic polynucleotide
caggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacacaaactataatggaaagttcaaaggcaaggctactctgaccgcagacgagtcaagctccactgcatatatgcagctgtctagtctggccagcgaggattccgctgtctacttttgcgcacggagagaaaccacaactgtgggcaggtactattacgccatggactactggggccaggggaccacagtcaccgtgtcaagc

SEQ ID NO: 269 - protein, 17 aa, Artificial Sequence
  Description of Artificial Sequence Synthetic peptide
AAEPKSSDKTHTCPPCP

SEQ ID NO: 270 - DNA, 51 bp, Artificial Sequence; identical to SEQ ID NO: 94
  Description of Artificial Sequence Synthetic oligonucleotide

SEQ ID NO: 271 - protein, 110 aa, Artificial Sequence; identical to SEQ ID NO: 81
  Description of Artificial Sequence Synthetic polypeptide

SEQ ID NO: 272 - DNA, 330 bp, Artificial Sequence
  Description of Artificial Sequence Synthetic polynucleotide
gctcctgagctgctgggaggaccaagcgtgttcctgtttccacctaaacctaaggacactctgatgatctctcggactcccgaagtcacctgtgtggtcgtggatgtgagccacgaggaccctgaagtcaaattcaactggtacgtggatggcgtcgaggtgcataatgccaaaacaaagcctagggaggaacagtataactccacataccgcgtcgtgtctgtcctgactgtgctgcatcaggactggctgaacggaaaggagtacaaatgcaaggtgagcaacaaggccctgccagctcccatcgagaagaccatttccaaagctaag

SEQ ID NO: 273 - protein, 106 aa, Artificial Sequence; identical to SEQ ID NO: 13
  Description of Artificial Sequence Synthetic polypeptide

SEQ ID NO: 274 - DNA, 318 bp, Artificial Sequence
  Description of Artificial Sequence Synthetic polynucleotide
ggccagcctcgagaaccacaggtctatgtgctgccacccagccgggacgagctgacaaaaaaccaggtctccctgctgtgtctggtgaagggattctacccttctgatattgcagtggagtgggaaagtaatggccagccagaaaacaattatctgacttggcctccagtgctggattctgacgggagtttctttctgtacagtaaactgaccgtggataagtcacggtggcagcagggaaacgtctttagttgttcagtgatgcacgaggccctgcacaatcattacacccagaaaagcctgtccctgtctcccggc

SEQ ID NO: 275 - protein, 473 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEINGGGGSGGGGSGGGGSQVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 276 - DNA, 1419 bp, Artificial Sequence
  Description of Artificial Sequence Synthetic polynucleotide
cagatcgtcctgactcagagccccgctattatgtccgcaagccctggagagaaagtgactatgacctgttccgcatctagttccgtgtcctacatgaactggtatcagcagaaatctggaacaagtcccaagcgatggatctacgacacttccaagctggcatctggagtgcctgcccacttccgaggcagcggctctgggacaagttattcactgactattagcggcatggaggccgaagatgccgctacatactattgccagcagtggagctccaacccattcacctttggatgtggcacaaagctggagatcaatggcggaggaggctccggaggaggagggtctggaggaggaggaagtcaggtccagctgcagcagtccggagcagaactggctagaccaggagccagtgtgaaaatgtcatgcaaggccagcggctacacattcactcggtataccatgcattgggtgaaacagagaccaggacagtgtctggagtggatcggctacattaatcccagcagggggtacacaaactacaaccagaagtttaaagacaaggcaaccctgaccaccgataagtctagttcaacagcttatatgcagctgagctccctgacttcagaagacagcgctgtgtactattgcgcacgctactatgacgatcactactccctggattattgggggcagggaactaccctgaccgtgtctagtgcagccgagcctaaatcaagcgacaagacccatacatgccccccttgtccggcgccagaagctgcaggcggaccaagtgtgttcctgtttccacccaaacctaaggatactctgatgatttctcgaactcctgaggtcacctgcgtggtcgtgagcgtgtcccacgaggacccagaagtcaagttcaactggtacgtggatggggtcgaagtgcataatgccaaaaccaagcccagggaggaacagtacaactcaacttatcgcgtcgtgtctgtcctgaccgtgctgcaccaggactggctgaatggcaaggagtacaaatgtaaggtctcaaataaggctctgcccgcccctatcgaaaaaactatctctaaggcaaaaggacagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 277 - protein, 106 aa, Artificial Sequence; identical to SEQ ID NO: 17
  Description of Artificial Sequence Synthetic polypeptide

SEQ ID NO: 278 - DNA, 318 bp, Artificial Sequence
  Description of Artificial Sequence Synthetic polynucleotide
cagatcgtcctgactcagagccccgctattatgtccgcaagccctggagagaaagtgactatgacctgttccgcatctagttccgtgtcctacatgaactggtatcagcagaaatctggaacaagtcccaagcgatggatctacgacacttccaagctggcatctggagtgcctgcccacttccgaggcagcggctctgggacaagttattcactgactattagcggcatggaggccgaagatgccgctacatactattgccagcagtggagctccaacccattcacctttggatgtggcacaaagctggagatcaat

SEQ ID NO: 279 - protein, 15 aa, Artificial Sequence
  Description of Artificial Sequence Synthetic peptide
GGGGSGGGGSGGGGS

SEQ ID NO: 280 - DNA, 45 bp, Artificial Sequence; identical to SEQ ID NO: 6
  Description of Artificial Sequence Synthetic oligonucleotide

SEQ ID NO: 281 - protein, 119 aa, Artificial Sequence; identical to SEQ ID NO: 161
  Description of Artificial Sequence Synthetic polypeptide

SEQ ID NO: 282 - DNA, 357 bp, Artificial Sequence
  Description of Artificial Sequence Synthetic polynucleotide
caggtccagctgcagcagtccggagcagaactggctagaccaggagccagtgtgaaaatgtcatgcaaggccagcggctacacattcactcggtataccatgcattgggtgaaacagagaccaggacagtgtctggagtggatcggctacattaatcccagcagggggtacacaaactacaaccagaagtttaaagacaaggcaaccctgaccaccgataagtctagttcaacagcttatatgcagctgagctccctgacttcagaagacagcgctgtgtactattgcgcacgctactatgacgatcactactccctggattattgggggcagggaactaccctgaccgtgtctagt

SEQ ID NO: 283 - protein, 17 aa, Artificial Sequence
  Description of Artificial Sequence Synthetic peptide
AAEPKSSDKTHTCPPCP

SEQ ID NO: 284 - DNA, 51 bp, Artificial Sequence; identical to SEQ ID NO: 24
  Description of Artificial Sequence Synthetic oligonucleotide

SEQ ID NO: 285 - protein, 110 aa, Artificial Sequence; identical to SEQ ID NO: 11
  Description of Artificial Sequence Synthetic polypeptide

SEQ ID NO: 286 - DNA, 330 bp, Artificial Sequence
  Description of Artificial Sequence Synthetic polynucleotide
gcgccagaagctgcaggcggaccaagtgtgttcctgtttccacccaaacctaaggatactctgatgatttctcgaactcctgaggtcacctgcgtggtcgtgagcgtgtcccacgaggacccagaagtcaagttcaactggtacgtggatggggtcgaagtgcataatgccaaaaccaagcccagggaggaacagtacaactcaacttatcgcgtcgtgtctgtcctgaccgtgctgcaccaggactggctgaatggcaaggagtacaaatgtaaggtctcaaataaggctctgcccgcccctatcgaaaaaactatctctaaggcaaaa

SEQ ID NO: 287 - protein, 106 aa, Artificial Sequence; identical to SEQ ID NO: 13
  Description of Artificial Sequence Synthetic polypeptide

SEQ ID NO: 288 - DNA, 318 bp, Artificial Sequence
  Description of Artificial Sequence Synthetic polynucleotide
ggacagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 289 - protein, 5 aa, Artificial Sequence
  Description of Artificial Sequence Synthetic peptide
SSVSY

SEQ ID NO: 290 - protein, 3 aa, Artificial Sequence
  Description of Artificial Sequence Synthetic peptide
DTS

SEQ ID NO: 291 - protein, 7 aa, Artificial Sequence
  Description of Artificial Sequence Synthetic peptide
QQWSSNP

SEQ ID NO: 292 - protein, 8 aa, Artificial Sequence
  Description of Artificial Sequence Synthetic peptide
GYTFTRYT

SEQ ID NO: 293 - protein, 8 aa, Artificial Sequence
  Description of Artificial Sequence Synthetic peptide
INPSRGYT

SEQ ID NO: 294 - protein, 12 aa, Artificial Sequence
  Description of Artificial Sequence Synthetic peptide
ARYYDDHYCLDY

SEQ ID NO: 295 - protein, 5 aa, Artificial Sequence
  Description of Artificial Sequence Synthetic peptide
SSVSY

SEQ ID NO: 296 - protein, 3 aa, Artificial Sequence
  Description of Artificial Sequence Synthetic peptide
DTS

SEQ ID NO: 297 - protein, 7 aa, Artificial Sequence
  Description of Artificial Sequence Synthetic peptide
QQWSSNP

SEQ ID NO: 298 - protein, 8 aa, Artificial Sequence
  Description of Artificial Sequence Synthetic peptide
GYTFTRYT

SEQ ID NO: 299 - protein, 8 aa, Artificial Sequence
  Description of Artificial Sequence Synthetic peptide
INPSRGYT

SEQ ID NO: 300 - protein, 12 aa, Artificial Sequence
  Description of Artificial Sequence Synthetic peptide
ARYYDDHYSLDY

SEQ ID NO: 301 - protein, 11 aa, Artificial Sequence
  Description of Artificial Sequence Synthetic peptide
QSVDYDGDSYL

SEQ ID NO: 302 - protein, 3 aa, Artificial Sequence
  Description of Artificial Sequence Synthetic peptide
DAS

SEQ ID NO: 303 - protein, 9 aa, Artificial Sequence
  Description of Artificial Sequence Synthetic peptide
QQSTEDPWT

SEQ ID NO: 304 - protein, 8 aa, Artificial Sequence
  Description of Artificial Sequence Synthetic peptide
GYAFSSYW

SEQ ID NO: 305 - protein, 8 aa, Artificial Sequence
  Description of Artificial Sequence Synthetic peptide
IWPGDGDT

SEQ ID NO: 306 - protein, 15 aa, Artificial Sequence
  Description of Artificial Sequence Synthetic peptide
RETTTVGRYYYAMDY

SEQ ID NO: 307 - protein, 11 aa, Artificial Sequence
  Description of Artificial Sequence Synthetic peptide
QSVDYEGDSYL

SEQ ID NO: 308 - protein, 3 aa, Artificial Sequence
  Description of Artificial Sequence Synthetic peptide
DAS

SEQ ID NO: 309 - protein, 9 aa, Artificial Sequence
  Description of Artificial Sequence Synthetic peptide
QQSTEDPWT

SEQ ID NO: 310 - protein, 8 aa, Artificial Sequence
  Description of Artificial Sequence Synthetic peptide
GYAFSSYW

SEQ ID NO: 311 - protein, 8 aa, Artificial Sequence
  Description of Artificial Sequence Synthetic peptide
IWPGDGDT

SEQ ID NO: 312 - protein, 15 aa, Artificial Sequence
  Description of Artificial Sequence Synthetic peptide
RETTTVGRYYYAMDY

SEQ ID NO: 313 - protein, 11 aa, Artificial Sequence
  Description of Artificial Sequence Synthetic peptide
QSVDYSGDSYL

SEQ ID NO: 314 - protein, 3 aa, Artificial Sequence
  Description of Artificial Sequence Synthetic peptide
DAS

SEQ ID NO: 315 - protein, 9 aa, Artificial Sequence
  Description of Artificial Sequence Synthetic peptide
QQSTEDPWT

SEQ ID NO: 316 - protein, 8 aa, Artificial Sequence
  Description of Artificial Sequence Synthetic peptide
GYAFSSYW

SEQ ID NO: 317 - protein, 8 aa, Artificial Sequence
  Description of Artificial Sequence Synthetic peptide
IWPGDGDT

SEQ ID NO: 318 - protein, 15 aa, Artificial Sequence
  Description of Artificial Sequence Synthetic peptide
RETTTVGRYYYAMDY

SEQ ID NO: 319 - protein, 14 aa, Artificial Sequence
  Description of Artificial Sequence Synthetic peptide
KASQSVDYDGDSYL

SEQ ID NO: 320 - protein, 7 aa, Artificial Sequence
  Description of Artificial Sequence Synthetic peptide
DASNLVS

SEQ ID NO: 321 - protein, 9 aa, Artificial Sequence
  Description of Artificial Sequence Synthetic peptide
QQSTEDPWT

SEQ ID NO: 322 - protein, 10 aa, Artificial Sequence
  Description of Artificial Sequence Synthetic peptide
GYAFSSYWMN

SEQ ID NO: 323 - protein, 10 aa, Artificial Sequence
  Description of Artificial Sequence Synthetic peptide
QIWPGDGDTN

SEQ ID NO: 324 - protein, 15 aa, Artificial Sequence
  Description of Artificial Sequence Synthetic peptide
RETTTVGRYYYAMDY

SEQ ID NO: 325 - protein, 14 aa, Artificial Sequence
  Description of Artificial Sequence Synthetic peptide
RASQSVDYEGDSYL

SEQ ID NO: 326 - protein, 7 aa, Artificial Sequence
  Description of Artificial Sequence Synthetic peptide
DASNLVS

SEQ ID NO: 327 - protein, 9 aa, Artificial Sequence
  Description of Artificial Sequence Synthetic peptide
QQSTEDPWT

SEQ ID NO: 328 - protein, 10 aa, Artificial Sequence
  Description of Artificial Sequence Synthetic peptide
GYAFSSYWMN

SEQ ID NO: 329 - protein, 10 aa, Artificial Sequence
  Description of Artificial Sequence Synthetic peptide
QIWPGDGDTN

SEQ ID NO: 330 - protein, 15 aa, Artificial Sequence
  Description of Artificial Sequence Synthetic peptide
RETTTVGRYYYAMDY

SEQ ID NO: 331 - protein, 14 aa, Artificial Sequence
  Description of Artificial Sequence Synthetic peptide
RASQSVDYSGDSYL

SEQ ID NO: 332 - protein, 7 aa, Artificial Sequence
  Description of Artificial Sequence Synthetic peptide
DASNLVS

SEQ ID NO: 333 - protein, 9 aa, Artificial Sequence
  Description of Artificial Sequence Synthetic peptide
QQSTEDPWT

SEQ ID NO: 334 - protein, 10 aa, Artificial Sequence
  Description of Artificial Sequence Synthetic peptide
GYAFSSYWMN

SEQ ID NO: 335 - protein, 10 aa, Artificial Sequence
  Description of Artificial Sequence Synthetic peptide
QIWPGDGDTN

SEQ ID NO: 336 - protein, 15 aa, Artificial Sequence
  Description of Artificial Sequence Synthetic peptide
RETTTVGRYYYAMDY

SEQ ID NO: 337 - protein, 111 aa, Artificial Sequence
  Description of Artificial Sequence Synthetic polypeptide
DIQLTQSPSSLSASVGDRATITCRASQSVDYDGDSYLNWYQQKPGKAPKLLIYDASNLVSGIPSRFSGSGSGTDFTLTISSVQPEDAATYYCQQSTEDPWTFGCGTKLEIK

SEQ ID NO: 338 - DNA, 333 bp, Artificial Sequence
  Description of Artificial Sequence Synthetic polynucleotide
gatattcagctgacccagagcccaagctccctgtctgccagtgtgggggatagggctacaatcacttgccgcgcatcacagagcgtggactatgagggcgattcctatctgaactggtaccagcagaagccagggaaagcacccaagctgctgatctacgacgcctctaatctggtgagtggcattccctcaaggttctccggatctggcagtgggactgactttaccctgacaatctctagtgtgcagcccgaggatgccgctacctactattgccagcagtctacagaagacccttggactttcggatgtggcaccaaactggagattaag

SEQ ID NO: 339 - protein, 124 aa, Artificial Sequence
  Description of Artificial Sequence Synthetic polypeptide
QVQLVQSGAEVKKPGASVKISCKASGYAFSSYWMNWVRQAPGQCLEWIGQIWPGDGDTNYAQKFQGRATLTADTSTSTAYMELSSLRSEDTAVYYCARRETTTVGRYYYAMDYWGQGTTVTVSS

SEQ ID NO: 340 - DNA, 372 bp, Artificial Sequence
  Description of Artificial Sequence Synthetic polynucleotide
caggtccagctggtgcagagcggagcagaggtcaagaaacccggagccagcgtgaaaatttcctgcaaggcctctggctatgctttctcaagctactggatgaactgggtgaggcaggcaccaggacagtgtctggaatggatcggacagatttggcctggggacggagataccaattatgctcagaagtttcagggacgcgcaactctgaccgccgatacatcaacaagcactgcatacatggagctgtcctctctgcgctccgaagacacagccgtgtactattgcgcacggagagaaaccacaactgtgggccgatactattacgcaatggattactggggccaggggaccacagtcactgtgagttca

SEQ ID NO: 341 - protein, 124 aa, Artificial Sequence
  Description of Artificial Sequence Synthetic polypeptide
QVQLVQSGAEVKKPGASVKISCKASGYAFSSYWMNWVRQAPGQCLEWIGQIWPGDGDTNYAQKFQGRATLTADESTSTAYMELSSLRSEDTAVYYCARRETTTVGRYYYAMDYWGQGTTVTVSS

SEQ ID NO: 342 - DNA, 372 bp, Artificial Sequence
  Description of Artificial Sequence Synthetic polynucleotide
caggtccagctggtgcagagcggagcagaggtcaagaaacccggagccagcgtgaaaatttcctgcaaggcctctggctatgctttctcaagctactggatgaactgggtgaggcaggcaccaggacagtgtctggaatggatcggacagatttggcctggggacggagataccaattatgctcagaagtttcagggacgcgcaactctgaccgccgatgagtcaacaagcactgcatacatggagctgtcctctctgcgctccgaagacacagccgtgtactattgcgcacggagagaaaccacaactgtgggccgatactattacgcaatggattactggggccaggggaccacagtcactgtgagttca

SEQ ID NO: 343 - protein, 15 aa, Artificial Sequence
  Description of Artificial Sequence Synthetic peptide
GGGGSGGGGSGGGGS

SEQ ID NO: 344 - protein, 20 aa, Artificial Sequence
  Description of Artificial Sequence Synthetic peptide
SSTGGGGSGGGGSGGGGSDI

SEQ ID NO: 345 - protein, 18 aa, Artificial Sequence
  Description of Artificial Sequence Synthetic peptide
VEGGSGGSGGSGGSGGVD

SEQ ID NO: 346 - protein, 15 aa, Artificial Sequence
  Description of Artificial Sequence Synthetic peptide
GGGGSGGGGSGGGGS

SEQ ID NO: 347 - protein, 20 aa, Artificial Sequence
  Description of Artificial Sequence Synthetic peptide
GGGGSGGGGSGGGGSGGGGS

SEQ ID NO: 348 - protein, 18 aa, Artificial Sequence
  Description of Artificial Sequence Synthetic peptide
GSTSGGGSGGGSGGGGSS

SEQ ID NO: 349 - protein, 18 aa, Artificial Sequence
  Description of Artificial Sequence Synthetic peptide
GSTSGSGKPGSGEGSTKG

SEQ ID NO: 350 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 351 - protein, 15 aa, Artificial Sequence
  Description of Artificial Sequence Synthetic peptide
EPKSCDKTHTCPPCP

SEQ ID NO: 352 - DNA, 45 bp, Artificial Sequence
  Description of Artificial Sequence Synthetic oligonucleotide
gagcccaagagctgtgataagacccacacctgccctccctgtcca

SEQ ID NO: 353 - protein, 17 aa, Artificial Sequence
  Description of Artificial Sequence Synthetic peptide
AAEPKSSDKTHTCPPCP

SEQ ID NO: 354 - DNA, 51 bp, Artificial Sequence; identical to SEQ ID NO: 66
  Description of Artificial Sequence Synthetic oligonucleotide

SEQ ID NO: 355 - protein, 15 aa, Artificial Sequence
  Description of Artificial Sequence Synthetic peptide
EPKSSDKTHTCPPCP

SEQ ID NO: 356 - DNA, 45 bp, Artificial Sequence
  Description of Artificial Sequence Synthetic oligonucleotide
gagcctaaaagctccgacaagacccacacatgcccaccttgtccg

SEQ ID NO: 357 - protein, 10 aa, Artificial Sequence
  Description of Artificial Sequence Synthetic peptide
DKTHTCPPCP

SEQ ID NO: 358 - DNA, 30 bp, Artificial Sequence
  Description of Artificial Sequence Synthetic oligonucleotide
gacaagacccacacatgcccaccttgtccg

SEQ ID NO: 359 - protein, 7 aa, Artificial Sequence
  Description of Artificial Sequence Synthetic peptide
GTCPPCP

SEQ ID NO: 360 - DNA, 21 bp, Artificial Sequence
  Description of Artificial Sequence Synthetic oligonucleotide
ggcacatgccctccatgtcca

SEQ ID NO: 361 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 15 aa, Artificial Sequence
  Description of Artificial Sequence Synthetic peptide
GGGGSGGGGSGGGGS

SEQ ID NO: 363 - protein, 50 aa, Artificial Sequence
  Description of Artificial Sequence Synthetic polypeptide
GGGGSGGGGSGGGGSGGGGSGGGGSGGGGSGGGGSGGGGSGGGGSGGGGS

SEQ ID NO: 364 - protein, 50 aa, Artificial Sequence
  Description of Artificial Sequence Synthetic polypeptide
SGGGGSGGGGSGGGGSGGGGSGGGGSGGGGSGGGGSGGGGSGGGGSGGGG

SEQ ID NO: 365 - protein, 54 aa, Artificial Sequence
  Description of Artificial Sequence Synthetic polypeptide
GGGGSGGGGSGGGGSGGGGSGGGGSGGGGSGGGGSGGGGSGGGGSGGGGSGGGG

SEQ ID NO: 366 - protein, 32 aa, Artificial Sequence
  Description of Artificial Sequence Synthetic polypeptide
GGSGGSGGSGGSGGSGGSGGSGGSGGSGGSGG

SEQ ID NO: 367 - protein, 111 aa, Mus musculus; identical to SEQ ID NO: 59

SEQ ID NO: 368 - protein, 111 aa, Artificial Sequence; identical to SEQ ID NO: 337
  Description of Artificial Sequence Synthetic polypeptide

SEQ ID NO: 369 - protein, 111 aa, Artificial Sequence
  Description of Artificial Sequence Synthetic polypeptide
DIQLTQSPSSLSASVGDRATITCRASQSVDYEGDSYLNWYQQKPGKAPKLLIYDASNLVSGIPSRFSGSGSGTDFTLTISSVQPEDAATYYCQQSTEDPWTFGCGTKLEIK

SEQ ID NO: 370 - protein, 111 aa, Artificial Sequence
  Description of Artificial Sequence Synthetic polypeptide
DIQLTQSPSSLSASVGDRATITCRASQSVDYSGDSYLNWYQQKPGKAPKLLIYDASNLVSGIPSRFSGSGSGTDFTLTISSVQPEDAATYYCQQSTEDPWTFGCGTKLEIK

SEQ ID NO: 371 - protein, 124 aa, Mus musculus; identical to SEQ ID NO: 63

SEQ ID NO: 372 - protein, 124 aa, Artificial Sequence; identical to SEQ ID NO: 339
  Description of Artificial Sequence Synthetic polypeptide

SEQ ID NO: 373 - protein, 124 aa, Artificial Sequence; identical to SEQ ID NO: 341
  Description of Artificial Sequence Synthetic polypeptide

(19) United States
(12) Patent Application Publication
(10) Pub. No.: US 2016/0326249 A1
(43) Pub. Date: Nov. 10, 2016

(54) BI-SPECIFIC CD3 AND CD19 ANTIGEN-BINDING CONSTRUCTS

(71) Applicant: ZYMEWORKS INC., Vancouver (CA)

(72) Inventors: Gordon Yiu Kon Ng, Vancouver (CA); Thomas Spreter von Kreudenstein, Vancouver (BC); Leonard G. Presta, San Francisco, CA (US)

(73) Assignee: Zymeworks Inc., Vancouver (CA)

(21) Appl. No.: 15/109,709

(22) PCT Filed: Jan. 15, 2015
(86) PCT No.: PCT/US2015/011664

§ 371 Date: Jul. 5, 2016

Related U.S. Application Data
(60) Provisional application No. 61/927,877, filed on Jan. 15, 2014.
(60) Provisional application No. 61/978,719, filed on Apr. 11, 2014.
(60) Provisional application No. 62/025,932, filed on Jul. 17, 2014.

(30) Foreign Application Priority Data
Jul. 11, 2014 (US) PCT/US2014/046436

(51) Int. Cl. C07K 16/28

(52) U.S. Cl. CPC C07K 16/2809; C07K 16/2803; C07K 2317/31; C07K 2317/526; C07K 2317/565; C07K 2317/622; C07K 2317/64; C07K 2317/71; A61K 2039/505

(57) ABSTRACT

Antigen-binding constructs, e.g., antibodies, which bind CD3 and CD 19 and methods of use are disclosed.

### CROSS-REFERENCE TO RELATED APPLICATIONS

This application claims the benefit of U.S. Provisional Application No. 61/927,877, filed on Jan. 15, 2014 and U.S. Provisional Application No. 61/978,719, filed on Apr. 11, 2014 and U.S. Provisional Application No. 62/025,932, filed on Jul. 17, 2014. This application also claims priority to International Application No. PCT/US2014/046436, filed on Jul. 11, 2014. Each of these applications are hereby incorporated in their entirety by reference.

### SEQUENCE LISTING

The instant application contains a Sequence Listing which has been submitted via EFS-Web and is hereby incorporated by reference in its entirety. Said ASCII copy, created on Month XX, 2015, is named XXXXX_CRF_sequencelisting.txt, and is XXX,XXX bytes in size.

### FIELD OF THE INVENTION

The field of the invention is bi-specific antigen-binding constructs, e.g., antibodies, comprising a CD3 antigen-binding polypeptide construct, e.g., a CD3 binding domain and a CD19 antigen-binding polypeptide construct, e.g., a CD19 binding domain.

### BACKGROUND OF THE INVENTION

In the realm of therapeutic proteins, antibodies with their multivalent target binding features are excellent scaffolds for the design of drug candidates. Advancing these features further, designed bi-specific antibodies and other fused multispecific therapeutics exhibit dual or multiple target specificities and an opportunity to create drugs with novel modes of action. The development of such multivalent and multispecific therapeutic proteins with favorable pharmacokinetics and functional activity has been a challenge.

Bi-specific antibodies capable of targeting T cells to tumor cells have been identified and tested for their efficacy in the treatment of cancers. Blinatumomab is an example of a bi-specific anti-CD3-CD19 antibody in a format called BiTE™ (Bi-specific T-cell Engager) that has been identified for the treatment of B-cell diseases such as relapsed B-cell non-Hodgkin lymphoma and chronic lymphocytic leukemia (Baeuerle et al (2009) 12:4941-4944). The BiTE™ format is a bi-specific single chain antibody construct that links variable domains derived from two different antibodies. Blinatumomab, however, possesses poor half-life in vivo, and is difficult to manufacture in terms of production and stability. Thus, there is a need for improved bi-specific antibodies, capable of targeting T-cells to tumor cells and having improved manufacturability.

Antigen binding constructs are described in the following: International application no. PCT/US2013/050411 filed on Jul. 13, 2013 and titled “Bispecific Asymmetric Heterodimers Comprising Anti-CD3 Constructs;” International application no. PCT/US2014/046436 filed on Jul. 11, 2014 and titled “Bispecific CD3 and CD19 Antigen Binding Constructs.”

### SUMMARY OF THE INVENTION

Described herein are antigen-binding constructs, each comprising a first antigen-binding polypeptide construct, a second antigen-binding polypeptide construct and a heterodimeric Fc. The first scFv comprises a first VL, a first scFv linker, and a first VH. The first scFv monovalently and specifically binds a CD19 antigen. The first scFv is selected from the group consisting of an anti-CD19 antibody HD37 scFv, a modified HD37 scFv, an HD37 blocking antibody scFv, and a modified HD37 blocking antibody scFv, wherein the HD37 blocking antibody blocks by 50% or greater the binding of HD37 to the CD19 antigen.

The second antigen-binding polypeptide construct comprises a second scFv comprising a second VL, a second scFv linker, and a second VH. The second scFv monovalently and specifically binding an epsilon subunit of a CD3 antigen. The second scFv is selected from the group consisting of the OKT3 scFv, a modified OKT3 scFv, an OKT3 blocking antibody scFv, and a modified OKT3 blocking antibody scFv, wherein the OKT3 blocking antibody blocks by 50% or greater the binding of OKT3 to the epsilon subunit of the CD3 antigen.

The heterodimeric Fc comprises first and second Fc polypeptides each comprising a modified CH3 sequence capable of forming a dimerized CH3 domain, wherein each modified CH3 sequence comprises asymmetric amino acid modifications that promote formation of a heterodimeric Fc and the dimerized CH3 domains have a melting temperature (Tm) of about 68° C. or higher. The first Fc polypeptide is linked to the first antigen-binding polypeptide construct with a first hinge linker, and the second Fc polypeptide is linked to the second antigen-binding polypeptide construct with a second hinge linker.

Also described are antigen-binding constructs polypeptide sequences and CDR sequences, nucleic acids encoding antigen-binding constructs, and vectors and cells. Also described are pharmaceutical compositions comprising the antigen-binding constructs and methods of treating a disorder, e.g., cancer, using the antigen-binding constructs described herein.

### BRIEF DESCRIPTION OF THE FIGURES

FIG. 1 depicts schematic representations of designs of antigen-binding constructs. FIG. 1A shows a representation of an exemplary CD3-CD19 antigen-binding construct with an Fc that is capable of mediating effector function. Both of the antigen-binding domains of the antigen-binding construct are scFvs, with the VH and VL regions of each scFv connected with a polypeptide linker. Each scFv is also connected to one polypeptide chain of a heterodimeric Fc with a hinge polypeptide linker. The two polypeptide chains of the antigen-binding construct are covalently linked together via disulphide bonds (depicted as dashed lines). FIG. 1B depicts a representation of an exemplary CD3-CD19 antigen-binding construct with an Fc knockout. This type of antigen-binding construct is similar to that shown in FIG. 1A, except that it includes modifications to the CH2 region of the Fc that ablate FcγR binding (denoted by “X”).

FIG. 2 shows the analysis of the purification procedure for selected variants. The upper panel in FIG. 2A depicts the preparative gel filtration (GFC) profile after protein A purification for variant 10149, while the lower panel shows the analytical SEC profile of the pooled GFC fractions. The upper panel of FIG. 2B shows the preparative gel filtration (GFC) profile after protein A purification for variant 1661, while the lower panel shows the analytical SEC profile of the pooled GFC fractions for 1661. FIG. 2C provides a summary of the biophysical characteristics of variants 875, 1661, 1653, 1666, 10149, and 12043.

FIG. 3 depicts the ability of variants 875 and 1661 to bridge B and T cells with the formation of pseudopodia. The table on the left provides a summary of B:T cell bridging analysis for these variants as measured by FACS bridging analysis and bridging microscopy; the image on the right shows the formation of pseudopodia for variant 875, as measured by bridging microscopy.

FIG. 4 depicts off-target cytotoxicity of variant 875 on non-CD19 expressing K562 cells in IL2-activated purified CD8+ T cells at 300 nM (average 4 donors).

FIG. 5 depicts the reduced or ablated ability of v1661 to mediate ADCC or CDC. FIG. 5A depicts the ability of variant 1661 to mediate ADCC of Raji cells compared to Rituximab control. FIG. 5B depicts the ability of variant 1661 to mediate CDC of Raji cells vs. Rituximab control.

FIG. 6 depicts the ability of selected variants to mediate autologous B cell depletion in a whole blood assay. The presence of CD20+B cells was determined following 48 h incubation in IL2 activated human whole blood (Average of 2 donors, n=4).

FIG. 7 depicts dose-dependent autologous B-cell depletion by v1661 in a concentration-dependent manner (EC50<0.01 nM) in IL-2 activated human whole blood after 48 h at an E:T ratio of 10:1.

FIG. 8 depicts a comparison of the ability of variants 1661 and 10149 to deplete autologous B cells in whole blood, in a dose-dependent manner, under resting conditions.

FIG. 9 depicts autologous B cell depletion by v1661 in primary patient human whole blood. FIG. 9A shows the effect of v1661 in blood from an MCL patient. FIG. 9B shows the effect of v1661 in blood from two CLL patients. The number of malignant B cells remaining are represented as a percentage of CD20+/CD5+ B cell normalization to media control.

FIG. 10 depicts the ability of v875, 1380 and controls to stimulate T cell proliferation in human PBMC (4 day incubation, average of 4 donors).

FIG. 11 depicts target B cell dependent T cell proliferation in human PBMC, variants at 100 nM (4 day incubation, average of 4 donors).

FIG. 12 depicts the ability of selected variants to bind to the human G2 ALL tumor cell line.

FIG. 13 depicts the efficacy of variant 875 compared to controls in an in vivo mouse leukemia model. FIG. 13A shows the amount of bioluminescence in the whole body in the prone position; FIG. 13B shows the amount of bioluminescence in the whole body in the supine position; FIG. 13C shows the amount of bioluminescence in the isolated spleen at Day 18.

FIG. 14 depicts the efficacy of variant 1661 (an FcγR knockout variant) compared to controls in an in vivo mouse leukemia model. FIG. 14A shows the amount of bioluminescence in the whole body in the prone position; FIG. 14B shows the amount of bioluminescence in the whole body in the supine position; FIG. 14C is an image of whole body bioluminescence; and FIG. 141) shows the amount of bioluminescence detected in the isolated spleen at Day 18.

FIG. 15 depicts the analysis of the serum concentration of bi-specific anti-CD3-CD19 variants at 24 h following 3 mg/kg IV injection in an in vivo mouse leukemia model.

FIG. 16 depicts humanized CD19 VL and VH sequences based on the mouse HD37 VL and VH sequences. Three humanized VL sequences have been provided: hVL2, hVL2 (D-E), and hVL2 (D-S). hVL2 (D-E) contains a D to E substitution in CDR L1, while hVL2 (D-S) contains a D to S substitution in CDR L1. Two humanized VH sequences have been provided: hVH2, and hVH3. The CDR sequences are identified by boxes. The CDRs identified in this figure are exemplary only. As is known in the art, the identification of CDRs may vary depending on the method used to identify them. Alternate CDR definitions for the anti-CD19 VL and VH sequences are shown in Table S1. Modifications to humanize these sequences with respect to the wild-type mouse HD37 antibody sequence are denoted by underlining.

FIG. 17 depicts a table showing the number according to Kabat for the anti-CD19 VH and VL sequences, based on the anti-CD19 HD37 antibody.

### DETAILED DESCRIPTION OF THE INVENTION

Described herein are bispecific antigen-binding constructs (e.g. antibodies) that bind to CD3 and CD19 (CD3-CD19 antigen-binding constructs). These CD3-CD19 antigen-binding constructs comprise an antigen-binding domain that monovalently binds to the CD3 epsilon subunit, an antigen-binding domain that monovalently binds to CD19, and a heterodimeric Fc region. Both antigen-binding domains are in the scFv format, and have been engineered in order to improve manufacturability, as assessed by yield, purity and stability of the antibodies when expressed and purified using standard antibody manufacturing protocols.

For successful development of a therapeutic antibody or antigen-binding construct as described herein, the construct must be produced with sufficiently high titer and the expressed product must be substantially pure. The post purification titer of an antibody or scFv construct is determined at least in part by protein folding and processing within the expression host cell, and the stability of the construct during the purification process, to minimize the formation of aggregates and protein degradation.

As described elsewhere herein, the antigen-binding constructs incorporate several modifications to optimize the specific aspects of folding, expression and stability. These modifications include, for example optimization of the linker and VHVL orientation to improve protein folding and expression; disulphide engineering of the VHVL to reduce the formation of misfolded aggregates during expression and purification; and CDR grafting to a known stable framework to optimize folding, expression, but also stability during the purification process.

The bispecific antigen-binding constructs described herein are able to bridge CD3-expressing T cells with CD19-expressing B cells, with the formation of immunological synapses. These antigen-binding constructs are able to mediate T cell directed B cell depletion as measured by in vitro and ex vivo assays, and as assessed in an in vivo model of disease. As such, the bispecific antigen-binding constructs described herein are useful in the treatment of diseases such as lymphomas and leukemias, in which it is advantageous to decrease the number of circulating B cells in a patient.

Also described herein are humanized anti-CD19 VL and VH (anti-CD19 huVLVH) sequences, based on the VL and VH sequences of the anti-CD19 HD37 antibody. These anti-CD19 huVLVH sequences can be used in the anti-CD19 antigen-binding domains of the bispecific CD3-CD19 antigen-binding constructs described herein.

### Bi-Specific Antigen-Binding Constructs

Provided herein are bi-specific antigen-binding constructs, e.g., antibodies, that bind CD3 and CD19. The bi-specific antigen-binding construct includes two antigen-binding polypeptide constructs, e.g., antigen binding domains, each an scFv and specifically binding either CD3 or CD19. In some embodiments, the antigen-binding construct is derived from known antibodies or antigen-binding constructs. As described in more detail below, the antigen-binding polypeptide constructs are scFv (single chain Fv) and includes an Fc.

The term “antigen-binding construct” refers to any agent, e.g., polypeptide or polypeptide complex capable of binding to an antigen. In some aspects an antigen-binding construct is a polypeptide that specifically binds to an antigen of interest. An antigen-binding construct can be a monomer, dimer, multimer, a protein, a peptide, or a protein or peptide complex; an antibody, an antibody fragment, or an antigen-binding fragment thereof; an scFv and the like. An antigen-binding construct can be a polypeptide construct that is monospecific, bi-specific, or multispecific. In some aspects, an antigen-binding construct can include, e.g., one or more antigen-binding components (e.g., Fabs or scFvs) linked to one or more Fc. Further examples of antigen-binding constructs are described below and provided in the Examples.

The term “bi-specific” is intended to include any agent, e.g., an antigen-binding construct, which has two antigen-binding moieties (e.g. antigen-binding polypeptide constructs), each with a unique binding specificity. For example, a first antigen-binding moiety binds to an epitope on a first antigen, and a second antigen-binding moiety binds to an epitope on a second antigen, where the first antigen is different from the second antigen.

For example, in some embodiments a bi-specific agent may bind to, or interact with, (a) a cell surface target molecule and (b) an Fc receptor on the surface of an effector cell. In another embodiment, the agent may bind to, or interact with (a) a first cell surface target molecule and (b) a second cell surface target molecule that is different from the first cells surface target molecule. In another embodiment, the agent may bind to and bridge two cells, i.e. interact with (a) a first cell surface target molecule on a first call and (b) a second cell surface target molecule on a second cell that is different from the first cell's surface target molecule.

In some embodiments, the bi-specific antigen-binding construct bridges CD3-expressing T cells with CD19-expressing B cells, with the formation of immunological synapses and/or mediation of T cell directed B cell depletion.

A monospecific antigen-binding construct refers to an antigen-binding construct with a single binding specificity. In other words, both antigen-binding moieties bind to the same epitope on the same antigen. Examples of monospecific antigen-binding constructs include the anti-CD19 antibody HD37 and the anti-CD3 antibody OKT3 for example.

An antigen-binding construct can be an antibody or antigen-binding portion thereof. As used herein, an “antibody” or “immunoglobulin” refers to a polypeptide substantially encoded by an immunoglobulin gene or immunoglobulin genes, or fragments thereof, which specifically bind and recognize an analyte (e.g., antigen). The recognized immunoglobulin genes include the kappa, lambda, alpha, gamma, delta, epsilon and mu constant region genes, as well as the myriad immunoglobulin variable region genes. Light chains are classified as either kappa or lambda. The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

An exemplary immunoglobulin (antibody) structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains respectively.

The IgG₁ heavy chain comprised of the VH, CH1, CH2 and CH3 domains respectively from the N to C-terminus. The light chain is comprised of the VL and CL domains from N to C terminus. The IgG₁ heavy chain comprises a hinge between the CH1 and CH2 domains.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The CDR regions of an antibody may be used to construct a binding protein, including without limitation, an antibody, a scFv, a diabody, and the like. In a certain embodiment, the antigen-binding constructs described herein will comprise at least one or all the CDR regions from an antibody. CDR sequences may be used on an antibody backbone, or fragment thereof, and likewise may include humanized antibodies, or antibodies containing humanized sequences. Methods of identifying CDR portions of an antibody are well known in the art. See, Shirai, H., Kidera, A., and Nakamura, H., H3-rules: Identification of CDR-H3 structures in antibodies, FEBS Lett., 455(1):188-197, 1999; and Almagro J C, Fransson, J. Front Biosci. 13:1619-33 (2008).

### Antigen-Binding Polypeptide Construct—Format

The bi-specific antigen-binding construct comprises two antigen-binding polypeptide constructs, e.g., antigen binding domains. The format of the antigen-binding polypeptide construct determines the functional characteristics of the bi-specific antigen-binding construct. In one embodiment, the bi-specific antigen-binding construct has an scFv-scFv format, i.e. both antigen-binding polypeptide constructs are scFvs.

The format “Single-chain Fv” or “scFv” includes the VH and VL domains of an antibody, wherein these domains are present in a single polypeptide chain. In some embodiments, the scFv polypeptide further comprises a polypeptide linker between the VH and VL domains. For a review of scFv see Pluckthun in The Pharmacology of Monoclonal Antibodies, vol. 113, Rosenburg and Moore eds., Springer-Verlag, New York, pp. 269-315 (1994).

Other antigen-binding polypeptide construct formats include a Fab fragment or sdAb.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

The “Single domain antibodies” or “sdAb” format is an individual immunoglobulin domain. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77(1): 13-22).

Format scFv

The antigen-binding constructs described herein are bi-specific, e.g., they comprise two antigen-binding polypeptide constructs each capable of specific binding to a distinct antigen. Each antigen-binding polypeptide construct is in an scFv format. (i.e., antigen-binding domains composed of a heavy chain variable domain and a light chain variable domain, connected with a polypeptide linker). In one embodiment said scFv are human. In another embodiment said scFv molecules are humanized. The scFvs are optimized for protein expression and yield by the modifications described below.

The scFv can be optimized by changing the order of the variable domains VL and VH in the scFv. In some embodiments of an scFv in a antigen-binding construct described herein, the C-terminus of the light chain variable region may be connected to the N-terminus of the heavy chain variable region, or the C-terminus of the heavy chain variable region may be connected to the N-terminus of the light chain variable region.

The variable regions may be connected via a linker peptide, or scFv linker, that allows the formation of a functional antigen-binding moiety. The scFv can be optimized for protein expression and yield by changing composition and/or length of the scFv linker polypeptide. Typical peptide linkers comprise about 2-20 amino acids, and are described herein or known in the art. Suitable, non-immunogenic linker peptides include, for example, (G₄S)ₙ, (SG₄)ₙ, (G₄S)ₙ, G₄(SG₄)ₙ or G₂(SG₂)ₙ linker peptides, wherein n is generally a number between 1 and 10, typically between 2 and 4.

In some embodiments, the scFv linker is selected from Table below:

TABLE B
scFv linker polypeptide sequences

| | SEQ ID NO: |
|---|---|
| CD19 | |
| GGGGSGGGGSGGGGS | 342 |
| CD3 | |
| GGGGSGGGGSGGGGS | 343 |
| SSTGGGGSGGGGSGGGGSDI | 344 |
| VEGGSGGSGGSGGSGGVD | 345 |
| Generic linkers: | |
| GGGGSGGGGSGGGGS | 346 |
| GGGGSGGGGSGGGGSGGGGS | 347 |
| GSTSGGGSGGGSGGGGSS | 348 |
| GSTSGSGKPGSGEGSTKG | 349 |

The scFv molecule may be optimized for protein expression and yield by including stabilizing disulfide bridges between the heavy and light chain variable domains, for example as described in Reiter et al. (Nat Biotechnol 14, 1239-1245 (1996)). Hence, in one embodiment the T cell activating bi-specific antigen-binding molecule of the invention comprises a scFv molecule wherein an amino acid in the heavy chain variable domain and an amino acid in the light chain variable domain have been replaced by cysteine so that a disulfide bridge can be formed between the heavy and light chain variable domain. In a specific embodiment the amino acid at position 44 of the light chain variable domain and the amino acid at position 100 of the heavy chain variable domain have been replaced by cysteine (Kabat numbering).

As is known in the art, scFvs can also be stabilized by mutation of CDR sequences, as described in [Miller et al., Protein Eng Des Sel. 2010 July; 23(7):549-57; Igawa et al., MAbs. 2011 May-June; 3(3):243-5; Perchiacca & Tessier, Annu Rev Chem Biomol Eng. 2012; 3:263-86.].

### Humanized CD19 VH and VL

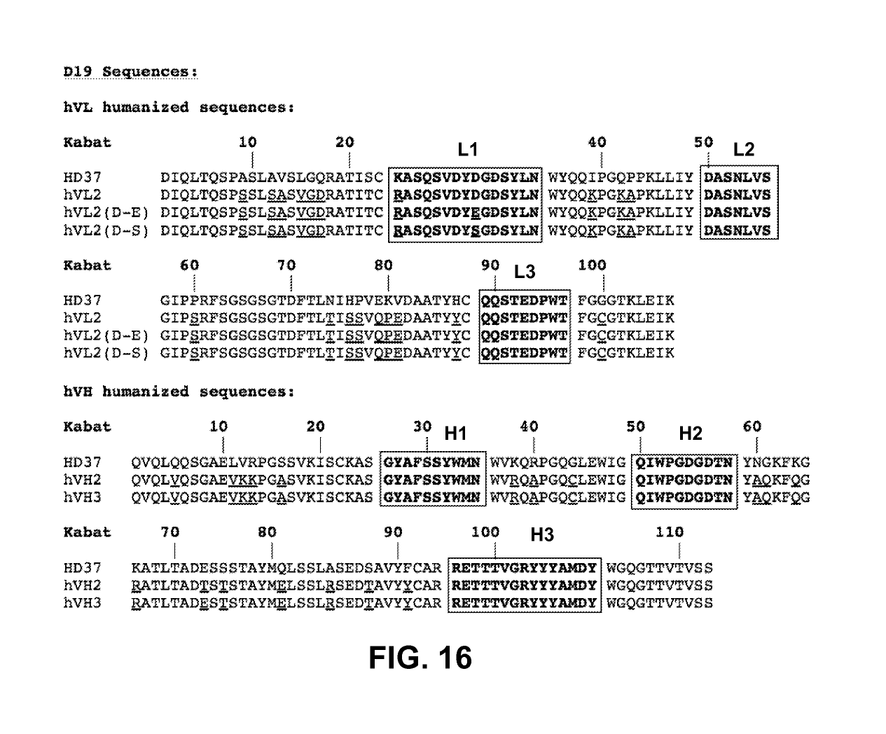

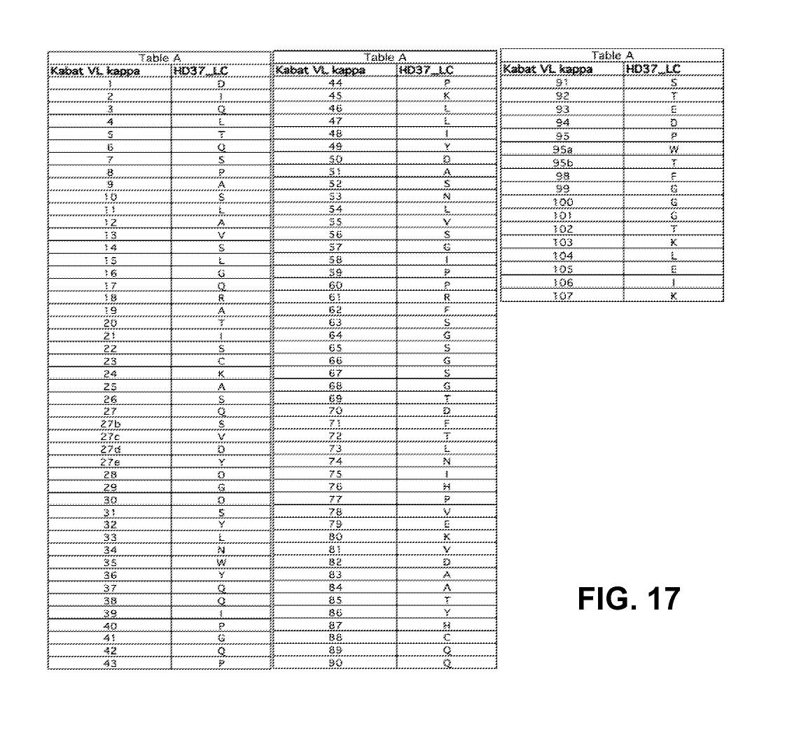

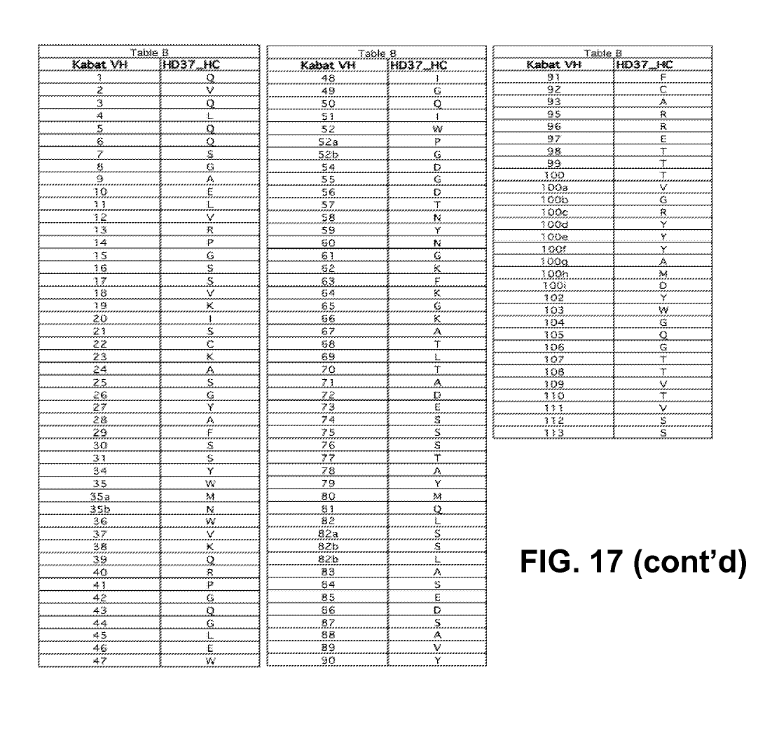

In some embodiments, and in order to further stabilize the antigen-binding constructs described herein, the wild-type sequences of the HD37 anti-CD19 antibody can be modified to generate humanized VH and VL polypeptide sequences. Modifications to both the framework regions and CDRs can be made in order to obtain VH and VL polypeptide sequences to be used in the CD19-binding scFv of the antigen-binding constructs. In some embodiments, the modifications are those depicted in FIG. 16, and the sequences of the modified CDRs, VH and VL polypeptide sequences are those shown in Tables S2 and S3

One or more of the above noted modifications to the format and sequence of the scFv may be applied to scFvs of the antigen-binding constructs.

### Antigen-Binding Polypeptide Construct—Antigens

The antigen-binding constructs described herein specifically bind a CD3 antigen and a CD19 antigen.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen-binding moiety binds, forming an antigen-binding moiety-antigen complex. An epitope typically includes at least 3, and more usually, at least 5 or 8-10 amino acids in a unique spatial conformation. The epitope may comprise amino acid residues directly involved in the binding and other amino acid residues, which are not directly involved in the binding, such as amino acid residues which are effectively blocked by the specifically antigen binding peptide; in other words, the amino acid residue is within the footprint of the specifically antigen binding peptide. Antibodies that recognize the same epitope can be verified in a simple immunoassay showing the ability of one antibody to block the binding of another antibody to a target antigen.

“Specifically binds”, “specific binding” or “selective binding” means that the binding is selective for the antigen and can be discriminated from unwanted or non-specific interactions. The ability of an antigen-binding construct to bind to a specific antigenic determinant can be measured either through an enzyme-linked immunosorbent assay (ELISA) or other techniques familiar to one of skill in the art, e.g. surface plasmon resonance (SPR) technique (analyzed on a BIAcore instrument) (Liljceblad et al, Glyco J 17, 323-329 (2000)), and traditional binding assays (Heeley, Endocr Res 28, 217-229 (2002)). In one embodiment, the extent of binding of an antigen-binding moiety to an unrelated protein is less than about 10% of the binding of the antigen-binding construct to the antigen as measured, e.g., by SPR.

In certain embodiments, an antigen-binding construct that binds to the antigen, or an antigen-binding molecule comprising that antigen-binding moiety, has a dissociation constant (K_{D}) of <1 μM, <100 nM, <10 nM, <1 nM, <0.1 nM, <0.01 nM, or <0.001 nM (e.g. 10⁻⁸ M or less, e.g. from 10⁻⁸ M to 10⁻¹³ M, e.g., from 10⁻⁹ M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen-binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR), or whole cell binding assays with cells that express the antigen of interest.

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

An “activating T cell antigen” as used herein refers to an antigenic determinant expressed on the surface of a T lymphocyte, particularly a cytotoxic T lymphocyte, which is capable of inducing T cell activation upon interaction with an antigen-binding molecule. Specifically, interaction of an antigen-binding molecule with an activating T cell antigen may induce T cell activation by triggering the signaling cascade of the T cell receptor complex. In a particular embodiment the activating T cell antigen is CD3.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating bi-specific antigen-binding molecules of the invention are capable of inducing T cell activation. Suitable assays to measure T cell activation are known in the art described herein.

A “target cell antigen” as used herein refers to an antigenic determinant presented on the surface of a target cell, for example a B cell in a tumor such as a cancer cell or a cell of the tumor stroma. As used herein, the terms “first” and “second” with respect to antigen-binding moieties etc., are used for convenience of distinguishing when there is more than one of each type of moiety. Use of these terms is not intended to confer a specific order or orientation of the T cell activating bi-specific antigen-binding molecule unless explicitly so stated.

The term “cross-species binding” or “interspecies binding” as used herein means binding of a binding domain described herein to the same target molecule in humans and other organisms for instance, but not restricted to non-chimpanzee primates. Thus, “cross-species binding” or “interspecies binding” is to be understood as an interspecies reactivity to the same molecule “X” (i.e. the homolog) expressed in different species, but not to a molecule other than “X”. Cross-species specificity of a monoclonal antibody recognizing e.g. human CD3 epsilon, to a non-chimpanzee primate CD3 epsilon, e.g. macaque CD3 epsilon, can be determined, for instance, by FACS analysis. The FACS analysis is carried out in a way that the respective monoclonal antibody is tested for binding to human and non-chimpanzee primate cells, e.g. macaque cells, expressing said human and non-chimpanzee primate CD3 epsilon antigens, respectively. An appropriate assay is shown in the following examples. The above-mentioned subject matter applies mutatis mutandis for the CD19. The FACS analysis is carried out in a way that the respective monoclonal antibody is tested for binding to human and non-chimpanzee primate cells, e.g. macaque cells, expressing said human and non-chimpanzee primate CD3 or CD19 antigens.

### CD3

The antigen-binding constructs described herein specifically bind a CD3 antigen.

“CD3” or “CD3 complex” as described herein is a complex of at least five membrane-bound polypeptides in mature T-lymphocytes that are non-covalently associated with one another and with the T-cell receptor. The CD3 complex includes the gamma, delta, epsilon, and zeta chains (also referred to as subunits). Non-human monoclonal antibodies have been developed against some of these chains, as exemplified by the murine antibodies OKT3, SP34, UCHT1 or 64.1. (See e.g., June, et al., J. Immunol. 136:3945-3952 (1986); Yang, et al., J. Immunol. 137:1097-1100 (1986); and Hayward, et al., Immunol. 64:87-92 (1988)). Clustering of CD3 on T cells, e.g., by immobilized anti-CD3-antibodies, leads to T cell activation similar to the engagement of the T cell receptor but independent from its clone typical specificity. Most anti-CD3-antibodies recognize the CD3ε-chain.

In some embodiments, the anti-CD3 scFv is an scFV of a known anti-CD3 antibody, or is derived from, e.g., is a modified version of the scFv of a known anti-CD3 antibody. Antibodies directed against human CD3 which provide for variable regions (VH and VL) to be employed in the bi-specific antigen-binding construct described herein are known in the art and include OKT3 (ORTHOCLONE-OKT3™ (muromonab-CD3). Additional anti-CD3 antibodies include “OKT3 blocking antibodies” that block by 50% or greater the binding of OKT3 to the epsilon subunit of the CD3 antigen. Examples include but are not limited to Teplizumab™ (MGA031, Eli Lilly); UCHT1 (Pollard et al. 1987 J Histochem Cytochem. 35(11):1329-38); N10401 (WO2007/033230); and visilizumab (US25834597).

In one embodiment, the bi-specific antigen-binding construct comprises a CD3 antigen-binding polypeptide construct which monovalently and specifically binds a CD3 antigen, where the CD3 antigen-binding polypeptide construct is derived from OKT3 (ORTHOCLONE-OKT3™ (muromonab-CD3). In one embodiment the bi-specific antigen-binding construct comprises a CD3 antigen-binding polypeptide construct which monovalently and specifically binds a CD3 antigen, the VH and VL regions of said CD3 antigen-binding polypeptide derived from the CD3 epsilon-specific antibody OKT3.

In some embodiments, the binding affinity of the first scFv for CD19 is between about 0.1 nM to about 5 nM or less than 5.0, 4.0, 3.0, 2.0, 1.0, 0.9, 0.09, 0.9, 0.7, 0.6, 0.5, 0.4, 0.3, or less than 0.2 nM.

The epitope on the CD3 epsilon subunit to which the OKT3 antibody binds is identified by analysis of the crystal structure of the OKT3 bound to CD3 epsilon (Kjer-Nielsen L. et al., (2004) Proc. Natl. Acad. Sci. USA 101: 7675-7680). The polypeptide sequence of CD3 epsilon is provided in the Table below.

TABLE F
CD3 Epsilon sequence

| | |
|---|---|
| Human T-cell | MQSGTHWRVLGLCLLSVGVWGQDGNEEMGGITQTPYK |
| surface | VSISGTTVILTCPQYPGSEILWQHNDKNIGGDEDDKN |
| glycoprotein | IGSDEDHLSLKEFSELEQSGYYVCYPRGSKPEDANFY |
| CD3 epsilon | LYLRARVCENCMEMDVMSVATIVIVDICITGGLLLLV |
| subunit, | YYWSKNRKAKAKPVTRGAGAGGRQRGQNKERPPPVPN |
| UniProt ID: | PDYEPIRKGQRDLYSGLNQRRI (SEQ ID NO: |
| P07766 (207 | 350) |
| amino acids) | |

Analysis of this structure indicates that the CDRs of the OKT3 antibody, with respect to the sequence in Table F, contact human CD3 epsilon at residues 56-57 (SE), 68-70 (GDE), and 101-107 (RGSKPED). The binding hotspots in these residues are underlined. These residues are considered to be the epitope to which OKT3 binds. Accordingly, the antigen-binding constructs described herein comprise an antigen-binding polypeptide construct that specifically binds to this epitope.

Provided herein are antigen-binding constructs comprising at least one CD3 binding polypeptide construct that binds to a CD3 complex on at least one CD3 expressing cell, where in the CD3 expressing cell is a T-cell. In certain embodiments, the CD3 expressing cell is a human cell. In some embodiments, the CD3 expressing cell is a non-human, mammalian cell. In some embodiments, the T cell is a cytotoxic T cell. In some embodiments the T cell is a CD4⁺ or a CD8⁺ T cell.

In certain embodiments of the antigen-binding constructs provided herein, the construct is capable of activating and redirecting cytotoxic activity of a T cell to a target cell such as a B cell. In a particular embodiment, said redirection is independent of MHC-mediated peptide antigen presentation by the target cell and and/or specificity of the T cell.

### CD19

The antigen-binding constructs described herein include an antigen-binding polypeptide construct that binds to a CD19 antigen (anti-CD19 scFv).

In some embodiments, the anti-CD19 scFv is an scFv of a known anti-CD19 antibody, or is derived from, e.g., is a modified version of the scFv of a known anti-CD19 antibody. Antibodies directed against CD19 which provide for variable regions (VH and VL) to be employed in the bi-specific antigen-binding construct described herein are known in the art and include HD37, provided by the HD37 hybridoma (Pezzutto (1997), J. Immunol. 138, 2793-9). Additional anti-CD19 antibodies include “HD37 blocking antibodies” that block by 50% or greater the binding of HD37 to the CD19 antigen. Examples include but are not limited to HD237 (IgG2b) (Fourth International Workshop on Human Leukocyte Differentiation Antigens, Vienna, Austria, 1989; and Pezzutto et al., J. Immunol., 138(9):2793-2799 (1987)); 4G7 (Meecker (1984) Hybridoma 3, 305-20); B4 (Freedman (1987) Blood 70, 418-27); B43 (Bejcek (1995) Cancer Res. 55, 2346-51) and Mor-208 (Hammer (2012) Mabs 4:5, 571-577).

In one embodiment said VH(CD19) and VL(CD19) regions (or parts, like CDRs, thereof) are derived from the anti-CD19 antibody HD37, provided by the HD37 hybridoma (Pezzutto (1997), J. Immunol. 138, 2793-9).

In some embodiments, the binding affinity of the second scFv for the epsilon subunit of CD3 is between about 1 nM to about 100 nM, or between about 20 nM to about 100 nM, or, e.g., greater than 2, 3, 4, 5, 6, 7, 8, 9, 10, 20, 30, 40, 50, 60, 70, 80, or greater than 90 nM.

In certain embodiments, the at least one antigen-binding polypeptide construct is scFv construct that binds CD19 on a B cell. In some embodiments said scFv construct is mammalian. In one embodiment said scFv construct is human. In another embodiment said scFv construct is humanized. In yet another embodiment said scFv construct comprises at least one of human heavy and light chain variable regions.

In certain embodiments, the antigen-binding polypeptide construct exhibits cross-species binding to a least one antigen expressed on the surface of a B cell. In some embodiments, the antigen-binding polypeptide construct of an antigen-binding construct described herein bind to at least one of mammalian CD19. In certain embodiments, the CD19 antigen-binding polypeptide construct binds a human CD19.

### Fc of Antigen-Binding Constructs.

The antigen-binding constructs described herein comprise an Fc, e.g., a dimeric Fc. The Fc is a heterodimeric Fc comprising first and second Fc polypeptides each comprising a modified CH3 sequence, wherein each modified CH3 sequence comprises asymmetric amino acid modifications that promote the formation of a heterodimeric Fc and the dimerized CH3 domains have a melting temperature (Tm) of about 68° C. or higher, and wherein the first Fc polypeptide is linked to the first antigen-binding polypeptide construct, with a first hinge linker, and the second Fc polypeptide is linked to the second antigen-binding polypeptide construct with a second hinge linker.

The term “Fc domain” or “Fc region” herein is used to define a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, Md., 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence.

An Fc domain comprises either a CH3 domain or a CH3 and a CH2 domain. The CH3 domain comprises two CH3 sequences, one from each of the two Fc polypeptides of the dimeric Fc. The CH2 domain comprises two CH2 sequences, one from each of the two Fc polypeptides of the dimeric Fc.

In some aspects, the Fc comprises at least one or two CH3 sequences. In some aspects, the Fc is coupled, with or without one or more linkers, to a first antigen-binding construct and/or a second antigen-binding construct. In some aspects, the Fc is a human Fc. In some aspects, the Fc is a human IgG or IgG1 Fc. In some aspects, the Fc is a heterodimeric Fc. In some aspects, the Fc comprises at least one or two CH2 sequences.

In some aspects, the Fc comprises one or more modifications in at least one of the CH3 sequences. In some aspects, the Fc comprises one or more modifications in at least one of the CH2 sequences. In some aspects, an Fc is a single polypeptide. In some aspects, an Fc is multiple peptides, e.g., two polypeptides.

In some aspects, the Fc is an Fc described in patent applications PCT/CA2011/001238, filed Nov. 4, 2011 or PCT/CA2012/050780, filed Nov. 2, 2012, the entire disclosure of each of which is hereby incorporated by reference in its entirety for all purposes.

Modified CH3 Domains

In some aspects, the antigen-binding construct described herein comprises a heterodimeric Fc comprising a modified CH3 domain that has been asymmetrically modified. The heterodimeric Fc can comprise two heavy chain constant domain polypeptides: a first Fc polypeptide and a second Fc polypeptide, which can be used interchangeably provided that Fc comprises one first Fc polypeptide and one second Fc polypeptide. Generally, the first Fc polypeptide comprises a first CH3 sequence and the second Fc polypeptide comprises a second CH3 sequence.

Two CH3 sequences that comprise one or more amino acid modifications introduced in an asymmetric fashion generally results in a heterodimeric Fc, rather than a homodimer, when the two CH3 sequences dimerize. As used herein, “asymmetric amino acid modifications” refers to any modification where an amino acid at a specific position on a first CH3 sequence is different from the amino acid on a second CH3 sequence at the same position, and the first and second CH3 sequence preferentially pair to form a heterodimer, rather than a homodimer. This heterodimerization can be a result of modification of only one of the two amino acids at the same respective amino acid position on each sequence; or modification of both amino acids on each sequence at the same respective position on each of the first and second CH3 sequences. The first and second CH3 sequence of a heterodimeric Fc can comprise one or more than one asymmetric amino acid modification.

Table A provides the amino acid sequence of the human IgG1 Fc sequence, corresponding to amino acids 231 to 447 of the full-length human IgG1 heavy chain. Amino acids 231-238 are also referred to as the lower hinge. The CH3 sequence comprises amino acid 341-447 of the full-length human IgG1 heavy chain.

Typically an Fc can include two contiguous heavy chain sequences (A and B) that are capable of dimerizing. With respect to the antigen binding constructs described herein, in some embodiments the first scFv is linked to chain A of the heterodimeric Fc and the second scFv is linked to chain B of the heterodimeric Fc. In some embodiments the second scFv is linked to chain A of the heterodimeric Fc and the first scFv is linked to chain B of the heterodimeric Fc.

In some aspects, one or both sequences of an Fc include one or more mutations or modifications at the following locations: L351, F405, Y407, T366, K392, T394, T350, S400, and/or N390, using EU numbering. In some aspects, an Fc includes a mutant sequence shown in Table X. In some aspects, an Fc includes the mutations of Variant 1 A-B. In some aspects, an Fc includes the mutations of Variant 2 A-B. In some aspects, an Fc includes the mutations of Variant 3 A-B. In some aspects, an Fc includes the mutations of Variant 4 A-B. In some aspects, an Fc includes the mutations of Variant 5 A-B.

TABLE A
IgG1 Fc sequence and variants

| Human IgG1 Fc | APELLGGPSVFLFPPKPKDTLMISRTPEVTCVVV |
|---|---|
| sequence 231-447 | DVSHEDPEVKFNWYVDGVEVHNAKTKPREEQYNS |
| (EU-numbering) | TYRVVSVLTVLHQDWLNGKEYKCKVSNKALPAPI |
| | EKTISKAKGQPREPQVYTLPPSRDELTKNQVS |
| | LTCLVKGFYPSDIAVEWESNGQPENNYKTTPPVL |
| | DSDGSFFLYSKLTVDKSRWQQGNVFSCSVMH |
| | EALHNHYTQKSLSLSPGK (SEQ ID NO: |
| | 361) |

| Variant IgG1 | | |
|---|---|---|
| Fc sequence | | |
| (231-447) | Chain | Mutations |
| 1 | A | L351Y_F405A_Y407V |
| 1 | B | T366L_K392M_T394W |
| 2 | A | L351Y_F405A_Y407V |
| 2 | B | T366L_K392L_T394W |
| 3 | A | T350V_L351Y_F405A_Y407V |
| 3 | B | T350V_T366L_K392L_T394W |
| 4 | A | T350V_L351Y_F405A_Y407V |
| 4 | B | T350V_T366L_K392M_T394W |
| 5 | A | T350V_L351Y_S400E_F405A_Y407V |
| 5 | B | T350V_T366L_N390R_K392M_T394W |

The first and second CH3 sequences can comprise amino acid mutations as described herein, with reference to amino acids 231 to 447 of the full-length human IgG1 heavy chain. In one embodiment, the heterodimeric Fc comprises a modified CH3 domain with a first CH3 sequence having amino acid modifications at positions F405 and Y407, and a second CH3 sequence having amino acid modifications at position T394. In one embodiment, the heterodimeric Fc comprises a modified CH3 domain with a first CH3 sequence having one or more amino acid modifications selected from L351Y, F405A, and Y407V, and the second CH3 sequence having one or more amino acid modifications selected from T366L, T366I, K392L, K392M, and T394W.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, and one of the first or second CH3 sequences further comprising amino acid modifications at position Q347, and the other CH3 sequence further comprising amino acid modification at position K360. In another embodiment, a heterodimeric Fc comprises a modified CH3 domain with a first CH3 sequence having amino acid modifications at positions L351. F405 and Y407, and a second CH3 sequence having amino acid modifications at position T366. K392, and T394, one of the first or second CH3 sequences further comprising amino acid modifications at position Q347, and the other CH3 sequence further comprising amino acid modification at position K360, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394 and one of said first and second CH3 sequences further comprising amino acid modification of D399R or D399K and the other CH3 sequence comprising one or more of T411E, T411D, K409E, K409D, K392E and K392D. In another embodiment, a heterodimeric Fc comprises a modified CH3 domain with a first CH3 sequence having amino acid modifications at positions L351. F405 and Y407, and a second CH3 sequence having amino acid modifications at positions T366, K392, and T394, one of said first and second CH3 sequences further comprises amino acid modification of D399R or D399K and the other CH3 sequence comprising one or more of T411E, T411 D, K409E, K409D, K392E and K392D, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, wherein one or both of said CH3 sequences further comprise the amino acid modification of T350V.

In one embodiment, a heterodimeric Fc comprises a modified CH3 domain comprising the following amino acid modifications, where “A” represents the amino acid modifications to the first CH3 sequence, and “B” represents the amino acid modifications to the second CH3 sequence: A: L351Y_F405A_Y407V, B: T366L_K392M_T394W, A: L351Y_F405A_Y407V, B: T366L_K392L_T394W, A: T350V_L351Y_F405A_Y407V, B: T350V_T366L_K392L_T394W, A: T350V_L351Y_F405A_Y407V, B: T350V_T366L_K392M_T394W, A: T350V_L351Y_S400E_F405A_Y407V, and/or B: T350V_T366L_N390R_K392M_T394W.

The one or more asymmetric amino acid modifications can promote the formation of a heterodimeric Fc in which the heterodimeric CH3 domain has a stability that is comparable to a wild-type homodimeric CH3 domain. In an embodiment, the one or more asymmetric amino acid modifications promote the formation of a heterodimeric Fc domain in which the heterodimeric Fc domain has a stability that is comparable to a wild-type homodimeric Fc domain. In an embodiment, the one or more asymmetric amino acid modifications promote the formation of a heterodimeric Fc domain in which the heterodimeric Fc domain has a stability observed via the melting temperature (Tm) in a differential scanning calorimetry study, and where the melting temperature is within 4° C. of that observed for the corresponding symmetric wild-type homodimeric Fc domain. In some aspects, the Fc comprises one or more modifications in at least one of the C_{H3} sequences that promote the formation of a heterodimeric Fc with stability comparable to a wild-type homodimeric Fc.

In one embodiment, the stability of the CH3 domain can be assessed by measuring the melting temperature of the CH3 domain, for example by differential scanning calorimetry (DSC). Thus, in a further embodiment, the CH3 domain has a melting temperature of about 68° C. or higher. In another embodiment, the CH3 domain has a melting temperature of about 70° C. or higher. In another embodiment, the CH3 domain has a melting temperature of about 72° C. or higher. In another embodiment, the CH3 domain has a melting temperature of about 73° C. or higher. In another embodiment, the CH3 domain has a melting temperature of about 75° C. or higher. In another embodiment, the CH3 domain has a melting temperature of about 78° C. or higher. In some aspects, the dimerized CH3 sequences have a melting temperature (Tm) of about 68, 69, 70, 71, 72, 73, 74, 75, 76, 77, 77.5, 78, 79, 80, 81, 82, 83, 84, or 85° C. or higher.

In some embodiments, a heterodimeric Fc comprising modified CH3 sequences can be formed with a purity of at least about 75% as compared to homodimeric Fc in the expressed product. In another embodiment, the heterodimeric Fc is formed with a purity greater than about 80%. In another embodiment, the heterodimeric Fc is formed with a purity greater than about 85%. In another embodiment, the heterodimeric Fc is formed with a purity greater than about 90%. In another embodiment, the heterodimeric Fc is formed with a purity greater than about 95%. In another embodiment, the heterodimeric Fc is formed with a purity greater than about 97%. In some aspects, the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when expressed. In some aspects, the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when expressed via a single cell.

Additional methods for modifying monomeric Fc polypeptides to promote heterodimeric Fc formation are described in International Patent Publication No. WO 96/027011 (knobs into holes), in Gunasekaran et al. (Gunasekaran K. et al. (2010) J Biol Chem. 285, 19637-46, electrostatic design to achieve selective heterodimerization), in Davis et al. (Davis, J H. et al. (2010) Prot Eng Des Sel; 23(4): 195-202, strand exchange engineered domain (SEED) technology), and in Labrijn et al [Efficient generation of stable bi-specific IgG1 by controlled Fab-arm exchange. Labrijn A F, Meesters J I, de Goeij B E, van den Bremer E T, Neijssen J, van Kampen M D, Strumane K, Verploegen S, Kundu A, Gramer M J, van Berkel P H, van de Winkel J G, Schuurman J, Parren P W. Proc Natl Acad Sci USA. 2013 Mar. 26; 110(13):5145-50.

CH2 Domains

As indicated above, in some embodiments, the Fc of the antigen-binding construct comprises a CH2 domain in addition to a CH3 domain. As an example, the amino acid sequence of the CH2 domain of an IgG1 Fc is identified as amino acids 239-340 of the sequence shown in Table A. The CH2 domain of the Fc binds to Fc receptors and complement and is thus involved in mediating effector cell functions.

The terms “Fc receptor” and “FcR” are used to describe a receptor that binds to the Fc region of an antibody, and includes Fc gamma receptors (FcγRs) and the neonatal receptor FcRn.

Generally, an FcγR is one which binds an IgG antibody (a gamma receptor) and includes receptors of the FcγRI, FcγRII, and FcγRIII subclasses in humans, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcγRs, including those to be identified in the future, are encompassed by the term “FcR” herein. An FcγR are also found in other organisms, including but not limited to mice, rats, rabbits, and monkeys. Mouse FcγRs include but are not limited to FcγRI (CD64), FcγRII (CD32), FcγRIII (CD 16), and FcγRIII-2 (CD 16-2). FcγRs are expressed by effector cells such as NK cells or B cells.

Complement activation requires binding of the complement protein C1q to antigen-antibody complexes. Residues in the CH2 domain of the Fc are involved in the interaction between C1q and the Fc.

The antigen-binding constructs described herein are able to bind FcRn. As is known in the art, binding to FcRn recycles endocytosed antibody from the endosome back to the bloodstream (Raghavan et al., 1996, Annu Rev Cell Dev Biol 12:181-220; Ghetie et al., 2000, Annu Rev Immunol 18:739-766). This process, coupled with preclusion of kidney filtration due to the large size of the full-length molecule, results in favorable antibody serum half-lives ranging from one to three weeks. Binding of Fc to FcRn also plays a key role in antibody transport. FcRn is responsible for the transfer of maternal IgGs to the fetus (Guyer et al., J. Immunol. 117:587 (1976); and Kim et al., J. Immunol. 24:249 (1994)). Binding of the FcRn to IgG involves residues in the CH2 and CH3 domains of the Fc.

Modifications in the CH2 domain can affect the binding of FcRs to the Fc. As indicated above, the CH2 domain of the Fc comprises two CH2 sequences, one on each of the two Fc polypeptides of the dimeric Fc. Typically, the modifications to the CH2 domain are symmetric and are thus the same on both CH2 sequences of the Fc polypeptides. However, asymmetric mutations are also possible in the presence of mutations on the CH3 domain that enhance heterodimerization. In one embodiment, the CH2 domain comprises modifications to reduce FcγR or C1q binding and/or effector function.

### Modifications to Reduce Effector Function:

Fc modifications reducing FcγR and/or complement binding and/or effector function are known in the art. Recent publications describe strategies that have been used to engineer antibodies with reduced or silenced effector activity (see Strohl, W R (2009), Curr Opin Biotech 20:685-691, and Strohl, W R and Strohl L M. “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, US Patent Publication No. 2011/0212087 (Strohl), International Patent Publication No. WO 2006/105338 (Xencor), US Patent Publication No. 2012/0225058 (Xencor), US Patent Publication No. 2012/0251531 (Genentech), and Strop et al ((2012) J. Mol. Biol. 420: 204-219) describe specific modifications to reduce FcγR or complement binding to the Fc.

Specific, non-limiting examples of known symmetric amino acid modifications to reduce FcγR or complement binding to the Fc include those identified in the following table:

TABLE C
modifications to reduce FcγR or complement binding to the Fc

| Company | Mutations |
|---|---|
| GSK | N297A |
| Ortho Biotech | L234A/L235A |
| Protein Design labs | IGG2 V234A/G237A |
| Wellcome Labs | IGG4 L235A/G237A/E318A |
| GSK | IGG4 S228P/L236E |
| Alexion | IGG2/IgG4 combination |
| Merck | IGG2 H268Q/V309L/A330S/A331S |
| Bristol-Myers | C220S/C226S/C229S/P238S |
| Seattle Genetics | C226S/C229S/E3233P/L235V/L235A |
| Amgen | E. coli production, non glycosylated |
| Medimune | L234F/L235E/P331S |
| Trubion | Hinge mutant, possibly C226S/P230S |

In one embodiment, the Fc comprises at least one amino acid modification identified in the above table. In another embodiment the Fc comprises amino acid modification of at least one of L234, L235, or D265. In another embodiment, the Fc comprises amino acid modification at L234, L235 and D265. In another embodiment, the Fc comprises the amino acid modifications L234A, L235A and D265S.

In some embodiments the Fc comprises one or more asymmetric amino acid modifications in the lower hinge region of the Fc as described in International Patent Application No. PCT/CA2014/050507. Examples of such asymmetric amino acid modifications that reduce FcγR binding are shown in Table D:

TABLE D
Asymmetric mutations that reduce FcγR binding

| Chain A | Chain B |
|---|---|
| L234D/L235E | L234K/L235K |
| E233A/L234D/L235E | E233A/L234R/L235R |
| L234D/L235E | E233K/L234R/L235R |
| E233A/L234K/L235A | E233K/L234A/L235K |

### Hinge Linkers

In the antigen-binding constructs described herein, the first Fc polypeptide is linked to the first antigen-binding polypeptide construct with a first hinge linker, and the second Fc polypeptide is linked to the second antigen-binding polypeptide construct with a second hinge linker. Examples of hinge linker sequences are well-known to one of skill in the art and can be used in the antigen-binding constructs described herein. Alternatively, modified versions of known hinge linkers can be used.

The hinge linker polypeptides are selected such that they maintain or optimize the functional activity of the antigen-binding construct. Suitable linker polypeptides include IgG hinge regions such as, for example those from IgG₁, IgG₂, or IgG₄, including the upper hinge sequences and core hinge sequences. The amino acid residues corresponding to the upper and core hinge sequences vary depending on the IgG type, as is known in the art and one of skill in the art would readily be able to identify such sequences for a given IgG type. Modified versions of these exemplary linkers can also be used. For example, modifications to improve the stability of the IgG4 hinge are known in the art (see for example, Labrijn et al. (2009) Nature Biotechnology 27, 767-771). Examples of hinge linker sequences are found in the following Table.

TABLE E
Hinge linker polypeptide sequences
(SEQ ID NOS: 351-360)

| SEQ ID NO: | | |
|---|---|---|
| 351 | IgG1 | EPKSCDKTHTCPPCP |
| 352 | IgG1 | GAGCCCAAGAGCTGTGATAAGACCCACACCT |
| | | GCCCTCCCTGTCCA |
| 353 | v1661 | AAEPKSSDKTHTCPPCP |
| 354 | v1661 | GCAGCCGAACCCAAATCCTCTGATAAGACCC |
| | | ACACATGCCCTCCATGTCCA |
| 355 | Hinge-1 | EPKSSDKTHTCPPCP |
| 356 | Hinge-1 | GAGCCTAAAAGCTCCGACAAGACCCACACAT |
| | | GCCCACCTTGTCCG |
| 357 | Hinge-2 | DKTHTCPPCP |
| 358 | Hinge-2 | GACAAGACCCACACATGCCCACCTTGTCCG |
| 359 | Hinge-3 | GTCPPCP |
| 360 | Hinge-3 | GGCACATGCCCTCCATGTCCA |

### Dissociation Constant (K_{D}) and Maximal Binding (Bmax)

In some embodiments, an antigen-binding construct is described by functional characteristics including but not limited to a dissociation constant and a maximal binding.

The term “dissociation constant (K_{D})” as used herein, is intended to refer to the equilibrium dissociation constant of a particular ligand-protein interaction. As used herein, ligand-protein interactions refer to, but are not limited to protein-protein interactions or antibody-antigen interactions. The K_{D} measures the propensity of two proteins (e.g. AB) to dissociate reversibly into smaller components (A+B), and is define as the ratio of the rate of dissociation, also called the “off-rate (k_{off})”, to the association rate, or “on-rate (kₒₙ)”. Thus, K_{D} equals k_{off}/kₒₙ and is expressed as a molar concentration (M). It follows that the smaller the K_{D}, the stronger the affinity of binding. Therefore, a K_{D} of 1 mM indicates weak binding affinity compared to a K_{D} of 1 nM. K_{D} values for antigen-binding constructs can be determined using methods well established in the art. One method for determining the K_{D} of an antigen-binding construct is by using surface plasmon resonance (SPR), typically using a biosensor system such as a Biacore® system. Isothermal titration calorimetry (ITC) is another method that can be used to determine.

The term “Bmax”, or maximal binding, refers to the maximum antigen-binding construct binding level on the cells at saturating concentrations of antigen-binding construct. This parameter can be reported in the arbitrary unit MFI for relative comparison, or converted into an absolute value corresponding to the number of antigen-binding constructs bound to the cell with the use of a standard curve.

The binding characteristics of an antigen-binding construct can be determined by various techniques. One of which is the measurement of binding to target cells expressing the antigen by flow cytometry (FACS, Fluorescence-activated cell sorting). Typically, in such an experiment, the target cells expressing the antigen of interest are incubated with antigen-binding constructs at different concentrations, washed, incubated with a secondary agent for detecting the antigen-binding construct, washed, and analyzed in the flow cytometer to measure the median fluorescent intensity (MFI) representing the strength of detection signal on the cells, which in turn is related to the number of antigen-binding constructs bound to the cells. The antigen-binding construct concentration vs. MFI data is then fitted into a saturation binding equation to yield two key binding parameters, Bmax and apparent K_{D}.

Apparent K_{D}, or apparent equilibrium dissociation constant, represents the antigen-binding construct concentration at which half maximal cell binding is observed. Evidently, the smaller the K_{D} value, the smaller antigen-binding construct concentration is required to reach maximum cell binding and thus the higher is the affinity of the antigen-binding construct. The apparent K_{D} is dependent on the conditions of the cell binding experiment, such as different receptor levels expressed on the cells and incubation conditions, and thus the apparent K_{D} is generally different from the K_{D} values determined from cell-free molecular experiments such as SPR and ITC. However, there is generally good agreement between the different methods.

### Methods of Preparation of Antigen-Binding Constructs

Antigen-binding constructs described herein may be produced using recombinant methods and compositions, e.g., as described in U.S. Pat. No. 4,816,567.

In one embodiment, an isolated nucleic acid encoding an antigen-binding construct described herein is provided. Such nucleic acid may encode an amino acid sequence comprising the VL and/or an amino acid sequence comprising the VH of the antigen-binding construct (e.g., the light and/or heavy chains of the antigen-binding construct). In a further embodiment, one or more vectors (e.g., expression vectors) comprising such nucleic acid are provided. In one embodiment, the nucleic acid is provided in a multicistronic vector. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antigen-binding construct and an amino acid sequence comprising the VH of the antigen-binding polypeptide construct, or (2) a first vector comprising a nucleic acid that encodes an amino acid sequence comprising the VL of the antigen-binding polypeptide construct and a second vector comprising a nucleic acid that encodes an amino acid sequence comprising the VH of the antigen-binding polypeptide construct. In one embodiment, the host cell is eukaryotic, e.g. a Chinese Hamster Ovary (CHO) cell, or human embryonic kidney (HEK) cell, or lymphoid cell (e.g., Y0, NS0, Sp20 cell). In one embodiment, a method of making an antigen-binding construct is provided, wherein the method comprises culturing a host cell comprising nucleic acid encoding the antigen-binding construct, as provided above, under conditions suitable for expression of the antigen-binding construct, and optionally recovering the antigen-binding construct from the host cell (or host cell culture medium).

For recombinant production of the antigen-binding construct, a nucleic acid encoding an antigen-binding construct, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antigen-binding construct).

Suitable host cells for cloning or expression of antigen-binding construct-encoding vectors include prokaryotic or eukaryotic cells described herein.

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

For example, antigen-binding constructs may be produced in bacteria, in particular when glycosylation and Fc effector function are not needed. For expression of antigen-binding construct fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antigen-binding construct may be isolated from the bacterial cell paste in a soluble fraction and can be further purified.

In addition to prokaryotes, eukaryotic microbes such as filamentous fungi or yeast are suitable cloning or expression hosts for antigen-binding construct-encoding vectors, including fungi and yeast strains whose glycosylation pathways have been “humanized,” resulting in the production of an antigen-binding construct with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antigen-binding constructs are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antigen-binding constructs in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antigen-binding construct production, see, e.g., Yazaki and Wu, Methods in Molecular Biology, Vol. 248 (B. K. C. Lo, ed., Humana Press, Totowa, N.J.), pp. 255-268 (2003).

In one embodiment, the antigen-binding constructs described herein are produced in stable mammalian cells, by a method comprising: transfecting at least one stable mammalian cell with: nucleic acid encoding the antigen-binding construct, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antigen-binding construct in the expressed product.

If required, the antigen-binding constructs can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of antigen-binding constructs. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni⁺² affinity chromatography if a His-tag is employed, or immobilized anti-flag antibody if a flag-tag is used. For general guidance in suitable purification techniques, see, e.g. incorporated entirely by reference Protein Purification: Principles and Practice, 3^{rd} Ed., Scopes, Springer-Verlag, NY, 1994, incorporated entirely by reference. The degree of purification necessary will vary depending on the use of the antigen-binding constructs. In some instances no purification is necessary.

In certain embodiments the antigen-binding constructs are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, antigen-binding constructs described herein can be chemically synthesized using techniques known in the art (e.g., see Creighton, 1983. Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, -alanine, fluoro-amino acids, designer amino acids such as -methyl amino acids, C-methyl amino acids, N-methyl amino acids, and amino acid analogs in general. Furthermore, the amino acid can be D (dextrorotary) or L (levorotary).

In some embodiments, the antigen-binding constructs described herein are substantially purified. The term “substantially purified” refers to a construct described herein, or variant thereof that may be substantially or essentially free of components that normally accompany or interact with the protein as found in its naturally occurring environment, i.e. a native cell, or host cell in the case of recombinantly produced antigen-binding construct that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein. When the antigen-binding construct or variant thereof is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the antigen-binding construct or variant thereof is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, a “substantially purified” antigen-binding construct produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Post-Translational Modifications:

In certain embodiments antigen-binding constructs described herein are differentially modified during or after translation.

The term “modified,” as used herein refers to any changes made to a given polypeptide, such as changes to the length of the polypeptide, the amino acid sequence, chemical structure, co-translational modification, or post-translational modification of a polypeptide. The form “(modified)” term means that the polypeptides being discussed are optionally modified, that is, the polypeptides under discussion can be modified or unmodified.

The term “post-translationally modified” refers to any modification of a natural or non-natural amino acid that occurs to such an amino acid after it has been incorporated into a polypeptide chain. The term encompasses, by way of example only, co-translational in vivo modifications, co-translational in vitro modifications (such as in a cell-free translation system), post-translational in vivo modifications, and post-translational in vitro modifications.

In some embodiments, the modification is at least one of: glycosylation, acetylation, phosphorylation, amidation, derivatization by known protecting/blocking groups, proteolytic cleavage and linkage to an antibody molecule or antigen-binding construct or other cellular ligand. In some embodiments, the antigen-binding construct is chemically modified by known techniques, including but not limited, to specific chemical cleavage by cyanogen bromide, trypsin, chymotrypsin, papain, V8 protease, NaBH₄; acetylation, formylation, oxidation, reduction; and metabolic synthesis in the presence of tunicamycin.

Additional post-translational modifications of antigen-binding constructs described herein include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The antigen-binding constructs described herein are modified with a detectable label, such as an enzymatic, fluorescent, isotopic or affinity label to allow for detection and isolation of the protein. In certain embodiments, examples of suitable enzyme labels include horseradish peroxidase, alkaline phosphatase, beta-galactosidase, or acetylcholinesterase; examples of suitable prosthetic group complexes include streptavidin biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include iodine, carbon, sulfur, tritium, indium, technetium, thallium, gallium, palladium, molybdenum, xenon, fluorine.

In some embodiments, antigen-binding constructs described herein are attached to macrocyclic chelators that associate with radiometal ions.

In some embodiments, the antigen-binding constructs described herein are modified by either natural processes, such as post-translational processing, or by chemical modification techniques which are well known in the art. In certain embodiments, the same type of modification may be present in the same or varying degrees at several sites in a given polypeptide. In certain embodiments, polypeptides from antigen-binding constructs described herein are branched, for example, as a result of ubiquitination, and in some embodiments are cyclic, with or without branching. Cyclic, branched, and branched cyclic polypeptides are a result from posttranslation natural processes or made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993), POST-TRANSLATIONAL COVALENT MODIFICATION OF PROTEINS, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:48-62 (1992)).

In certain embodiments, antigen-binding constructs described herein are attached to solid supports, which are particularly useful for immunoassays or purification of polypeptides that are bound by, that bind to, or associate with proteins described herein. Such solid supports include, but are not limited to, glass, cellulose, polyacrylamide, nylon, polystyrene, polyvinyl chloride or polypropylene.

### Assaying Functional Activity of Antigen-Binding Constructs

The antigen-binding constructs described herein can be assayed for functional activity (e.g., biological activity) using or routinely modifying assays known in the art, as well as assays described herein.

Methods of testing the biological activity of the antigen-binding constructs described herein can be measured by various assays as described in the Examples. Such methods include in vitro assays measuring T cell-mediated killing of target CD19+ B cells in comprising human whole blood, or PBMCs. Such assays may also be carried out using purified T cell cultures and autologous target B cells or tumor B cells.

In some embodiments, the antigen-binding constructs described herein are capable of synapse formation and bridging between CD19+ Raji B-cells and Jurkat T-cells as assayed by FACS and/or microscopy. In some embodiments, the antigen-binding constructs described herein mediate T-cell directed killing of CD20+ B cells in human whole blood. In some embodiments, the antigen-binding constructs described herein display improved biophysical properties compared to v875 and/or v1661; and/or displays improved yield compared to v875 and/or v1661, e.g., expressed at >10 mg/L after SEC (size exclusion chromatography); and/or displays heterodimer purity, e.g., >95%. In one embodiment, the assays are those described in the examples below.

In some embodiments, the functional characteristics of the bi-specific antigen-binding constructs described herein are compared to those of a reference antigen-binding construct. The identity of the reference antigen-binding construct depends on the functional characteristic being measured or the distinction being made. For example, when comparing the functional characteristics of exemplary bi-specific antigen-binding constructs, the reference antigen-binding construct may be the anti CD19 antibody HD37 and/or the anti CD3 antibody OKT3. In other embodiment, the reference antigen-binding construct is a construct described herein, e.g., v v875 and v1661.

The degree to which an antibody blocks binding to OKT3 or HD37 can be assessed using a competition assay in which the test antibody is able to inhibit or block specific binding of the OKT3 or HD37 antibody (reference antibody) to its target antigen (see, e.g., Junghans et al., Cancer Res. 50:1495, 1990; Fendly et al. Cancer Research 50: 1550-1558; U.S. Pat. No. 6,949,245 for examples of assays). A test antibody competes with a reference antibody if an excess of a test antibody (e.g., at least 2×, 5×, 10×, 20×, or 100×) inhibits or blocks binding of the reference antibody by, e.g., at least 50%, 60%, 70%, 75%, 80%, 85%, 90%, 95%, or 99% as measured in a competitive binding assay. Test antibodies identified by competition assay (blocking antibodies) include those binding to the same epitope as the reference antibody and antibodies binding to an adjacent epitope sufficiently proximal to the epitope bound by the reference antibody for steric hindrance to occur.

For example, in one embodiment where one is assaying for the ability of a antigen-binding construct described herein to bind an antigen or to compete with another polypeptide for binding to an antigen, or bind to an Fc receptor and/or anti-albumin antibody, various immunoassays known in the art can be used, including but not limited to, competitive and non-competitive assay systems using techniques such as radioimmunoassays. ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In certain embodiments, where a binding partner (e.g., a receptor or a ligand) is identified for an antigen-binding domain comprised by a antigen-binding construct described herein, binding to that binding partner by an antigen-binding construct described herein is assayed, e.g., by means well-known in the art, such as, for example, reducing and non-reducing gel chromatography, protein affinity chromatography, and affinity blotting. See generally. Phizicky et al., Microbiol. Rev. 59:94-123 (1995). In another embodiment, the ability of physiological correlates of a antigen-binding construct protein to bind to a substrate(s) of antigen-binding polypeptide constructs of the antigen-binding constructs described herein can be routinely assayed using techniques known in the art.

### Antigen-Binding Constructs and Antibody Drug Conjugates (ADC)

In certain embodiments an antigen-binding construct described herein is conjugated to a drug, e.g., a toxin, a chemotherapeutic agent, an immune modulator, or a radioisotope. Several methods of preparing ADCs (antibody drug conjugates or antigen-binding construct drug conjugates) are known in the art and are described in U.S. Pat. No. 8,624,003 (pot method), U.S. Pat. No. 8,163,888 (one-step), and U.S. Pat. No. 5,208,020 (two-step method) for example.

In some embodiments, the drug is selected from a maytansine, auristatin, calicheamicin, or derivative thereof. In other embodiments, the drug is a maytansine selected from DM1 and DM4.

In some embodiments the drug is conjugated to the antigen-binding construct with an SMCC linker (DM1), or an SPDB linker (DM4).

In some embodiments the antigen-binding construct is conjugated to a cytotoxic agent. The term “cytotoxic agent” as used herein refers to a substance that inhibits or prevents the function of cells and/or causes destruction of cells. The term is intended to include radioactive isotopes (e.g. At211, I131, I125, Y90, Re186, Re188, Sm153, Bi212, P32, and Lu177), chemotherapeutic agents, and toxins such as small molecule toxins or enzymatically active toxins of bacterial, fungal, plant or animal origin, including fragments and/or variants thereof.

Conjugate Linkers

In some embodiments, the drug is linked to the antigen-binding construct, e.g., antibody, by a linker. Attachment of a linker to an antibody can be accomplished in a variety of ways, such as through surface lysines, reductive-coupling to oxidized carbohydrates, and through cysteine residues liberated by reducing interchain disulfide linkages. A variety of ADC linkage systems are known in the art, including hydrazone-, disulfide- and peptide-based linkages.

Suitable linkers include, for example, cleavable and non-cleavable linkers. A cleavable linker is typically susceptible to cleavage under intracellular conditions. Suitable cleavable linkers include, for example, a peptide linker cleavable by an intracellular protease, such as lysosomal protease or an endosomal protease. The linker may be covalently bound to the antibody to such an extent that the antibody must be degraded intracellularly in order for the drug to be released e.g. the MC linker and the like.

### Pharmaceutical Compositions

Also provided herein are pharmaceutical compositions comprising an antigen-binding construct described herein. Pharmaceutical compositions comprise the construct and a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. In some aspects, the carrier is a man-made carrier not found in nature. Water can be used as a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In certain embodiments, the composition comprising the construct is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, etc.

### Methods of Treatment

Also described herein are methods of treating a disease or disorder comprising administering to a subject in which such treatment, prevention or amelioration is desired, an antigen-binding construct described herein, in an amount effective to treat, prevent or ameliorate the disease or disorder.

Disorder and disease are used interchangeably and refer to any condition that would benefit from treatment with an antigen-binding construct or method described herein. This includes chronic and acute disorders or diseases including those pathological conditions which predispose the mammal to the disorder in question. In some embodiments, the disorder is cancer.

The term “subject” refers to an animal which is the object of treatment, observation or experiment. An animal may be a human, a non-human primate, a companion animal (e.g., dogs, cats, and the like), farm animal (e.g., cows, sheep, pigs, horses, and the like) or a laboratory animal (e.g., rats, mice, guinea pigs, and the like).

The term “mammal” as used herein includes but is not limited to humans, non-human primates, canines, felines, murines, bovines, equines, and porcines.

“Treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishing of any direct or indirect pathological consequences of the disease, preventing metastasis, decreasing the rate of disease progression, amelioration or palliation of the disease state, and remission or improved prognosis. In some embodiments, antigen-binding constructs described herein are used to delay development of a disease or disorder. In one embodiment, antigen-binding constructs and methods described herein effect tumor regression. In one embodiment, antigen-binding constructs and methods described herein effect inhibition of tumor/cancer growth.

Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, construct constructs described herein are used to delay development of a disease or to slow the progression of a disease.

The term “effective amount” as used herein refers to that amount of construct being administered, which will accomplish the goal of the recited method, e.g., relieve to some extent one or more of the symptoms of the disease, condition or disorder being treated. The amount of the composition described herein which will be effective in the treatment, inhibition and prevention of a disease or disorder associated with aberrant expression and/or activity of a therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

### Therapeutic Uses:

In an aspect, the antigen-binding constructs described herein are used in antibody-based therapies which involve administering the antigen-binding constructs, or nucleic acids encoding antigen-binding constructs to a patient for treating one or more diseases, disorders, or conditions.

In certain embodiments is provided a method for the prevention, treatment or amelioration of cancer, said method comprising administering to a subject in need of such prevention, treatment or amelioration a pharmaceutical composition comprising an antigen-binding construct described herein.

In certain embodiments is a method of treating cancer in a mammal in need thereof, comprising administering to the mammal a composition comprising an effective amount of the pharmaceutical composition described herein, optionally in combination with other pharmaceutically active molecules. In certain embodiments, the cancer is a lymphoma or leukemia.

In one embodiment, the cancer is a lymphoma or leukemia or a B cell malignancy, or a cancer that expresses CD19, or non-Hodgkin's lymphoma (NHL) or mantle cell lymphoma (MCL) or acute lymphoblastic leukemia (ALL) or chronic lymphocytic leukemia (CLL) or rituximab- or CHOP (Cytoxan™/Adriamycin™vincristine/prednisone therapy)-resistant B cell cancer.

In a further aspect, the antigen-binding constructs described herein are for use in the manufacture or preparation of a medicament. In one embodiment, the medicament is for treatment of cancer. In certain embodiments, the medicament is for the treatment of lymphoma or leukemia. In other embodiments, the medicament is for the treatment of cancer described above. In another embodiment, the medicament is for use in a method of treating cancer comprising administering to patient having cancer, an effective amount of the medicament.

In certain embodiments, the methods and uses described herein further comprise administering to the patient an effective amount of at least one additional therapeutic agent. e.g., cytotoxic agents, chemotherapeutic agents, cytokines, growth inhibitory agents, kinase inhibitors, anti-angiogenic agents, cardioprotectants, immunostimulatory agents, immunosuppressive agents, protein tyrosine kinase (PTK) inhibitors, other antibodies, Fc fusions, or immunoglobulins, or other therapeutic agents.

In certain embodiments, the additional therapeutic agent is for preventing and/or treating cancer. Such combination therapy encompasses combined administration (where two or more therapeutic agents are included in the same or separate formulations), and separate administration, in which case, administration of the antigen-binding construct described herein can occur prior to, simultaneously, and/or following, administration of the additional therapeutic agent and/or adjuvant.

The antigen-binding constructs described herein may be administered alone or in combination with other types of treatments (e.g., radiation therapy, chemotherapy, hormonal therapy, immunotherapy and anti-tumor agents).

Demonstration of Therapeutic or Prophylactic Activity:

The antigen-binding constructs or pharmaceutical compositions described herein are tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays.

Therapeutic/Prophylactic Administration and Composition:

Provided are methods of treatment, inhibition and prophylaxis by administration to a subject of an effective amount of an antigen-binding construct or pharmaceutical composition described herein. In an embodiment, the antigen-binding construct is substantially purified (e.g., substantially free from substances that limit its effect or produce undesired side-effects). In certain embodiments, the subject is an animal, including but not limited to animals such as cows, pigs, horses, chickens, cats, dogs, etc., and in certain embodiments, a mammal, and most preferably human.

Various delivery systems are known and can be used to administer an antigen-binding construct formulation described herein, e.g., encapsulation in liposomes, microparticles, microcapsules, recombinant cells capable of expressing the antigen-binding constructs, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The antigen-binding constructs may be administered by any convenient route, for example by infusion or bolus injection, by absorption through epithelial or mucocutaneous linings (e.g., oral mucosa, rectal and intestinal mucosa, etc.) and may be administered together with other therapeutic agents. Administration can be systemic or local. Suitable routes of administration include intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it is desirable to administer the antigen-binding constructs, or compositions described herein locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the antigen-binding constructs or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the antigen-binding constructs or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance. Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

### Kits and Articles of Manufacture

Also described herein are kits comprising one or more antigen-binding constructs described herein. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the antigen-binding construct.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilized form and the kit can additionally contain a suitable solvent for reconstitution of the lyophilized components. Irrespective of the number or type of containers, the kits described herein also may comprise an instrument for assisting with the administration of the composition to a patient. Such an instrument may be an inhalant, nasal spray device, syringe, pipette, forceps, measured spoon, eye dropper or similar medically approved delivery vehicle.

In another aspect described herein, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a T cell activating antigen-binding construct described herein. The label or package insert indicates that the composition is used for treating the condition of choice. Moreover, the article of manufacture may comprise (a) a first container with a composition contained therein, wherein the composition comprises an antigen-binding construct described herein; and (b) a second container with a composition contained therein, wherein the composition comprises a further cytotoxic or otherwise therapeutic agent. The article of manufacture in this embodiment described herein may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Polypeptides and Polynucleotides

The antigen-binding constructs described herein comprise at least one polypeptide. Also described are polynucleotides encoding the polypeptides described herein. The polypeptides and polynucleotides are typically isolated.

As used herein, “isolated” means an agent (e.g., a polypeptide or polynucleotide) that has been identified and separated and/or recovered from a component of its natural cell culture environment. Contaminant components of its natural environment are materials that would interfere with diagnostic or therapeutic uses for the antigen-binding construct, and may include enzymes, hormones, and other proteinaceous or non-proteinaceous solutes. Isolated also refers to an agent that has been synthetically produced, e.g., via human intervention.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full length proteins, wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, etc.; and chemically synthesized compounds having properties known in the art to be characteristic of amino acids. Examples of non-naturally occurring amino acids include, but are not limited to, α-methyl amino acids (e.g. α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the proteins of the present invention may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Also described herein are polynucleotides encoding polypeptides of the antigen-binding constructs. The term “polynucleotide” or “nucleotide sequence” is intended to indicate a consecutive stretch of two or more nucleotide molecules. The nucleotide sequence may be of genomic, cDNA, RNA, semisynthetic or synthetic origin, or any combination thereof.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid. Conservative substitution tables providing functionally similar amino acids are known to those of ordinary skill in the art. Such conservatively modified variants are in addition to and do not exclude polymorphic variants, interspecies homologs, and alleles described herein.

Conservative substitution tables providing functionally similar amino acids are known to those of ordinary skill in the art. The following eight groups each contain amino acids that are conservative substitutions for one another; 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins: Structures and Molecular Properties (W H Freeman & Co.; 2nd edition (December 1993)

The terms “identical” or percent “identity,” in the context of two or more nucleic acids or polypeptide sequences, refer to two or more sequences or subsequences that are the same. Sequences are “substantially identical” if they have a percentage of amino acid residues or nucleotides that are the same (i.e., about 60% identity, about 65%, about 70%, about 75%, about 80%, about 85%, about 90%, or about 95% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide of the present invention, including homologs from species other than human, may be obtained by a process comprising the steps of screening a library under stringent hybridization conditions with a labeled probe having a polynucleotide sequence described herein or a fragment thereof, and isolating full-length cDNA and genomic clones containing said polynucleotide sequence. Such hybridization techniques are well known to the skilled artisan.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988 Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of an algorithm that is suitable for determining percent sequence identity and sequence similarity are the BLAST and BLAST 2.0 algorithms, which are described in Altschul et al. (1997) Nuc. Acids Res. 25:3389-3402, and Altschul et al. (1990) J. Mol. Biol. 215:403-410, respectively. Software for performing BLAST analyses is publicly available through the National Center for Biotechnology Information available at the World Wide Web at ncbi.nlm.nih.gov. The BLAST algorithm parameters W. T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1992) Proc. Natl. Acad. Sci. USA 89:10915) alignments (B) of 50, expectation (E) of 10, M=5, N=−4, and a comparison of both strands. The BLAST algorithm is typically performed with the “low complexity” filter turned off.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

The phrase “selectively (or specifically) hybridizes to” refers to the binding, duplexing, or hybridizing of a molecule only to a particular nucleotide sequence under stringent hybridization conditions when that sequence is present in a complex mixture (including but not limited to, total cellular or library DNA or RNA).

The phrase “stringent hybridization conditions” refers to hybridization of sequences of DNA, RNA, or other nucleic acids, or combinations thereof under conditions of low ionic strength and high temperature as is known in the art. Typically, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. Longer sequences hybridize specifically at higher temperatures. An extensive guide to the hybridization of nucleic acids is found in Tijssen, Laboratory Techniques in Biochemistry and Molecular Biology—Hybridization with Nucleic Probes, “Overview of principles of hybridization and the strategy of nucleic acid assays” (1993).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. The engineered proteins are expressed and produced by standard molecular biology techniques.

By “isolated nucleic acid molecule or polynucleotide” is intended a nucleic acid molecule, DNA or RNA, which has been removed from its native environment. For example, a recombinant polynucleotide encoding a polypeptide contained in a vector is considered isolated. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts, as well as positive and negative strand forms, and double-stranded forms. Isolated polynucleotides or nucleic acids described herein, further include such molecules produced synthetically, e.g., via PCR or chemical synthesis. In addition, a polynucleotide or a nucleic acid, in certain embodiments, include a regulatory element such as a promoter, ribosome binding site, or a transcription terminator.

The term “polymerase chain reaction” or “PCR” generally refers to a method for amplification of a desired nucleotide sequence in vitro, as described, for example, in U.S. Pat. No. 4,683,195. In general, the PCR method involves repeated cycles of primer extension synthesis, using oligonucleotide primers capable of hybridising preferentially to a template nucleic acid.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

A derivative, or a variant of a polypeptide is said to share “homology” or be “homologous” with the peptide if the amino acid sequences of the derivative or variant has at least 50% identity with a 100 amino acid sequence from the original peptide. In certain embodiments, the derivative or variant is at least 75% the same as that of either the peptide or a fragment of the peptide having the same number of amino acid residues as the derivative. In certain embodiments, the derivative or variant is at least 85% the same as that of either the peptide or a fragment of the peptide having the same number of amino acid residues as the derivative. In certain embodiments, the amino acid sequence of the derivative is at least 90% the same as the peptide or a fragment of the peptide having the same number of amino acid residues as the derivative. In some embodiments, the amino acid sequence of the derivative is at least 95% the same as the peptide or a fragment of the peptide having the same number of amino acid residues as the derivative. In certain embodiments, the derivative or variant is at least 99% the same as that of either the peptide or a fragment of the peptide having the same number of amino acid residues as the derivative.

The term “modified,” as used herein refers to any changes made to a given polypeptide, such as changes to the length of the polypeptide, the amino acid sequence, chemical structure, co-translational modification, or post-translational modification of a polypeptide. The form “(modified)” term means that the polypeptides being discussed are optionally modified, that is, the polypeptides under discussion can be modified or unmodified.

In some aspects, an antigen-binding construct comprises an amino acids sequence that is at least 80, 85, 90, 91, 92, 93, 94, 95, 96, 97, 98, 99, or 100% identical to a relevant amino acid sequence or fragment thereof set forth in the Table(s) or accession number(s) disclosed herein. In some aspects, an isolated antigen-binding construct comprises an amino acids sequence encoded by a polynucleotide that is at least 80, 85, 90, 91, 92, 93, 94, 95, 96, 97, 98, 99, or 100% identical to a relevant nucleotide sequence or fragment thereof set forth in Table(s) or accession number(s) disclosed herein.

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information. Terms understood by those in the art of antibody technology are each given the meaning acquired in the art, unless expressly defined differently herein.

It is to be understood that the general description and following detailed description are exemplary and explanatory only and are not restrictive of any subject matter claimed.

In this application, the use of the singular includes the plural unless specifically stated otherwise.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means ±10% of the indicated range, value, sequence, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously. In addition, it should be understood that the individual single chain polypeptides or immunoglobulin constructs derived from various combinations of the structures and substituents described herein are disclosed by the present application to the same extent as if each single chain polypeptide or heterodimer were set forth individually. Thus, selection of particular components to form individual single chain polypeptides or heterodimers is within the scope of the present disclosure.

The section headings used herein are for organizational purposes only and are not to be construed as limiting the subject matter described.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All documents, or portions of documents, cited in the application including, but not limited to, patents, patent applications, articles, books, manuals, and treatises are hereby expressly incorporated by reference in their entirety for any purpose. All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

### EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

It is understood that the examples and embodiments described herein are for illustrative purposes only and that various modifications or changes in light thereof will be suggested to persons skilled in the art and are to be included within the spirit and purview of this application and scope of the appended claims.

### Example 1

### Design, Expression and Purification of Antigen-Binding Constructs and Controls

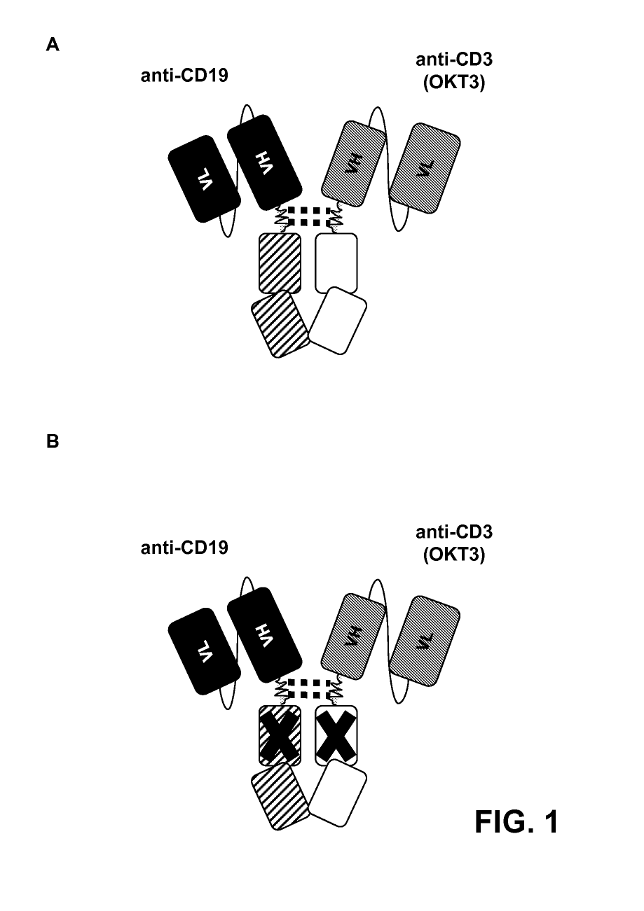

FIG. 1 depicts schematic representations of designs of antigen-binding constructs. FIG. 1A shows a representation of an exemplary CD3-CD19 antigen-binding construct with an Fc that is capable of mediating effector function. Both of the antigen-binding domains of the antigen-binding construct are scFvs, with the VH and VL regions of each scFv connected with a polypeptide linker. Each scFv is also connected to one polypeptide chain of a heterodimeric Fc with a hinge polypeptide. The two polypeptide chains of the antigen-binding construct are covalently linked together via disulphide bonds (depicted as dashed lines). FIG. 1B depicts a representation of an exemplary CD3-CD19 antigen-binding construct with an Fc knockout. This type of antigen-binding construct is similar to that shown in FIG. 1A, except that it includes modifications to the CH2 region of the Fc that ablate FcγR binding. These construct are thus unable to mediate Fc effector functions at therapeutically relevant concentrations.

A number of bispecific anti-CD3-CD19 antibodies were prepared as described in Table 1. Where the description of the anti-CD3 or anti-CD19 scFv includes a reference to BiTE, this indicates that anti-CD3 or anti-CD19 scFv has an amino acid sequence identical to the sequence of the VH and VL of the anti-CD3 anti-CD19 BiTE™ molecule (blinatumomab) with or without modifications to variable heavy and light chain orientation (e.g. VH-VL) as indicated below. Unless otherwise indicated, for αCD19_HD37 scFv and αCD3_OKT3 scFv, the order of the VL and VH regions from N-terminus to C-terminus is VLVH.

TABLE 1
Variants, Chain A, Chain B, Fc

| Variant | Chain A | Chain B | Fc |
|---|---|---|---|
| 875 | αCD19_HD37 scFv | αCD3_OKT3 scFv | Het Fc 1 |
| 1661 | αCD19_HD37 scFv | αCD3_OKT3 scFv | Het Fc 2; |
| | | | FcγR KO 2 |
| 1653 | αCD19_HD37 scFv | αCD3_OKT3 scFv | Het Fc 2 |
| | | (CDR C−>S) | |
| 1662 | αCD19_HD37 scFv | αCD3_OKT3 scFv | Het Fc 2; |
| | | (CDR C−>S) | FcγR KO 2 |
| 1660 | αCD3_OKT3 scFv | αCD19_HD37 scFv | Het Fc 2 |
| | (VHVL linker) | | |
| 1666 | αCD3_OKT3 scFv | αCD19_HD37 scFv | Het Fc 2; |
| | (VHVL linker) | | FcγR KO 2 |
| 1801 | αCD19_HD37 scFv | αCD3_OKT3 scFv | Het Fc 2 |
| | | (VLVH SS) | |
| N1 | αCD19_HD37 scFv | αCD3_OKT3 scFv | Het Fc 2; |
| | | (VLVH SS) | FcγR KO 2 |
| 6747 | αCD19_HD37 scFv | αCD3_OKT3 scFv | Het Fc 2 |
| | (VLVH SS) | (VLVH SS) | |
| 10149 | αCD19_HD37 scFv | αCD3_OKT3 scFv | Het Fc 2; |
| | (VLVH SS) | (VLVH SS) | FcγR KO 2 |
| N3 | αCD19_HD37 scFv | αCD3_OKT3 scFv | Het Fc 2 |
| | (VLVH SS) | (CDR C−>S) | |
| | | (VLVH SS) | |
| 10150 | αCD19_HD37 scFv | αCD3_OKT3 scFv | Het Fc 2; |
| | (VLVH SS) | (CDR C−>S) | FcγR KO 2 |
| | | (VLVH SS) | |
| 1380 | αCD19_HD37 scFv | αCD3_BiTE scFv | Het Fc 2; |
| | | | FcγR KO 1 |
| N10 | αCD19_HD37 scFv, | αCD3_OKT3 scFv | Het Fc 2 |
| | humanized (VLVH SS) | (VLVH SS) | |
| 12043 | αCD19_HD37 scFv, | αCD3_OKT3 scFv | Het Fc 2; |
| | humanized (VLVH SS) | (VLVH SS) | FcγR KO 1 |

Het Fc 1=Chain A: L351Y_F405A_Y407V; Chain B: T366L_K392M_T394W (EU numbering system for IgG1 Fc)
Het Fc 2=Chain A: T350V_L351Y_F405A_Y407V; Chain B: T350V_T366L_K392L_T394W
FcγR KO 1=Chain A: L234A_L235A; Chain B: L234A_L235A
FcγR KO 2=Chain A: D265S_L234A_L235A; Chain B: D265S_L234A_L235A
αCD19_HD37 scFv—N- to C-terminal order of variable regions is VLVH unless otherwise indicated
αCD3_OKT3 scFv—N- to C-terminal order of variable regions is VLVH unless otherwise indicated. The VLVH are connected by a (GGGGS)3 linker.
αCD3_BiTE scFv—N- to C-terminal order of variable regions is VH/VL and linker and composition is identical to blinatumomab.
(VLVH SS) or (VHVL SS) indicates disulfide stabilized scFv utilizing the published positions VH 44 and VL 100, according to the Kabat numbering system, to introduce a disulphide link between the VH and VL of the scFv [Reiter et al., Nat. Biotechnol. 14:1239-1245 (1996)].
(CDR C->S)—indicates a mutation in the H3 CDR of OKT3 as referenced below
(VHVL linker)—indicates VH and VL connected by the linker SSTGGGGSGGGG SGGGGSDI.

Fc numbering is according to EU index as in Kabat referring to the numbering of the EU antibody (Edelman et al., 1969, Proc Natl Acad Sci USA 63:78-85); Fab or variable domain numbering is according to Kabat (Kabat and Wu, 1991; Kabat et al, Sequences of proteins of immunological interest. 5th Edition—US Department of Health and Human Services, NIH publication no. 91-3242, p 647 (1991)).

The variants described in Table 1 include variant 875, a preliminary design, which was used as a starting point to generate antigen-binding constructs with improved yield and biophysical properties. The modifications include stabilization of the scFv by VLVH disulfide engineering and/or adding stabilizing CDR mutations. All variants include a heterodimeric Fc (Het Fc 1 or Het Fc 2) and can be expressed with or without mutations in the CH2 domain (FcγR KO 1 or FcγR KO 2) to abolish Fc effector activity. Variants including this modification to the Fc are referred to as having an Fc knockout or Fc KO.

Variants 875, 1661, 1653, 1662, 1660, 1666, 1801, and 1380 are initial designs of the CD3-CD19 antigen-binding constructs developed, while variants 6747, 10149, and 12043 exemplify designs that include modifications designed to further improve yield and biophysical properties of the CD3-CD19 antigen-binding constructs. Variants N1, N3 and N10 have also been designed and the biophysical and functional characteristics of these variants can be predicted from the data provided herein.

The VHVL disulfide engineering strategy for both the CD3 and CD19 scFvs utilized the published positions VH 44 and VL 100, according to the Kabat numbering system, to introduce a disulphide link between the VH and VL of the scFv [Reiter et al., Nat. Biotechnol. 14:1239-1245 (1996)]. The mutation of C to S in the 1-13 CDR of αCD3 OKT3 scFv was generated as described in Kipryanov et al., in Protein Engineering 10: 445-453 (1997).

Selected variants from Table 1 were prepared and the corresponding sequence composition of these variants is shown in Table 2.

TABLE 2
Sequence composition of bispecific CD3-CD19
antigen-binding constructs and controls

| | Chain A | Chain B |
|---|---|---|
| Variant Number | (clone #) | (clone #) |
| 875 | 1064 | 1067 |
| 1661 | 2183 | 2176 |
| 6747 | 5243 | 2227 |
| 10149 | 6692 | 6689 |
| 12043 | 7239 | 6689 |
| 891 | 1109 | |
| 1653 | 1842 | 2167 |
| 1662 | 2183 | 2177 |
| 1660 | 2174 | 2175 |
| 1666 | 2184 | 2185 |
| 1801 | 1842 | 2228 |
| 1380 | 1844 | 1890 |
| 10150 | 6692 | 6690 |

Cloning and Expression

The antibodies and antibody controls were cloned and expressed as follows. The genes encoding the antibody heavy and light chains were constructed via gene synthesis using codons optimized for human/mammalian expression. The scFv-Fc sequences were generated from a known anti-CD3 and CD19 scFv BiTE™ antibody (Kipriyanov et. al., 1998, Int. J Cancer: 77,763-772), anti-CD3 monoclonal antibody OKT3 (Drug Bank reference: DB00075).

The final gene products were sub-cloned into the mammalian expression vector pTT5 (NRC-BRI, Canada) and expressed in CHO cells (Durocher, Y., Perret, S. & Kamen, A. High-level and high-throughput recombinant protein production by transient transfection of suspension-growing CHO cells. Nucleic acids research 30, E9 (2002)).

The CHO cells were transfected in exponential growth phase (1.5 to 2 million cells/mL) with aqueous 1 mg/mL 25 kDa polyethylenimine (PEI, Polysciences) at a PEI:DNA ratio of 2.5:1. (Raymond C. et al. A simplified polyethylenimine-mediated transfection process for large-scale and high-throughput applications. Methods. 55(1):44-51 (2011)). In order to determine the optimal concentration range for forming heterodimers, the DNA was transfected in optimal DNA ratios of the heavy chain A (HC-A), and heavy chain B (HC-B) that allow for heterodimer formation (e.g. HC-A/HC-B/ratios=50:50%). Transfected cells were harvested after 5-6 days with the culture medium collected after centrifugation at 4000 rpm and clarified using a 0.45 μm filter.

The clarified culture medium was loaded onto a MabSelect SuRe (GE Healthcare) protein-A column and washed with 10 column volumes of PBS buffer at pH 7.2. The antibody was eluted with 10 column volumes of citrate buffer at pH 3.6 with the pooled fractions containing the antibody neutralized with TRIS at pH 11. The protein was desalted using an Econo-Pac 10DG column (Bio-Rad).

In some cases, the protein was further purified by gel filtration, 3.5 mg of the antibody mixture was concentrated to 1.5 mL and loaded onto a Superdex 200 HiLoad 16/600 200 pg column (GE Healthcare) via an AKTA Express FPLC at a flow-rate of 1 mL/min. PBS buffer at pH 7.4 was used at a flow-rate of 1 mL/min. Fractions corresponding to the purified antibody were collected, concentrated to 1 mg/mL and stored at −80° C.

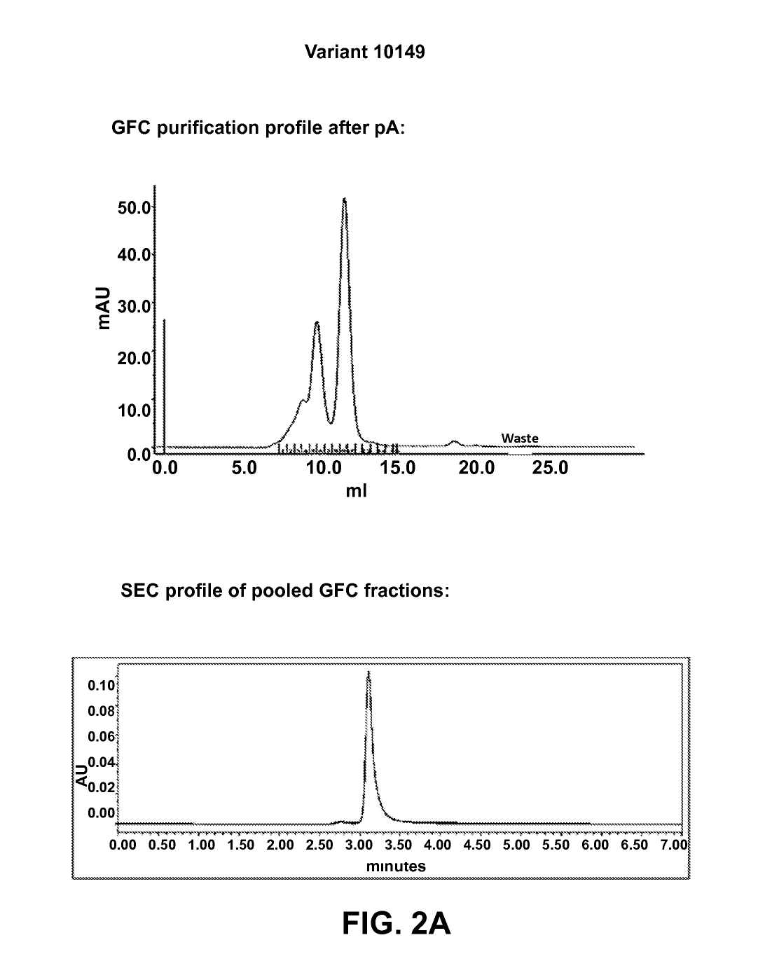

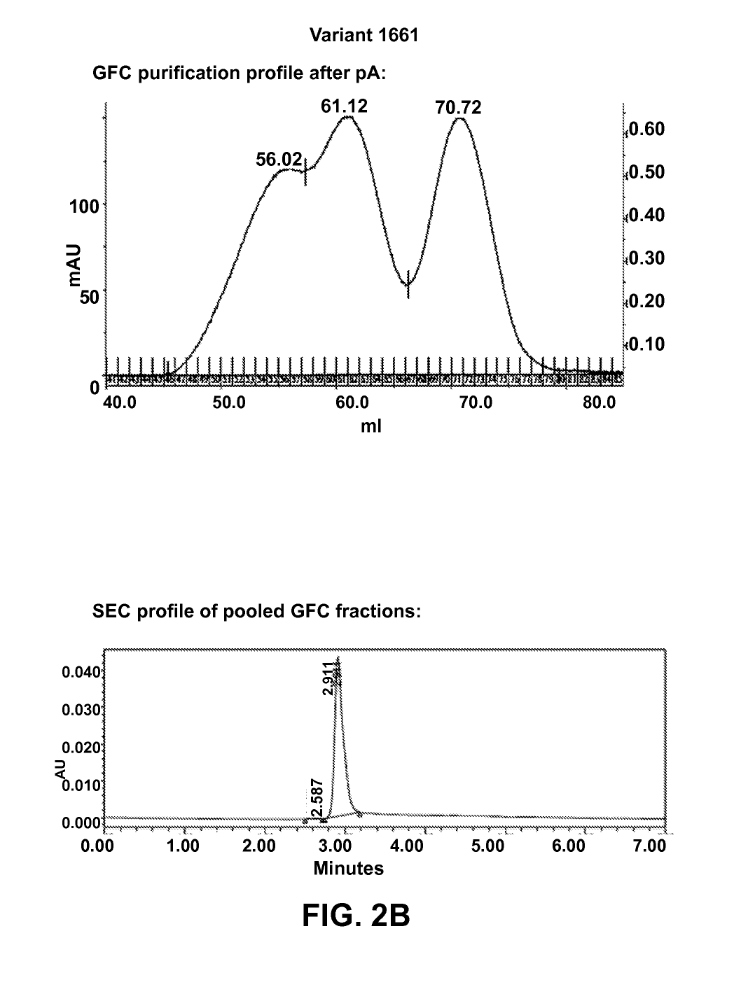

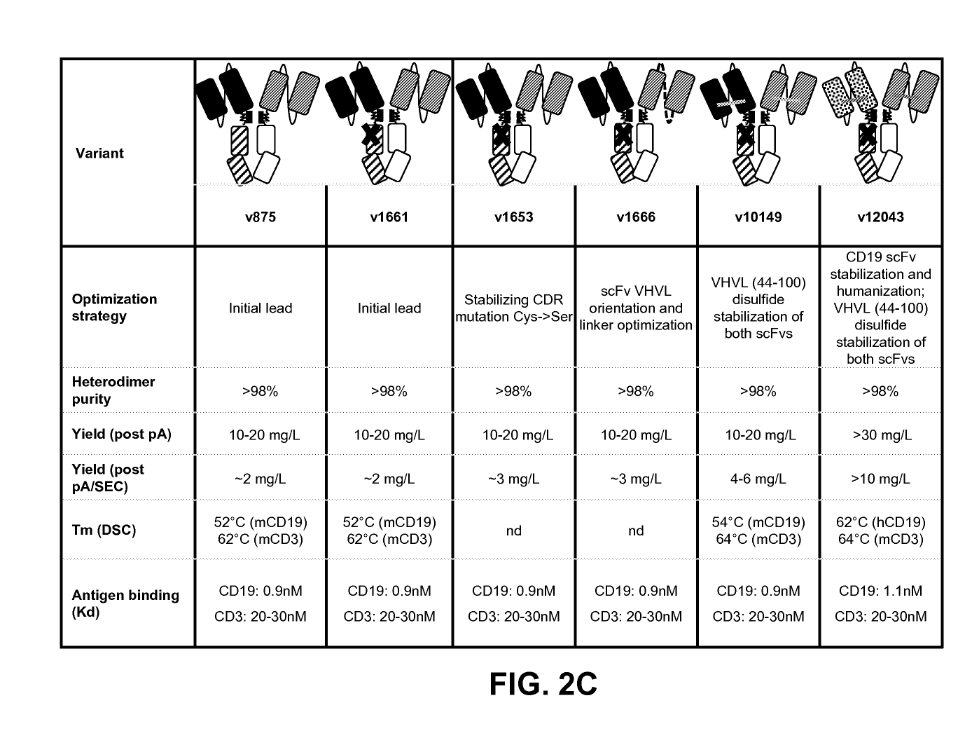

An additional purification step using, protein L chromatography after protein a purification could be carried out by the method as follows. Capto L resin was equilibrated with PBS and the variant was added to the resin and incubated at RT for 30 min. The resin was washed with PBS, and bound protein was eluted with 0.5 ml 0.1 M Glycine, pH 3. This additional step was not included in the production method used to generate the results in FIG. 2C.

The purity and yield of the final product was estimated by LC/MS and UPLC-SEC as described below.

LC-MS Analysis for Heterodimer Purity.

The purified samples were de-glycosylated with PNGase F for 6 hr at 37° C. Prior to MS analysis the samples were injected onto a Poros R2 column and eluted in a gradient with 20-90% ACN, 0.1% FA in 3 minutes, resulting in one single peak.

The peak of the LC column was analyzed with a LTQ-Orbitrap XL mass spectrometer using the following setup: Cone Voltage: 50 V′ Tube lens: 215 V; FT Resolution: 7,500. The mass spectrum was integrated with the software Promass or Max Ent. to generate molecular weight profiles.

UPLC-SEC Analysis

UPLC-SEC analysis was performed using a Waters BEH200 SEC column set to 30° C. (2.5 mL, 4.6×150 mm, stainless steel, 1.7 μm particles) at 0.4 ml/min. Run times consisted of 7 min and a total volume per injection of 2.8 mL with running buffers of 25 mM sodium phosphate, 150 mM sodium acetate, pH 7.1; and, 150 mM sodium phosphate, pH 6.4-7.1. Detection by absorbance was facilitated at 190-400 nm and by fluorescence with excitation at 280 nm and emission collected from 300-360 nm. Peak integration was analyzed by Empower 3 software.

All variants were expressed and purified to >95% heterodimer purity without contaminating homodimers.

The yield and heterodimer purity of variants 875, 1661, 1653, 1666, 10149, and 12043 are shown in FIG. 2C.

The gel filtration (GFC) profile after protein A purification for variant 10149 is shown in the upper panel of FIG. 2A, while the lower panel shows the SEC profile of the pooled GFC fractions. The upper panel of FIG. 2B shows the gel filtration (GFC) profile after protein A purification for variant 1661, while the lower panel shows the SEC profile of the pooled GFC fractions for 1661. FIG. 2C shows the improved yield and heterodimer purity of 10149 compared to 1661.

Assessment of Stability by Differential Scanning Calorimetry.

The stability of the CD3-CD19 antigen-binding constructs was assessed by determining the melting temperature (Tm) by differential scanning calorimetry (DSC). All DSC experiments were carried out using a GE VP-Capillary instrument. The proteins were buffer-exchanged into PBS (pH 7.4) and diluted to 0.3 to 0.7 mg/mL with 0.137 mL loaded into the sample cell and measured with a scan rate of 1° C./min from 20 to 100° C. Data was analyzed using the Origin software (GE Healthcare) with the PBS buffer background subtracted.

The results for variants 875, 1661, 1666, 10149, and 12043 are shown in FIG. 2C.

The initial variant 1661 showed low expression and post Protein A yield, and a large amount of high molecular weight aggregates as evident in the GFC post pA profile (FIGS. 2B and 2C). The lower expression and tendency of high molecular weight aggregates was optimized by scFv stability engineering using a variety of methods, including linker optimization, VHVL orientation, disulfide engineering and scFv stabilization by CDR grafting, that address different aspects of scFv expression and stability.

Variation of the scFv linker and VHVL orientations as exemplified in variant 1666 and 1380 did not yield significant improvement in expression and yield. Stabilization of the scFv by disulfide engineering did not improve the expression and post Protein A yield, but significantly reduced the amount of high molecular weight aggregates as shown in the GFC profile for variant 10149 (FIGS. 2B and 2C) and increased the final yield.

Stabilization by CDR grafting and humanization of the CD19 scFv yielded overall improved expression and post Protein A titer and scFv thermal stability and shown by the data for variant 12043 shown in FIG. 2C.

The initial variant 1661 showed low expression and post Protein A yield, and a large amount of high molecular weight aggregates as evident in the GFC post pA profile (FIGS. 2B and 2C). The lower expression and tendency of high molecular weight aggregates was optimized by scFv stability engineering using a variety of methods, including linker optimization, VHVL orientation, disulfide engineering and scFv stabilization by CDR grafting, that address different aspects of scFv expression and stability.

Variation of the scFv linker and VHVL orientations as exemplified in variant 1666 and 1380 did not yield significant improvement in expression and yield. Stabilization of the scFv by disulfide engineering did not improve the expression and post Protein A yield, but significantly reduced the amount of high molecular weight aggregates as shown in the GFC profile for variant 10149 (FIGS. 2B and 2C) and increased the final yield.

Stabilization by CDR grafting and humanization of the CD19 scFv yielded overall improved expression and post Protein A titer and scFv thermal stability and shown by the data for variant 12043 shown in FIG. 2C.

The analysis of post purification yield, heterodimer purity and thermal stability of scFvs as summarized in FIG. 2C shows that stabilization by disulfide engineering (v10149) and the humanization and stabilization of the CD19 scFv (v12043) yielded significant improvement in yield and thermal stability, while changing the VL-VH orientation and linker composition had no effect.

### Example 2

### Binding of CD3-CD19 Antigen-Binding Constructs to Rail and Jurkat Cells

The ability of the bispecific variants 875 and 1661 to bind to CD19- and CD3-expressing cells was assessed by FACS as described below.

Whole Cell Binding by FACS Protocol:

2×10⁶ cells/ml cells (>80% viability) were resuspended in L10+GS1 media, mixed with antibody dilutions, and incubated on ice for 1 h. Cells were washed by adding 10 ml of cold R-2 buffer, and centrifuging at 233×g for 10 min at 4° C. The cell pellet was resuspended with 100 μl (1/100 dilution in L10+GS1 media) of fluorescently labeled anti-mouse or anti-human IgG and incubated for 1 hour at RT. Cells were then washed by adding 10 ml of cold R-2 as described above, and the cell pellet resuspended with 400 μl of cold L-2 and the sample was filtered through Nitex and added to a tube containing 4 μl of propidium iodide.

Samples were analyzed by flow cytometry.

Table 3 provides a summary of the results indicating that all variants tested in this assay bind to CD19+ Raji B cells with comparable affinity, and to CD3+ Jurkat T cells with comparable affinity. All variants bound with high affinity to the Raji B cells, and with lower affinity to the Jurkat T cells. The low T cell affinity is most likely important for a serial TCR trigger process, allowing one T cell to kill multiple target cells.

### Example 3

### Analysis of T Cell and B Cell Bridging and Synapse (Pseudopodia) Formation by FACS and Microscopy

The ability of exemplary variants to mediate the formation of T cell synapses and pseudopodia was assessed as follows. The variants tested in this assay included 875 and 1661.

Whole Cell Bridging by FACS:

1×10⁶ cells/ml suspended in RPMI were labeled with 0.3 μM of the appropriate CellTrace label and mixed and incubated at 37° C. in a water bath for 25 minutes.

Pellets were resuspended in 2 ml of L10+GS1+NaN3 to a final concentration 5×106 cells/ml. Cell suspensions were analyzed (1/5 dilution) by flow cytometry to verify the appropriate cell labeling and laser settings. Flow-check and flow-set Fluorospheres were used to verify instrument standardization, optical alignment and fluidics. After flow cytometry verification, and prior to bridging, each cell line was mixed together at the desired ratio, at a final concentration of 1×10⁶ cells/ml. T:B bridging was assessed with Jurkat-violet+RAJI-FarRed.

Antibodies were diluted to 2× in L10+GS1+NaN3 at room temperature then added to cells followed by gentle mixing and a 30 min incubation. Following the 30 min incubation 2 μl of propidium iodide was added and slowly mixed and immediately analyze by flow cytometry. % Bridging B:T was calculated as the percentage of events that are simultaneously labeled violet and Far-red and the fold over background is calculated as ration % bridged of variants by % bridged of media only.

Analysis of Synapse (Pseudopodia) Formation by Microscopy:

Labeled Raji B cells and labeled Jurkat T cells were incubated for 30 min at room temperature with 3 nM of human IgG or variant. The cell suspension was concentrated by centrifugation, followed by removal of 180 μl of supernatant. Cell were resuspended in the remaining volume and imaged at 200× and 400×. Microscopy images (200×) were acquired, pseudo colored, overlaid and converted to TIFF using Openlab software. The cells were then counted using the cell counter in Image J software and binned into 5 different populations:

1. T alone (also include T:T)
2. T associated with B (no pseudopodia)
3. T associated with B (with pseudopodia, i.e. T-cells that showed a crescent-like structure)
4. B alone (also include B:B)
5. B associated with T

For some cells, a review of original and phase images in Openlab software was necessary for proper binning. Then, % of total T-cell associated with B-cells, % of total T-cell associated with B-cells that have pseudopodia, % of T-cell associated with B-cells that have pseudopodia, % of B-cells associated with T-cells and overall B:T (%) could be determined.

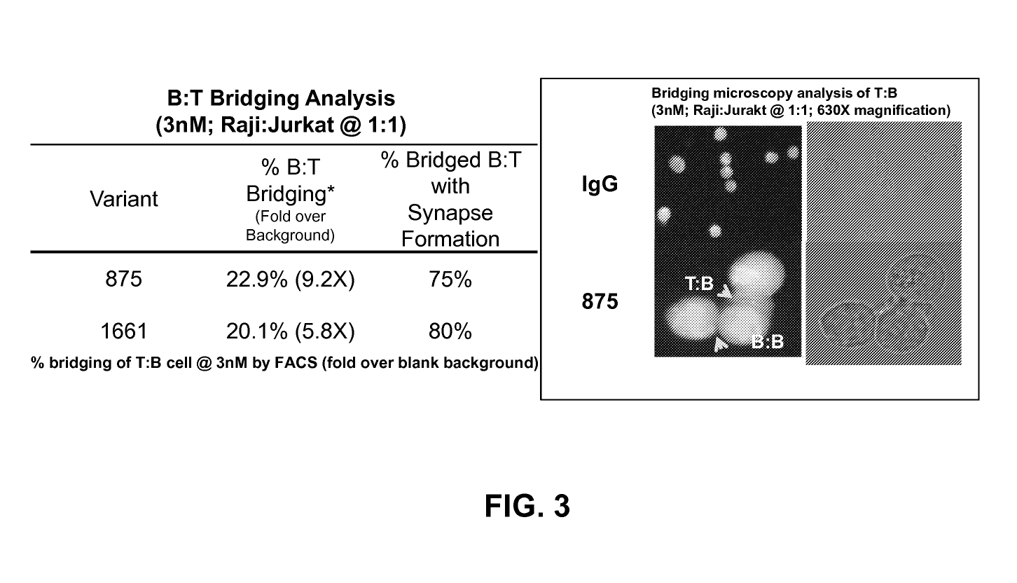

The results are shown in FIG. 3 and demonstrate that at 3 nM, variants 875 and 1661 were able to bridge CD19⁺ Raji B cells and Jurkat T cells with the formation of T cell synapses (pseudopodia) at a 1:1 stoichiometry. Over 80% of bridged T:B cells display pseudopodia indicative of synapse formation. This data indicates that variants 875 and 1661 are able to bridge Raji lymphoma B cells and Jurkat T cells, and elicit T:B cell synapses as a prerequisite and indication of T cell mediated target cell lysis.

### Example 4

### Determination of Off-Target Cytotoxicity of Activated Human CD8+ T-Cells in the Presence of a CD3-CD19 Antigen-Binding Construct

Potential off-target cytotoxicity of activated human CD8+ T cells in the presence of a CD3-CD19 antigen-binding construct was measured against the target cell line, K562 which does not express CD19 or CD3. The variant 875 was tested in this case, and the assay was carried out as follows.

Human blood (120-140 mL) for individual studies was collected from selected donors. PBMC were freshly isolated from donors using lymphocyte gradient separation (Cedarlane, Cat No. CL5020) For IL2 activation PBMCs were activated with 1000-3000 units/mL of IL-2 with an overnight incubation. Resting and IL-2 activated PBMCs were passed through EasySep (STEMCELL Technologies Inc.) columns for CD4+ and CD8+ enrichment. IL-2 activated CD8+ were used as effector cells and K562 erythroleukemia cells as target cells at an E:T ratio of 15:1. After incubating the cells with test articles for 20-26 hours, 50 microL of cell culture supernatant was collected for LDH analysis using a Promega LDH enzyme kit. Optical densities (OD) at 490 nm were determined for each well using a Molecular Devices Emax. Data analysis was performed using LibreOffice Calc software.

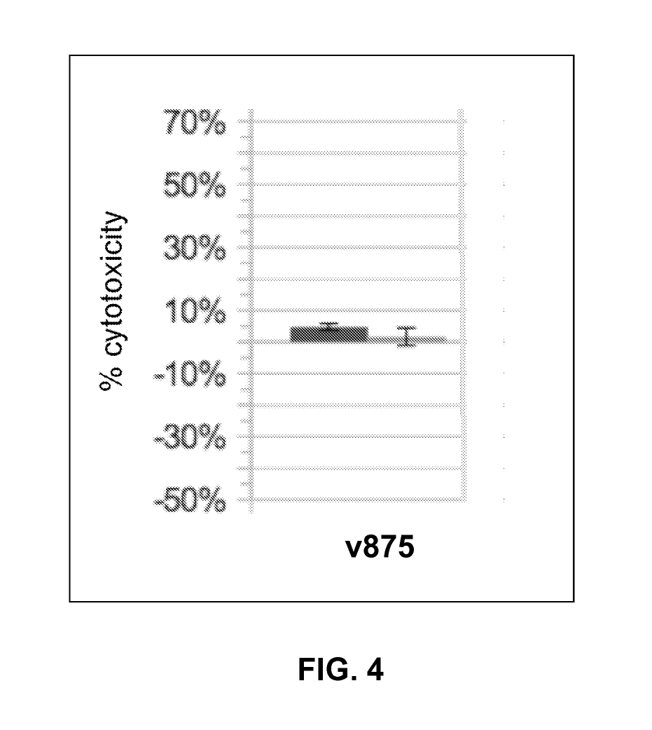

The results are shown in Table 3 and FIG. 4. Table 3 shows the percentage of activated T cell in purified CD8+ T cells at Day 0. FIG. 4 shows that no depletion of K562 erythroleukemia cells with IL-2 activated human CD8+ T cells was observed at 300 nM and a E:T ratio of 15:1. Thus, no off-target bystander cytotoxicity of K562 erythroleukemia cells with IL-2 activated human CD8+ T cells was observed at a saturating concentration and a high target to effector cell ratio.

TABLE 3
Percentage of activated T cell in purified CD8+ T cells at Day 0.

| | % CD69 cells | % CD69+ cells in |
|---|---|---|
| | in PBMCs | CD8+ enriched fractions |
| Donor 1 | 49 | 97 |
| Donor 2 | 52 | 96 |
| Donor 3 | 45 | 92 |
| Donor 4 | 62 | 95 |

### Example 5

### Ability of Variant 1661 to Mediate Dose-Dependent ADCC and CDC in Rail Cells

As described in Example 1, variant 1661 includes an Fc with CH2 mutations that abolish Fc mediated effector activity (Fc KO). In order to confirm lack of effector function for this variant it was tested in ADCC and CDC assays as described below.

Dose-response studies were performed at antibody concentration range of 1000-0.01 nM. Rituximab was used as a positive control. The ADCC assay was carried out as follows. Target Raji cells were pre-incubated with test antibodies for 30 min followed by adding effector cells with NK effector cell to target cell ratio of 5:1 and the incubation continued for 6 hours at 37° C. in 5% CO₂ incubators. LDH release and % target lysis was measured using LDH assay kit. For the CDC assay, normal human serum (NHS) at 10% final concentration was incubated with Raji target cells and respective antibody for 2 hours at 37° C. in 5% CO₂ incubators. LDH release and % target lysis was measured using LDH assay kit.

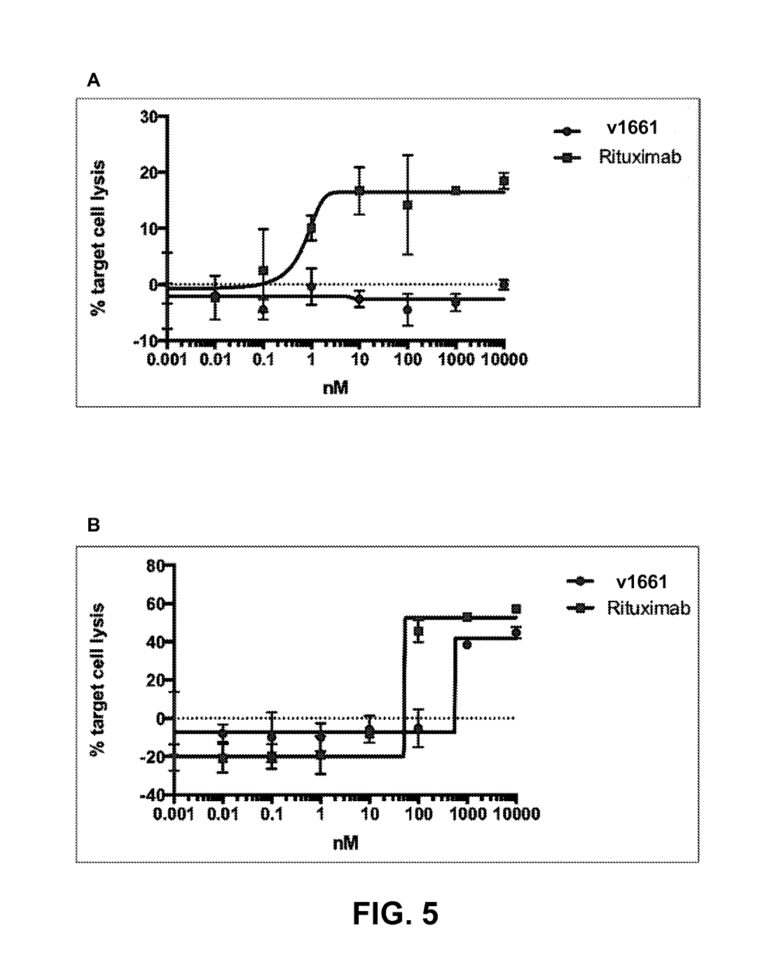

The results are shown in FIG. 5. FIG. 5A shows that variant 1661 was not able to mediate ADCC at concentrations up to 10 μM, as expected. By comparison, the positive control Rituximab did mediate ADCC. FIG. 5B shows that variant 1661 was more than 10-fold less potent than rituximab at eliciting CDC, also as expected, with an observed EC₅₀ of >500 nM. These results indicate that 1661 is unlikely to mediate ADCC and CDC at concentrations that mediate maximal target B cell killing (see subsequent examples).

### Example 6

### Autologous B Cell Depletion in Human Whole Blood

Bi-specific anti-CD19-CD3 antigen-binding constructs were analyzed for their ability to deplete autologous B cells in human whole blood primary cell culture under IL2 activation. The variants tested in this assay were 875, 1661, and 10149. As a nonspecific control, a homodimeric Fc without Fab binding arms (Fc block) was used.

Briefly, variants were incubated in heparinized human whole blood in the presence of IL2 for 2 days. Quadruplicate wells were plated for each control and experimental condition and cultures are incubated in 5% CO₂, 37° C. and stopped at 48 hours. The red blood cells were lysed after harvesting of the cultures and the collected primary cells were stained for CD45, CD20 and 7-AAD FACS detection. FACS analysis of the CD45+, CD45+/CD20+ and CD45+/CD20+/7AAD+/− populations was carried out by InCyte/FlowJo as follows: Between 5,000 event for FSC/SSC and compensation wells, and 30,000 events for experimental wells were analyzed by cytometry. A threshold was set to skip debris and RBCs. Gating was performed on lymphocytes, CD45+, CD20+, and 7AAD+ cells.

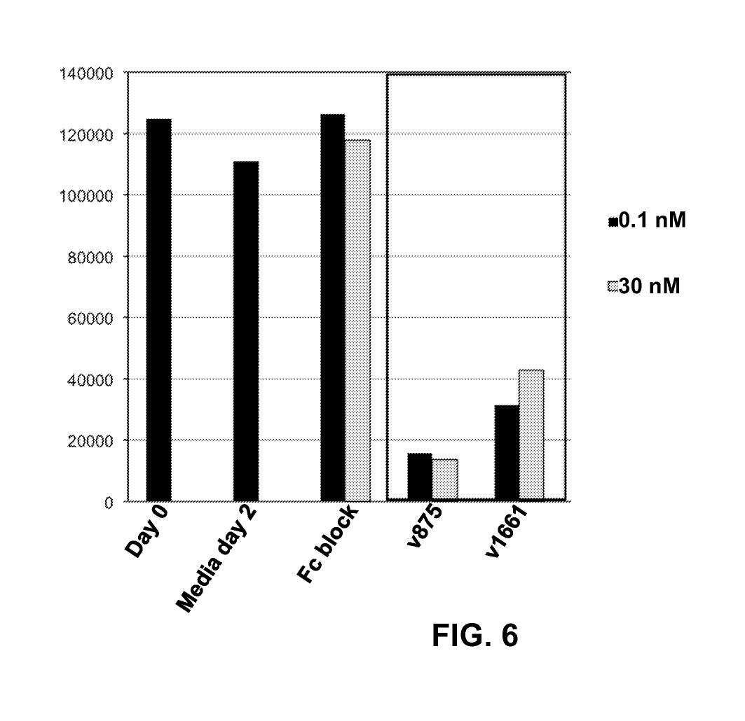

FIG. 6 shows the cytotoxic effect of the variants 875 and 1661 on the autologous B cell concentration in human whole blood under IL2 activation. Both variants were able to deplete CD20+ B cells in this assay. Maximal in vitro efficacy was observed at less than 0.1 nM, and there was a potent concentration-dependent effect with the EC₅₀ of about 0.001 nM.

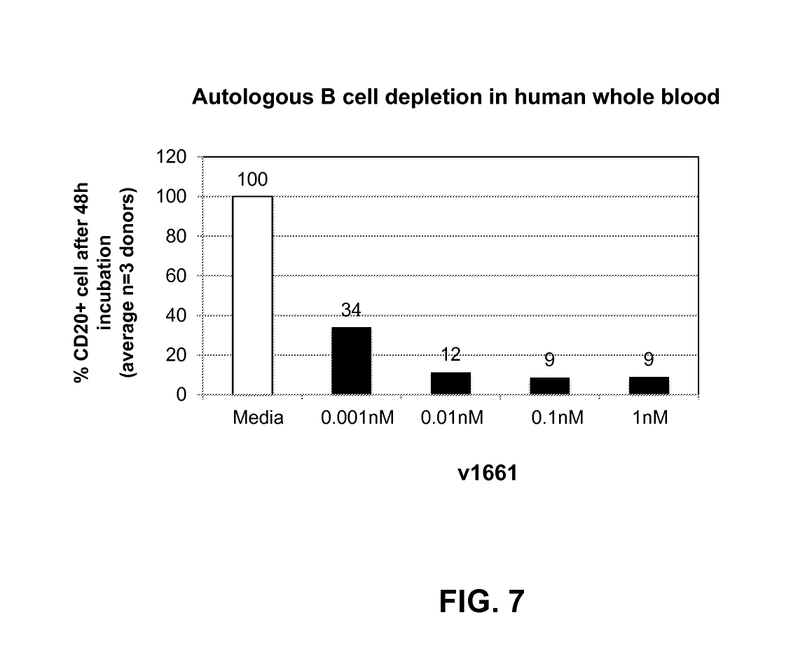

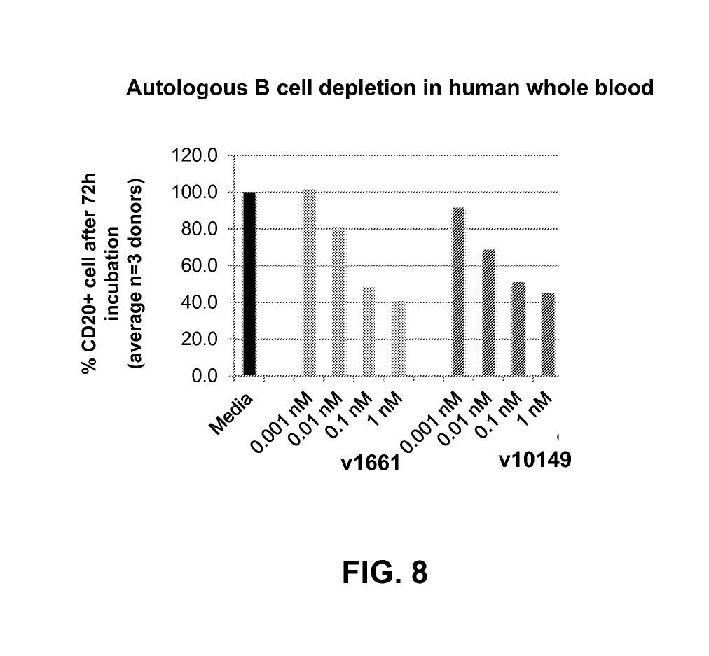

FIG. 7 shows that variant 1661 was able to mediate dose-dependent autologous B-cell depletion in a concentration-dependent manner (EC50<0.01 nM) in IL-2 activated human whole blood after 48 h at an E:T ratio of 10:1. The results are shown as the % of CD20+ B cells normalized to media control. FIG. 8 shows a comparison between variants 1661 and 10149, under resting conditions (i.e. in the absence of IL2 stimulation), indicating that both variants were able to deplete B cells in a dose-dependent manner. The disulfide stabilized variant 10149 showed equivalent potency to the parental variant v1661 in resting whole blood.

### Example 7

### Ability of an Exemplary CD3-CD19 Antigen-Binding Construct to Deplete Autologous B Cells in Primary CLL (Chronic Lymphocytic Leukemia and MCL (Mantle Cell Lymphoma) Patient Samples

The ability of variant 1661 to deplete autologous B cells in primary CLL and MCL patient whole blood samples was determined as follows.

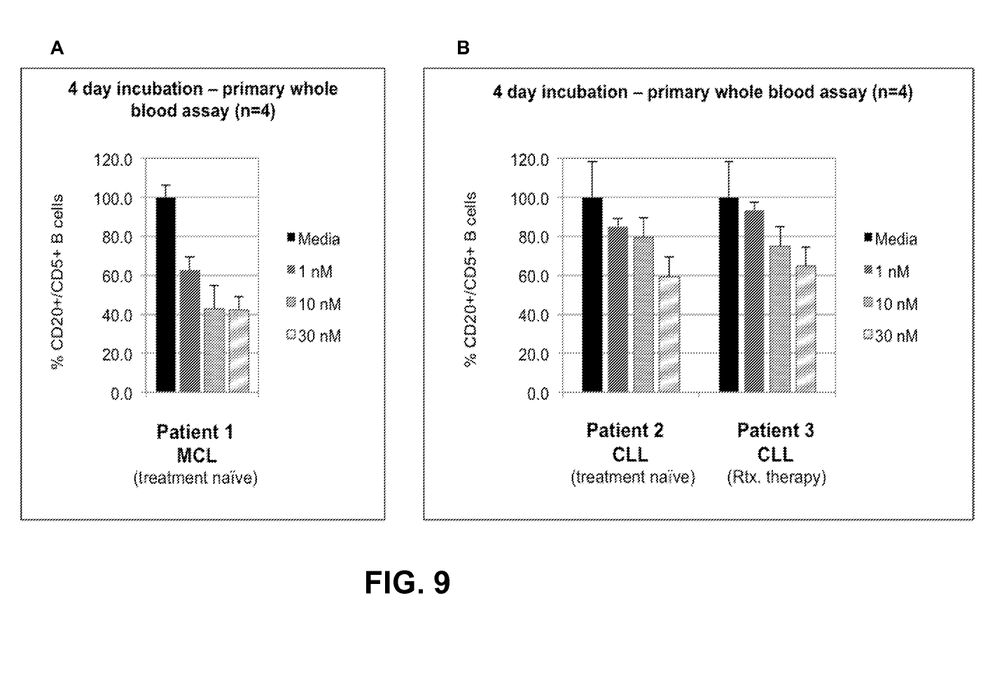

Primary patient blood samples were collected from 3 patients. The blood samples were treated on the day of blood collection as follows: Variants were directly incubated in heparinized patient whole blood. Quadruplicate wells were plated for each control and experimental condition and cultures are incubated in 5% CO₂, 37° C. and stopped at day 4. Red blood cells were lysed after harvesting of the cultures and the collected primary cells were stained for CD45, CD20, CD5, CD3, CD19 and 7-AAD FACS detection. FACS analysis was carried out in InCyte/FlowJo. Prior to carrying out the assay, basal lymphocyte counts for each patient were also determined by staining for CD45, CD20, CD5, CD3, CD19 and 7-AAD. The basal lymphocyte counts are shown in Table 4 below. FIGS. 9A and B show the results of the depletion assay. The results are shown as % of CD20+/CD5+ B cells normalized to media control.

TABLE 4
Basal Lymphocyte counts: Percentage of T and B cells
in patient whole blood before Z34 KO incubation.

| | Stage of | | | % CD20+/ | |
|---|---|---|---|---|---|
| Patient | disease | % CD19+ | % CD20+ | CD5+ | % CD3+ |
| profile | (RAI^{$}) | B cells | B cells | B cells | T cells |
| Patient 1 | 0 | 0.5 | 0.53 | 0.07 | 0.4 |
| (naïve MCL) | | | | | |
| Patient 2 | 0 | 0.82 | 0.83 | 0.81 | 0.17 |
| (naïve CLL) | | | | | |
| Patient 3 | 3 | 0.47 | 0.46 | 0.44 | 0.49 |
| (Rx treatment* | | | | | |
| CLL) | | | | | |

*Patient was receiving standard Rituxan plus Prednisone treatment at time of sampling
^{$}RAI: International RAI system for staging and diagnosis of CLL

The E:T ratio in MCL patient whole blood was 1:1.3 T cells to B cells. The E:T ratio in CCL patient whole blood was between 1:1 to 1:5 T cells to B cells. Variant 1661 was able to activate T cells in CLL primary patient whole blood, shown by elevated levels of CD69+ T cells after a 4 day incubation (data not shown). FIG. 9B shows that variant 1661 depleted CLL B cells in a concentration-dependent manner and to comparable extent in treatment naive and Rituxan pretreated primary patient whole blood samples. FIG. 9A shows that variant 1661 demonstrated concentration-dependent MCL B cell depletion in the treatment-naive primary patient whole blood sample.

### Example 8

### Assessment of Autologous T Cell Proliferation in Human PBMCs in the Presence of an Exemplary CD3-CD19 Antigen-Binding Construct

The ability of an exemplary CD3-CD19 antigen-binding construct to stimulate autologous T cell proliferation in human PBMCs was assessed. The variants tested were 875 and 1380 (with an Fc KO, similar to variant 1661). The controls tested were the wild-type OKT3 antibody, human IgG, and blinatumomab (variant 891). The assay was carried out as described below.

Cell proliferation assay: On Day 1, blood was collected from each of 4 donors and PBMCs were freshly isolated. The donor lymphocyte profile was determined by FACS as described in Example 6. The donor profiles of the 4 donors are shown in Table 5 below.

TABLE 5
Donor PBMC profile.

| | % live | % CD8+ | %CD19+ | % CD20+ | % CD56+ |
|---|---|---|---|---|---|
| | lymphocytes | T cells | B cells | B cells | NK cells |
| Donor 1 | 94 | 22 | 4.5 | 5.3 | 3 |
| Donor 2 | 95 | 25.4 | 2.9 | 4 | 4.2 |
| Donor 3 | 93.4 | 23.6 | 7.8 | 7.2 | 3.4 |
| Donor 4 | 88.2 | 18.2 | 10.9 | 6.9 | 3.8 |

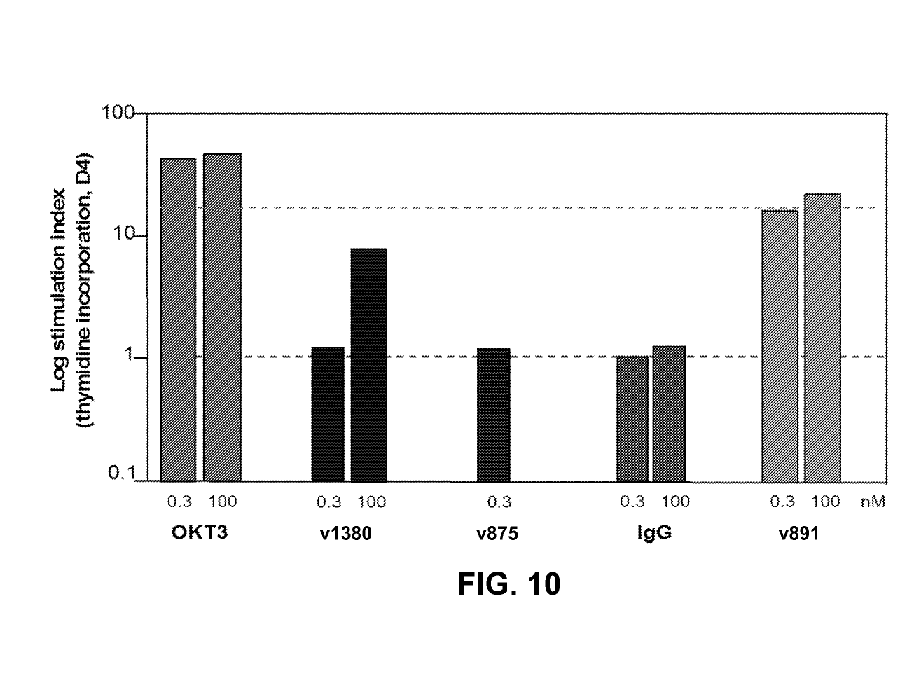

For the proliferation assay, the test items were prepared for a final concentration of 0.3 and 100 nM, combined with the PBMCs, and plated at 250,000 cells well. The mixtures were incubated for 3 days, after which tritiated thymidine was added to the cell-containing wells for a final concentration of 0.5 μCi thymidine/well; the plates were incubated for an additional 18 hours, after which the plates were frozen. Total incubation time was 4 days. The plates were filtered and counted (CPMs) using a β-counter. From the averages, a Stimulation Index (SI) was calculated as follows and the data was tabulated: average CPM of test item/average CPM of media only. The results of the assay are shown in FIG. 10, which shows that OKT3 mediated maximum T cell proliferation at 0.3 nM followed in descending rank order: v891 (blinatumomab)>v875 and v1380. At a concentration of 0.3 nM in serum of patients, OKT3 and blinatumomab are associated with adverse effects [Bargou et al. Science (2008); Klinger et al. Blood (2010)]. v1380 induced T cell proliferation to a significantly lower extent than OKT3 and blinatumomab. V1380, a variant which does not mediate Fc effector functions, like variant 1661, was able to induce sufficient T cell proliferation (but at much lower levels than benchmarks) for maximal B cell depletion (see Examples 5 and 6).

### Example 9

### Determination of Target B Cell Dependence for T Cell Proliferation in Human PBMC Mediated by an Exemplary CD3-CD19 Antigen-Binding Construct

Confirmation that the T cell proliferation mediated by the CD3-CD19 antigen-binding constructs is dependent on the presence of target B cells was obtained by assessing the ability of the CD3-CD19 antigen-binding constructs to stimulate T cell proliferation in PBMCs in the absence or presence of B cells and/or NK effector cells. The assay was carried out as described below, using variant 1380, the control blinatumomab (v891), and human IgG.

Cell proliferation assay: The PBMC derived subpopulations included PBMC, PBMC without B cells (PBMC-B), PBMC without NK cells (PBMC-NK), PBMC without NK and B cells (PBMC-NK-B). On Day 1, about 135 mL of blood was collected from each of 4 donors. PBMCs were freshly isolated and the PMBCs were passed through EasySep columns (STEMCELL Technologies Inc.) for CD19 and/or CD56 depletion by positive selection (day 1). The leukocyte profile of the PBMCs was determined by FACS as described in Example 6. The PBMC profiles are shown in Table 6.

TABLE 6
PBMC profile.

| | % live | % CD8+ | % CD19+ | % CD20+ | % CD56+ |
|---|---|---|---|---|---|
| | lymphocytes | T cells | B cells | B cells | NK cells |
| Donor 1 | 94 | 22 | 4.5 | 5.3 | 3 |
| Donor 2 | 95 | 25.4 | 2.9 | 4 | 4.2 |
| Donor 3 | 93.4 | 23.6 | 7.8 | 7.2 | 3.4 |
| Donor 4 | 88.2 | 18.2 | 10.9 | 6.9 | 3.8 |

The T cell proliferation assay was carried out as follows. The test items were prepared for a final concentration of 100 nM and combined with the PBMCs, plated at 250,000 cells/well. The mixtures were incubated for 3 days, after which tritiated thymidine was added to the cell-containing wells for a final of 0.5 μCi thymidine/well; the plates were incubated for an additional 18 hours, after which the plates were frozen. Total incubation time was 4 days. The plates were filtered and counted (CPMs) using a β-counter. From the averages, a Stimulation Index (SI) was calculated as follows and the data was tabulated: average CPM of test item/average CPM of media only.

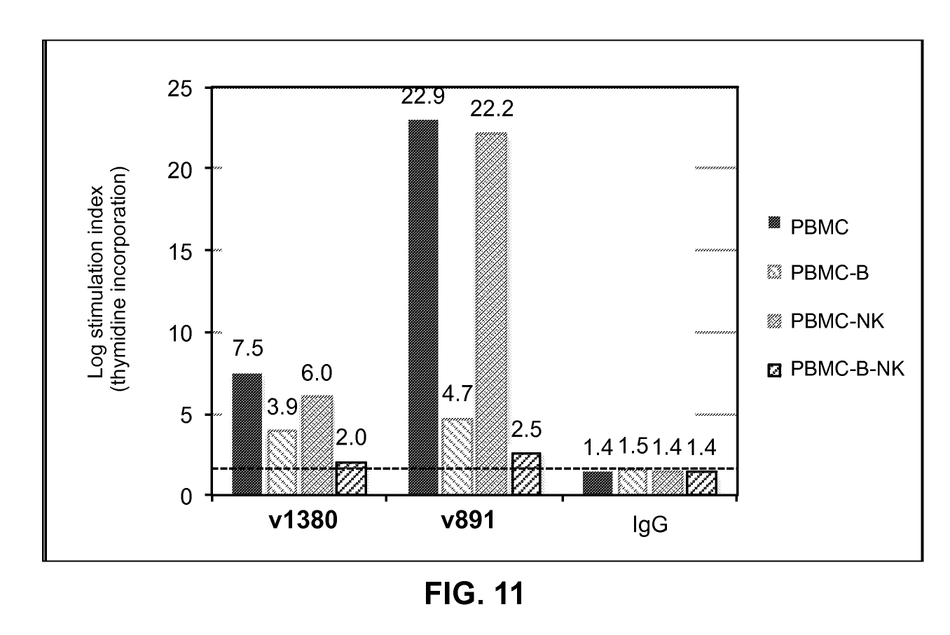

The results are shown in FIG. 11. The average E:T ratio in human PBMC collected from healthy donors was ˜10:1 CD3 T cells to CD19+ B cells (data not shown).

FIG. 11 shows that variant 1380 showed T cell proliferation in PBMCs, and PBMC-NK cells (PBMCs minus NK cells), but little to no T cell proliferation in PBMC lacking B cells and PBMC lacking B cells and NK cells, indicating target B cell dependence. Blinatumomab showed similar target B cell dependence for T cell activation, but induced higher T cell proliferation than 1380.

These results indicate that variant 1380 exhibits strictly target-dependent T cell proliferation at concentrations mediating maximal B cell depletion (see examples 5 and 6). These results also indicate that variant 1380 and other CD3-CD19 antigen-binding constructs with an Fc that is unable to mediate effector functions is likely to have a higher therapeutic index than blinatumomab. 1380 has identical CDR sequences to 1661 and equivalent T and B cell affinities and only differs from 1661 in the anti-CD3 scFv VH-VL orientations and scFv linker (see Table 1).

### Example 10

### In Vivo Efficacy of CD3-CD19 Antigen-Binding Constructs in NSG Mice Engrafted with IL2 Activated Human PBMC and G2 Leukemia Cells

The efficacy of exemplary CD3-CD19 antigen-binding constructs in an in vivo mouse leukemia model was determined. In this model, PBMC humanized NSG (NOD) scid gamma) mice were engrafted with chemo resistant G2 ALL (Acute lymphoblastic leukemia) cells, and the effect of CD3-CD19 antigen-binding constructs 875 and 1661 on the level of the G2 leukemia cell engraftment was observed. This model is described in Ishii et al. Leukemia 9(1):175-84 (1995), and Nervi et al, Exp Hematol 35: 1823-1838 (2007).

As a preliminary experiment the ability of selected variants to bind to the G2 leukemia cell line was tested.

In Vitro FACS Binding to Human G2 ALL Tumor Cell Line:

Pre-chilled G2 cells (1×10⁶ viable cells/tube) were incubated in triplicate on ice for 2 h in the absence of CO₂ with ice cold bispecific reagent huCD3×huCD19 at concentrations of 0, 0.1, 0.3, 1, 3, 10, 30, and 100 nM in Leibovitz L15 buffer containing 10% heat inactivated fetal bovine serum and 1% goat serum (L-10+GS1) in a final volume of 200 microL/tube. After the incubation, cells were washed in 4 ml ice cold Leibovitz L15, and the pellet resuspended in 100 microL ice cold Alexa fluor 488-tagged anti-human antibody (Jackson Immunoresearch) diluted 1/100 in L-10+GS1. After ≧15 min in the dark, 4 ml Leibovitz L15 was added, cells were pelleted, and then resuspended in 200 microL ice cold flow cytometry running buffer containing 2 ug/ml 7AAD before analysis by flow cytometry. Mean fluorescence intensity was used to establish binding curves from which the Kd was determined for each bispecific reagent for each cell line.

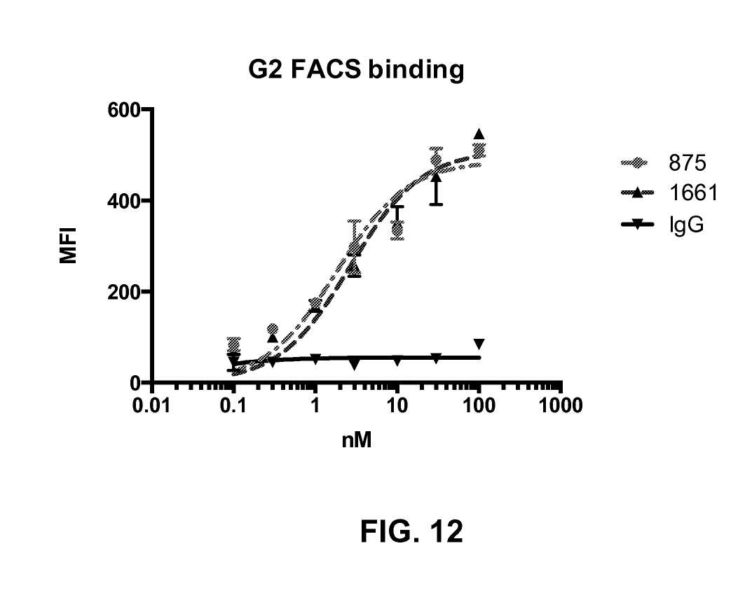

FIG. 12 shows that the exemplary variants, 875, and 1661 were able to bind to G2 ALL cells with a Kd of 1.9 nM for 875, and a Kd of 2.6 nM for 1661.

In vivo efficacy in NSG mice engrafted with IL2 activated human PBMC and G2 leukemia cells:

NOD/SCID/_{c}^{null} (NSG) mice (n=5/group) were implanted intravenously with 1×10⁵ G2-CBRluc/eGFP cells mixed with 3×10⁶ activated (anti-CD3/antiCD28 s [1 bead/CD3+ cell]+50 U IL2/ml for 5 d) human PBMC using a single donor as the source of cells for all groups of mice. The ratio of human T cells:G2 B cells was 10:1. Flow cytometry was used to assess the activation state (CD3, CD4, CD8, CD25, CD69, CD45RO, CD62L, and CCR7) and viability (7AAD) of the T cells.

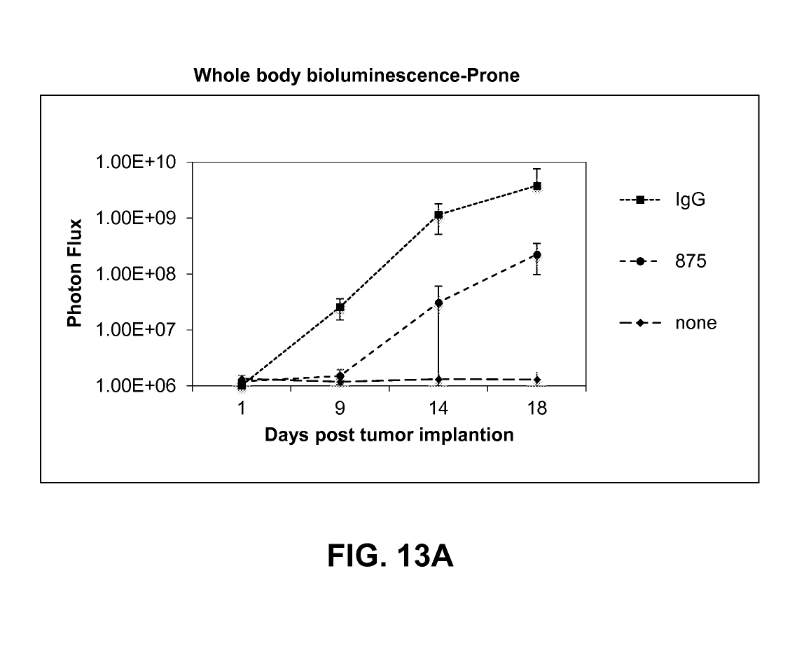

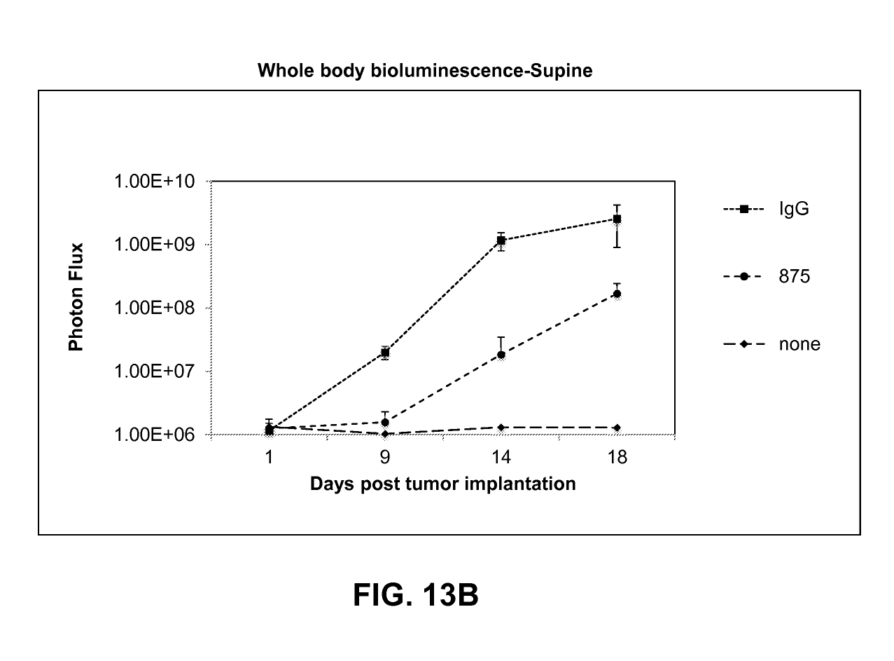

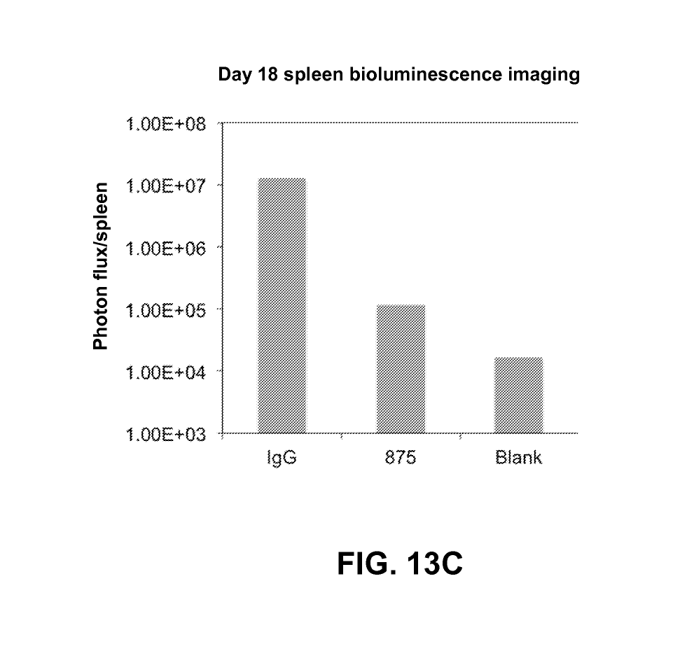

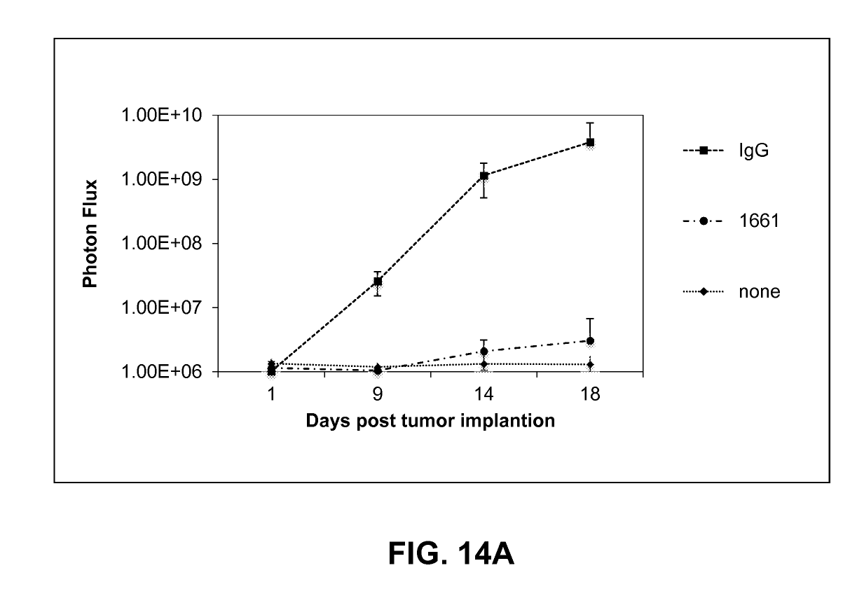

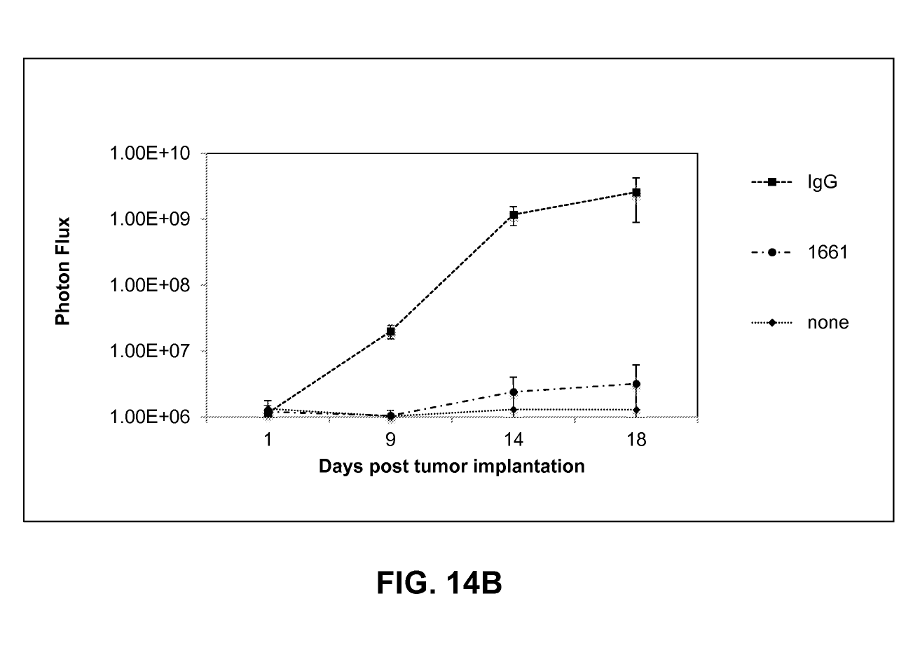

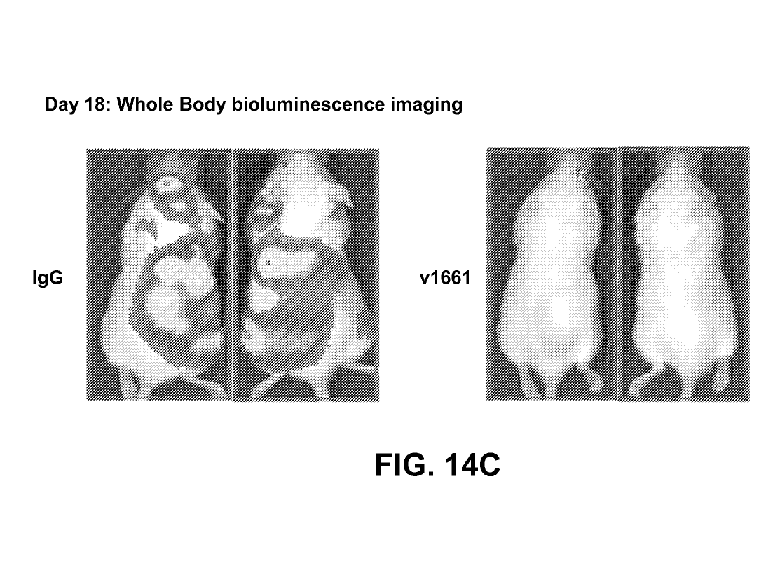

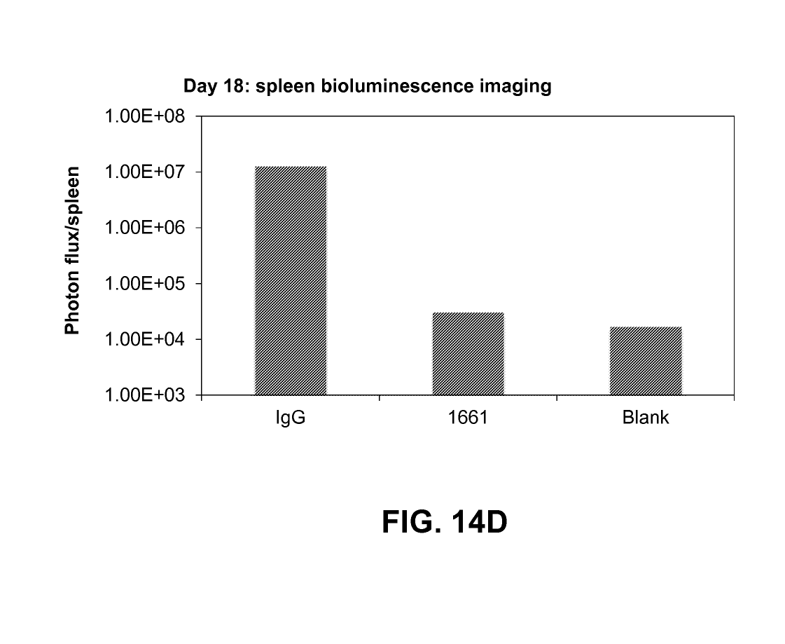

1 h after PBMC and G2 engraftment the mice received the first dose (n−5/group) of the bispecific variants with dosing at 3 mg/kg on day 0, 2, and 4, ending at Day 5. Tumor progression was followed by injecting mice with D-luciferin (150 micrograms/g body weight) followed by whole body bioluminescence imaging (BLI) 10 min later at baseline and on days 9, 14 and 18 post-implant. On day 18 animals were terminated and the spleen harvested for ex vivo BLI (bioluminescence imaging). The results are shown in FIGS. 13 and 14. ‘Blank’ indicates the control group without G2 engraftment.

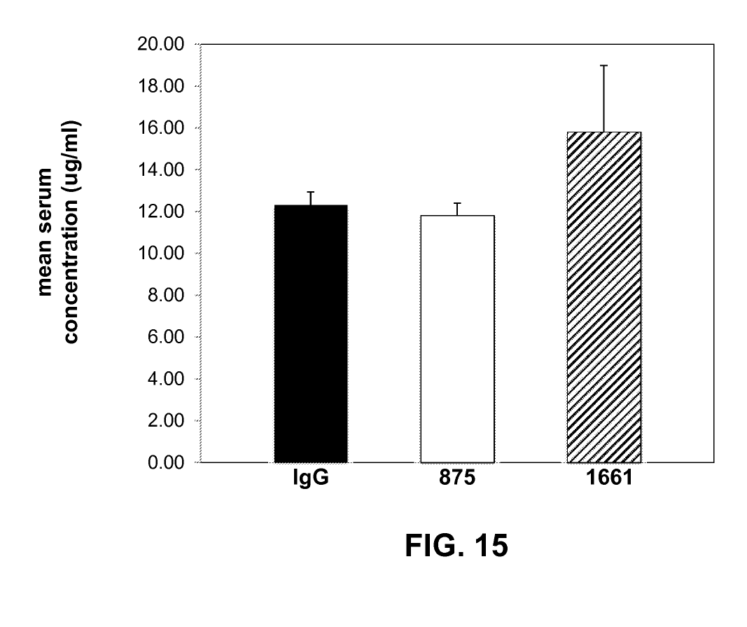

In addition, blood samples were collected for 2 animals per cohort at 24 hours after the first 3 mg/kg i.v. dose in order to determine mean serum concentrations in micrograms per mL. The results are shown in FIG. 15.

FIG. 13A shows the whole body BLI for variant 875 when measured in the prone position, while FIG. 13B shows the whole body BLI for the same variant in the supine position over 18 days. FIG. 13C shows the spleen BLI for variant 875 and controls at day 18.

FIG. 14A shows the whole body BLI for variant 1661 when measured in the prone position, while FIG. 14B shows the whole body BLI for the same variant in the supine position over 18 days. FIG. 14C shows an image of the whole body scan of the two representative mice from the IgG treated control group and the group treated with v1661. The figure shows no G2 engraftment for the v1661 treated animals and high engraftment and ALL disease progression in the IgG treated group. FIG. 14D shows the spleen BLI for variant 1661 and controls at day 18.

FIG. 15 shows the mean serum concentrations of variants 875 and 1661 achieved 24 hours after a 3 mg/kg i.v. dose.

These results indicate that the Fc knock-out variant 1661 shows complete depletion of the G2 ALL cells and no significant G2 engraftment. Under these conditions variant 875, which contains an active Fc, shows a similar, but reduced level of G2 depletion compared to the variant 1661.

TABLE S1
CDR sequences CD3 and CD19 antigen
binding constructs (289-386)

| | |
|---|---|
| Antigen binding constructs CDR sequence | SEQ ID NO: |

Wild-type OKT3 (CD3 binding)

| | |
|---|---|
| L1: SSVSY | 289 |
| L2: DTS | 290 |
| L3: QQWSSNP | 291 |
| H1: GYTFTRYT | 292 |
| H2: INPSRGYT | 293 |
| H3: ARYYDDHYCLDY | 294 |

Stabilized VARIANT of OKT3 (CD3 binding)

| | |
|---|---|
| L1: SSVSY | 295 |
| L2: DTS | 296 |
| L3: QQWSSNP | 297 |
| H1: GYTFTRYT | 298 |
| H2: INPSRGYT | 299 |
| H3: ARYYDDHYSLDY | 300 |

HD37 (CD19 binding) short

| | |
|---|---|
| L1: QSVDYDGDSYL | 301 |
| L2: DAS | 302 |
| L3: QQSTEDPWT | 303 |
| H1: GYAFSSYW | 304 |
| H2: IWPGDGDT | 305 |
| H3: RETTTVGRYYYAMDY | 306 |

Humanized VARIANT of HD37 (CD19 binding) short

| | |
|---|---|
| L1: QSVDYEGDSYL | 307 |
| L2: DAS | 308 |
| L3: QQSTEDPWT | 309 |
| H1: GYAFSSYW | 310 |
| H2: IWPGDGDT | 311 |
| H3: RETTTVGRYYYAMDY | 312 |

Humanized VARIANT of HD37 (CD19 binding)short

| | |
|---|---|
| L1: QSVDYSGDSYL | 313 |
| L2: DAS | 314 |
| L3: QQSTEDPWT | 315 |
| H1: GYAFSSYW | 316 |
| H2: IWPGDGDT | 317 |
| H3: RETTTVGRYYYAMDY | 318 |

HD37 (CD19 binding) long

| | |
|---|---|
| L1: KASQSVDYDGDSYL | 319 |
| L2: DASNLVS | 320 |
| L3: QQSTEDPWT | 321 |
| H1: GYAFSSYWMN | 322 |
| H2: QIWPGDGDTN | 323 |
| H3: RETTTVGRYYYAMDY | 324 |

Humanized VARIANT of HD37 (CD19 binding) long

| | |
|---|---|
| L1: RASQSVDYEGDSYL | 325 |
| L2: DASNLVS | 326 |
| L3: QQSTEDPWT | 327 |
| H1: GYAFSSYWMN | 328 |
| H2: QIWPGDGDTN | 329 |
| H3: RETTTVGRYYYAMDY | 330 |

Humanized VARIANT of HD37 (CD19 binding)long

| | |
|---|---|
| L1: RASQSVDYSGDSYL | 331 |
| L2: DASNLVS | 332 |
| L3: QQSTEDPWT | 333 |
| H1: GYAFSSYWMN | 334 |
| H2: QIWPGDGDTN | 335 |
| H3: RETTTVGRYYYAMDY | 336 |

TABLE S2
CD19 humanized VL sequences (SEQ ID NOS: 337, 338)

| SEQ | | |
|---|---|---|
| ID | | |
| NO: | Desc. | Sequence |
| 337 | hVL2 | DIQLTQSPSSLSASVGDRATITCRASQSVDYDGDSYLNWYQQKPGKAPKLLIYDASNLVSG |
| | wild- | IPSRFSGSGSGTDFTLTISSVQPEDAATYYCQQSTEDPWTFGCGTKLEIK |
| | type | |
| | CDRs | |
| 338 | hVL2 | GATATTCAGCTGACCCAGAGCCCAAGCTCCCTGTCTGCCAGTGTGGGGGATAGGGCTACAA |
| | wild- | TCACTTGCCGCGCATCACAGAGCGTGGACTATGAGGGCGATTCCTATCTGAACTGGTACCA |
| | type | GCAGAAGCCAGGGAAAGCACCCAAGCTGCTGATCTACGACGCCTCTAATCTGGTGAGTGGC |
| | CDRs | ATTCCCTCAAGGTTCTCCGGATCTGGCAGTGGGACTGACTTTACCCTGACAATCTCTAGTG |
| | | TGCAGCCCGAGGATGCCGCTACCTACTATTGCCAGCAGTCTACAGAAGACCCTTGGACTTT |
| | | CGGATGTGGCACCAAACTGGAGATTAAG |

TABLE S3
CD19 humanized VH sequences(SEQ ID NOS: 339-342)

| SEQ | | |
|---|---|---|
| ID | | |
| NO: | Desc. | Sequence |
| 339 | hVH2 | QVQLVQSGAEVKKPGASVKISCKASGYAFSSYWMNWVRQAPGQCLEWIGQIWPGDGDTN |
| | wild- | YAQKFQGRATLTADTSTSTAYMELSSLRSEDTAVYYCARRETTTVGRYYYAMDYWGQGTTVT |
| | type | VSS |
| | CDRs | |
| 340 | hVH2 | CAGGTCCAGCTGGTGCAGAGCGGAGCAGAGGTCAAGAAACCCGGAGCCAGCGTGAAAATTTC |
| | wild- | CTGCAAGGCCTCTGGCTATGCTTTCTCAAGCTACTGGATGAACTGGGTGAGGCAGGCACCAG |
| | type | GACAGTGTCTGGAATGGATCGGACAGATTTGGCCTGGGGACGGAGATACCAATTATGCTCAG |
| | CDRs | AAGTTTCAGGGACGCGCAACTCTGACCGCCGATACATCAACAAGCACTGCATACATGGAGCT |
| | | GTCCTCTCTGCGCTCCGAAGACACAGCCGTGTACTATTGCGCACGGAGAGAAACCACAACTG |
| | | TGGGCCGATACTATTACGCAATGGATTACTGGGGCCAGGGGACCACAGTCACTGTGAGTTCA |
| 341 | hVH3 | QVQLVQSGAEVKKPGASVKISCKASGYAFSSYWMNWVRQAPGQCLEWIGQIWPGDGDTNYAQ |
| | wild- | KFQGRATLTADESTSTAYMELSSLRSEDTAVYYCARRETTTVGRYYYAMDYWGQGTTVTVSS |
| | type | |
| | CDRs | |
| 342 | hVH3 | CAGGTCCAGCTGGTGCAGAGCGGAGCAGAGGTCAAGAAACCCGGAGCCAGCGTGAAAATTTC |
| | wild- | CTGCAAGGCCTCTGGCTATGCTTTCTCAAGCTACTGGATGAACTGGGTGAGGCAGGCACCAG |
| | type | GACAGTGTCTGGAATGGATCGGACAGATTTGGCCTGGGGACGGAGATACCAATTATGCTCAG |
| | CDRs | AAGTTTCAGGGACGCGCAACTCTGACCGCCGATGAGTCAACAAGCACTGCATACATGGAGCT |
| | | GTCCTCTCTGCGCTCCGAAGACACAGCCGTGTACTATTGCGCACGGAGAGAAACCACAACTG |
| | | TGGGCCGATACTATTACGCAATGGATTACTGGGGCCAGGGGACCACAGTCACTGTGAGTTCA |

TABLE S4
Variants and clones

| Variant Number | H1 (clone) | H2 (clone) |
|---|---|---|
| 875 | 1064 | 1067 |
| 1661 | 2183 | 2176 |
| 6747 | 5243 | 2227 |
| 10149 | 6692 | 6689 |
| 12043 | 7239 | 6689 |
| 891 | 1109 | n/a |
| 1653 | 1842 | 2167 |
| 1662 | 2183 | 2177 |
| 1660 | 2174 | 2175 |
| 1666 | 2184 | 2185 |
| 1801 | 1842 | 2228 |
| 1380 | 1844 | 1890 |
| 10150 | 6692 | 6690 |

TABLE S5
Sequences of clones by SEQ ID NO (1-288) (Desc. = description)

| SEQ | | | |
|---|---|---|---|
| ID | | | |
| NO: | Clone | Desc. | Sequence |
| 1 | 2176 | Full | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGG |
| | | | SQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS |
| | | | AAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIE |
| | | | KTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK |
| 2 | 2176 | Full | CAGATCGTCCTGACACAGAGCCCAGCTATCATGTCAGCAAGCCCCGGCGAGAAAGTCACAATGACTTGCTCAGCCAGCTCCTCTGTGAGCTACATGAACTGGTATCAGCAGAAAAGCGGA |
| | | | ACCTCCCCCAAGAGATGGATCTACGACACATCCAAGCTGGCCTCTGGAGTGCCTGCTCACTTCAGGGGCAGCGGCTCTGGGACCAGTTATTCACTGACAATTTCCGGCATGGAGGCCGAA |
| | | | GATGCCGCTACCTACTATTGCCAGCAGTGGAGTTCAAACCCATTCACTTTTGGATCTGGCACCAAGCTGGAAATTAATGGCGGAGGAGGCTCCGGAGGAGGAGGGTCTGGAGGAGGAGGA |
| | | | AGTCAGGTGCAGCTGCAGCAGTCCGGAGCAGAGCTGGCTCGACCAGGAGCTAGTGTGAAAATGTCCTGTAAGGCAAGCGGCTACACCTTCACACGGTATACCATGCATTGGGTGAAACAG |
| | | | AGACCCGGGCAGGGACTGGAATGGATCGGGTACATTAATCCTAGCCGAGGATACACAAACTACAACCAGAAGTTTAAAGACAAGGCCACTCTGACCACAGATAAGAGCTCCTCTACCGCT |
| | | | TATATGCAGCTGAGTTCACTGACATCTGAGGACAGTGCAGTGTACTATTGCGCCAGGTACTATGACGATCACTACTGTCTGGATTATTGGGGCCAGGGGACTACCCTGACAGTGAGCTCC |
| | | | GCAGCCGAACCTAAATCTAGTGACAAGACTCATACCTGCCCCCCTTGTCCAGCACCAGAGGCTGCAGGAGGACCTTCCGTGTTCCTGTTTCCACCCAAACCAAAGGATACTCTGATGATC |
| | | | TCCCGGACACCTGAAGTCACTTGCGTGGTCGTGAGCGTGTCTCACGAGGACCCCGAAGTCAAGTTTAACTGGTACGTGGACGGCGTCGAGGTGCATAATGCCAAAACCAAGCCCAGGGAG |
| | | | GAACAGTACAACTCCACATATCGCGTCGTGTCTGTCCTGACTGTGCTGCACCAGGATTGGCTGAACGGCAAGGAGTACAAATGCAAGGTGAGCAACAAGGCACTGCCTGCCCCAATCGAG |
| | | | AAGACAATTAGCAAAGCAAAGGGGCAGCCCCGAGAACCTCAGGTCTACGTGCTGCCTCCATCTCGGGACGAGCTGACTAAAAACCAGGTCAGTCTGCTGTGTCTGGTGAAGGGCTTCTAT |
| | | | CCAAGCGATATTGCTGTGGAGTGGGAATCCAATGGGCAGCCCGAAAACAATTACCTGACTTGGCCCCCTGTCCTGGACTCAGATGGGAGCTTCTTTCTGTATAGTAAACTGACCGTGGAC |
| | | | AAGTCACGGTGGCAGCAGGGAAACGTCTTTAGCTGTTCCGTGATGCATGAGGCCCTGCACAATCATTACACCCAGAAATCTCTGAGTCTGTCACCCGGCAAG |
| 3 | 2176 | VL | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN |
| 4 | 2176 | VL | CAGATCGTCCTGACACAGAGCCCAGCTATCATGTCAGCAAGCCCCGGCGAGAAAGTCACAATGACTTGCTCAGCCAGCTCCTCTGTGAGCTACATGAACTGGTATCAGCAGAAAAGCGGA |
| | | | ACCTCCCCCAAGAGATGGATCTACGACACATCCAAGCTGGCCTCTGGAGTGCCTGCTCACTTCAGGGGCAGCGGCTCTGGGACCAGTTATTCACTGACAATTTCCGGCATGGAGGCCGAA |
| | | | GATGCCGCTACCTACTATTGCCAGCAGTGGAGTTCAAACCCATTCACTTTTGGATCTGGCACCAAGCTGGAAATTAAT |
| 5 | 2176 | linker | GGGGSGGGGSGGGGS |
| 6 | 2176 | linker | GGCGGAGGAGGCTCCGGAGGAGGAGGGTCTGGAGGAGGAGGAAGT |
| 7 | 2176 | VH | QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS |
| 8 | 2176 | VH | CAGGTGCAGCTGCAGCAGTCCGGAGCAGAGCTGGCTCGACCAGGAGCTAGTGTGAAAATGTCCTGTAAGGCAAGCGGCTACACCTTCACACGGTATACCATGCATTGGGTGAAACAGAGA |
| | | | CCCGGGCAGGGACTGGAATGGATCGGGTACATTAATCCTAGCCGAGGATACACAAACTACAACCAGAAGTTTAAAGACAAGGCCACTCTGACCACAGATAAGAGCTCCTCTACCGCTTAT |
| | | | ATGCAGCTGAGTTCACTGACATCTGAGGACAGTGCAGTGTACTATTGCGCCAGGTACTATGACGATCACTACTGTCTGGATTATTGGGGCCAGGGGACTACCCTGACAGTGAGCTCC |
| 9 | 2176 | hinge | AAEPKSSDKTHTCPPCP |
| 10 | 2176 | hinge | GCAGCCGAACCTAAATCTAGTGACAAGACTCATACCTGCCCCCCTTGTCCA |
| 11 | 2176 | CH2 | APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK |
| 12 | 2176 | CH2 | GCACCAGAGGCTGCAGGAGGACCTTCCGTGTTCCTGTTTCCACCCAAACCAAAGGATACTCTGATGATCTCCCGGACACCTGAAGTCACTTGCGTGGTCGTGAGCGTGTCTCACGAGGAC |
| | | | CCCGAAGTCAAGTTTAACTGGTACGTGGACGGCGTCGAGGTGCATAATGCCAAAACCAAGCCCAGGGAGGAACAGTACAACTCCACATATCGCGTCGTGTCTGTCCTGACTGTGCTGCAC |
| | | | CAGGATTGGCTGAACGGCAAGGAGTACAAATGCAAGGTGAGCAACAAGGCACTGCCTGCCCCAATCGAGAAGACAATTAGCAAAGCAAAG |
| 13 | 2176 | CH3 | GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 14 | 2176 | CH3 | GGGCAGCCCCGAGAACCTCAGGTCTACGTGCTGCCTCCATCTCGGGACGAGCTGACTAAAAACCAGGTCAGTCTGCTGTGTCTGGTGAAGGGCTTCTATCCAAGCGATATTGCTGTGGAG |
| | | | TGGGAATCCAATGGGCAGCCCGAAAACAATTACCTGACTTGGCCCCCTGTCCTGGACTCAGATGGGAGCTTCTTTCTGTATAGTAAACTGACCGTGGACAAGTCACGGTGGCAGCAGGGA |
| | | | AACGTCTTTAGCTGTTCCGTGATGCATGAGGCCCTGCACAATCATTACACCCAGAAATCTCTGAGTCTGTCACCCGGC |
| 15 | 6689 | Full | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEINGGGGSGGGGSGGGG |
| | | | SQVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSS |
| | | | AAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIE |
| | | | KTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 16 | 6689 | Full | CAGATCGTCCTGACTCAGAGCCCCGCTATTATGTCCGCTTCCCCTGGAGAAAAGGTCACTATGACTTGTTCCGCCTCTAGTTCCGTCTCCTACATGAACTGGTATCAGCAGAAATCTGGA |
| | | | ACAAGTCCCAAGCGATGGATCTACGACACTTCCAAGCTGGCATCTGGAGTGCCTGCCCACTTCCGAGGCAGCGGCTCTGGGACAAGTTATTCACTGACTATTTCTGGCATGGAGGCCGAA |
| | | | GATGCCGCTACATACTATTGCCAGCAGTGGAGCTCCAACCCATTCACCTTTGGATGTGGCACAAAGCTGGAGATCAATGGCGGAGGAGGCTCCGGAGGAGGAGGGTCTGGAGGAGGAGGA |
| | | | AGTCAGGTCCAGCTGCAGCAGAGCGGAGCAGAACTGGCTAGACCAGGAGCCAGTGTGAAAATGTCATGCAAGGCCAGCGGCTACACATTCACTCGGTATACCATGCATTGGGTGAAACAG |
| | | | AGACCAGGACAGTGTCTGGAGTGGATCGGCTACATTAATCCCAGCAGGGGGTACACAAACTACAACCAGAAGTTTAAAGACAAGGCAACCCTGACCACCGATAAGTCTAGTTCAACAGCT |
| | | | TATATGCAGCTGAGCTCCCTGACTTCAGAAGACAGCGCTGTGTACTATTGCGCACGCTACTATGACGATCACTACTGTCTGGATTATTGGGGGCAGGGAACTACCCTGACCGTGTCTAGT |
| | | | GCAGCCGAGCCTAAATCAAGCGACAAGACCCATACATGCCCCCCTTGTCCGGCGCCAGAAGCTGCAGGCGGACCAAGCGTGTTCCTGTTTCCACCCAAACCTAAGGATACTCTGATGATT |
| | | | AGCCGAACTCCTGAGGTCACCTGCGTGGTCGTGAGCGTGTCCCACGAGGACCCAGAAGTCAAGTTCAACTGGTACGTGGATGGGGTCGAAGTGCATAATGCCAAAACCAAGCCCAGGGAG |
| | | | GAACAGTACAACTCCACTTATCGCGTCGTGTCTGTCCTGACCGTGCTGCACCAGGACTGGCTGAATGGCAAGGAGTACAAATGTAAGGTCTCAAATAAGGCTCTGCCCGCCCCTATCGAA |
| | | | AAAACTATCTCAAAGGCAAAAGGCCAGCCTCGCGAACCACAGGTCTACGTGCTGCCCCCTAGCCGCGACGAACTGACTAAAAATCAGGTCTCTCTGCTGTGTCTGGTCAAAGGATTCTAC |
| | | | CCTTCCGACATCGCCGTGGAGTGGGAAAGTAACGGCCAGCCCGAGAACAATTACCTGACCTGGCCCCCTGTGCTGGACTCTGATGGGAGTTTCTTTCTGTATTCAAAGCTGACAGTCGAT |
| | | | AAAAGCCGGTGGCAGCAGGGCAATGTGTTCAGCTGCTCCGTCATGCACGAAGCACTGCACAACCATTACACTCAGAAGTCCCTGTCCCTGTCACCTGGC |
| 17 | 6689 | VL | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEIN |
| 18 | 6689 | VL | CAGATCGTCCTGACTCAGAGCCCCGCTATTATGTCCGCTTCCCCTGGAGAAAAGGTCACTATGACTTGTTCCGCCTCTAGTTCCGTCTCCTACATGAACTGGTATCAGCAGAAATCTGGA |
| | | | ACAAGTCCCAAGCGATGGATCTACGACACTTCCAAGCTGGCATCTGGAGTGCCTGCCCACTTCCGAGGCAGCGGCTCTGGGACAAGTTATTCACTGACTATTTCTGGCATGGAGGCCGAA |
| | | | GATGCCGCTACATACTATTGCCAGCAGTGGAGCTCCAACCCATTCACCTTTGGATGTGGCACAAAGCTGGAGATCAAT |
| 19 | 6689 | linker | GGGGSGGGGSGGGGS |
| 20 | 6689 | linker | GGCGGAGGAGGCTCCGGAGGAGGAGGGTCTGGAGGAGGAGGAAGT |
| 21 | 6689 | VH | QVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSS |
| 22 | 6689 | VH | CAGGTCCAGCTGCAGCAGAGCGGAGCAGAACTGGCTAGACCAGGAGCCAGTGTGAAAATGTCATGCAAGGCCAGCGGCTACACATTCACTCGGTATACCATGCATTGGGTGAAACAGAGA |
| | | | CCAGGACAGTGTCTGGAGTGGATCGGCTACATTAATCCCAGCAGGGGGTACACAAACTACAACCAGAAGTTTAAAGACAAGGCAACCCTGACCACCGATAAGTCTAGTTCAACAGCTTAT |
| | | | ATGCAGCTGAGCTCCCTGACTTCAGAAGACAGCGCTGTGTACTATTGCGCACGCTACTATGACGATCACTACTGTCTGGATTATTGGGGGCAGGGAACTACCCTGACCGTGTCTAGT |
| 23 | 6689 | hinge | AAEPKSSDKTHTCPPCP |
| 24 | 6689 | hinge | GCAGCCGAGCCTAAATCAAGCGACAAGACCCATACATGCCCCCCTTGTCCG |
| 25 | 6689 | CH2 | APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK |
| 26 | 6689 | CH2 | GCGCCAGAAGCTGCAGGCGGACCAAGCGTGTTCCTGTTTCCACCCAAACCTAAGGATACTCTGATGATTAGCCGAACTCCTGAGGTCACCTGCGTGGTCGTGAGCGTGTCCCACGAGGAC |
| | | | CCAGAAGTCAAGTTCAACTGGTACGTGGATGGGGTCGAAGTGCATAATGCCAAAACCAAGCCCAGGGAGGAACAGTACAACTCCACTTATCGCGTCGTGTCTGTCCTGACCGTGCTGCAC |
| | | | CAGGACTGGCTGAATGGCAAGGAGTACAAATGTAAGGTCTCAAATAAGGCTCTGCCCGCCCCTATCGAAAAAACTATCTCAAAGGCAAAA |
| 27 | 6689 | CH3 | GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 28 | 6689 | CH3 | GGCCAGCCTCGCGAACCACAGGTCTACGTGCTGCCCCCTAGCCGCGACGAACTGACTAAAAATCAGGTCTCTCTGCTGTGTCTGGTCAAAGGATTCTACCCTTCCGACATCGCCGTGGAG |
| | | | TGGGAAAGTAACGGCCAGCCCGAGAACAATTACCTGACCTGGCCCCCTGTGCTGGACTCTGATGGGAGTTTCTTTCTGTATTCAAAGCTGACAGTCGATAAAAGCCGGTGGCAGCAGGGC |
| | | | AATGTGTTCAGCTGCTCCGTCATGCACGAAGCACTGCACAACCATTACACTCAGAAGTCCCTGTCCCTGTCACCTGGC |
| 29 | 1890 | Full | DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSV |
| | | | EGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKL |
| | | | ELKAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPA |
| | | | PIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK |
| 30 | 1890 | Full | GACATCAAACTGCAGCAGAGCGGAGCAGAGCTGGCTCGACCAGGAGCCAGTGTGAAAATGTCATGCAAGACCAGCGGCTACACATTCACTCGGTATACAATGCACTGGGTGAAGCAGAGA |
| | | | CCAGGACAGGGACTGGAATGGATCGGATATATTAACCCTTCCCGAGGCTACACAAACTACAACCAGAAGTTTAAAGACAAGGCAACTCTGACCACAGATAAGAGCTCCTCTACCGCCTAC |
| | | | ATGCAGCTGAGTTCACTGACAAGTGAGGACTCAGCCGTGTACTATTGCGCTAGGTACTATGACGATCATTACTGTCTGGATTATTGGGGACAGGGCACTACCCTGACTGTCAGCTCCGTG |
| | | | GAAGGAGGGAGCGGAGGCTCCGGAGGATCTGGCGGGAGTGGAGGCGTGGACGATATCCAGCTGACCCAGTCCCCAGCTATTATGTCCGCATCTCCCGGCGAGAAAGTCACCATGACATGC |
| | | | CGCGCCTCTAGTTCAGTGAGCTACATGAACTGGTATCAGCAGAAATCAGGCACTAGCCCCAAGAGATGGATCTACGACACCTCCAAGGTCGCTTCTGGGGTGCCTTATAGGTTCAGTGGG |
| | | | TCAGGAAGCGGCACCTCCTACTCTCTGACAATTAGCTCCATGGAGGCTGAAGATGCCGCTACCTACTATTGTCAGCAGTGGTCTAGTAATCCACTGACTTTTGGGGCAGGAACCAAACTG |
| | | | GAGCTGAAGGCAGCCGAACCCAAATCAAGCGACAAGACTCACACCTGCCCACCTTGTCCAGCACCAGAAGCTGCAGGAGGACCTAGCGTGTTCCTGTTTCCACCCAAACCAAAGGATACA |
| | | | CTGATGATCAGCCGGACACCTGAGGTCACTTGCGTGGTCGTGGACGTGAGCCACGAGGACCCCGAAGTCAAGTTCAACTGGTACGTGGACGGCGTCGAAGTGCATAATGCCAAAACCAAG |
| | | | CCTAGGGAGGAACAGTACAATAGTACATATAGAGTCGTGTCAGTGCTGACCGTCCTGCATCAGGATTGGCTGAACGGGAAGGAGTACAAATGCAAGGTGTCCAACAAGGCACTGCCTGCC |
| | | | CCAATCGAGAAGACCATTTCTAAAGCAAAGGGCCAGCCCCGAGAACCTCAGGTCTATGTGCTGCCTCCATCCCGGGACGAGCTGACAAAAAACCAGGTCTCTCTGCTGTGTCTGGTGAAG |
| | | | GGGTTCTACCCATCTGATATTGCTGTGGAGTGGGAAAGTAATGGACAGCCCGAGAACAATTATCTGACATGGCCCCCTGTGCTGGACTCCGATGGATCTTTCTTTCTGTACAGCAAACTG |
| | | | ACTGTGGACAAGTCCAGATGGCAGCAGGGCAACGTCTTTAGTTGTTCAGTGATGCACGAGGCCCTGCACAATCATTACACCCAGAAAAGCCTGTCCCTGTCTCCCGGCAAG |
| 31 | 1890 | VH | DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS |
| 32 | 1890 | VH | GACATCAAACTGCAGCAGAGCGGAGCAGAGCTGGCTCGACCAGGAGCCAGTGTGAAAATGTCATGCAAGACCAGCGGCTACACATTCACTCGGTATACAATGCACTGGGTGAAGCAGAGA |
| | | | CCAGGACAGGGACTGGAATGGATCGGATATATTAACCCTTCCCGAGGCTACACAAACTACAACCAGAAGTTTAAAGACAAGGCAACTCTGACCACAGATAAGAGCTCCTCTACCGCCTAC |
| | | | ATGCAGCTGAGTTCACTGACAAGTGAGGACTCAGCCGTGTACTATTGCGCTAGGTACTATGACGATCATTACTGTCTGGATTATTGGGGACAGGGCACTACCCTGACTGTCAGCTCC |
| 33 | 1890 | linker | GGSGGSGGSGGSGG |
| 34 | 1890 | linker | GGAGGGAGCGGAGGCTCCGGAGGATCTGGCGGGAGTGGAGGC |
| 35 | 1890 | VL | DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK |
| 36 | 1890 | VL | GATATCCAGCTGACCCAGTCCCCAGCTATTATGTCCGCATCTCCCGGCGAGAAAGTCACCATGACATGCCGCGCCTCTAGTTCAGTGAGCTACATGAACTGGTATCAGCAGAAATCAGGC |
| | | | ACTAGCCCCAAGAGATGGATCTACGACACCTCCAAGGTCGCTTCTGGGGTGCCTTATAGGTTCAGTGGGTCAGGAAGCGGCACCTCCTACTCTCTGACAATTAGCTCCATGGAGGCTGAA |
| | | | GATGCCGCTACCTACTATTGTCAGCAGTGGTCTAGTAATCCACTGACTTTTGGGGCAGGAACCAAACTGGAGCTGAAG |
| 37 | 1890 | hinge | AAEPKSSDKTHTCPPCP |
| 38 | 1890 | hinge | GCAGCCGAACCCAAATCAAGCGACAAGACTCACACCTGCCCACCTTGTCCA |
| 39 | 1890 | CH2 | APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK |
| 40 | 1890 | CH2 | GCACCAGAAGCTGCAGGAGGACCTAGCGTGTTCCTGTTTCCACCCAAACCAAAGGATACACTGATGATCAGCCGGACACCTGAGGTCACTTGCGTGGTCGTGGACGTGAGCCACGAGGAC |
| | | | CCCGAAGTCAAGTTCAACTGGTACGTGGACGGCGTCGAAGTGCATAATGCCAAAACCAAGCCTAGGGAGGAACAGTACAATAGTACATATAGAGTCGTGTCAGTGCTGACCGTCCTGCAT |
| | | | CAGGATTGGCTGAACGGGAAGGAGTACAAATGCAAGGTGTCCAACAAGGCACTGCCTGCCCCAATCGAGAAGACCATTTCTAAAGCAAAG |
| 41 | 1890 | CH3 | GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 42 | 1890 | CH3 | GGCCAGCCCCGAGAACCTCAGGTCTATGTGCTGCCTCCATCCCGGGACGAGCTGACAAAAAACCAGGTCTCTCTGCTGTGTCTGGTGAAGGGGTTCTACCCATCTGATATTGCTGTGGAG |
| | | | TGGGAAAGTAATGGACAGCCCGAGAACAATTATCTGACATGGCCCCCTGTGCTGGACTCCGATGGATCTTTCTTTCTGTACAGCAAACTGACTGTGGACAAGTCCAGATGGCAGCAGGGC |
| | | | AACGTCTTTAGTTGTTCAGTGATGCACGAGGCCCTGCACAATCATTACACCCAGAAAAGCCTGTCCCTGTCTCCCGGC |
| 43 | 6692 | Full | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGCGTKLEIKGGGGSGGGG |
| | | | SGGGGSQVQLQQSGAELVRPGSSVKISCKASGYAFSSYWMNWVKQRPGQCLEWIGQIWPGDGDTNYNGKFKGKATLTADESSSTAYMQLSSLASEDSAVYFCARRETTTVGRYYYAMDYW |
| | | | GQGTTVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKV |
| | | | SNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSL |
| | | | SPG |
| 44 | 6692 | Full | GACATCCAGCTGACACAGAGCCCCGCAAGCCTGGCCGTGAGCCTGGGACAGAGAGCCACTATTTCATGCAAAGCCTCACAGAGCGTGGACTATGATGGAGACAGCTATCTGAACTGGTAC |
| | | | CAGCAGATCCCAGGCCAGCCCCCTAAACTGCTGATCTACGACGCCAGCAATCTGGTGTCCGGCATCCCACCCAGGTTCAGTGGATCAGGCAGCGGGACCGATTTTACACTGAACATTCAC |
| | | | CCTGTCGAGAAGGTGGACGCCGCTACCTACCATTGCCAGCAGTCCACAGAGGACCCCTGGACTTTCGGATGTGGCACCAAACTGGAAATCAAGGGCGGGGGAGGCTCAGGAGGAGGAGGG |
| | | | AGCGGAGGAGGAGGCAGCCAGGTGCAGCTGCAGCAGAGCGGAGCAGAACTGGTCCGACCTGGAAGCTCCGTGAAAATTTCTTGCAAGGCCAGTGGCTATGCTTTTTCTAGTTACTGGATG |
| | | | AATTGGGTGAAGCAGCGACCAGGACAGTGTCTGGAGTGGATCGGGCAGATTTGGCCTGGGGATGGAGACACCAACTATAATGGAAAGTTCAAAGGCAAGGCAACTCTGACCGCCGACGAA |
| | | | TCAAGCTCCACAGCTTATATGCAGCTGTCTAGTCTGGCTAGTGAGGATTCAGCAGTGTACTTTTGCGCCCGGAGAGAAACCACAACTGTGGGCAGATACTATTACGCAATGGACTACTGG |
| | | | GGCCAGGGGACCACAGTCACCGTGTCAAGCGCAGCCGAGCCCAAATCCTCTGATAAGACACACACTTGCCCTCCATGTCCGGCGCCAGAAGCTGCAGGCGGACCTTCCGTGTTCCTGTTT |
| | | | CCCCCTAAACCAAAGGACACTCTGATGATCTCTCGCACTCCAGAGGTCACCTGCGTGGTCGTGTCCGTGTCTCACGAGGACCCCGAAGTCAAATTCAACTGGTATGTGGACGGGGTCGAA |
| | | | GTGCATAATGCCAAAACAAAGCCTAGGGAGGAACAGTATAACTCTACATACCGCGTCGTGAGTGTCCTGACTGTGCTGCATCAGGATTGGCTGAATGGCAAGGAGTACAAATGTAAGGTG |
| | | | AGCAACAAAGCACTGCCCGCCCCTATCGAAAAAACTATTAGCAAAGCAAAAGGACAGCCTCGCGAACCACAGGTCTACGTCTACCCCCCATCAAGAGATGAACTGACAAAAAATCAGGTC |
| | | | TCTCTGACATGCCTGGTCAAAGGATTCTACCCTTCCGACATCGCCGTGGAGTGGGAAAGTAACGGCCAGCCCGAGAACAATTACAAGACCACACCCCCTGTCCTGGACTCTGATGGGAGT |
| | | | TTCGCTCTGGTGTCAAAGCTGACCGTCGATAAAAGCCGGTGGCAGCAGGGCAATGTGTTTAGCTGCTCCGTCATGCACGAAGCCCTGCACAATCACTACACACAGAAGTCCCTGAGCCTG |
| | | | AGCCCTGGC |
| 45 | 6692 | VL | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGCGTKLEIK |
| 46 | 6692 | VL | GACATCCAGCTGACACAGAGCCCCGCAAGCCTGGCCGTGAGCCTGGGACAGAGAGCCACTATTTCATGCAAAGCCTCACAGAGCGTGGACTATGATGGAGACAGCTATCTGAACTGGTAC |
| | | | CAGCAGATCCCAGGCCAGCCCCCTAAACTGCTGATCTACGACGCCAGCAATCTGGTGTCCGGCATCCCACCCAGGTTCAGTGGATCAGGCAGCGGGACCGATTTTACACTGAACATTCAC |
| | | | CCTGTCGAGAAGGTGGACGCCGCTACCTACCATTGCCAGCAGTCCACAGAGGACCCCTGGACTTTCGGATGTGGCACCAAACTGGAAATCAAG |
| 47 | 6692 | linker | GGGGSGGGGSGGGGS |
| 48 | 6692 | linker | GGCGGGGGAGGCTCAGGAGGAGGAGGGAGCGGAGGAGGAGGCAGC |
| 49 | 6692 | VH | QVQLQQSGAELVRPGSSVKISCKASGYAFSSYWMNWVKQRPGQCLEWIGQIWPGDGDTNYNGKFKGKATLTADESSSTAYMQLSSLASEDSAVYFCARRETTTVGRYYYAMDYWGQGTTV |
| | | | TVSS |
| 50 | 6692 | VH | CAGGTGCAGCTGCAGCAGAGCGGAGCAGAACTGGTCCGACCTGGAAGCTCCGTGAAAATTTCTTGCAAGGCCAGTGGCTATGCTTTTTCTAGTTACTGGATGAATTGGGTGAAGCAGCGA |
| | | | CCAGGACAGTGTCTGGAGTGGATCGGGCAGATTTGGCCTGGGGATGGAGACACCAACTATAATGGAAAGTTCAAAGGCAAGGCAACTCTGACCGCCGACGAATCAAGCTCCACAGCTTAT |
| | | | ATGCAGCTGTCTAGTCTGGCTAGTGAGGATTCAGCAGTGTACTTTTGCGCCCGGAGAGAAACCACAACTGTGGGCAGATACTATTACGCAATGGACTACTGGGGCCAGGGGACCACAGTC |
| | | | ACCGTGTCAAGC |
| 51 | 6692 | hinge | AAEPKSSDKTHTCPPCP |
| 52 | 6692 | hinge | GCAGCCGAGCCCAAATCCTCTGATAAGACACACACTTGCCCTCCATGTCCG |
| 53 | 6692 | CH2 | APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK |
| 54 | 6692 | CH2 | GCGCCAGAAGCTGCAGGCGGACCTTCCGTGTTCCTGTTTCCCCCTAAACCAAAGGACACTCTGATGATCTCTCGCACTCCAGAGGTCACCTGCGTGGTCGTGTCCGTGTCTCACGAGGAC |
| | | | CCCGAAGTCAAATTCAACTGGTATGTGGACGGGGTCGAAGTGCATAATGCCAAAACAAAGCCTAGGGAGGAACAGTATAACTCTACATACCGCGTCGTGAGTGTCCTGACTGTGCTGCAT |
| | | | CAGGATTGGCTGAATGGCAAGGAGTACAAATGTAAGGTGAGCAACAAAGCACTGCCCGCCCCTATCGAAAAAACTATTAGCAAAGCAAAA |
| 55 | 6692 | CH3 | GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG |
| 56 | 6692 | CH3 | GGACAGCCTCGCGAACCACAGGTCTACGTCTACCCCCCATCAAGAGATGAACTGACAAAAAATCAGGTCTCTCTGACATGCCTGGTCAAAGGATTCTACCCTTCCGACATCGCCGTGGAG |
| | | | TGGGAAAGTAACGGCCAGCCCGAGAACAATTACAAGACCACACCCCCTGTCCTGGACTCTGATGGGAGTTTCGCTCTGGTGTCAAAGCTGACCGTCGATAAAAGCCGGTGGCAGCAGGGC |
| | | | AATGTGTTTAGCTGCTCCGTCATGCACGAAGCCCTGCACAATCACTACACACAGAAGTCCCTGAGCCTGAGCCCTGGC |
| 57 | 2183 | Full | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGG |
| | | | SGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYW |
| | | | GQGTTVTVSSAAEPKSSDKTHTCPPCPAPEAAGGPSVFLFPPKPKDTLMISRIPEVICVVVSVSHEDPEVKFNWEVDGVEVHNAKTKPREEQYNSTYRVVSVLTVLHQDWLNGKEYKCKV |
| | | | SNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSL |
| | | | SPGK |
| 58 | 2183 | Full | GATATTCAGCTGACACAGAGTCCTGCATCACTGGCTGTGAGCCTGGGACAGCGAGCAACTATCTCCTGCAAAGCCAGTCAGTCAGTGGACTATGATGGCGACTCCTATCTGAACTGGTAC |
| | | | CAGCAGATCCCAGGGCAGCCCCCTAAGCTGCTGATCTACGACGCCTCAAATCTGGTGAGCGGCATCCCACCACGATTCAGCGGCAGCGGCTCTGGGACTGATTTTACCCTGAACATTCAC |
| | | | CCAGTCGAGAAGGTGGACGCCGCTACCTACCATTGCCAGCAGTCTACCGAGGACCCCTGGACATTCGGCGGGGGAACTAAACTGGAAATCAAGGGAGGAGGAGGCAGTGGCGGAGGAGGG |
| | | | TCAGGAGGAGGAGGAAGCCAGGTGCAGCTGCAGCAGAGCGGAGCAGAGCTGGTCAGACCAGGAAGCTCCGTGAAAATTTCCTGTAAGGCTTCTGGCTATGCATTTTCTAGTTACTGGATG |
| | | | AATTGGGTGAAGCAGAGGCCAGGACAGGGCCTGGAATGGATCGGGCAGATTTGGCCCGGGGATGGAGACACCAACTATAATGGAAAGTTCAAAGGCAAGGCCACACTGACTGCTGACGAG |
| | | | TCAAGCTCCACAGCCTATATGCAGCTGTCTAGTCTGGCAAGCGAGGATTCCGCCGTGTACTTTTGCGCTCGGAGAGAAACCACAACTGTGGGCAGGTACTATTACGCTATGGACTACTGG |
| | | | GGCCAGGGGACCACAGTCACCGTGTCAAGCGCAGCCGAACCCAAATCCTCTGATAAGACCCACACATGCCCTCCATGTCCAGCTCCTGAGGCTGCAGGAGGACCAAGCGTGTTCCTGTTT |
| | | | CCCCCTAAACCTAAGGACACACTGATGATCTCTCGGACACCCGAAGTCACTTGTGTGGTCGTGAGCGTGAGCCACGAGGACCCTGAAGTCAAATTCAACTGGTACGTGGATGGCGTCGAG |
| | | | GTGCATAATGCCAAAACTAAGCCTAGGGAGGAACAGTATAACTCCACTTACCGCGTCGTGTCTGTCCTGACCGTGCTGCATCAGGACTGGCTGAACGGAAAGGAGTACAAATGCAAGGTG |
| | | | AGCAACAAGGCACTGCCAGCCCCCATCGAGAAGACAATTTCCAAAGCAAAGGGCCAGCCTCGAGAACCACAGGTCTATGTGTACCCACCCAGCCGGGACGAGCTGACCAAAAACCAGGTC |
| | | | TCCCTGACATGTCTGGTGAAGGGATTTTATCCTTCTGATATTGCCGTGGAGTGGGAAAGTAATGGCCAGCCAGAAAACAATTACAAGACTACCCCTCCAGTGCTGGATTCTGACGGGAGT |
| | | | TTCGCTCTGGTCAGTAAACTGACTGTGGATAAGTCACGGTGGCAGCAGGGAAACGTCTTTAGTTGTTCAGTGATGCACGAGGCACTGCACAATCATTACACCCAGAAAAGCCTGTCCCTG |
| | | | TCTCCCGGCAAG |
| 59 | 2183 | VL | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK |
| 60 | 2183 | VL | GATATTCAGCTGACACAGAGTCCTGCATCACTGGCTGTGAGCCTGGGACAGCGAGCAACTATCTCCTGCAAAGCCAGTCAGTCAGTGGACTATGATGGCGACTCCTATCTGAACTGGTAC |
| | | | CAGCAGATCCCAGGGCAGCCCCCTAAGCTGCTGATCTACGACGCCTCAAATCTGGTGAGCGGCATCCCACCACGATTCAGCGGCAGCGGCTCTGGGACTGATTTTACCCTGAACATTCAC |
| | | | CCAGTCGAGAAGGTGGACGCCGCTACCTACCATTGCCAGCAGTCTACCGAGGACCCCTGGACATTCGGCGGGGGAACTAAACTGGAAATCAAG |
| 61 | 2183 | linker | GGGGSGGGGSGGGGS |
| 62 | 2183 | linker | GGAGGAGGAGGCAGTGGCGGAGGAGGGTCAGGAGGAGGAGGAAGC |
| 63 | 2183 | VH | QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTV |
| | | | TVSS |
| 64 | 2183 | VH | CAGGTGCAGCTGCAGCAGAGCGGAGCAGAGCTGGTCAGACCAGGAAGCTCCGTGAAAATTTCCTGTAAGGCTTCTGGCTATGCATTTTCTAGTTACTGGATGAATTGGGTGAAGCAGAGG |
| | | | CCAGGACAGGGCCTGGAATGGATCGGGCAGATTTGGCCCGGGGATGGAGACACCAACTATAATGGAAAGTTCAAAGGCAAGGCCACACTGACTGCTGACGAGTCAAGCTCCACAGCCTAT |
| | | | ATGCAGCTGTCTAGTCTGGCAAGCGAGGATTCCGCCGTGTACTTTTGCGCTCGGAGAGAAACCACAACTGTGGGCAGGTACTATTACGCTATGGACTACTGGGGCCAGGGGACCACAGTC |
| | | | ACCGTGTCAAGC |
| 65 | 2183 | hinge | AAEPKSSDKTHTCPPCP |
| 66 | 2183 | hinge | GCAGCCGAACCCAAATCCTCTGATAAGACCCACACATGCCCTCCATGTCCA |
| 67 | 2183 | CH2 | APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK |
| 68 | 2183 | CH2 | GCTCCTGAGGCTGCAGGAGGACCAAGCGTGTTCCTGTTTCCCCCTAAACCTAAGGACACACTGATGATCTCTCGGACACCCGAAGTCACTTGTGTGGTCGTGAGCGTGAGCCACGAGGAC |
| | | | CCTGAAGTCAAATTCAACTGGTACGTGGATGGCGTCGAGGTGCATAATGCCAAAACTAAGCCTAGGGAGGAACAGTATAACTCCACTTACCGCGTCGTGTCTGTCCTGACCGTGCTGCAT |
| | | | CAGGACTGGCTGAACGGAAAGGAGTACAAATGCAAGGTGAGCAACAAGGCACTGCCAGCCCCCATCGAGAAGACAATTTCCAAAGCAAAG |
| 69 | 2183 | CH3 | GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG |
| 70 | 2183 | CH3 | GGCCAGCCTCGAGAACCACAGGTCTATGTGTACCCACCCAGCCGGGACGAGCTGACCAAAAACCAGGTCTCCCTGACATGTCTGGTGAAGGGATTTTATCCTTCTGATATTGCCGTGGAG |
| | | | TGGGAAAGTAATGGCCAGCCAGAAAACAATTACAAGACTACCCCTCCAGTGCTGGATTCTGACGGGAGTTTCGCTCTGGTCAGTAAACTGACTGTGGATAAGTCACGGTGGCAGCAGGGA |
| | | | AACGTCTTTAGTTGTTCAGTGATGCACGAGGCACTGCACAATCATTACACCCAGAAAAGCCTGTCCCTGTCTCCCGGC |
| 71 | 1064 | Full | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGG |
| | | | SGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYW |
| | | | GQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKV |
| | | | SNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSL |
| | | | SPGK |
| 72 | 1064 | Full | GACATTCAGCTGACACAGAGTCCTGCTTCACTGGCAGTGAGCCTGGGACAGCGAGCAACTATCTCCTGCAAAGCTAGTCAGTCAGTGGACTATGATGGCGACTCCTATCTGAACTGGTAC |
| | | | CAGCAGATCCCAGGGCAGCCCCCTAAGCTGCTGATCTACGACGCCTCAAATCTGGTGAGCGGCATCCCACCACGATTCAGCGGCAGCGGCTCTGGGACTGATTTTACCCTGAACATTCAC |
| | | | CCAGTCGAGAAGGTGGACGCCGCTACCTACCATTGCCAGCAGTCTACCGAGGACCCCTGGACATTCGGCGGGGGAACTAAACTGGAAATCAAGGGAGGAGGAGGCAGTGGCGGAGGAGGG |
| | | | TCAGGAGGAGGAGGAAGCCAGGTGCAGCTGCAGCAGAGCGGAGCAGAGCTGGTCAGACCAGGAAGCTCCGTGAAAATTTCCTGTAAGGCATCTGGCTATGCCTTTTCTAGTTACTGGATG |
| | | | AATTGGGTGAAGCAGAGGCCAGGACAGGGCCTGGAATGGATCGGGCAGATTTGGCCCGGGGATGGAGACACTAACTATAATGGAAAGTTCAAAGGCAAGGCTACACTGACTGCAGACGAG |
| | | | TCAAGCTCCACCGCTTATATGCAGCTGTCTAGTCTGGCCAGCGAGGATTCCGCTGTCTACTTTTGCGCACGGAGAGAAACCACAACTGTGGGCAGGTACTATTACGCAATGGACTACTGG |
| | | | GGCCAGGGGACCACAGTCACCGTGTCAAGCGCAGCCGAACCCAAATCCTCTGATAAGACCCACACATGCCCTCCATGTCCAGCACCTGAGCTGCTGGGAGGACCAAGCGTGTTCCTGTTT |
| | | | CCACCTAAACCTAAGGACACCCTGATGATCTCTCGGACACCCGAAGTCACTTGTGTGGTCGTGGATGTGAGCCACGAGGACCCTGAAGTCAAATTCAACTGGTACGTGGATGGCGTCGAG |
| | | | GTGCATAATGCCAAAACAAAGCCTAGGGAGGAACAGTATAACTCCACTTACCGCGTCGTGTCTGTCCTGACCGTGCTGCATCAGGACTGGCTGAACGGAAAGGAGTACAAATGCAAGGTG |
| | | | AGCAACAAGGCCCTGCCAGCTCCCATCGAGAAGACCATTTCCAAAGCTAAGGGCCAGCCTCGAGAACCACAGGTGTATACATACCCACCCAGCCGGGACGAGCTGACCAAAAACCAGGTC |
| | | | TCCCTGACATGTCTGGTGAAGGGATTTTATCCTTCTGATATTGCCGTGGAGTGGGAAAGTAATGGCCAGCCAGAAAACAATTACAAGACTACCCCTCCAGTGCTGGATTCTGACGGGAGT |
| | | | TTCGCACTGGTCAGTAAACTGACAGTGGATAAGTCACGGTGGCAGCAGGGAAACGTCTTTAGTTGTTCAGTGATGCACGAGGCCCTGCACAATCATTACACTCAGAAAAGCCTGTCCCTG |
| | | | TCTCCCGGCAAG |
| 73 | 1064 | VL | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK |
| 74 | 1064 | VL | GACATTCAGCTGACACAGAGTCCTGCTTCACTGGCAGTGAGCCTGGGACAGCGAGCAACTATCTCCTGCAAAGCTAGTCAGTCAGTGGACTATGATGGCGACTCCTATCTGAACTGGTAC |
| | | | CAGCAGATCCCAGGGCAGCCCCCTAAGCTGCTGATCTACGACGCCTCAAATCTGGTGAGCGGCATCCCACCACGATTCAGCGGCAGCGGCTCTGGGACTGATTTTACCCTGAACATTCAC |
| | | | CCAGTCGAGAAGGTGGACGCCGCTACCTACCATTGCCAGCAGTCTACCGAGGACCCCTGGACATTCGGCGGGGGAACTAAACTGGAAATCAAG |
| 75 | 1064 | linker | GGGGSGGGGSGGGGS |
| 76 | 1064 | linker | GGAGGAGGAGGCAGTGGCGGAGGAGGGTCAGGAGGAGGAGGAAGC |
| 77 | 1064 | VH | QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTV |
| | | | TVSS |
| 78 | 1064 | VH | CAGGTGCAGCTGCAGCAGAGCGGAGCAGAGCTGGTCAGACCAGGAAGCTCCGTGAAAATTTCCTGTAAGGCATCTGGCTATGCCTTTTCTAGTTACTGGATGAATTGGGTGAAGCAGAGG |
| | | | CCAGGACAGGGCCTGGAATGGATCGGGCAGATTTGGCCCGGGGATGGAGACACTAACTATAATGGAAAGTTCAAAGGCAAGGCTACACTGACTGCAGACGAGTCAAGCTCCACCGCTTAT |
| | | | ATGCAGCTGTCTAGTCTGGCCAGCGAGGATTCCGCTGTCTACTTTTGCGCACGGAGAGAAACCACAACTGTGGGCAGGTACTATTACGCAATGGACTACTGGGGCCAGGGGACCACAGTC |
| | | | ACCGTGTCAAGC |
| 79 | 1064 | hinge | AAEPKSSDKTHTCPPCP |
| 80 | 1064 | hinge | GCAGCCGAACCCAAATCCTCTGATAAGACCCACACATGCCCTCCATGTCCA |
| 81 | 1064 | CH2 | APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK |
| 82 | 1064 | CH2 | GCACCTGAGCTGCTGGGAGGACCAAGCGTGTTCCTGTTTCCACCTAAACCTAAGGACACCCTGATGATCTCTCGGACACCCGAAGTCACTTGTGTGGTCGTGGATGTGAGCCACGAGGAC |
| | | | CCTGAAGTCAAATTCAACTGGTACGTGGATGGCGTCGAGGTGCATAATGCCAAAACAAAGCCTAGGGAGGAACAGTATAACTCCACTTACCGCGTCGTGTCTGTCCTGACCGTGCTGCAT |
| | | | CAGGACTGGCTGAACGGAAAGGAGTACAAATGCAAGGTGAGCAACAAGGCCCTGCCAGCTCCCATCGAGAAGACCATTTCCAAAGCTAAG |
| 83 | 1064 | CH3 | GQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG |
| 84 | 1064 | CH3 | GGCCAGCCTCGAGAACCACAGGTGTATACATACCCACCCAGCCGGGACGAGCTGACCAAAAACCAGGTCTCCCTGACATGTCTGGTGAAGGGATTTTATCCTTCTGATATTGCCGTGGAG |
| | | | TGGGAAAGTAATGGCCAGCCAGAAAACAATTACAAGACTACCCCTCCAGTGCTGGATTCTGACGGGAGTTTCGCACTGGTCAGTAAACTGACAGTGGATAAGTCACGGTGGCAGCAGGGA |
| | | | AACGTCTTTAGTTGTTCAGTGATGCACGAGGCCCTGCACAATCATTACACTCAGAAAAGCCTGTCCCTGTCTCCCGGC |
| 85 | 2185 | Full | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGG |
| | | | SGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYW |
| | | | GQGTTVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSL |
| | | | SPGK |
| 86 | 2185 | Full | GATATTCAGCTGACCCAGAGTCCTGCATCACTGGCTGTGAGCCTGGGACAGCGAGCAACAATCTCCTGCAAAGCCAGTCAGTCAGTGGACTATGATGGCGACTCCTATCTGAACTGGTAC |
| | | | CAGCAGATCCCAGGGCAGCCCCCTAAGCTGCTGATCTACGACGCTTCAAATCTGGTGAGCGGCATCCCACCACGATTCAGCGGCAGCGGCTCTGGAACCGATTTTACACTGAACATTCAC |
| | | | CCAGTCGAGAAGGTGGACGCCGCTACCTACCATTGCCAGCAGTCTACAGAGGACCCCTGGACTTTCGGCGGGGGAACCAAACTGGAAATCAAGGGAGGAGGAGGCAGTGGCGGAGGAGGG |
| | | | TCAGGAGGAGGAGGAAGCCAGGTGCAGCTGCAGCAGAGCGGAGCAGAGCTGGTCAGACCAGGAAGCTCCGTGAAAATTTCCTGTAAGGCTTCTGGCTATGCATTTTCTAGTTACTGGATG |
| | | | AATTGGGTGAAGCAGAGGCCAGGACAGGGCCTGGAATGGATCGGGCAGATTTGGCCCGGGGATGGAGACACAAACTATAATGGAAAGTTCAAAGGCAAGGCCACTCTGACCGCTGACGAG |
| | | | TCAAGCTCCACTGCTTATATGCAGCTGTCTAGTCTGGCAAGCGAGGATTCCGCCGTCTACTTTTGCGCTCGGAGAGAAACCACAACTGTGGGCAGGTACTATTACGCAATGGACTACTGG |
| | | | GGCCAGGGGACCACAGTCACCGTGTCAAGCGCAGCCGAACCCAAATCCTCTGATAAGACACACACTTGCCCTCCATGTCCAGCACCTGAGGCTGCAGGAGGACCAAGCGTGTTCCTGTTT |
| | | | CCCCCTAAACCTAAGGACACTCTGATGATCTCTCGGACTCCCGAAGTCACCTGTGTGGTCGTGAGCGTGAGCCACGAGGACCCTGAAGTCAAATTCAACTGGTACGTGGATGGCGTCGAG |
| | | | GTGCATAATGCCAAAACAAAGCCTAGGGAGGAACAGTATAACTCCACATACCGCGTCGTGTCTGTCCTGACTGTGCTGCATCAGGACTGGCTGAACGGAAAGGAGTACAAATGCAAGGTG |
| | | | AGCAACAAGGCACTGCCAGCCCCCATCGAGAAGACCATTTCCAAAGCCAAGGGCCAGCCTCGAGAACCACAGGTCTATGTGCTGCCACCCAGCCGGGACGAGCTGACAAAAAACCAGGTC |
| | | | TCCCTGCTGTGTCTGGTGAAGGGATTCTACCCTTCTGATATTGCTGTGGAGTGGGAAAGTAATGGCCAGCCAGAAAACAATTATCTGACTTGGCCTCCAGTGCTGGATTCTGACGGGAGT |
| | | | TTCTTTCTGTACAGTAAACTGACCGTGGATAAGTCACGGTGGCAGCAGGGAAACGTCTTTAGTTGTTCAGTGATGCACGAGGCCCTGCACAATCATTACACCCAGAAAAGCCTGTCCCTG |
| | | | TCTCCCGGCAAG |
| 87 | 2185 | VL | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK |
| 88 | 2185 | VL | GATATTCAGCTGACCCAGAGTCCTGCATCACTGGCTGTGAGCCTGGGACAGCGAGCAACAATCTCCTGCAAAGCCAGTCAGTCAGTGGACTATGATGGCGACTCCTATCTGAACTGGTAC |
| | | | CAGCAGATCCCAGGGCAGCCCCCTAAGCTGCTGATCTACGACGCTTCAAATCTGGTGAGCGGCATCCCACCACGATTCAGCGGCAGCGGCTCTGGAACCGATTTTACACTGAACATTCAC |
| | | | CCAGTCGAGAAGGTGGACGCCGCTACCTACCATTGCCAGCAGTCTACAGAGGACCCCTGGACTTTCGGCGGGGGAACCAAACTGGAAATCAAG |
| 89 | 2185 | linker | GGGGSGGGGSGGGGS |
| 90 | 2185 | linker | GGAGGAGGAGGCAGTGGCGGAGGAGGGTCAGGAGGAGGAGGAAGC |
| 91 | 2185 | VH | QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTV |
| | | | TVSS |
| 92 | 2185 | VH | CAGGTGCAGCTGCAGCAGAGCGGAGCAGAGCTGGTCAGACCAGGAAGCTCCGTGAAAATTTCCTGTAAGGCTTCTGGCTATGCATTTTCTAGTTACTGGATGAATTGGGTGAAGCAGAGG |
| | | | CCAGGACAGGGCCTGGAATGGATCGGGCAGATTTGGCCCGGGGATGGAGACACAAACTATAATGGAAAGTTCAAAGGCAAGGCCACTCTGACCGCTGACGAGTCAAGCTCCACTGCTTAT |
| | | | ATGCAGCTGTCTAGTCTGGCAAGCGAGGATTCCGCCGTCTACTTTTGCGCTCGGAGAGAAACCACAACTGTGGGCAGGTACTATTACGCAATGGACTACTGGGGCCAGGGGACCACAGTC |
| | | | ACCGTGTCAAGC |
| 93 | 2185 | hinge | AAEPKSSDKTHTCPPCP |
| 94 | 2185 | hinge | GCAGCCGAACCCAAATCCTCTGATAAGACACACACTTGCCCTCCATGTCCA |
| 95 | 2185 | CH2 | APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK |
| 96 | 2185 | CH2 | GCACCTGAGGCTGCAGGAGGACCAAGCGTGTTCCTGTTTCCCCCTAAACCTAAGGACACTCTGATGATCTCTCGGACTCCCGAAGTCACCTGTGTGGTCGTGAGCGTGAGCCACGAGGAC |
| | | | CCTGAAGTCAAATTCAACTGGTACGTGGATGGCGTCGAGGTGCATAATGCCAAAACAAAGCCTAGGGAGGAACAGTATAACTCCACATACCGCGTCGTGTCTGTCCTGACTGTGCTGCAT |
| | | | CAGGACTGGCTGAACGGAAAGGAGTACAAATGCAAGGTGAGCAACAAGGCACTGCCAGCCCCCATCGAGAAGACCATTTCCAAAGCCAAG |
| 97 | 2185 | CH3 | GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 98 | 2185 | CH3 | GGCCAGCCTCGAGAACCACAGGTCTATGTGCTGCCACCCAGCCGGGACGAGCTGACAAAAAACCAGGTCTCCCTGCTGTGTCTGGTGAAGGGATTCTACCCTTCTGATATTGCTGTGGAG |
| | | | TGGGAAAGTAATGGCCAGCCAGAAAACAATTATCTGACTTGGCCTCCAGTGCTGGATTCTGACGGGAGTTTCTTTCTGTACAGTAAACTGACCGTGGATAAGTCACGGTGGCAGCAGGGA |
| | | | AACGTCTTTAGTTGTTCAGTGATGCACGAGGCCCTGCACAATCATTACACCCAGAAAAGCCTGTCCCTGTCTCCCGGC |
| 99 | 1067 | Full | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGG |
| | | | SQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS |
| | | | AAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIE |
| | | | KTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK |
| 100 | 1067 | Full | CAGATCGTCCTGACACAGAGCCCAGCAATCATGTCAGCCAGCCCCGGCGAGAAAGTCACAATGACTTGCTCAGCAAGCTCCTCTGTGAGCTACATGAACTGGTATCAGCAGAAAAGCGGA |
| | | | ACCTCCCCCAAGAGATGGATCTACGACACATCCAAGCTGGCTTCTGGAGTGCCTGCACACTTCAGGGGCAGCGGCTCTGGGACCAGTTATTCACTGACAATTTCCGGCATGGAGGCTGAA |
| | | | GATGCCGCTACCTACTATTGCCAGCAGTGGAGTTCAAACCCATTCACTTTTGGATCTGGCACCAAGCTGGAAATTAATGGCGGAGGAGGCTCCGGAGGAGGAGGGTCTGGAGGAGGAGGA |
| | | | AGTCAGGTCCAGCTGCAGCAGTCCGGAGCTGAGCTGGCACGACCAGGAGCAAGTGTGAAAATGTCCTGTAAGGCCAGCGGCTACACCTTCACACGGTATACCATGCATTGGGTGAAACAG |
| | | | AGACCCGGGCAGGGACTGGAATGGATCGGGTACATTAATCCTAGCCGAGGATACACAAACTACAACCAGAAGTTTAAAGACAAGGCTACTCTGACCACAGATAAGAGCTCCTCTACCGCA |
| | | | TATATGCAGCTGAGTTCACTGACATCTGAGGACAGTGCCGTGTACTATTGCGCTAGGTACTATGACGATCACTACTGTCTGGATTATTGGGGCCAGGGGACTACCCTGACCGTGAGCTCC |
| | | | GCAGCCGAACCTAAATCTAGTGACAAGACTCATACCTGCCCCCCTTGTCCAGCACCAGAGCTGCTGGGAGGACCTTCCGTGTTCCTGTTTCCACCCAAACCAAAGGATACTCTGATGATC |
| | | | TCCCGGACACCTGAAGTCACTTGCGTGGTCGTGGACGTGTCTCACGAGGACCCCGAAGTCAAGTTTAACTGGTACGTGGACGGCGTCGAGGTGCATAATGCCAAAACCAAGCCCAGGGAG |
| | | | GAACAGTACAACTCCACATATCGCGTCGTGTCTGTCCTGACTGTGCTGCACCAGGATTGGCTGAACGGCAAGGAGTACAAATGCAAGGTGAGCAACAAGGCCCTGCCTGCTCCAATCGAG |
| | | | AAGACAATTAGCAAAGCCAAGGGGCAGCCCCGAGAACCTCAGGTGTACACTCTGCCTCCATCTCGGGACGAGCTGACCAAAAACCAGGTCAGTCTGCTGTGTCTGGTGAAGGGCTTCTAT |
| | | | CCAAGCGATATTGCTGTGGAGTGGGAATCCAATGGGCAGCCCGAAAACAATTACATGACATGGCCCCCTGTCCTGGACTCAGATGGGAGCTTCTTTCTGTATAGTAAACTGACTGTGGAC |
| | | | AAGTCACGGTGGCAGCAGGGAAACGTCTTTAGCTGTTCCGTGATGCATGAGGCCCTGCACAATCATTACACCCAGAAATCTCTGAGTCTGTCACCCGGCAAG |
| 101 | 1067 | VL | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN |
| 102 | 1067 | VL | CAGATCGTCCTGACACAGAGCCCAGCAATCATGTCAGCCAGCCCCGGCGAGAAAGTCACAATGACTTGCTCAGCAAGCTCCTCTGTGAGCTACATGAACTGGTATCAGCAGAAAAGCGGA |
| | | | ACCTCCCCCAAGAGATGGATCTACGACACATCCAAGCTGGCTTCTGGAGTGCCTGCACACTTCAGGGGCAGCGGCTCTGGGACCAGTTATTCACTGACAATTTCCGGCATGGAGGCTGAA |
| | | | GATGCCGCTACCTACTATTGCCAGCAGTGGAGTTCAAACCCATTCACTTTTGGATCTGGCACCAAGCTGGAAATTAAT |
| 103 | 1067 | linker | GGGGSGGGGSGGGGS |
| 104 | 1067 | linker | GGCGGAGGAGGCTCCGGAGGAGGAGGGTCTGGAGGAGGAGGAAGT |
| 105 | 1067 | VH | QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS |
| 106 | 1067 | VH | CAGGTCCAGCTGCAGCAGTCCGGAGCTGAGCTGGCACGACCAGGAGCAAGTGTGAAAATGTCCTGTAAGGCCAGCGGCTACACCTTCACACGGTATACCATGCATTGGGTGAAACAGAGA |
| | | | CCCGGGCAGGGACTGGAATGGATCGGGTACATTAATCCTAGCCGAGGATACACAAACTACAACCAGAAGTTTAAAGACAAGGCTACTCTGACCACAGATAAGAGCTCCTCTACCGCATAT |
| | | | ATGCAGCTGAGTTCACTGACATCTGAGGACAGTGCCGTGTACTATTGCGCTAGGTACTATGACGATCACTACTGTCTGGATTATTGGGGCCAGGGGACTACCCTGACCGTGAGCTCC |
| 107 | 1067 | hinge | AAEPKSSDKTHTCPPCP |
| 108 | 1067 | hinge | GCAGCCGAACCTAAATCTAGTGACAAGACTCATACCTGCCCCCCTTGTCCA |
| 109 | 1067 | CH2 | APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK |
| 110 | 1067 | CH2 | GCACCAGAGCTGCTGGGAGGACCTTCCGTGTTCCTGTTTCCACCCAAACCAAAGGATACTCTGATGATCTCCCGGACACCTGAAGTCACTTGCGTGGTCGTGGACGTGTCTCACGAGGAC |
| | | | CCCGAAGTCAAGTTTAACTGGTACGTGGACGGCGTCGAGGTGCATAATGCCAAAACCAAGCCCAGGGAGGAACAGTACAACTCCACATATCGCGTCGTGTCTGTCCTGACTGTGCTGCAC |
| | | | CAGGATTGGCTGAACGGCAAGGAGTACAAATGCAAGGTGAGCAACAAGGCCCTGCCTGCTCCAATCGAGAAGACAATTAGCAAAGCCAAG |
| 111 | 1067 | CH3 | GQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPG |
| 112 | 1067 | CH3 | GGGCAGCCCCGAGAACCTCAGGTGTACACTCTGCCTCCATCTCGGGACGAGCTGACCAAAAACCAGGTCAGTCTGCTGTGTCTGGTGAAGGGCTTCTATCCAAGCGATATTGCTGTGGAG |
| | | | TGGGAATCCAATGGGCAGCCCGAAAACAATTACATGACATGGCCCCCTGTCCTGGACTCAGATGGGAGCTTCTTTCTGTATAGTAAACTGACTGTGGACAAGTCACGGTGGCAGCAGGGA |
| | | | AACGTCTTTAGCTGTTCCGTGATGCATGAGGCCCTGCACAATCATTACACCCAGAAATCTCTGAGTCTGTCACCCGGC |
| 113 | 2184 | Full | QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSS |
| | | | STGGGGSGGGGSGGGGSDIQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGT |
| | | | KLEINRAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKA |
| | | | LPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK |
| 114 | 2184 | Full | CAGGTCCAGCTGCAGCAGAGCGGAGCAGAGCTGGCTCGACCAGGAGCTAGTGTGAAAATGTCATGCAAGGCAAGCGGCTACACCTTCACACGGTATACTATGCACTGGGTGAAACAGAGA |
| | | | CCCGGACAGGGCCTGGAATGGATCGGGTACATTAACCCTAGCCGAGGATACACCAACTACAACCAGAAGTTTAAAGACAAGGCCACCCTGACCACAGATAAGAGCTCCTCTACAGCTTAT |
| | | | ATGCAGCTGAGTTCACTGACTTCTGAGGACAGTGCCGTGTACTATTGTGCTCGGTACTATGACGATCATTACTCCCTGGATTATTGGGGGCAGGGAACTACCCTGACCGTGAGCTCCTCT |
| | | | AGTACAGGAGGAGGAGGCAGTGGAGGAGGAGGGTCAGGCGGAGGAGGAAGCGACATCCAGATTGTGCTGACACAGTCTCCAGCTATCATGTCCGCATCTCCCGGCGAGAAAGTCACTATG |
| | | | ACCTGCTCCGCCTCAAGCTCCGTGTCTTACATGAATTGGTATCAGCAGAAATCAGGAACCAGCCCCAAGAGATGGATCTACGACACATCCAAGCTGGCATCTGGAGTGCCTGCACACTTC |
| | | | AGGGGCAGTGGGTCAGGAACTAGCTATTCCCTGACCATTAGCGGCATGGAGGCCGAAGATGCCGCTACCTACTATTGTCAGCAGTGGTCTAGTAACCCATTCACATTTGGCAGCGGGACT |
| | | | AAGCTGGAGATCAATAGGGCAGCCGAACCCAAATCAAGCGACAAGACACATACTTGCCCCCCTTGTCCAGCTCCAGAAGCTGCAGGAGGACCTTCCGTGTTCCTGTTTCCACCCAAACCA |
| | | | AAGGATACACTGATGATTAGCCGCACCCCTGAGGTCACATGCGTGGTCGTGAGCGTGAGCCACGAGGACCCCGAAGTCAAGTTCAACTGGTACGTGGACGGCGTCGAAGTGCATAATGCC |
| | | | AAAACCAAGCCTAGGGAGGAACAGTACAACAGTACATATAGAGTCGTGTCAGTGCTGACCGTCCTGCACCAGGATTGGCTGAACGGCAAGGAGTACAAATGCAAGGTGTCCAACAAGGCA |
| | | | CTGCCTGCCCCAATCGAGAAGACCATTTCTAAAGCTAAGGGGCAGCCCCGAGAACCTCAGGTCTACGTGTATCCTCCATCCCGGGACGAGCTGACTAAAAACCAGGTCTCTCTGACCTGT |
| | | | CTGGTGAAGGGCTTTTACCCATCTGATATTGCAGTCGAGTGGGAAAGTAATGGGCAGCCCGAGAACAATTATAAGACAACTCCCCCTGTGCTGGACTCCGATGGGTCTTTCGCACTGGTC |
| | | | AGCAAACTGACAGTGGATAAGTCCAGATGGCAGCAGGGAAACGTCTTTTCTTGTAGTGTGATGCATGAAGCCCTGCACAATCATTACACTCAGAAATCACTGAGCCTGTCCCCCGGCAAG |
| 115 | 2184 | VH | QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS |
| 116 | 2184 | VH | CAGGTCCAGCTGCAGCAGAGCGGAGCAGAGCTGGCTCGACCAGGAGCTAGTGTGAAAATGTCATGCAAGGCAAGCGGCTACACCTTCACACGGTATACTATGCACTGGGTGAAACAGAGA |
| | | | CCCGGACAGGGCCTGGAATGGATCGGGTACATTAACCCTAGCCGAGGATACACCAACTACAACCAGAAGTTTAAAGACAAGGCCACCCTGACCACAGATAAGAGCTCCTCTACAGCTTAT |
| | | | ATGCAGCTGAGTTCACTGACTTCTGAGGACAGTGCCGTGTACTATTGTGCTCGGTACTATGACGATCATTACTCCCTGGATTATTGGGGGCAGGGAACTACCCTGACCGTGAGCTCC |
| 117 | 2184 | linker | GGGGSGGGGSGGGGS |
| 118 | 2184 | linker | GGAGGAGGAGGCAGTGGAGGAGGAGGGTCAGGCGGAGGAGGAAGC |
| 119 | 2184 | VL | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN |
| 120 | 2184 | VL | CAGATTGTGCTGACACAGTCTCCAGCTATCATGTCCGCATCTCCCGGCGAGAAAGTCACTATGACCTGCTCCGCCTCAAGCTCCGTGTCTTACATGAATTGGTATCAGCAGAAATCAGGA |
| | | | ACCAGCCCCAAGAGATGGATCTACGACACATCCAAGCTGGCATCTGGAGTGCCTGCACACTTCAGGGGCAGTGGGTCAGGAACTAGCTATTCCCTGACCATTAGCGGCATGGAGGCCGAA |
| | | | GATGCCGCTACCTACTATTGTCAGCAGTGGTCTAGTAACCCATTCACATTTGGCAGCGGGACTAAGCTGGAGATCAAT |
| 121 | 2184 | hinge | AAEPKSSDKTHTCPPCP |
| 122 | 2184 | hinge | GCAGCCGAACCCAAATCAAGCGACAAGACACATACTTGCCCCCCTTGTCCA |
| 123 | 2184 | CH2 | APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK |
| 124 | 2184 | CH2 | GCTCCAGAAGCTGCAGGAGGACCTTCCGTGTTCCTGTTTCCACCCAAACCAAAGGATACACTGATGATTAGCCGCACCCCTGAGGTCACATGCGTGGTCGTGAGCGTGAGCCACGAGGAC |
| | | | CCCGAAGTCAAGTTCAACTGGTACGTGGACGGCGTCGAAGTGCATAATGCCAAAACCAAGCCTAGGGAGGAACAGTACAACAGTACATATAGAGTCGTGTCAGTGCTGACCGTCCTGCAC |
| | | | CAGGATTGGCTGAACGGCAAGGAGTACAAATGCAAGGTGTCCAACAAGGCACTGCCTGCCCCAATCGAGAAGACCATTTCTAAAGCTAAG |
| 125 | 2184 | CH3 | GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG |
| 126 | 2184 | CH3 | GGGCAGCCCCGAGAACCTCAGGTCTACGTGTATCCTCCATCCCGGGACGAGCTGACTAAAAACCAGGTCTCTCTGACCTGTCTGGTGAAGGGCTTTTACCCATCTGATATTGCAGTCGAG |
| | | | TGGGAAAGTAATGGGCAGCCCGAGAACAATTATAAGACAACTCCCCCTGTGCTGGACTCCGATGGGTCTTTCGCACTGGTCAGCAAACTGACAGTGGATAAGTCCAGATGGCAGCAGGGA |
| | | | AACGTCTTTTCTTGTAGTGTGATGCATGAAGCCCTGCACAATCATTACACTCAGAAATCACTGAGCCTGTCCCCCGGC |
| 127 | 1842 | Full | DIQLTQSPASLAVSLGQRATISCKASQSVDYDGDSYLNWYQQIPGQPPKLLIYDASNLVSGTPPRFSGSGSGTDFTLNIHPVEKVDAATYHCQQSTEDPWTFGGGTKLEIKGGGGSGGGG |
| | | | SGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYW |
| | | | GQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKV |
| | | | SNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSL |
| | | | SPGK |
| 128 | 1842 | Full | GATATTCAGCTGACACAGAGTCCTGCTTCACTGGCAGTGAGCCTGGGACAGCGAGCAACTATCTCCTGCAAAGCTAGTCAGTCAGTGGACTATGATGGCGACTCCTATCTGAACTGGTAC |
| | | | CAGCAGATCCCAGGGCAGCCCCCTAAGCTGCTGATCTACGACGCCTCAAATCTGGTGAGCGGCATCCCACCACGATTCAGCGGCAGCGGCTCTGGGACTGATTTTACCCTGAACATTCAC |
| | | | CCAGTCGAGAAGGTGGACGCCGCTACCTACCATTGCCAGCAGTCTACCGAGGACCCCTGGACATTCGGCGGGGGAACTAAACTGGAAATCAAGGGAGGAGGAGGCAGTGGCGGAGGAGGG |
| | | | TCAGGAGGAGGAGGAAGCCAGGTGCAGCTGCAGCAGAGCGGAGCAGAGCTGGTCAGACCAGGAAGCTCCGTGAAAATTTCCTGTAAGGCATCTGGCTATGCCTTTTCTAGTTACTGGATG |
| | | | AATTGGGTGAAGCAGAGGCCAGGACAGGGCCTGGAATGGATCGGGCAGATTTGGCCCGGGGATGGAGACACCAACTATAATGGAAAGTTCAAAGGCAAGGCTACACTGACTGCAGACGAG |
| | | | TCAAGCTCCACAGCTTATATGCAGCTGTCTAGTCTGGCCAGCGAGGATTCCGCTGTGTACTTTTGCGCACGGAGAGAAACCACAACTGTGGGCAGGTACTATTACGCAATGGACTACTGG |
| | | | GGCCAGGGGACCACAGTCACCGTGTCAAGCGCAGCCGAACCCAAATCCTCTGATAAGACCCACACATGCCCTCCATGTCCAGCACCTGAGCTGCTGGGAGGACCAAGCGTGTTCCTGTTT |
| | | | CCACCTAAACCTAAGGACACACTGATGATCTCTCGGACACCCGAAGTCACTTGTGTGGTCGTGGATGTGAGCCACGAGGACCCTGAAGTCAAATTCAACTGGTACGTGGATGGCGTCGAG |
| | | | GTGCATAATGCCAAAACTAAGCCTAGGGAGGAACAGTATAACTCCACTTACCGCGTCGTGTCTGTCCTGACCGTGCTGCATCAGGACTGGCTGAACGGAAAGGAGTACAAATGCAAGGTG |
| | | | AGCAACAAGGCCCTGCCAGCTCCCATCGAGAAGACAATTTCCAAAGCTAAGGGCCAGCCTCGAGAACCACAGGTCTATGTGTACCCACCCAGCCGGGACGAGCTGACCAAAAACCAGGTC |
| | | | TCCCTGACATGTCTGGTGAAGGGATTTTATCCTTCTGATATTGCCGTGGAGTGGGAAAGTAATGGCCAGCCAGAAAACAATTACAAGACTACCCCTCCAGTGCTGGATTCTGACGGGAGT |
| | | | TTCGCACTGGTCAGTAAACTGACTGTGGATAAGTCACGGTGGCAGCAGGGAAACGTCTTTAGTTGTTCAGTGATGCACGAGGCCCTGCACAATCATTACACCCAGAAAAGCCTGTCCCTG |
| | | | TCTCCCGGCAAG |
| 129 | 1842 | VL | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK |
| 130 | 1842 | VL | GATATTCAGCTGACACAGAGTCCTGCTTCACTGGCAGTGAGCCTGGGACAGCGAGCAACTATCTCCTGCAAAGCTAGTCAGTCAGTGGACTATGATGGCGACTCCTATCTGAACTGGTAC |
| | | | CAGCAGATCCCAGGGCAGCCCCCTAAGCTGCTGATCTACGACGCCTCAAATCTGGTGAGCGGCATCCCACCACGATTCAGCGGCAGCGGCTCTGGGACTGATTTTACCCTGAACATTCAC |
| | | | CCAGTCGAGAAGGTGGACGCCGCTACCTACCATTGCCAGCAGTCTACCGAGGACCCCTGGACATTCGGCGGGGGAACTAAACTGGAAATCAAG |
| 131 | 1842 | linker | GGGGSGGGGSGGGGS |
| 132 | 1842 | linker | GGAGGAGGAGGCAGTGGCGGAGGAGGGTCAGGAGGAGGAGGAAGC |
| 133 | 1842 | VH | QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTV |
| | | | TVSS |
| 134 | 1842 | VH | CAGGTGCAGCTGCAGCAGAGCGGAGCAGAGCTGGTCAGACCAGGAAGCTCCGTGAAAATTTCCTGTAAGGCATCTGGCTATGCCTTTTCTAGTTACTGGATGAATTGGGTGAAGCAGAGG |
| | | | CCAGGACAGGGCCTGGAATGGATCGGGCAGATTTGGCCCGGGGATGGAGACACCAACTATAATGGAAAGTTCAAAGGCAAGGCTACACTGACTGCAGACGAGTCAAGCTCCACAGCTTAT |
| | | | ATGCAGCTGTCTAGTCTGGCCAGCGAGGATTCCGCTGTGTACTTTTGCGCACGGAGAGAAACCACAACTGTGGGCAGGTACTATTACGCAATGGACTACTGGGGCCAGGGGACCACAGTC |
| | | | ACCGTGTCAAGC |
| 135 | 1842 | hinge | AAEPKSSDKTHTCPPCP |
| 136 | 1842 | hinge | GCAGCCGAACCCAAATCCTCTGATAAGACCCACACATGCCCTCCATGTCCA |
| 137 | 1842 | CH2 | APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK |
| 138 | 1842 | CH2 | GCACCTGAGCTGCTGGGAGGACCAAGCGTGTTCCTGTTTCCACCTAAACCTAAGGACACACTGATGATCTCTCGGACACCCGAAGTCACTTGTGTGGTCGTGGATGTGAGCCACGAGGAC |
| | | | CCTGAAGTCAAATTCAACTGGTACGTGGATGGCGTCGAGGTGCATAATGCCAAAACTAAGCCTAGGGAGGAACAGTATAACTCCACTTACCGCGTCGTGTCTGTCCTGACCGTGCTGCAT |
| | | | CAGGACTGGCTGAACGGAAAGGAGTACAAATGCAAGGTGAGCAACAAGGCCCTGCCAGCTCCCATCGAGAAGACAATTTCCAAAGCTAAG |
| 139 | 1842 | CH3 | GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG |
| 140 | 1842 | CH3 | GGCCAGCCTCGAGAACCACAGGTCTATGTGTACCCACCCAGCCGGGACGAGCTGACCAAAAACCAGGTCTCCCTGACATGTCTGGTGAAGGGATTTTATCCTTCTGATATTGCCGTGGAG |
| | | | TGGGAAAGTAATGGCCAGCCAGAAAACAATTACAAGACTACCCCTCCAGTGCTGGATTCTGACGGGAGTTTCGCACTGGTCAGTAAACTGACTGTGGATAAGTCACGGTGGCAGCAGGGA |
| | | | AACGTCTTTAGTTGTTCAGTGATGCACGAGGCCCTGCACAATCATTACACCCAGAAAAGCCTGTCCCTGTCTCCCGGC |
| 141 | 2227 | Full | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEINGGGGSGGGGSGGGG |
| | | | SQVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSS |
| | | | AAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIE |
| | | | KTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK |
| 142 | 2227 | Full | CAGATCGTCCTGACACAGTCCCCAGCAATCATGTCAGCCAGCCCCGGGGAGAAAGTCACAATGACTTGCTCAGCAAGCTCCTCTGTGAGCTACATGAACTGGTATCAGCAGAAAAGCGGG |
| | | | ACCTCCCCCAAGAGATGGATCTACGACACATCCAAGCTGGCTTCTGGAGTGCCTGCACACTTCAGGGGCAGCGGCTCTGGGACCAGTTATTCACTGACAATTAGCGGCATGGAGGCTGAA |
| | | | GATGCCGCTACCTACTATTGCCAGCAGTGGAGTTCAAACCCATTCACTTTTGGATGTGGCACCAAGCTGGAAATTAATGGCGGAGGAGGCTCCGGAGGAGGAGGGTCTGGAGGAGGAGGA |
| | | | AGTCAGGTGCAGCTGCAGCAGTCCGGAGCTGAGCTGGCACGACCAGGAGCAAGTGTGAAAATGTCATGCAAGGCCAGCGGCTACACCTTCACACGGTATACCATGCATTGGGTGAAACAG |
| | | | AGACCCGGACAGTGTCTGGAATGGATCGGCTACATTAATCCTTCTCGAGGGTACACAAACTACAACCAGAAGTTTAAAGACAAGGCTACTCTGACCACAGATAAGAGCTCCTCTACCGCA |
| | | | TATATGCAGCTGAGTTCACTGACATCTGAGGACAGTGCCGTGTACTATTGCGCTAGGTACTATGACGATCACTACTGTCTGGATTATTGGGGGCAGGGAACTACCCTGACAGTGAGCTCC |
| | | | GCAGCCGAACCTAAATCTAGTGACAAGACTCATACCTGCCCCCCTTGTCCAGCACCAGAGCTGCTGGGAGGACCTAGCGTGTTCCTGTTTCCACCCAAACCAAAGGATACTCTGATGATC |
| | | | TCCCGGACACCTGAAGTCACTTGCGTGGTCGTGGACGTGTCTCACGAGGACCCCGAAGTCAAGTTTAACTGGTACGTGGACGGCGTCGAGGTGCATAATGCCAAAACCAAGCCCAGGGAG |
| | | | GAACAGTACAACTCCACATATCGCGTCGTGTCTGTCCTGACTGTGCTGCACCAGGATTGGCTGAACGGAAAGGAGTACAAATGCAAGGTGAGCAACAAGGCCCTGCCTGCTCCAATCGAG |
| | | | AAGACAATTAGCAAAGCCAAGGGCCAGCCCCGAGAACCTCAGGTCTACGTGCTGCCTCCATCTCGGGACGAGCTGACTAAAAACCAGGTCAGTCTGCTGTGTCTGGTGAAGGGATTCTAT |
| | | | CCAAGCGATATTGCTGTGGAGTGGGAATCCAATGGCCAGCCCGAAAACAATTACCTGACTTGGCCCCCTGTCCTGGACTCAGATGGCAGCTTCTTTCTGTATAGTAAACTGACCGTGGAC |
| | | | AAGTCACGGTGGCAGCAGGGGAACGTCTTTAGCTGTTCCGTGATGCATGAGGCCCTGCACAATCATTACACCCAGAAATCTCTGAGTCTGTCACCCGGCAAG |
| 143 | 2227 | VL | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEIN |
| 144 | 2227 | VL | CAGATCGTCCTGACACAGTCCCCAGCAATCATGTCAGCCAGCCCCGGGGAGAAAGTCACAATGACTTGCTCAGCAAGCTCCTCTGTGAGCTACATGAACTGGTATCAGCAGAAAAGCGGG |
| | | | ACCTCCCCCAAGAGATGGATCTACGACACATCCAAGCTGGCTTCTGGAGTGCCTGCACACTTCAGGGGCAGCGGCTCTGGGACCAGTTATTCACTGACAATTAGCGGCATGGAGGCTGAA |
| | | | GATGCCGCTACCTACTATTGCCAGCAGTGGAGTTCAAACCCATTCACTTTTGGATGTGGCACCAAGCTGGAAATTAAT |
| 145 | 2227 | linker | GGGGSGGGGSGGGGS |
| 146 | 2227 | linker | GGCGGAGGAGGCTCCGGAGGAGGAGGGTCTGGAGGAGGAGGAAGT |
| 147 | 2227 | VH | QVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYCLDYWGQGTTLTVSS |
| 148 | 2227 | VH | CAGGTGCAGCTGCAGCAGTCCGGAGCTGAGCTGGCACGACCAGGAGCAAGTGTGAAAATGTCATGCAAGGCCAGCGGCTACACCTTCACACGGTATACCATGCATTGGGTGAAACAGAGA |
| | | | CCCGGACAGTGTCTGGAATGGATCGGCTACATTAATCCTTCTCGAGGGTACACAAACTACAACCAGAAGTTTAAAGACAAGGCTACTCTGACCACAGATAAGAGCTCCTCTACCGCATAT |
| | | | ATGCAGCTGAGTTCACTGACATCTGAGGACAGTGCCGTGTACTATTGCGCTAGGTACTATGACGATCACTACTGTCTGGATTATTGGGGGCAGGGAACTACCCTGACAGTGAGCTCC |
| 149 | 2227 | hinge | AAEPKSSDKTHTCPPCP |
| 150 | 2227 | hinge | GCAGCCGAACCTAAATCTAGTGACAAGACTCATACCTGCCCCCCTTGTCCA |
| 151 | 2227 | CH2 | APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK |
| 152 | 2227 | CH2 | GCACCAGAGCTGCTGGGAGGACCTAGCGTGTTCCTGTTTCCACCCAAACCAAAGGATACTCTGATGATCTCCCGGACACCTGAAGTCACTTGCGTGGTCGTGGACGTGTCTCACGAGGAC |
| | | | CCCGAAGTCAAGTTTAACTGGTACGTGGACGGCGTCGAGGTGCATAATGCCAAAACCAAGCCCAGGGAGGAACAGTACAACTCCACATATCGCGTCGTGTCTGTCCTGACTGTGCTGCAC |
| | | | CAGGATTGGCTGAACGGAAAGGAGTACAAATGCAAGGTGAGCAACAAGGCCCTGCCTGCTCCAATCGAGAAGACAATTAGCAAAGCCAAG |
| 153 | 2227 | CH3 | GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 154 | 2227 | CH3 | GGCCAGCCCCGAGAACCTCAGGTCTACGTGCTGCCTCCATCTCGGGACGAGCTGACTAAAAACCAGGTCAGTCTGCTGTGTCTGGTGAAGGGATTCTATCCAAGCGATATTGCTGTGGAG |
| | | | TGGGAATCCAATGGCCAGCCCGAAAACAATTACCTGACTTGGCCCCCTGTCCTGGACTCAGATGGCAGCTTCTTTCTGTATAGTAAACTGACCGTGGACAAGTCACGGTGGCAGCAGGGG |
| | | | AACGTCTTTAGCTGTTCCGTGATGCATGAGGCCCTGCACAATCATTACACCCAGAAATCTCTGAGTCTGTCACCCGGC |
| 155 | 2228 | Full | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEINGGGGSGGGGSGGGG |
| | | | SQVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYSLDYWGQGTTLTVSS |
| | | | AAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIE |
| | | | KTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK |
| 156 | 2228 | Full | CAGATCGTCCTGACACAGAGCCCAGCAATCATGTCAGCCAGCCCCGGGGAGAAAGTCACAATGACTTGCTCAGCAAGCTCCTCTGTGAGCTACATGAACTGGTATCAGCAGAAAAGCGGG |
| | | | ACCTCCCCCAAGAGATGGATCTACGACACATCCAAGCTGGCTTCTGGAGTGCCTGCACACTTCAGGGGCAGCGGCTCTGGGACCAGTTATTCACTGACAATTTCCGGCATGGAGGCTGAA |
| | | | GATGCCGCTACCTACTATTGCCAGCAGTGGAGTTCAAACCCATTCACTTTTGGATGTGGCACCAAGCTGGAAATTAATGGCGGAGGAGGCTCCGGAGGAGGAGGGTCTGGAGGAGGAGGA |
| | | | AGTCAGGTGCAGCTGCAGCAGTCCGGAGCTGAGCTGGCACGACCAGGAGCAAGTGTGAAAATGTCATGCAAGGCCAGCGGCTACACCTTCACACGGTATACCATGCATTGGGTGAAACAG |
| | | | AGACCCGGACAGTGTCTGGAATGGATCGGCTACATTAATCCTAGCCGAGGGTACACAAACTACAACCAGAAGTTTAAAGACAAGGCTACTCTGACCACAGATAAGAGCTCCTCTACCGCA |
| | | | TATATGCAGCTGAGTTCACTGACATCTGAGGACAGTGCCGTGTACTATTGCGCTAGGTACTATGACGATCACTACTCCCTGGATTATTGGGGGCAGGGAACTACCCTGACAGTGAGCTCC |
| | | | GCAGCCGAACCTAAATCTAGTGACAAGACTCATACCTGCCCACCTTGTCCAGCACCAGAGCTGCTGGGCGGGCCTTCTGTGTTCCTGTTTCCACCCAAACCAAAGGATACTCTGATGATC |
| | | | TCCCGGACACCTGAAGTCACTTGTGTGGTCGTGGACGTGTCTCACGAGGACCCCGAAGTCAAGTTTAACTGGTACGTGGACGGCGTCGAGGTGCATAATGCCAAAACCAAGCCCAGGGAG |
| | | | GAACAGTACAACTCCACATATCGCGTCGTGTCTGTCCTGACTGTGCTGCACCAGGATTGGCTGAACGGAAAGGAGTACAAATGCAAGGTGAGCAACAAGGCCCTGCCTGCTCCAATCGAG |
| | | | AAGACAATTAGCAAAGCCAAGGGCCAGCCCCGAGAACCTCAGGTCTACGTGCTGCCTCCATCTCGGGACGAGCTGACTAAAAACCAGGTCAGTCTGCTGTGTCTGGTGAAGGGATTCTAT |
| | | | CCAAGCGATATTGCTGTGGAGTGGGAATCCAATGGCCAGCCCGAAAACAATTACCTGACTTGGCCCCCTGTCCTGGACTCAGATGGCAGCTTCTTTCTGTATAGTAAACTGACCGTGGAC |
| | | | AAGTCACGGTGGCAGCAGGGGAACGTCTTTAGCTGTTCCGTGATGCATGAGGCCCTGCACAATCATTACACCCAGAAATCTCTGAGTCTGTCACCCGGCAAG |
| 157 | 2228 | VL | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEIN |
| 158 | 2228 | VL | CAGATCGTCCTGACACAGAGCCCAGCAATCATGTCAGCCAGCCCCGGGGAGAAAGTCACAATGACTTGCTCAGCAAGCTCCTCTGTGAGCTACATGAACTGGTATCAGCAGAAAAGCGGG |
| | | | ACCTCCCCCAAGAGATGGATCTACGACACATCCAAGCTGGCTTCTGGAGTGCCTGCACACTTCAGGGGCAGCGGCTCTGGGACCAGTTATTCACTGACAATTTCCGGCATGGAGGCTGAA |
| | | | GATGCCGCTACCTACTATTGCCAGCAGTGGAGTTCAAACCCATTCACTTTTGGATGTGGCACCAAGCTGGAAATTAAT |
| 159 | 2228 | linker | GGGGSGGGGSGGGGS |
| 160 | 2228 | linker | GGCGGAGGAGGCTCCGGAGGAGGAGGGTCTGGAGGAGGAGGAAGT |
| 161 | 2228 | VH | QVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYSLDYWGQGTTLTVSS |
| 162 | 2228 | VH | CAGGTGCAGCTGCAGCAGTCCGGAGCTGAGCTGGCACGACCAGGAGCAAGTGTGAAAATGTCATGCAAGGCCAGCGGCTACACCTTCACACGGTATACCATGCATTGGGTGAAACAGAGA |
| | | | CCCGGACAGTGTCTGGAATGGATCGGCTACATTAATCCTAGCCGAGGGTACACAAACTACAACCAGAAGTTTAAAGACAAGGCTACTCTGACCACAGATAAGAGCTCCTCTACCGCATAT |
| | | | ATGCAGCTGAGTTCACTGACATCTGAGGACAGTGCCGTGTACTATTGCGCTAGGTACTATGACGATCACTACTCCCTGGATTATTGGGGGCAGGGAACTACCCTGACAGTGAGCTCC |
| 163 | 2228 | hinge | AAEPKSSDKTHTCPPCP |
| 164 | 2228 | hinge | GCAGCCGAACCTAAATCTAGTGACAAGACTCATACCTGCCCACCTTGTCCA |
| 165 | 2228 | CH2 | APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK |
| 166 | 2228 | CH2 | GCACCAGAGCTGCTGGGCGGGCCTTCTGTGTTCCTGTTTCCACCCAAACCAAAGGATACTCTGATGATCTCCCGGACACCTGAAGTCACTTGTGTGGTCGTGGACGTGTCTCACGAGGAC |
| | | | CCCGAAGTCAAGTTTAACTGGTACGTGGACGGCGTCGAGGTGCATAATGCCAAAACCAAGCCCAGGGAGGAACAGTACAACTCCACATATCGCGTCGTGTCTGTCCTGACTGTGCTGCAC |
| | | | CAGGATTGGCTGAACGGAAAGGAGTACAAATGCAAGGTGAGCAACAAGGCCCTGCCTGCTCCAATCGAGAAGACAATTAGCAAAGCCAAG |
| 167 | 2228 | CH3 | GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 168 | 2228 | CH3 | GGCCAGCCCCGAGAACCTCAGGTCTACGTGCTGCCTCCATCTCGGGACGAGCTGACTAAAAACCAGGTCAGTCTGCTGTGTCTGGTGAAGGGATTCTATCCAAGCGATATTGCTGTGGAG |
| | | | TGGGAATCCAATGGCCAGCCCGAAAACAATTACCTGACTTGGCCCCCTGTCCTGGACTCAGATGGCAGCTTCTTTCTGTATAGTAAACTGACCGTGGACAAGTCACGGTGGCAGCAGGGG |
| | | | AACGTCTTTAGCTGTTCCGTGATGCATGAGGCCCTGCACAATCATTACACCCAGAAATCTCTGAGTCTGTCACCCGGC |
| 169 | 1109 | Full | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGG |
| | | | SGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYW |
| | | | GQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYC |
| | | | LDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQ |
| | | | QWSSNPLTFGAGTKLELKHHHHHH |
| 170 | 1109 | Full | GATATTCAGCTGACACAGTCTCCAGCTAGTCTGGCAGTGAGCCTGGGCCAGCGGGCTACTATCAGCTGCAAGGCAAGCCAGTCCGTCGACTACGATGGGGACAGCTATCTGAACTGGTAC |
| | | | CAGCAGATCCCCGGACAGCCCCCTAAACTGCTGATCTACGACGCCTCAAATCTGGTGAGCGGCATCCCACCCAGATTCTCTGGAAGTGGCTCAGGGACCGATTTTACACTGAACATTCAC |
| | | | CCCGTGGAGAAGGTCGACGCCGCTACCTACCATTGCCAGCAGTCCACTGAGGACCCCTGGACCTTCGGAGGAGGAACAAAGCTGGAAATCAAAGGCGGAGGAGGCAGTGGAGGAGGAGGG |
| | | | AGCGGAGGAGGAGGAAGCCAGGTGCAGCTGCAGCAGAGCGGAGCAGAACTGGTGAGACCTGGAAGCTCCGTCAAGATTTCCTGTAAAGCATCTGGCTATGCCTTTTCTAGTTACTGGATG |
| | | | AATTGGGTGAAGCAGAGGCCAGGACAGGGACTGGAGTGGATCGGACAGATTTGGCCTGGGGATGGAGACACCAACTACAATGGAAAGTTCAAAGGCAAGGCTACCCTGACAGCAGACGAA |
| | | | TCAAGCTCCACAGCTTACATGCAGCTGTCTAGTCTGGCATCAGAGGATAGCGCCGTGTATTTTTGCGCTCGGAGAGAAACCACAACTGTCGGCCGCTACTATTACGCCATGGACTACTGG |
| | | | GGCCAGGGGACCACAGTGACAGTCTCAAGCGGCGGGGGAGGCTCCGATATCAAGCTGCAGCAGTCTGGAGCAGAGCTGGCTCGACCAGGAGCCAGTGTGAAGATGTCATGTAAAACCAGC |
| | | | GGCTATACTTTCACCAGGTACACAATGCACTGGGTGAAACAGCGCCCAGGACAGGGCCTGGAATGGATCGGATACATTAACCCCTCCAGGGGCTATACCAACTACAATCAGAAGTTCAAG |
| | | | GATAAAGCCACTCTGACTACCGACAAGTCCTCTAGTACCGCTTATATGCAGCTGTCAAGCCTGACATCCGAGGACTCTGCAGTGTATTACTGCGCCCGCTATTACGACGATCATTATTGT |
| | | | CTGGATTACTGGGGGCAGGGAACAACTCTGACTGTGTCCTCTGTCGAAGGGGGAAGTGGAGGGTCAGGAGGCAGCGGAGGCAGCGGAGGGGTGGACGATATCCAGCTGACCCAGTCCCCT |
| | | | GCCATTATGAGCGCTTCCCCAGGCGAGAAGGTGACAATGACTTGCAGGGCTAGTTCAAGCGTCTCTTATATGAATTGGTATCAGCAGAAGTCTGGCACTAGTCCTAAACGATGGATCTAT |
| | | | GACACCTCCAAAGTGGCATCTGGGGTCCCATACCGGTTCTCTGGCAGTGGGTCAGGAACTAGCTATTCCCTGACCATTTCCTCTATGGAGGCAGAAGATGCAGCCACCTATTACTGTCAG |
| | | | CAGTGGAGTTCAAATCCCCTGACATTTGGCGCCGGGACTAAGCTGGAGCTGAAACACCATCACCATCACCAT |
| 171 | 1109 | VL | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK |
| 172 | 1109 | VL | GATATTCAGCTGACACAGTCTCCAGCTAGTCTGGCAGTGAGCCTGGGCCAGCGGGCTACTATCAGCTGCAAGGCAAGCCAGTCCGTCGACTACGATGGGGACAGCTATCTGAACTGGTAC |
| | | | CAGCAGATCCCCGGACAGCCCCCTAAACTGCTGATCTACGACGCCTCAAATCTGGTGAGCGGCATCCCACCCAGATTCTCTGGAAGTGGCTCAGGGACCGATTTTACACTGAACATTCAC |
| | | | CCCGTGGAGAAGGTCGACGCCGCTACCTACCATTGCCAGCAGTCCACTGAGGACCCCTGGACCTTCGGAGGAGGAACAAAGCTGGAAATCAAA |
| 173 | 1109 | linker | GGGGSGGGGSGGGGS |
| 174 | 1109 | linker | GGCGGAGGAGGCAGTGGAGGAGGAGGGAGCGGAGGAGGAGGAAGC |
| 175 | 1109 | VH | QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTV |
| | | | TVSS |
| 176 | 1109 | VH | CAGGTGCAGCTGCAGCAGAGCGGAGCAGAACTGGTGAGACCTGGAAGCTCCGTCAAGATTTCCTGTAAAGCATCTGGCTATGCCTTTTCTAGTTACTGGATGAATTGGGTGAAGCAGAGG |
| | | | CCAGGACAGGGACTGGAGTGGATCGGACAGATTTGGCCTGGGGATGGAGACACCAACTACAATGGAAAGTTCAAAGGCAAGGCTACCCTGACAGCAGACGAATCAAGCTCCACAGCTTAC |
| | | | ATGCAGCTGTCTAGTCTGGCATCAGAGGATAGCGCCGTGTATTTTTGCGCTCGGAGAGAAACCACAACTGTCGGCCGCTACTATTACGCCATGGACTACTGGGGCCAGGGGACCACAGTG |
| | | | ACAGTCTCAAGC |
| 177 | 1109 | VH | DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS |
| 178 | 1109 | VH | GATATCAAGCTGCAGCAGTCTGGAGCAGAGCTGGCTCGACCAGGAGCCAGTGTGAAGATGTCATGTAAAACCAGCGGCTATACTTTCACCAGGTACACAATGCACTGGGTGAAACAGCGC |
| | | | CCAGGACAGGGCCTGGAATGGATCGGATACATTAACCCCTCCAGGGGCTATACCAACTACAATCAGAAGTTCAAGGATAAAGCCACTCTGACTACCGACAAGTCCTCTAGTACCGCTTAT |
| | | | ATGCAGCTGTCAAGCCTGACATCCGAGGACTCTGCAGTGTATTACTGCGCCCGCTATTACGACGATCATTATTGTCTGGATTACTGGGGGCAGGGAACAACTCTGACTGTGTCCTCT |
| 179 | 1109 | linker | GGSGGSGGSGGSGG |
| 180 | 1109 | linker | GGGGGAAGTGGAGGGTCAGGAGGCAGCGGAGGCAGCGGAGGG |
| 181 | 1109 | VL | DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK |
| 182 | 1109 | VL | GATATCCAGCTGACCCAGTCCCCTGCCATTATGAGCGCTTCCCCAGGCGAGAAGGTGACAATGACTTGCAGGGCTAGTTCAAGCGTCTCTTATATGAATTGGTATCAGCAGAAGTCTGGC |
| | | | ACTAGTCCTAAACGATGGATCTATGACACCTCCAAAGTGGCATCTGGGGTCCCATACCGGTTCTCTGGCAGTGGGTCAGGAACTAGCTATTCCCTGACCATTTCCTCTATGGAGGCAGAA |
| | | | GATGCAGCCACCTATTACTGTCAGCAGTGGAGTTCAAATCCCCTGACATTTGGCGCCGGGACTAAGCTGGAGCTGAAA |
| 183 | 2167 | Full | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGG |
| | | | SQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS |
| | | | AAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIE |
| | | | KTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK |
| 184 | 2167 | Full | CAGATCGTCCTGACACAGAGCCCAGCAATCATGTCAGCCAGCCCCGGCGAGAAAGTCACAATGACTTGCTCAGCAAGCTCCTCTGTGAGCTACATGAACTGGTATCAGCAGAAAAGCGGA |
| | | | ACCTCCCCCAAGAGATGGATCTACGACACATCCAAGCTGGCTTCTGGAGTGCCTGCACACTTCAGGGGCAGCGGCTCTGGGACCAGTTATTCACTGACAATTTCCGGCATGGAGGCTGAA |
| | | | GATGCCGCTACCTACTATTGCCAGCAGTGGAGTTCAAACCCATTCACTTTTGGATCTGGCACCAAGCTGGAAATTAATGGCGGAGGAGGCTCCGGAGGAGGAGGGTCTGGAGGAGGAGGA |
| | | | AGTCAGGTGCAGCTGCAGCAGAGCGGAGCTGAGCTGGCACGACCAGGAGCAAGTGTGAAAATGTCCTGTAAGGCCAGCGGCTACACCTTCACACGGTATACCATGCATTGGGTGAAACAG |
| | | | AGACCCGGGCAGGGACTGGAATGGATCGGGTACATTAATCCTTCCCGAGGATACACAAACTACAACCAGAAGTTTAAAGACAAGGCTACTCTGACCACAGATAAGAGCTCCTCTACCGCA |
| | | | TATATGCAGCTGAGTTCACTGACATCTGAGGACAGTGCCGTGTACTATTGCGCTAGGTACTATGACGATCACTACTCCCTGGATTATTGGGGCCAGGGGACTACCCTGACAGTGAGCTCC |
| | | | GCAGCCGAACCTAAATCTAGTGACAAGACTCATACCTGCCCCCCTTGTCCAGCACCAGAGCTGCTGGGAGGACCTAGCGTGTTCCTGTTTCCACCCAAACCAAAGGATACTCTGATGATC |
| | | | TCCCGGACACCTGAAGTCACTTGTGTGGTCGTGGACGTGTCTCACGAGGACCCCGAAGTCAAGTTTAACTGGTACGTGGACGGCGTCGAGGTGCATAATGCCAAAACCAAGCCCAGGGAG |
| | | | GAACAGTACAACTCCACATATCGCGTCGTGTCTGTCCTGACTGTGCTGCACCAGGATTGGCTGAACGGCAAGGAGTACAAATGCAAGGTGAGCAACAAGGCCCTGCCTGCTCCAATCGAG |
| | | | AAGACAATTAGCAAAGCCAAGGGGCAGCCCCGAGAACCTCAGGTCTACGTGCTGCCTCCATCTCGGGACGAGCTGACTAAAAACCAGGTCAGTCTGCTGTGTCTGGTGAAGGGCTTCTAT |
| | | | CCAAGCGATATTGCTGTGGAGTGGGAATCCAATGGGCAGCCCGAAAACAATTACCTGACTTGGCCCCCTGTCCTGGACTCAGATGGGAGCTTCTTTCTGTATAGTAAACTGACCGTGGAC |
| | | | AAGTCACGGTGGCAGCAGGGAAACGTCTTTAGCTGTTCCGTGATGCATGAGGCCCTGCACAATCATTACACCCAGAAATCTCTGAGTCTGTCACCCGGCAAG |
| 185 | 2167 | VL | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN |
| 186 | 2167 | VL | CAGATCGTCCTGACACAGAGCCCAGCAATCATGTCAGCCAGCCCCGGCGAGAAAGTCACAATGACTTGCTCAGCAAGCTCCTCTGTGAGCTACATGAACTGGTATCAGCAGAAAAGCGGA |
| | | | ACCTCCCCCAAGAGATGGATCTACGACACATCCAAGCTGGCTTCTGGAGTGCCTGCACACTTCAGGGGCAGCGGCTCTGGGACCAGTTATTCACTGACAATTTCCGGCATGGAGGCTGAA |
| | | | GATGCCGCTACCTACTATTGCCAGCAGTGGAGTTCAAACCCATTCACTTTTGGATCTGGCACCAAGCTGGAAATTAAT |
| 187 | 2167 | linker | GGGGSGGGGSGGGGS |
| 188 | 2167 | linker | GGCGGAGGAGGCTCCGGAGGAGGAGGGTCTGGAGGAGGAGGAAGT |
| 189 | 2167 | VH | QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS |
| 190 | 2167 | VH | CAGGTGCAGCTGCAGCAGAGCGGAGCTGAGCTGGCACGACCAGGAGCAAGTGTGAAAATGTCCTGTAAGGCCAGCGGCTACACCTTCACACGGTATACCATGCATTGGGTGAAACAGAGA |
| | | | CCCGGGCAGGGACTGGAATGGATCGGGTACATTAATCCTTCCCGAGGATACACAAACTACAACCAGAAGTTTAAAGACAAGGCTACTCTGACCACAGATAAGAGCTCCTCTACCGCATAT |
| | | | ATGCAGCTGAGTTCACTGACATCTGAGGACAGTGCCGTGTACTATTGCGCTAGGTACTATGACGATCACTACTCCCTGGATTATTGGGGCCAGGGGACTACCCTGACAGTGAGCTCC |
| 191 | 2167 | hinge | AAEPKSSDKTHTCPPCP |
| 192 | 2167 | hinge | GCAGCCGAACCTAAATCTAGTGACAAGACTCATACCTGCCCCCCTTGTCCA |
| 193 | 2167 | CH2 | APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK |
| 194 | 2167 | CH2 | GCACCAGAGCTGCTGGGAGGACCTAGCGTGTTCCTGTTTCCACCCAAACCAAAGGATACTCTGATGATCTCCCGGACACCTGAAGTCACTTGTGTGGTCGTGGACGTGTCTCACGAGGAC |
| | | | CCCGTAAGTCTAAGTTTTAACTGGTACGTGGACGGCGTCGAGGTGCATTAATGCCTATATAACCTAAGCCCAGGGAGGTAACAGTACTAACTCCACATATCGCGTCGTGTCTGTCCTGACTGTGCTGCAC |
| | | | CAGGATTGGCTGAACGGCAAGGAGTACAAATGCAAGGTGAGCAACAAGGCCCTGCCTGCTCCAATCGAGAAGACAATTAGCAAAGCCAAG |
| 195 | 2167 | CH3 | GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 196 | 2167 | CH3 | GGGCAGCCCCGAGAACCTCAGGTCTACGTGCTGCCTCCATCTCGGGACGAGCTGACTAAAAACCAGGTCAGTCTGCTGTGTCTGGTGAAGGGCTTCTATCCAAGCGATATTGCTGTGGAG |
| | | | TGGGAATCCAATGGGCAGCCCGAAAACAATTACCTGACTTGGCCCCCTGTCCTGGACTCAGATGGGAGCTTCTTTCTGTATAGTAAACTGACCGTGGACAAGTCACGGTGGCAGCAGGGA |
| | | | AACGTCTTTAGCTGTTCCGTGATGCATGAGGCCCTGCACAATCATTACACCCAGAAATCTCTGAGTCTGTCACCCGGC |
| 197 | 2177 | Full | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGG |
| | | | SQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS |
| | | | AAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIE |
| | | | KTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK |
| 198 | 2177 | Full | CAGATCGTCCTGACACAGAGCCCAGCTATCATGTCAGCAAGCCCCGGCGAGAAAGTCACAATGACTTGCTCAGCCAGCTCCTCTGTGAGCTACATGAACTGGTATCAGCAGAAAAGCGGA |
| | | | ACCTCCCCCAAGAGATGGATCTACGACACATCCAAGCTGGCCTCTGGAGTGCCTGCTCACTTCAGGGGCAGCGGCTCTGGGACCAGTTATTCACTGACAATTTCCGGCATGGAGGCCGAA |
| | | | GATGCCGCTACCTACTATTGCCAGCAGTGGAGTTCAAACCCATTCACTTTTGGATCTGGCACCAAGCTGGAAATTAATGGCGGAGGAGGCTCCGGAGGAGGAGGGTCTGGAGGAGGAGGA |
| | | | AGTCAGGTGCAGCTGCAGCAGAGCGGAGCAGAGCTGGCTCGACCAGGAGCTAGTGTGAAAATGTCCTGTAAGGCAAGCGGCTACACCTTCACACGGTATACCATGCATTGGGTGAAACAG |
| | | | AGACCCGGGCAGGGACTGGAATGGATCGGGTACATTAATCCTTCCCGAGGATACACAAACTACAACCAGAAGTTTAAAGACAAGGCCACTCTGACCACAGATAAGAGCTCCTCTACCGCT |
| | | | TATATGCAGCTGAGTTCACTGACATCTGAGGACAGTGCAGTGTACTATTGCGCCAGGTACTATGACGATCACTACTCCCTGGATTATTGGGGCCAGGGGACTACCCTGACAGTGAGCTCC |
| | | | GCAGCCGAACCTAAATCTAGTGACAAGACTCATACCTGCCCCCCTTGTCCAGCACCAGAGGCTGCAGGAGGACCTAGCGTGTTCCTGTTTCCACCCAAACCAAAGGATACTCTGATGATC |
| | | | TCCCGGACACCTGAAGTCACTTGTGTGGTCGTGAGCGTGTCTCACGAGGACCCCGAAGTCAAGTTTAACTGGTACGTGGACGGCGTCGAGGTGCATAATGCCAAAACCAAGCCCAGGGAG |
| | | | GAACAGTACAACTCCACATATCGCGTCGTGTCTGTCCTGACTGTGCTGCACCAGGATTGGCTGAACGGCAAGGAGTACAAATGCAAGGTGAGCAACAAGGCACTGCCTGCCCCAATCGAG |
| | | | AAGACAATTAGCAAAGCAAAGGGGCAGCCCCGAGAACCTCAGGTCTACGTGCTGCCTCCATCTCGGGACGAGCTGACTAAAAACCAGGTCAGTCTGCTGTGTCTGGTGAAGGGCTTCTAT |
| | | | CCAAGCGATATTGCTGTGGAGTGGGAATCCAATGGGCAGCCCGAAAACAATTACCTGACTTGGCCCCCTGTCCTGGACTCAGATGGGAGCTTCTTTCTGTATAGTAAACTGACCGTGGAC |
| | | | AAGTCACGGTGGCAGCAGGGAAACGTCTTTAGCTGTTCCGTGATGCATGAGGCCCTGCACAATCATTACACCCAGAAATCTCTGAGTCTGTCACCCGGCAAG |
| 199 | 2177 | VL | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN |
| 200 | 2177 | VL | CAGATCGTCCTGACACAGAGCCCAGCTATCATGTCAGCAAGCCCCGGCGAGAAAGTCACAATGACTTGCTCAGCCAGCTCCTCTGTGAGCTACATGAACTGGTATCAGCAGAAAAGCGGA |
| | | | ACCTCCCCCAAGAGATGGATCTACGACACATCCAAGCTGGCCTCTGGAGTGCCTGCTCACTTCAGGGGCAGCGGCTCTGGGACCAGTTATTCACTGACAATTTCCGGCATGGAGGCCGAA |
| | | | GATGCCGCTACCTACTATTGCCAGCAGTGGAGTTCAAACCCATTCACTTTTGGATCTGGCACCAAGCTGGAAATTAAT |
| 201 | 2177 | linker | GGGGSGGGGSGGGGS |
| 202 | 2177 | linker | GGCGGAGGAGGCTCCGGAGGAGGAGGGTCTGGAGGAGGAGGAAGT |
| 203 | 2177 | VH | QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS |
| 204 | 2177 | VH | CAGGTGCAGCTGCAGCAGAGCGGAGCAGAGCTGGCTCGACCAGGAGCTAGTGTGAAAATGTCCTGTAAGGCAAGCGGCTACACCTTCACACGGTATACCATGCATTGGGTGAAACAGAGA |
| | | | CCCGGGCAGGGACTGGAATGGATCGGGTACATTAATCCTTCCCGAGGATACACAAACTACAACCAGAAGTTTAAAGACAAGGCCACTCTGACCACAGATAAGAGCTCCTCTACCGCTTAT |
| | | | ATGCAGCTGAGTTCACTGACATCTGAGGACAGTGCAGTGTACTATTGCGCCAGGTACTATGACGATCACTACTCCCTGGATTATTGGGGCCAGGGGACTACCCTGACAGTGAGCTCC |
| 205 | 2177 | hinge | AAEPKSSDKTHTCPPCP |
| 206 | 2177 | hinge | GCAGCCGAACCTAAATCTAGTGACAAGACTCATACCTGCCCCCCTTGTCCA |
| 207 | 2177 | CH2 | APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK |
| 208 | 2177 | CH2 | GCACCAGAGGCTGCAGGAGGACCTAGCGTGTTCCTGTTTCCACCCAAACCAAAGGATACTCTGATGATCTCCCGGACACCTGAAGTCACTTGTGTGGTCGTGAGCGTGTCTCACGAGGAC |
| | | | CCCGAAGTCAAGTTTAACTGGTACGTGGACGGCGTCGAGGTGCATAATGCCAAAACCAAGCCCAGGGAGGAACAGTACAACTCCACATATCGCGTCGTGTCTGTCCTGACTGTGCTGCAC |
| | | | CAGGATTGGCTGAACGGCAAGGAGTACAAATGCAAGGTGAGCAACAAGGCACTGCCTGCCCCAATCGAGAAGACAATTAGCAAAGCAAAG |
| 209 | 2177 | CH3 | GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 210 | 2177 | CH3 | GGGCAGCCCCGAGAACCTCAGGTCTACGTGCTGCCTCCATCTCGGGACGAGCTGACTAAAAACCAGGTCAGTCTGCTGTGTCTGGTGAAGGGCTTCTATCCAAGCGATATTGCTGTGGAG |
| | | | TGGGAATCCAATGGGCAGCCCGAAAACAATTACCTGACTTGGCCCCCTGTCCTGGACTCAGATGGGAGCTTCTTTCTGTATAGTAAACTGACCGTGGACAAGTCACGGTGGCAGCAGGGA |
| | | | AACGTCTTTAGCTGTTCCGTGATGCATGAGGCCCTGCACAATCATTACACCCAGAAATCTCTGAGTCTGTCACCCGGC |
| 211 | 1844 | Full | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGG |
| | | | SGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYW |
| | | | GQGTTVTVSSAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKV |
| | | | SNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSL |
| | | | SPGK |
| 212 | 1844 | Full | GATATTCAGCTGACACAGAGTCCTGCATCACTGGCTGTGAGCCTGGGACAGCGAGCAACTATCTCCTGCAAAGCCAGTCAGTCAGTGGACTATGATGGCGACTCCTATCTGAACTGGTAC |
| | | | CAGCAGATCCCAGGGCAGCCCCCTAAGCTGCTGATCTACGACGCCTCAAATCTGGTGAGCGGCATCCCACCACGATTCAGCGGCAGCGGCTCTGGGACTGATTTTACCCTGAACATTCAC |
| | | | CCAGTCGAGAAGGTGGACGCCGCTACCTACCATTGCCAGCAGTCTACCGAGGACCCCTGGACATTCGGCGGGGGAACTAAACTGGAAATCAAGGGAGGAGGAGGCAGTGGCGGAGGAGGG |
| | | | TCAGGAGGAGGAGGAAGCCAGGTGCAGCTGCAGCAGAGCGGAGCAGAGCTGGTCAGACCAGGAAGCTCCGTGAAAATTTCCTGTAAGGCTTCTGGCTATGCATTTTCTAGTTACTGGATG |
| | | | AATTGGGTGAAGCAGAGGCCAGGACAGGGCCTGGAATGGATCGGGCAGATTTGGCCCGGGGATGGAGACACCAACTATAATGGAAAGTTCAAAGGCAAGGCCACACTGACTGCTGACGAG |
| | | | TCAAGCTCCACAGCCTATATGCAGCTGTCTAGTCTGGCAAGCGAGGATTCCGCCGTGTACTTTTGCGCTCGGAGAGAAACCACAACTGTGGGCAGGTACTATTACGCTATGGACTACTGG |
| | | | GGCCAGGGGACCACAGTCACCGTGTCAAGCGCAGCCGAACCCAAATCCTCTGATAAGACCCACACATGCCCTCCATGTCCAGCTCCTGAGGCTGCAGGAGGACCAAGCGTGTTCCTGTTT |
| | | | CCCCCTAAACCTAAGGACACACTGATGATCTCTCGGACACCCGAAGTCACTTGTGTGGTCGTGGATGTGAGCCACGAGGACCCTGAAGTCAAATTCAACTGGTACGTGGATGGCGTCGAG |
| | | | GTGCATAATGCCAAAACTAAGCCTAGGGAGGAACAGTATAACTCCACTTACCGCGTCGTGTCTGTCCTGACCGTGCTGCATCAGGACTGGCTGAACGGAAAGGAGTACAAATGCAAGGTG |
| | | | AGCAACAAGGCACTGCCAGCCCCCATCGAGAAGACAATTTCCAAAGCAAAGGGCCAGCCTCGAGAACCACAGGTCTATGTGTACCCACCCAGCCGGGACGAGCTGACCAAAAACCAGGTC |
| | | | TCCCTGACATGTCTGGTGAAGGGATTTTATCCTTCTGATATTGCCGTGGAGTGGGAAAGTAATGGCCAGCCAGAAAACAATTACAAGACTACCCCTCCAGTGCTGGATTCTGACGGGAGT |
| | | | TTCGCTCTGGTCAGTAAACTGACTGTGGATAAGTCACGGTGGCAGCAGGGAAACGTCTTTAGTTGTTCAGTGATGCACGAGGCACTGCACAATCATTACACCCAGAAAAGCCTGTCCCTG |
| | | | TCTCCCGGCAAG |
| 213 | 1844 | VL | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK |
| 214 | 1844 | VL | GATATTCAGCTGACACAGAGTCCTGCATCACTGGCTGTGAGCCTGGGACAGCGAGCAACTATCTCCTGCAAAGCCAGTCAGTCAGTGGACTATGATGGCGACTCCTATCTGAACTGGTAC |
| | | | CAGCAGATCCCAGGGCAGCCCCCTAAGCTGCTGATCTACGACGCCTCAAATCTGGTGAGCGGCATCCCACCACGATTCAGCGGCAGCGGCTCTGGGACTGATTTTACCCTGAACATTCAC |
| | | | CCAGTCGAGAAGGTGGACGCCGCTACCTACCATTGCCAGCAGTCTACCGAGGACCCCTGGACATTCGGCGGGGGAACTAAACTGGAAATCAAG |
| 215 | 1844 | linker | GGGGSGGGGSGGGGS |
| 216 | 1844 | linker | GGAGGAGGAGGCAGTGGCGGAGGAGGGTCAGGAGGAGGAGGAAGC |
| 217 | 1844 | VH | QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTV |
| | | | TVSS |
| 218 | 1844 | VH | CAGGTGCAGCTGCAGCAGAGCGGAGCAGAGCTGGTCAGACCAGGAAGCTCCGTGAAAATTTCCTGTAAGGCTTCTGGCTATGCATTTTCTAGTTACTGGATGAATTGGGTGAAGCAGAGG |
| | | | CCAGGACAGGGCCTGGAATGGATCGGGCAGATTTGGCCCGGGGATGGAGACACCAACTATAATGGAAAGTTCAAAGGCAAGGCCACACTGACTGCTGACGAGTCAAGCTCCACAGCCTAT |
| | | | ATGCAGCTGTCTAGTCTGGCAAGCGAGGATTCCGCCGTGTACTTTTGCGCTCGGAGAGAAACCACAACTGTGGGCAGGTACTATTACGCTATGGACTACTGGGGCCAGGGGACCACAGTC |
| | | | ACCGTGTCAAGC |
| 219 | 1844 | hinge | AAEPKSSDKTHTCPPCP |
| 220 | 1844 | hinge | GCAGCCGAACCCAAATCCTCTGATAAGACCCACACATGCCCTCCATGTCCA |
| 221 | 1844 | CH2 | APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK |
| 222 | 1844 | CH2 | GCTCCTGAGGCTGCAGGAGGACCAAGCGTGTTCCTGTTTCCCCCTAAACCTAAGGACACACTGATGATCTCTCGGACACCCGAAGTCACTTGTGTGGTCGTGGATGTGAGCCACGAGGAC |
| | | | CCTGAAGTCAAATTCAACTGGTACGTGGATGGCGTCGAGGTGCATAATGCCAAAACTAAGCCTAGGGAGGAACAGTATAACTCCACTTACCGCGTCGTGTCTGTCCTGACCGTGCTGCAT |
| | | | CAGGACTGGCTGAACGGAAAGGAGTACAAATGCAAGGTGAGCAACAAGGCACTGCCAGCCCCCATCGAGAAGACAATTTCCAAAGCAAAG |
| 223 | 1844 | CH3 | GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG |
| 224 | 1844 | CH3 | GGCCAGCCTCGAGAACCACAGGTCTATGTGTACCCACCCAGCCGGGACGAGCTGACCAAAAACCAGGTCTCCCTGACATGTCTGGTGAAGGGATTTTATCCTTCTGATATTGCCGTGGAG |
| | | | TGGGAAAGTAATGGCCAGCCAGAAAACAATTACAAGACTACCCCTCCAGTGCTGGATTCTGACGGGAGTTTCGCTCTGGTCAGTAAACTGACTGTGGATAAGTCACGGTGGCAGCAGGGA |
| | | | AACGTCTTTAGTTGTTCAGTGATGCACGAGGCACTGCACAATCATTACACCCAGAAAAGCCTGTCCCTGTCTCCCGGC |
| 225 | 7239 | Full | DIQLTQSPSSLSASVGDRATITCRASQSVDYEGDSYLNWYQQKPGKAPKLLIYDASNLVSGIPSRFSGSGSGTDFTLTISSVQPEDAATYYCQQSTEDPWTFGCGTKLEIKGGGGSGGGG |
| | | | SGGGGSQVQLVQSGAEVKKPGASVKISCKASGYAFSSYWMNWVRQAPGQCLEWIGQIWPGDGDTNYAQKFQGRATLTADESTSTAYMELSSLRSEDTAVYYCARRETTTVGRYYYAMDYW |
| | | | GQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSN |
| | | | KALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSP |
| | | | G |
| 226 | 7239 | Full | GATATTCAGCTGACCCAGAGCCCAAGCTCCCTGTCTGCCAGTGTGGGGGATAGGGCTACAATCACTTGCCGCGCATCACAGAGCGTGGACTATGAGGGCGATTCCTATCTGAACTGGTAC |
| | | | CAGCAGAAGCCAGGGAAAGCACCCAAGCTGCTGATCTACGACGCCTCTAATCTGGTGAGTGGCATTCCCTCAAGGTTCTCCGGATCTGGCAGTGGGACTGACTTTACCCTGACAATCTCT |
| | | | AGTGTGCAGCCCGAGGATGCCGCTACCTACTATTGCCAGCAGTCTACAGAAGACCCTTGGACTTTCGGATGTGGCACCAAACTGGAGATTAAGGGAGGAGGAGGCAGTGGCGGAGGAGGG |
| | | | TCAGGAGGAGGAGGAAGCCAGGTCCAGCTGGTGCAGAGCGGAGCAGAGGTCAAGAAACCCGGAGCCAGCGTGAAAATTTCCTGCAAGGCCTCTGGCTATGCTTTCTCAAGCTACTGGATG |
| | | | AACTGGGTGAGGCAGGCACCAGGACAGTGTCTGGAATGGATCGGACAGATTTGGCCTGGGGACGGAGATACCAATTATGCTCAGAAGTTTCAGGGACGCGCAACTCTGACCGCCGATGAG |
| | | | TCAACAAGCACTGCATACATGGAGCTGTCCTCTCTGCGCTCCGAAGACACAGCCGTGTACTATTGCGCACGGAGAGAAACCACAACTGTGGGCCGATACTATTACGCAATGGATTACTGG |
| | | | GGCCAGGGGACCACAGTCACTGTGAGTTCAGAGCCTAAAAGCTCCGACAAGACCCACACATGCCCACCTTGTCCGGCGCCAGAAGCAGCCGGAGGGCCTAGCGTGTTCCTGTTTCCACCC |
| | | | AAGCCAAAAGATACCCTGATGATCAGCCGGACTCCTGAGGTCACCTGCGTGGTCGTGTCCGTGTCTCACGAGGACCCAGAAGTCAAATTCAACTGGTATGTGGATGGCGTCGAAGTGCAT |
| | | | AATGCTAAGACAAAACCCCGAGAGGAACAGTATAACTCCACCTACCGGGTCGTGTCTGTCCTGACAGTGCTGCATCAGGACTGGCTGAACGGCAAGGAGTACAAGTGCAAAGTGAGCAAC |
| | | | AAGGCCCTGCCCGCCCCAATCGAAAAGACCATTTCCAAGGCCAAAGGGCAGCCTCGCGAACCTCAGGTCTACGTGTACCCTCCATCTAGGGATGAACTGACAAAAAACCAGGTCAGTCTG |
| | | | ACTTGTCTGGTGAAGGGCTTCTACCCAAGCGACATTGCCGTGGAGTGGGAATCCAATGGCCAGCCCGAGAACAATTACAAGACTACCCCCCCTGTGCTGGACAGCGATGGGTCCTTCGCT |
| | | | CTGGTCAGTAAACTGACAGTGGATAAGTCAAGATGGCAGCAGGGAAATGTCTTTAGTTGTTCAGTGATGCACGAGGCACTGCACAACCACTACACCCAGAAGTCACTGTCCCTGTCACCC |
| | | | GGC |
| 227 | 7239 | hinge | GGGGSGGGGSGGGGS |
| 228 | 7239 | hinge | GGAGGAGGAGGCAGTGGCGGAGGAGGGTCAGGAGGAGGAGGAAGC |
| 229 | 7239 | CH2 | APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK |
| 230 | 7239 | CH2 | GCGCCAGAAGCAGCCGGAGGGCCTAGCGTGTTCCTGTTTCCACCCAAGCCAAAAGATACCCTGATGATCAGCCGGACTCCTGAGGTCACCTGCGTGGTCGTGTCCGTGTCTCACGAGGAC |
| | | | CCAGAAGTCAAATTCAACTGGTATGTGGATGGCGTCGAAGTGCATAATGCTAAGACAAAACCCCGAGAGGAACAGTATAACTCCACCTACCGGGTCGTGTCTGTCCTGACAGTGCTGCAT |
| | | | CAGGACTGGCTGAACGGCAAGGAGTACAAGTGCAAAGTGAGCAACAAGGCCCTGCCCGCCCCAATCGAAAAGACCATTTCCAAGGCCAAA |
| 231 | 7239 | CH3 | GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG |
| 232 | 7239 | CH3 | GGGCAGCCTCGCGAACCTCAGGTCTACGTGTACCCTCCATCTAGGGATGAACTGACAAAAAACCAGGTCAGTCTGACTTGTCTGGTGAAGGGCTTCTACCCAAGCGACATTGCCGTGGAG |
| | | | TGGGAATCCAATGGCCAGCCCGAGAACAATTACAAGACTACCCCCCCTGTGCTGGACAGCGATGGGTCCTTCGCTCTGGTCAGTAAACTGACAGTGGATAAGTCAAGATGGCAGCAGGGA |
| | | | AATGTCTTTAGTTGTTCAGTGATGCACGAGGCACTGCACAACCACTACACCCAGAAGTCACTGTCCCTGTCACCCGGC |
| 233 | 5243 | Full | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGCGTKLEIKGGGGSGGGG |
| | | | SGGGGSQVQLQQSGAELVRPGSSVKISCKASGYAFSSYWMNWVKQRPGQCLEWIGQIWPGDGDTNYNGKFKGKATLTADESSSTAYMQLSSLASEDSAVYFCARRETTTVGRYYYAMDYW |
| | | | GQGTTVIVSSAAEPKSSDKTHTCPPCPAPELLGGPSVFLFPPKPKDTLMISRTPEVICVVVDVSHEDPEVKFNWYVDGVEVHNAKTKPREEQYNSTYRVVSVLTVLHQDWLNGKEYKCKV |
| | | | SNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSL |
| | | | SPG |
| 234 | 5243 | Full | GATATTCAGCTGACTCAGAGTCCTGCTTCACTGGCAGTGAGCCTGGGACAGCGAGCAACCATCTCCTGCAAAGCTAGTCAGTCAGTGGACTATGATGGAGACTCCTATCTGAACTGGTAC |
| | | | CAGCAGATCCCAGGCCAGCCCCCTAAGCTGCTGATCTACGACGCCTCAAATCTGGTGAGCGGCATCCCACCACGATTCAGCGGCAGCGGCTCTGGGACTGATTTTACCCTGAACATTCAC |
| | | | CCAGTCGAGAAGGTGGACGCCGCTACATACCATTGCCAGCAGTCTACCGAGGACCCCTGGACATTCGGATGTGGCACTAAACTGGAAATCAAGGGAGGAGGAGGCAGTGGCGGAGGAGGG |
| | | | TCAGGAGGAGGAGGAAGCCAGGTGCAGCTGCAGCAGAGCGGAGCAGAGCTGGTCAGACCAGGAAGCTCCGTGAAAATTTCCTGCAAGGCATCTGGCTATGCCTTTTCTAGTTACTGGATG |
| | | | AATTGGGTGAAGCAGAGGCCAGGCCAGTGTCTGGAATGGATCGGGCAGATTTGGCCCGGGGATGGAGACACAAACTATAATGGAAAGTTCAAAGGCAAGGCTACACTGACTGCAGACGAG |
| | | | TCAAGCTCCACTGCTTATATGCAGCTGTCTAGTCTGGCCAGCGAGGATTCCGCTGTGTACTTTTGCGCACGGAGAGAAACCACAACTGTGGGCAGGTACTATTACGCAATGGACTACTGG |
| | | | GGCCAGGGGACCACAGTCACCGTGTCAAGCGCAGCCGAACCCAAATCCTCTGATAAGACCCACACATGCCCTCCATGTCCAGCACCTGAGCTGCTGGGAGGACCAAGCGTGTTCCTGTTT |
| | | | CCACCTAAACCTAAGGACACTCTGATGATCTCTCGGACACCCGAAGTCACTTGTGTGGTCGTGGATGTGAGCCACGAGGACCCTGAAGTCAAATTCAACTGGTACGTGGATGGCGTCGAG |
| | | | GTGCATAATGCCAAAACAAAGCCTAGGGAGGAACAGTATAACTCCACTTACCGCGTCGTGTCTGTCCTGACCGTGCTGCATCAGGACTGGCTGAACGGAAAGGAGTACAAATGCAAGGTG |
| | | | AGCAACAAGGCCCTGCCAGCTCCCATCGAGAAGACCATTTCCAAAGCTAAGGGCCAGCCTCGAGAACCACAGGTCTATGTGTACCCACCCAGCCGGGACGAGCTGACCAAAAACCAGGTC |
| | | | TCCCTGACATGTCTGGTGAAGGGGTTTTATCCTTCTGATATTGCCGTGGAGTGGGAAAGTAATGGACAGCCAGAAAACAATTACAAAACTACCCCTCCAGTGCTGGATTCTGACGGCAGT |
| | | | TTCGCACTGGTCAGTAAACTGACCGTGGATAAGTCACGGTGGCAGCAGGGGAACGTCTTTAGTTGTTCAGTGATGCACGAGGCCCTGCACAATCATTACACACAGAAGAGCCTGTCCCTG |
| | | | TCTCCCGGC |
| 235 | 5243 | VL | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGCGTKLEIK |
| 236 | 5243 | VL | GATATTCAGCTGACTCAGAGTCCTGCTTCACTGGCAGTGAGCCTGGGACAGCGAGCAACCATCTCCTGCAAAGCTAGTCAGTCAGTGGACTATGATGGAGACTCCTATCTGAACTGGTAC |
| | | | CAGCAGATCCCAGGCCAGCCCCCTAAGCTGCTGATCTACGACGCCTCAAATCTGGTGAGCGGCATCCCACCACGATTCAGCGGCAGCGGCTCTGGGACTGATTTTACCCTGAACATTCAC |
| | | | CCAGTCGAGAAGGTGGACGCCGCTACATACCATTGCCAGCAGTCTACCGAGGACCCCTGGACATTCGGATGTGGCACTAAACTGGAAATCAAG |
| 237 | 5243 | linker | GGGGSGGGGSGGGGS |
| 238 | 5243 | linker | GGAGGAGGAGGCAGTGGCGGAGGAGGGTCAGGAGGAGGAGGAAGC |
| 239 | 5243 | VH | QVQLQQSGAELVRPGSSVKISCKASGYAFSSYWMNWVKQRPGQCLEWIGQIWPGDGDTNYNGKFKGKATLTADESSSTAYMQLSSLASEDSAVYFCARRETTTVGRYYYAMDYWGQGTTV |
| | | | TVSS |
| 240 | 5243 | VH | CAGGTGCAGCTGCAGCAGAGCGGAGCAGAGCTGGTCAGACCAGGAAGCTCCGTGAAAATTTCCTGCAAGGCATCTGGCTATGCCTTTTCTAGTTACTGGATGAATTGGGTGAAGCAGAGG |
| | | | CCAGGCCAGTGTCTGGAATGGATCGGGCAGATTTGGCCCGGGGATGGAGACACAAACTATAATGGAAAGTTCAAAGGCAAGGCTACACTGACTGCAGACGAGTCAAGCTCCACTGCTTAT |
| | | | ATGCAGCTGTCTAGTCTGGCCAGCGAGGATTCCGCTGTGTACTTTTGCGCACGGAGAGAAACCACAACTGTGGGCAGGTACTATTACGCAATGGACTACTGGGGCCAGGGGACCACAGTC |
| | | | ACCGTGTCAAGC |
| 241 | 5243 | hinge | AAEPKSSDKTHTCPPCP |
| 242 | 5243 | hinge | GCAGCCGAACCCAAATCCTCTGATAAGACCCACACATGCCCTCCATGTCCA |
| 243 | 5243 | CH2 | APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK |
| 244 | 5243 | CH2 | GCACCTGAGCTGCTGGGAGGACCAAGCGTGTTCCTGTTTCCACCTAAACCTAAGGACACTCTGATGATCTCTCGGACACCCGAAGTCACTTGTGTGGTCGTGGATGTGAGCCACGAGGAC |
| | | | CCTGAAGTCAAATTCAACTGGTACGTGGATGGCGTCGAGGTGCATAATGCCAAAACAAAGCCTAGGGAGGAACAGTATAACTCCACTTACCGCGTCGTGTCTGTCCTGACCGTGCTGCAT |
| | | | CAGGACTGGCTGAACGGAAAGGAGTACAAATGCAAGGTGAGCAACAAGGCCCTGCCAGCTCCCATCGAGAAGACCATTTCCAAAGCTAAG |
| 245 | 5243 | CH3 | GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG |
| 246 | 5243 | CH3 | GGCCAGCCTCGAGAACCACAGGTCTATGTGTACCCACCCAGCCGGGACGAGCTGACCAAAAACCAGGTCTCCCTGACATGTCTGGTGAAGGGGTTTTATCCTTCTGATATTGCCGTGGAG |
| | | | TGGGAAAGTAATGGACAGCCAGAAAACAATTACAAAACTACCCCTCCAGTGCTGGATTCTGACGGCAGTTTCGCACTGGTCAGTAAACTGACCGTGGATAAGTCACGGTGGCAGCAGGGG |
| | | | AACGTCTTTAGTTGTTCAGTGATGCACGAGGCCCTGCACAATCATTACACACAGAAGAGCCTGTCCCTGTCTCCCGGC |
| 247 | 2174 | Full | QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSS |
| | | | STGGGGSGGGGSGGGGSDIQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGT |
| | | | KLEINRAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKA |
| | | | LPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK |
| 248 | 2174 | Full | CAGGTCCAGCTGCAGCAGAGCGGAGCTGAGCTGGCACGACCAGGAGCAAGTGTGAAAATGTCATGCAAGGCCAGCGGCTACACCTTCACACGGTATACTATGCACTGGGTGAAACAGAGA |
| | | | CCCGGACAGGGCCTGGAATGGATCGGGTACATTAACCCTAGCCGAGGATACACCAACTACAACCAGAAGTTTAAAGACAAGGCTACCCTGACCACAGATAAGAGCTCCTCTACAGCATAT |
| | | | ATGCAGCTGAGTTCACTGACTTCTGAGGACAGTGCTGTGTACTATTGTGCACGGTACTATGACGATCATTACTCCCTGGATTATTGGGGGCAGGGAACTACCCTGACCGTGAGCTCCTCT |
| | | | AGTACAGGAGGAGGAGGCAGTGGAGGAGGAGGGTCAGGCGGAGGAGGAAGCGACATCCAGATTGTGCTGACACAGTCTCCAGCAATCATGTCCGCCTCTCCCGGCGAGAAAGTCACTATG |
| | | | ACCTGCTCCGCCTCAAGCTCCGTGTCTTACATGAATTGGTATCAGCAGAAATCAGGAACCAGCCCCAAGAGATGGATCTACGACACATCCAAGCTGGCCTCTGGCGTGCCTGCTCACTTC |
| | | | AGGGGCAGTGGGTCAGGAACTAGCTATTCCCTGACCATTAGCGGCATGGAGGCCGAAGATGCCGCTACCTACTATTGTCAGCAGTGGTCTAGTAACCCATTCACATTTGGCAGCGGGACT |
| | | | AAGCTGGAGATCAATAGGGCAGCCGAACCCAAATCAAGCGACAAGACACATACTTGCCCCCCTTGTCCAGCACCAGAACTGCTGGGAGGACCTTCCGTGTTCCTGTTTCCACCCAAACCA |
| | | | AAGGATACACTGATGATTAGCCGCACCCCTGAGGTCACATGCGTGGTCGTGGACGTGAGCCACGAGGACCCCGAAGTCAAGTTCAACTGGTACGTGGACGGCGTCGAAGTGCATAATGCC |
| | | | AAAACCAAGCCTAGGGAGGAACAGTACAACAGTACATATAGAGTCGTGTCAGTGCTGACCGTCCTGCACCAGGATTGGCTGAACGGCAAGGAGTACAAATGCAAGGTGTCCAACAAGGCC |
| | | | CTGCCTGCTCCAATCGAGAAGACCATTTCTAAAGCAAAGGGGCAGCCCCGAGAACCTCAGGTCTACGTGTATCCTCCATCCCGGGACGAGCTGACTAAAAACCAGGTCTCTCTGACCTGT |
| | | | CTGGTGAAGGGCTTTTACCCATCTGATATTGCTGTCGAGTGGGAAAGTAATGGGCAGCCCGAGAACAATTATAAGACAACTCCCCCTGTGCTGGACTCCGATGGGTCTTTCGCCCTGGTC |
| | | | AGCAAACTGACAGTGGATAAGTCCAGATGGCAGCAGGGAAACGTCTTTTCTTGTAGTGTGATGCATGAAGCTCTGCACAATCATTACACTCAGAAATCACTGAGCCTGTCCCCCGGCAAG |
| 249 | 2174 | VH | QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS |
| 250 | 2174 | VH | CAGGTCCAGCTGCAGCAGAGCGGAGCTGAGCTGGCACGACCAGGAGCAAGTGTGAAAATGTCATGCAAGGCCAGCGGCTACACCTTCACACGGTATACTATGCACTGGGTGAAACAGAGA |
| | | | CCCGGACAGGGCCTGGAATGGATCGGGTACATTAACCCTAGCCGAGGATACACCAACTACAACCAGAAGTTTAAAGACAAGGCTACCCTGACCACAGATAAGAGCTCCTCTACAGCATAT |
| | | | ATGCAGCTGAGTTCACTGACTTCTGAGGACAGTGCTGTGTACTATTGTGCACGGTACTATGACGATCATTACTCCCTGGATTATTGGGGGCAGGGAACTACCCTGACCGTGAGCTCC |
| 251 | 2174 | linker | GGGGSGGGGSGGGGS |
| 252 | 2174 | linker | GGAGGAGGAGGCAGTGGAGGAGGAGGGTCAGGCGGAGGAGGAAGC |
| 253 | 2174 | VL | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN |
| 254 | 2174 | VL | CAGATTGTGCTGACACAGTCTCCAGCAATCATGTCCGCCTCTCCCGGCGAGAAAGTCACTATGACCTGCTCCGCCTCAAGCTCCGTGTCTTACATGAATTGGTATCAGCAGAAATCAGGA |
| | | | ACCAGCCCCAAGAGATGGATCTACGACACATCCAAGCTGGCCTCTGGCGTGCCTGCTCACTTCAGGGGCAGTGGGTCAGGAACTAGCTATTCCCTGACCATTAGCGGCATGGAGGCCGAA |
| | | | GATGCCGCTACCTACTATTGTCAGCAGTGGTCTAGTAACCCATTCACATTTGGCAGCGGGACTAAGCTGGAGATCAAT |
| 255 | 2174 | hinge | AAEPKSSDKTHTCPPCP |
| 256 | 2174 | hinge | GCAGCCGAACCCAAATCAAGCGACAAGACACATACTTGCCCCCCTTGTCCA |
| 257 | 2174 | CH2 | APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK |
| 258 | 2174 | CH2 | GCACCAGAACTGCTGGGAGGACCTTCCGTGTTCCTGTTTCCACCCAAACCAAAGGATACACTGATGATTAGCCGCACCCCTGAGGTCACATGCGTGGTCGTGGACGTGAGCCACGAGGAC |
| | | | CCCGAAGTCAAGTTCAACTGGTACGTGGACGGCGTCGAAGTGCATAATGCCAAAACCAAGCCTAGGGAGGAACAGTACAACAGTACATATAGAGTCGTGTCAGTGCTGACCGTCCTGCAC |
| | | | CAGGATTGGCTGAACGGCAAGGAGTACAAATGCAAGGTGTCCAACAAGGCCCTGCCTGCTCCAATCGAGAAGACCATTTCTAAAGCAAAG |
| 259 | 2174 | CH3 | GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG |
| 260 | 2174 | CH3 | GGGCAGCCCCGAGAACCTCAGGTCTACGTGTATCCTCCATCCCGGGACGAGCTGACTAAAAACCAGGTCTCTCTGACCTGTCTGGTGAAGGGCTTTTACCCATCTGATATTGCTGTCGAG |
| | | | TGGGAAAGTAATGGGCAGCCCGAGAACAATTATAAGACAACTCCCCCTGTGCTGGACTCCGATGGGTCTTTCGCCCTGGTCAGCAAACTGACAGTGGATAAGTCCAGATGGCAGCAGGGA |
| | | | AACGTCTTTTCTTGTAGTGTGATGCATGAAGCTCTGCACAATCATTACACTCAGAAATCACTGAGCCTGTCCCCCGGC |
| 261 | 2175 | Full | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGG |
| | | | SGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYW |
| | | | GQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSL |
| | | | SPGK |
| 262 | 2175 | Full | GACATTCAGCTGACCCAGAGTCCTGCTTCACTGGCAGTGAGCCTGGGACAGCGAGCAACAATCTCCTGCAAAGCTAGTCAGTCAGTGGACTATGATGGCGACTCCTATCTGAACTGGTAC |
| | | | CAGCAGATCCCAGGGCAGCCCCCTAAGCTGCTGATCTACGACGCCTCAAATCTGGTGAGCGGCATCCCACCACGATTCAGCGGCAGCGGCTCTGGAACCGATTTTACACTGAACATTCAC |
| | | | CCAGTCGAGAAGGTGGACGCCGCTACCTACCATTGCCAGCAGTCTACAGAGGACCCCTGGACTTTCGGCGGGGGAACCAAACTGGAAATCAAGGGAGGAGGAGGCAGTGGCGGAGGAGGG |
| | | | TCAGGAGGAGGAGGAAGCCAGGTGCAGCTGCAGCAGAGCGGAGCAGAGCTGGTCAGACCAGGAAGCTCCGTGAAAATTTCCTGTAAGGCATCTGGCTATGCCTTTTCTAGTTACTGGATG |
| | | | AATTGGGTGAAGCAGAGGCCAGGACAGGGCCTGGAATGGATCGGGCAGATTTGGCCCGGGGATGGAGACACAAACTATAATGGAAAGTTCAAAGGCAAGGCTACTCTGACCGCAGACGAG |
| | | | TCAAGCTCCACTGCATATATGCAGCTGTCTAGTCTGGCCAGCGAGGATTCCGCTGTCTACTTTTGCGCACGGAGAGAAACCACAACTGTGGGCAGGTACTATTACGCCATGGACTACTGG |
| | | | GGCCAGGGGACCACAGTCACCGTGTCAAGCGCAGCCGAACCCAAATCCTCTGATAAGACACACACTTGCCCTCCATGTCCAGCTCCTGAGCTGCTGGGAGGACCAAGCGTGTTCCTGTTT |
| | | | CCACCTAAACCTAAGGACACTCTGATGATCTCTCGGACTCCCGAAGTCACCTGTGTGGTCGTGGATGTGAGCCACGAGGACCCTGAAGTCAAATTCAACTGGTACGTGGATGGCGTCGAG |
| | | | GTGCATAATGCCAAAACAAAGCCTAGGGAGGAACAGTATAACTCCACATACCGCGTCGTGTCTGTCCTGACTGTGCTGCATCAGGACTGGCTGAACGGAAAGGAGTACAAATGCAAGGTG |
| | | | AGCAACAAGGCCCTGCCAGCTCCCATCGAGAAGACCATTTCCAAAGCTAAGGGCCAGCCTCGAGAACCACAGGTCTATGTGCTGCCACCCAGCCGGGACGAGCTGACAAAAAACCAGGTC |
| | | | TCCCTGCTGTGTCTGGTGAAGGGATTCTACCCTTCTGATATTGCAGTGGAGTGGGAAAGTAATGGCCAGCCAGAAAACAATTATCTGACTTGGCCTCCAGTGCTGGATTCTGACGGGAGT |
| | | | TTCTTTCTGTACAGTAAACTGACCGTGGATAAGTCACGGTGGCAGCAGGGAAACGTCTTTAGTTGTTCAGTGATGCACGAGGCCCTGCACAATCATTACACCCAGAAAAGCCTGTCCCTG |
| | | | TCTCCCGGCAAG |
| 263 | 2175 | VL | DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK |
| 264 | 2175 | VL | GACATTCAGCTGACCCAGAGTCCTGCTTCACTGGCAGTGAGCCTGGGACAGCGAGCAACAATCTCCTGCAAAGCTAGTCAGTCAGTGGACTATGATGGCGACTCCTATCTGAACTGGTAC |
| | | | CAGCAGATCCCAGGGCAGCCCCCTAAGCTGCTGATCTACGACGCCTCAAATCTGGTGAGCGGCATCCCACCACGATTCAGCGGCAGCGGCTCTGGAACCGATTTTACACTGAACATTCAC |
| | | | CCAGTCGAGAAGGTGGACGCCGCTACCTACCATTGCCAGCAGTCTACAGAGGACCCCTGGACTTTCGGCGGGGGAACCAAACTGGAAATCAAG |
| 265 | 2175 | linker | GGGGSGGGGSGGGGS |
| 266 | 2175 | linker | GGAGGAGGAGGCAGTGGCGGAGGAGGGTCAGGAGGAGGAGGAAGC |
| 267 | 2175 | VH | QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTV |
| | | | TVSS |
| 268 | 2175 | VH | CAGGTGCAGCTGCAGCAGAGCGGAGCAGAGCTGGTCAGACCAGGAAGCTCCGTGAAAATTTCCTGTAAGGCATCTGGCTATGCCTTTTCTAGTTACTGGATGAATTGGGTGAAGCAGAGG |
| | | | CCAGGACAGGGCCTGGAATGGATCGGGCAGATTTGGCCCGGGGATGGAGACACAAACTATAATGGAAAGTTCAAAGGCAAGGCTACTCTGACCGCAGACGAGTCAAGCTCCACTGCATAT |
| | | | ATGCAGCTGTCTAGTCTGGCCAGCGAGGATTCCGCTGTCTACTTTTGCGCACGGAGAGAAACCACAACTGTGGGCAGGTACTATTACGCCATGGACTACTGGGGCCAGGGGACCACAGTC |
| | | | ACCGTGTCAAGC |
| 269 | 2175 | hinge | AAEPKSSDKTHTCPPCP |
| 270 | 2175 | hinge | GCAGCCGAACCCAAATCCTCTGATAAGACACACACTTGCCCTCCATGTCCA |
| 271 | 2175 | CH2 | APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK |
| 272 | 2175 | CH2 | GCTCCTGAGCTGCTGGGAGGACCAAGCGTGTTCCTGTTTCCACCTAAACCTAAGGACACTCTGATGATCTCTCGGACTCCCGAAGTCACCTGTGTGGTCGTGGATGTGAGCCACGAGGAC |
| | | | CCTGAAGTCAAATTCAACTGGTACGTGGATGGCGTCGAGGTGCATAATGCCAAAACAAAGCCTAGGGAGGAACAGTATAACTCCACATACCGCGTCGTGTCTGTCCTGACTGTGCTGCAT |
| | | | CAGGACTGGCTGAACGGAAAGGAGTACAAATGCAAGGTGAGCAACAAGGCCCTGCCAGCTCCCATCGAGAAGACCATTTCCAAAGCTAAG |
| 273 | 2175 | CH3 | GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 274 | 2175 | CH3 | GGCCAGCCTCGAGAACCACAGGTCTATGTGCTGCCACCCAGCCGGGACGAGCTGACAAAAAACCAGGTCTCCCTGCTGTGTCTGGTGAAGGGATTCTACCCTTCTGATATTGCAGTGGAG |
| | | | TGGGAAAGTAATGGCCAGCCAGAAAACAATTATCTGACTTGGCCTCCAGTGCTGGATTCTGACGGGAGTTTCTTTCTGTACAGTAAACTGACCGTGGATAAGTCACGGTGGCAGCAGGGA |
| | | | AACGTCTTTAGTTGTTCAGTGATGCACGAGGCCCTGCACAATCATTACACCCAGAAAAGCCTGTCCCTGTCTCCCGGC |
| 275 | 6690 | Full | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEINGGGGSGGGGSGGGG |
| | | | SQVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYSLDYWGQGTTLTVSS |
| | | | AAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIE |
| | | | KTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 276 | 6690 | Full | CAGATCGTCCTGACTCAGAGCCCCGCTATTATGTCCGCAAGCCCTGGAGAGAAAGTGACTATGACCTGTTCCGCATCTAGTTCCGTGTCCTACATGAACTGGTATCAGCAGAAATCTGGA |
| | | | ACAAGTCCCAAGCGATGGATCTACGACACTTCCAAGCTGGCATCTGGAGTGCCTGCCCACTTCCGAGGCAGCGGCTCTGGGACAAGTTATTCACTGACTATTAGCGGCATGGAGGCCGAA |
| | | | GATGCCGCTACATACTATTGCCAGCAGTGGAGCTCCAACCCATTCACCTTTGGATGTGGCACAAAGCTGGAGATCAATGGCGGAGGAGGCTCCGGAGGAGGAGGGTCTGGAGGAGGAGGA |
| | | | AGTCAGGTCCAGCTGCAGCAGTCCGGAGCAGAACTGGCTAGACCAGGAGCCAGTGTGAAAATGTCATGCAAGGCCAGCGGCTACACATTCACTCGGTATACCATGCATTGGGTGAAACAG |
| | | | AGACCAGGACAGTGTCTGGAGTGGATCGGCTACATTAATCCCAGCAGGGGGTACACAAACTACAACCAGAAGTTTAAAGACAAGGCAACCCTGACCACCGATAAGTCTAGTTCAACAGCT |
| | | | TATATGCAGCTGAGCTCCCTGACTTCAGAAGACAGCGCTGTGTACTATTGCGCACGCTACTATGACGATCACTACTCCCTGGATTATTGGGGGCAGGGAACTACCCTGACCGTGTCTAGT |
| | | | GCAGCCGAGCCTAAATCAAGCGACAAGACCCATACATGCCCCCCTTGTCCGGCGCCAGAAGCTGCAGGCGGACCAAGTGTGTTCCTGTTTCCACCCAAACCTAAGGATACTCTGATGATT |
| | | | TCTCGAACTCCTGAGGTCACCTGCGTGGTCGTGAGCGTGTCCCACGAGGACCCAGAAGTCAAGTTCAACTGGTACGTGGATGGGGTCGAAGTGCATAATGCCAAAACCAAGCCCAGGGAG |
| | | | GAACAGTACAACTCAACTTATCGCGTCGTGTCTGTCCTGACCGTGCTGCACCAGGACTGGCTGAATGGCAAGGAGTACAAATGTAAGGTCTCAAATAAGGCTCTGCCCGCCCCTATCGAA |
| | | | AAAACTATCTCTAAGGCAAAAGGACAGCCTCGCGAACCACAGGTCTACGTGCTGCCCCCTAGCCGCGACGAACTGACTAAAAATCAGGTCTCTCTGCTGTGTCTGGTCAAAGGATTCTAC |
| | | | CCTTCCGACATCGCCGTGGAGTGGGAAAGTAACGGCCAGCCCGAGAACAATTACCTGACCTGGCCCCCTGTGCTGGACTCTGATGGGAGTTTCTTTCTGTATTCAAAGCTGACAGTCGAT |
| | | | AAAAGCCGGTGGCAGCAGGGCAATGTGTTCAGCTGCTCCGTCATGCACGAAGCACTGCACAACCATTACACTCAGAAGTCCCTGTCCCTGTCACCTGGC |
| 277 | 6690 | VL | QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGCGTKLEIN |
| 278 | 6690 | VL | CAGATCGTCCTGACTCAGAGCCCCGCTATTATGTCCGCAAGCCCTGGAGAGAAAGTGACTATGACCTGTTCCGCATCTAGTTCCGTGTCCTACATGAACTGGTATCAGCAGAAATCTGGA |
| | | | ACAAGTCCCAAGCGATGGATCTACGACACTTCCAAGCTGGCATCTGGAGTGCCTGCCCACTTCCGAGGCAGCGGCTCTGGGACAAGTTATTCACTGACTATTAGCGGCATGGAGGCCGAA |
| | | | GATGCCGCTACATACTATTGCCAGCAGTGGAGCTCCAACCCATTCACCTTTGGATGTGGCACAAAGCTGGAGATCAAT |
| 279 | 6690 | linker | GGGGSGGGGSGGGGS |
| 280 | 6690 | linker | GGCGGAGGAGGCTCCGGAGGAGGAGGGTCTGGAGGAGGAGGAAGT |
| 281 | 6690 | VH | QVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYSLDYWGQGTTLTVSS |
| 282 | 6690 | VH | CAGGTCCAGCTGCAGCAGTCCGGAGCAGAACTGGCTAGACCAGGAGCCAGTGTGAAAATGTCATGCAAGGCCAGCGGCTACACATTCACTCGGTATACCATGCATTGGGTGAAACAGAGA |
| | | | CCAGGACAGTGTCTGGAGTGGATCGGCTACATTAATCCCAGCAGGGGGTACACAAACTACAACCAGAAGTTTAAAGACAAGGCAACCCTGACCACCGATAAGTCTAGTTCAACAGCTTAT |
| | | | ATGCAGCTGAGCTCCCTGACTTCAGAAGACAGCGCTGTGTACTATTGCGCACGCTACTATGACGATCACTACTCCCTGGATTATTGGGGGCAGGGAACTACCCTGACCGTGTCTAGT |
| 283 | 6690 | hinge | AAEPKSSDKTHTCPPCP |
| 284 | 6690 | hinge | GCAGCCGAGCCTAAATCAAGCGACAAGACCCATACATGCCCCCCTTGTCCG |
| 285 | 6690 | CH2 | APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK |
| 286 | 6690 | CH2 | GCGCCAGAAGCTGCAGGCGGACCAAGTGTGTTCCTGTTTCCACCCAAACCTAAGGATACTCTGATGATTTCTCGAACTCCTGAGGTCACCTGCGTGGTCGTGAGCGTGTCCCACGAGGAC |
| | | | CCAGAAGTCAAGTTCAACTGGTACGTGGATGGGGTCGAAGTGCATAATGCCAAAACCAAGCCCAGGGAGGAACAGTACAACTCAACTTATCGCGTCGTGTCTGTCCTGACCGTGCTGCAC |
| | | | CAGGACTGGCTGAATGGCAAGGAGTACAAATGTAAGGTCTCAAATAAGGCTCTGCCCGCCCCTATCGAAAAAACTATCTCTAAGGCAAAA |
| 287 | 6690 | CH3 | GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| 288 | 6690 | CH3 | GGACAGCCTCGCGAACCACAGGTCTACGTGCTGCCCCCTAGCCGCGACGAACTGACTAAAAATCAGGTCTCTCTGCTGTGTCTGGTCAAAGGATTCTACCCTTCCGACATCGCCGTGGAG |
| | | | TGGGAAAGTAACGGCCAGCCCGAGAACAATTACCTGACCTGGCCCCCTGTGCTGGACTCTGATGGGAGTTTCTTTCTGTATTCAAAGCTGACAGTCGATAAAAGCCGGTGGCAGCAGGGC |
| | | | AATGTGTTCAGCTGCTCCGTCATGCACGAAGCACTGCACAACCATTACACTCAGAAGTCCCTGTCCCTGTCACCTGGC |

1. An antigen-binding construct comprising
a first antigen-binding polypeptide construct comprising a first scFv comprising a first VL, a first scFv linker, and a first VH, the first scFv monovalently and specifically binding a CD19 antigen, the first scFv selected from the group consisting of an anti-CD19 antibody HD37 scFv, a modified HD37 scFv, an HD37 blocking antibody scFv, and a modified HD37 blocking antibody scFv, wherein the HD37 blocking antibody blocks by 50% or greater the binding of HD37 to the CD19 antigen;
a second antigen-binding polypeptide construct comprising a second scFv comprising a second VL, a second scFv linker, and a second VH, the second scFv monovalently and specifically binding an epsilon subunit of a CD3 antigen, the second scFv selected from the group consisting of the OKT3 scFv, a modified OKT3 scFv, an OKT3 blocking antibody scFv, and a modified OKT3 blocking antibody scFv, wherein the OKT3 blocking antibody blocks by 50% or greater the binding of OKT3 to the epsilon subunit of the CD3 antigen;
a heterodimeric Fc comprising first and second Fc polypeptides each comprising a modified CH3 sequence capable of forming a dimerized CH3 domain, wherein each modified CH3 sequence comprises asymmetric amino acid modifications that promote formation of a heterodimeric Fc and the dimerized CH3 domains have a melting temperature (Tm) of about 68° C. or higher, and wherein the first Fc polypeptide is linked to the first antigen-binding polypeptide construct with a first hinge linker, and the second Fc polypeptide is linked to the second antigen-binding polypeptide construct with a second hinge linker.

2. The antigen-binding construct of claim 1, consisting of v12043, v10149, or v1661.

3. The antigen-binding construct of claim 1, wherein the first scFv comprises CDR sequences 100% identical to a set of CDR sequences at selected from
a) L1:
(SEQ ID NO:)
QSVDYDGDSYL,
L2:
(SEQ ID NO:)
DAS,
L3:
(SEQ ID NO:)
QQSTEDPWT,
H1:
(SEQ ID NO:)
GYAFSSYW,
H2:
(SEQ ID NO:)
IWPGDGDT,
H3:
(SEQ ID NO:)
RETTTVGRYYYAMDY;
b) L1:
(SEQ ID NO:)
QSVDYEGDSYL,
L2:
(SEQ ID NO:)
DAS,
L3:
(SEQ ID NO:)
QQSTEDPWT,
H1:
(SEQ ID NO:)
GYAFSSYW,
H2:
(SEQ ID NO:)
IWPGDGDT,
H3:
(SEQ ID NO:)
RETTTVGRYYYAMDY;
c) L1:
(SEQ ID NO:)
QSVDYSGDSYL,
L2:
(SEQ ID NO:)
DAS,
L3:
(SEQ ID NO:)
QQSTEDPWT,
H1:
(SEQ ID NO:)
GYAFSSYW,
H2:
(SEQ ID NO:)
IWPGDGDT,
H3:
(SEQ ID NO:)
RETTTVGRYYYAMDY
d) L1:
(SEQ ID NO:)
KASQSVDYDGDSYL,
L2:
(SEQ ID NO:)
DASNLVS,
L3:
(SEQ ID NO:)
QQSTEDPWT,
H1:
(SEQ ID NO:)
GYAFSSYWMN,
H2:
(SEQ ID NO:)
QIWPGDGDTN,
H3:
(SEQ ID NO:)
RETTTVGRYYYAMDY
e) L1:
(SEQ ID NO:)
RASQSVDYEGDSYL,
L2:
(SEQ ID NO:)
DASNLVS,
L3:
(SEQ ID NO:)
QQSTEDPWT,
H1:
(SEQ ID NO:)
GYAFSSYWMN,
H2:
(SEQ ID NO:)
QIWPGDGDTN,
H3:
(SEQ ID NO:)
RETTTVGRYYYAMDY
and
f) L1:
(SEQ ID NO:)
RASQSVDYSGDSYL,
L2:
(SEQ ID NO:)
DASNLVS,
L3:
(SEQ ID NO:)
QQSTEDPWT,
H1:
(SEQ ID NO:)
GYAFSSYWMN,
H2:
(SEQ ID NO:)
QIWPGDGDTN,
H3:
(SEQ ID NO:)
RETTTVGRYYYAMDY.

4. The antigen-binding construct of claim 3, wherein the first scFv comprises CDR sequences 95% identical to the set of CDRs according to claim 3.

5. The antigen-binding construct of claim 1, wherein the first VH polypeptide sequence is selected from a wild-type HD37 VH polypeptide sequence, an hVH2 polypeptide sequence, and an hVH3 polypeptide sequence, and the first VL polypeptide sequence is selected from a wild-type HD37 VL polypeptide sequence and an hVL2 polypeptide sequence.

6. The antigen-binding construct of claim 1, wherein the first VH polypeptide sequence is 95% identical to a wild-type HD37 VH polypeptide sequence, an hVH2 polypeptide sequence, or an hVH3 polypeptide sequence, and the first VL polypeptide sequences are 95% identical to wild-type HD37 VL polypeptide sequence or an hVL2 polypeptide sequence.

7. The antigen-binding construct of claim 1, the HD37 blocking antibody selected from 4G7, B4, B3, HD237, and Mor-208.

8. The antigen-binding construct of claim 1, wherein the second scFv comprises a set of CDRs selected from:
a) L1:
(SEQ ID NO:)
SSVSY,
L2:
(SEQ ID NO:)
DTS,
L3:
(SEQ ID NO:)
QQWSSNP,
H1:
(SEQ ID NO:)
GYTFTRYT,
H2:
(SEQ ID NO:)
INPSRGYT,
H3:
(SEQ ID NO:)
ARYYDDHYCLDY
and
b) L1:
(SEQ ID NO:)
SSVSY,
L2:
(SEQ ID NO:)
DTS,
L3:
(SEQ ID NO:)
QQWSSNP,
H1:
(SEQ ID NO:)
GYTFTRYT,
H2:
(SEQ ID NO:)
INPSRGYT,
H3:
(SEQ ID NO:)
ARYYDDHYSLDY

9. The antigen-binding construct of claim 1, wherein the second scFv comprises a set of CDRs at least 95% identical to the set of CDRs according to claim 8.

10. The antigen-binding construct of claim 1, wherein the second VH polypeptide sequence is a wild-type OKT3 VH polypeptide sequence, or a polypeptide sequence 95% identical to a wild-type OKT3 VH polypeptide sequence, and the second VL polypeptide sequence is a wild-type OKT3 VL polypeptide sequence, or a polypeptide sequence 95% identical to a wild-type OKT3 VL polypeptide sequence.

11. The antigen-binding construct of claim 1, the OKT3 blocking antibody selected from Teplizumab™, UCHT1, and visilizumab.

12. The antigen-binding construct of claim 1, the second scFv binding to the OKT3 CD3 epitope.

13. The antigen-binding construct of any one of claims 1 to 12, wherein the first VL, first scFv linker polypeptide sequence and first VH polypeptide sequences are arranged from N-terminus to C-terminus as VL-linker-VH.

14. The antigen-binding construct of any one of claims 1 to 12, wherein the first VL, first scFv linker polypeptide sequence and first VH polypeptide sequences are arranged from N-terminus to C-terminus as VH-linker-VL.

15. The antigen-binding construct of any one of claims 1 to 14, wherein the second VL, second scFv linker polypeptide sequence and second VH polypeptide sequences are arranged from N-terminus to C-terminus as VL-linker-VH.

16. The antigen-binding construct of any one of claims 1 to 14, wherein the second VL, second scFv linker polypeptide sequence and second VH polypeptide sequences are arranged from N-terminus to C-terminus as VH-linker-VL.

17. The antigen-binding construct of any of claims 1 to 16, wherein one or both scFv comprise a disulphide bond between VL and VH polypeptide sequences.

18. The antigen-binding construct of any of claims 1 and 3 to 17, wherein the first or second scFv linker is selected from Table B.

19. The antigen-binding construct of any of claims 1 and 3 to 18, wherein the first or second hinge polypeptide linker is selected from Table E.

20. The antigen-binding construct of claim 1, wherein the first VL, scFv linker and VH polypeptide sequences are arranged from N-terminus to C-terminus as VL-linker-VH comprising a disulphide bond between the first VL and VH polypeptide sequences, and the second VL, scFv linker and VH polypeptide sequences are arranged from N-terminus to C-terminus as VH-linker-VL comprising a disulphide bond between the second VL and VH polypeptide sequences.

21. The antigen-binding construct of claim 1, wherein the first VL, scFv linker and VH polypeptide sequences are arranged from N-terminus to C-terminus as VL-linker-VH comprising a disulphide bond between the VL and VH polypeptide sequences, and the second VL, scFv linker and VH polypeptide sequences are arranged from N-terminus to C-terminus as VL-linker-VH, and a disulphide bond between the VL and VH polypeptide sequences.

22. The antigen-binding construct of claim 20 or 21, the heterodimeric Fc comprising at least one CH2 domain comprising one or more amino acid substitutions that reduce the ability of the heterodimeric Fc to bind to FcγRs or complement.

23. The antigen-binding construct of any one of claims 1 to 22, wherein the binding affinity of the first scFv for CD19 is between about 0.1 nM to about 5 nM, and the binding affinity of the second scFv for the epsilon subunit of CD3 is between about 1 nM to about 100 nM.

24. The antigen-binding construct of any one of claims 1 to 23, wherein the heterodimeric Fc
a. is a human Fc; and/or
b. is a human IgG1 Fc; and/or
c. comprises one or more modifications in at least one of the CH3 domains as described in Table A; and/or
d. further comprises at least one CH2 domain; and/or
e. further comprises at least one CH2 domain comprising one or more modifications; and/or
f. further comprises at least one CH2 domain comprising one or more modifications in at least one of the CH2 domains as described in Table B; and/or
g. further comprises at least one CH2 domain comprising one or more amino acid substitutions that reduce the ability of the heterodimeric Fc to bind to FcγRs or complement as described in Table C; and/or
h. further comprises at least one CH2 domain comprising amino acid substitutions N297A or L234A_L235A, or L234A_L235A_D265S.

25. The antigen-binding construct of any one of claims 1 to 24; wherein the dimerized CH3 domains have a melting temperature (Tm) of 68, 69, 70, 71, 72, 73, 74, 75, 76, 77, 77.5, 78, 79, 80, 81, 82, 83, 84, or 85° C. or higher.

26. The antigen-binding construct of any one of claims 1 to 25, wherein the antigen-binding construct
a) is capable of synapse formation and bridging between CD19+ Raji B-cells and Jurkat T-cells as assayed by FACS and/or microscopy; and/or
b) mediates T-cell directed killing of CD19-expressing B cells in human whole blood or PBMCs; and/or
c) displays improved biophysical properties compared to v875 or v1661; and/or
d) displays improved protein expression and yield compared to v875 or v1661, e.g., expressed at >4-10 mg/L after SEC (size exclusion chromatography) when expressed and purified under similar conditions; and/or
e) displays heterodimer purity, e.g., >95%.

27. The antigen-binding construct of any of claims 1 through 26, wherein the antigen-binding construct is conjugated to a drug.

28. A pharmaceutical composition the antigen-binding construct of any of claims 1 through 27 and a pharmaceutical carrier.

29. The pharmaceutical composition of claim 28, the carrier comprising a buffer, an antioxidant, a low molecular weight molecule, a drug, a protein, an amino acid, a carbohydrate, a lipid, a chelating agent, a stabilizer, or an excipient.

30. A pharmaceutical composition for use in medicine comprising the antigen-binding construct of any of claims 1 through 27.

31. A pharmaceutical composition for use in treatment of cancer comprising the antigen-binding construct of any of claims 1 through 27.

32. A method of treating a cancer in a subject, the method comprising administering an effective amount of the antigen-binding construct of any of claims 1 through 27 to the subject.

33. The method of claim 32, wherein the subject is a human.

34. The method of claim 32, wherein the cancer is a lymphoma or leukemia or a B cell malignancy, or a cancer that expresses CD19, or non-Hodgkin's lymphoma (NHL) or mantle cell lymphoma (MCL) or acute lymphoblastic leukemia (ALL) or chronic lymphocytic leukemia (CLL) or rituximab- or CHOP (Cytoxan™/Adriamycin™vincristine/prednisone therapy)-resistant B cell cancers.

35. A method of producing the antigen-binding construct of any of claims 1 through 27, comprising culturing a host cell under conditions suitable for expressing the antigen-binding construct wherein the host cell comprises a polynucleotide encoding the antigen-binding construct of any of claims 1 through 27, and purifying the antigen-binding construct.

36. An isolated polynucleotide or set of isolated polynucleotides comprising at least one nucleic acid sequence that encodes at least one polypeptide of the antigen-binding construct any of claims 1 through 27.

37. The isolated polynucleotide of claim 36, wherein the polynucleotide or set of polynucleotides is cDNA.

38. A vector or set of vectors comprising one or more of the polynucleotides or sets of polynucleotides according to claim 36, optionally selected from the group consisting of a plasmid, a viral vector, a non-episomal mammalian vector, an expression vector, and a recombinant expression vector.

39. An isolated cell comprising a polynucleotide or set of polynucleotides according to claim 36, or a vector or set of vectors of claim 38, optionally selected from a hybridoma, a Chinese Hamster Ovary (CHO) cell, or a HEK293 cell.

40. A kit comprising the antigen-binding construct any of claims 1 through 27 and instructions for use.
